(19) United States
(12) Patent Application Publication
(10) Pub. No.: US 2005/0208509 A1
(43) Pub. Date: Sep. 22, 2005

(54) Nucleic acid sequences encoding type III tenebrio antifreeze proteins and method for assaying activity

(76) Inventors: Kathleen L. Horwath, Endwell, NY (US); Christopher M. Easton, Ithaca, NY (US)

Correspondence Address: MARK LEVY & ASSOCIATES, PLLC, PRESS BUILDING, SUITE 902, 19 CHENANGO STREET, BINGHAMTON, NY, 13901, US

(21) Appl. No.: 10/917,030

(22) Filed: Aug. 12, 2004

Related U.S. Application Data
(62) Division of application No. 09/876,796, filed on Jun. 7, 2001.
(60) Provisional application No. 60/210,446, filed on Jun. 8, 2000.

(51) Int. Cl. C12Q 1/68; C07H 21/04; C12P 21/06; C12N 1/21; C07K 14/195; C12N 15/74

(52) U.S. Cl. 435/6; 435/69.1; 435/252.3; 435/320.1; 530/350; 536/23.2; 530/388.4

(57) ABSTRACT

Thermal hysteresis proteins and their nucleotide sequences derived from the Tenebrionoidea Superfamily which lower the freezing point of a solution without effecting the melting point. Related methods for preparing said proteins and for providing antifreeze or recrystallization inhibition properties to a subject formulation.

This application claims the benefit of U.S. provisional application Ser. No. 60/210,446 filed Jun. 8, 2000.

### FIELD OF THE INVENTION

The present invention generally relates to a family of insect derived peptides which lower the freezing point of water and, to the corresponding family of synthetically cloned nucleotide sequences for encoding the peptide family expressed in bacteria. Included particularly is a novel set of Tenebrio molitor Type III thermal hysteresis proteins (“THPs”) and the nucleic acid sequences coding them. These THPs, also known as antifreeze proteins, prevent ice formation and/or limit ice growth by lowering the noncolligative freezing point of water without lowering the melting point. Antibodies and activators reactive to them are also included herein. Further, the invention provides for the development of quantitative assessment of THP induced recrystallization inhibition.

### BACKGROUND OF THE INVENTION

The freezing of water can have unpleasant or even hazardous consequences especially when ice forms in uncontrolled conditions. An example of an especially hazardous situation is when ice forms on roadways and bridges creating the potential for vehicular accidents. Road surfaces are typically subjected to applications of salts or glycols to alleviate icing conditions, however these solutions will have an environmental impact as they leach into the soil and, over time, cause rusting and physical damage to vehicles and to the road surfaces to which they are applied. Another especially hazardous situation is when ice forms on aircraft wings. This will deleteriously affect the lift of the aircraft. In order to reduce the formation of ice, airplanes are routinely de-iced using chemicals, like ethylene glycol that are environmentally toxic.

In the food industry, one of the principle means for storage of food products is in the frozen state, and frozen foods have become a mainstay of the human diet. Additionally, some items, like frozen desserts, require continual shelving in the frozen-state. Ice crystal formation may either cause separation of incompatible materials such as oil and water, or compromises cell membrane integrity and osmotic balance, leading to destruction of cells. If freezing is improperly performed or if numerous freeze-thaw cycles occur (common with frost-free freezers) it is common for the taste or physical texture to be adversely affected thereby reducing consumer appeal. Duration of freezing is also detrimental. Recrystallization, the process of larger crystals growing at the expense of smaller ones, occurs in a frozen sample over time, and has substantial negative impact on taste and texture of frozen foods.

The cryogenic storage of biological samples, cells, tissues and organs, requires cryoprotection for the maintenance of viable cells and cell membranes, that would otherwise be deleteriously affected by the freezing process and storage and recovery from the frozen state. One type of damage that occurs is that cell membranes are susceptible to penetration by ice crystals thereby destroying their function and utility upon warmup. Such freezing damage may in part be attributed to recrystallization. Likewise, in the area of agriculture, crop yield loss due to frost or freezing can be significant, resulting in the loss of millions of dollars of crops such as oranges and grapefruit. To prevent frost damage, plants may be artificially heated or chemically sprayed resulting in waste of energy or application of chemicals that ordinarily would not have to be applied.

While ultra low temperature storage would effectively limit recrystallization damage, this is hardly practical for the consumer targeted frozen food industry that relies on refrigerator/freezers capable of only −20° C. storage. Likewise, roadways, aircrafts and other mechanical and industrial situations where ice is problematic need less toxic and environmentally friendly alternatives than those being used right now for ice prevention, and ice growth suppression. Improvements are also needed in cryogenic storage solutions to eliminate freezing damage during tissue cryopreservation and more effective ways to limit/prevent frost damage to plant crops need to be sought.

The advance of molecular biology techniques have facilitated more biologically based strategies for ice prevention/suppression in a wide variety of applications. For example, U.S. Pat. Nos. 4,045,910 and 4,161,084 to Amy and Undow disclose protecting plants from frost damage by applying non-ice nucleating bacteria to the plants before the onset of freezing cold. The non-ice nucleating bacteria are supposed to compete with native ice nucleating bacteria and prevent ice formation by reducing the number of potential “triggers” to crystazation. Following this, Lindow and coworkers (Lindow et al., [1988] Appl. Env. Microbiol. 54:1557-1563) genetically engineered ice minus bacterial mutants for aerial disperal and competitive exclusion of naturally occurring ice nucleating bacteria for enhance frost protection. One drawback of this ice prevention method is that it involves the release of genetically modified bacteria into the environment Alternatively, U.S. Pat. No. 4,834,899 to Kievecz discusses applying a bactericide to plants to prevent frost damage by killing the ice nucleating bacteria while U.S. Pat. No. 4,484,409 to Caple et al. discloses chemically synthesizing polymeric ice nucleation inhibitors via free radical polymerization. The polymers produced in Caple et al. have a tightly controlled spacing of about 15 Angstroms between the hydrophobic and hydrophilic groups. The polymers are sprayed on the plants and are designed to inhibit ice formation. U.S. Pat. No. 4,601,842 to Caple et al. discloses applying naturally occurring biogenic ice nucleation inhibitors, proteinaceous though not characterized further, obtained from cold weather plants to growing crops for protection from frost damage. However, it remains to be seen whether the active agent in these plant extracts is a member of what is now referred to as “antifreeze proteins”.

The existence of naturally occurring macromolecular species known as “antifreeze proteins” or thermal hysteresis proteins (referred to herein as “THP”) and the subclassifications antifreeze glycoproteins (referred to herein as “AFGPs”), and antifreeze peptides, (referred to herein as “AFPs”), is well known and widely reported in the literature. (Throughout this specification, the terms THP and AFP may be used interchangeably and mean the same unless specified otherwise.) THPs are believed to play an important role in many plant and animal species' ability to survive exposure to subzero temperatures. By definition, the equilibrium melting and freezing points of water are identical. This means that in the absence of THPs, a small (about 0.25 mm diameter) ice crystal that is about to melt at the melting point temperature will normally grow noticeably if the temperature is lowered by 0.01 to 0.02° C. However, the presence of thermal hysteresis proteins lowers the non-equilibrium freezing point of water without lowering the melting point (equilibrium freezing point). If THPs are present, the temperature may be lowered as much as 5 to 6° C. below the melting point (depending upon the specific activity and concentration of the proteins present) before noticeable crystal growth occurs. Thus, when THPs are added to a solution they produce a difference between the freezing and melting temperatures of the solution, and this difference has been termed “thermal hysteresis”. This unusual property is attributed only to antifreeze proteins, since all proteins other than antifreeze proteins, or other solutes do not cause a thermal hysteretic effect. The antifreeze protein compounds achieve freezing point depression in a non-colligative manner that does not depress the vapor pressure or raise the osmotic pressure of water, as is the case with colligative type antifreezes such as glycerol or ethylene glycol. Therefore, antifreeze proteins cause a freezing point depression to a far greater degree than one would expect on the basis of the osmolality of the solution containing the molecules. This non-colligative freezing point depression means that antifreeze proteins are more efficient antifreezes on a molar basis i.e., very low concentrations of AFP in pure solutions are known to have approximately five hundred times greater freezing point depression than colligative processes would predict. Given this, and their proteinaceous nature, they are an attractive alternative to the currently used de-icing solutions, since they are inherently environmentally friendly, non-toxic, biodegradable, and unlike the low molecular weight polyols, do not need to rely on high concentrations and colligative means to effect freezing point depression.

This thermal hysteretic behavior of antifreeze proteins is attributed to a specific protein-ice interaction that restricts ice growth, but not ice melt, hence creating a difference between the freezing and melting point of a solution. THPs are believed to create this thermal hysteretic effect via an adsorption-inhibition method (Raymond, J. A. and A. L. DeVries [1977] Proc. Nat'lAcad Sci. USA 74:2589-2593). The protein adsorbs (through hydrogen bonding and or hydrophobic interactions) to the surface of the ice crystal. This effectively raises the curvature growth steps on the ice surface thereby slowing or stopping the growth of ice until the temperature is significantly lowered. It is believed that the mechanism of ice crystal growth suppression by AFPs via this “adsorption inhibition” allows ice crystals to only grow in unblocked regions because the presence of the AFP ties up the potential insertion sites for new water to come into the lattice. Thus since all water adds to the lattice by hydrogen bonding too, if all possible active sites are bound up, the AFPs must be moved out of the way before new water can be added. Attachment of the AFP to the ice crystal increases the ratio of molecular volume to surface area, and in order for freezing to occur, more energy must be removed from the system than would be required in the absence of the AFPs. Hence, the freezing point of water is lowered by the binding action of AFPs (i.e., growth of the crystal requires the temperature to be further lowered to allow crystal growth to proceed.) The mechanism of action of AFPs is somewhat analogous to “poisoning” the growth of a crystal by the presence of an impurity where the AFP acts as the impurity. Melting point, however, is not lowered, therefore generating a thermal hysteretic gap.

By far, the vast majority of prior art studies relating to thermal hysteresis proteins, their biological functions and utilities of the proteins have focused on AFPs and AFGPs from various species of salt water fishes. These studies have focused on: (a) the isolation and characterization of the proteins, (b) the conformations of the protein molecules, including second and higher order conformations, (c) the interaction of the protein molecules with ice, which includes studies addressing such matters as the thermodynamics and surface kinetics of the ice crystal, the direction of crystal growth, and the modes and directions by which the proteins block the crystal growth and (d) means of preparing the proteins synthetically including methods involving the use of recombinant DNA.

The discovery of antifreeze glycoproteins, for example, was first reported in Antarctic fish by DeVries, in 1969 (A. L., DeVries and D. E. Wohlschlag [1969] Science 163:1073-1075). Water temperatures in McMurdo Sound, Antarctica, average −1.87° C. over the year, and various species of fish survive in these conditions despite the fact that the total concentration of sodium chloride and other low molecular weight substances present in their blood sera could only produce a freezing point depression of less than half that needed for survival under these conditions. These early studies indicated that the survival of these fish was attributed to the presence of certain macromolecular antifreeze compounds in the blood, and DeVries and his coworkers were the first to establish the nature and composition of these macromolecular species. The AFGP's isolated have typical molecular weights ranging from about 2,500 to 34,000 Dalton, and the AFP's (containing no sugar moieties) have molecular weights ranging from about 3,300 to 12,000 Dalton. The AFP's and AFGP's are present in relatively large concentrations in fish blood (about 10 to 40 mg/mL).

Following the discovery of these antifreeze proteins, attempts have been made to exploit the proteins' antifreeze character by using them in biological materials other than those of the fish from which they were derived. As an example, red blood cells were treated with the proteins using standard cryopreservation procedures and exposed to freezing conditions (Carpenter, J. F. and T. N. Hansen [1992] Proc. Nat'l Acad. Sci. 89:8953-8957). The results were highly dependent on concentration of the AFP and in certain concentration ranges actually caused the complete destruction of the cells rather than their preservation, presumably through the spicular ice growth that occurs in AFP solutions upon reaching the freezing point of the solution. U.S. Pat. No. 5,358,931 to Rubinsky et al., discloses the interaction of AFPs and AFGPs with cell membranes facilitate their cryoprotective effects, while U.S. Pat. No. 5,654,279 to Rubinsky and Koushafar exploit the injurous effects of more elevated titers of AFPs and AFGPs for use in a cryoablation technique of selective tissues damage during cryosurgery.

As seen, their utility as efficient ice supressors, and their proteinaceous nature make THPs an ideal, environmentally friendly material to suppress ice formation in a wide variety of circumstances. Also, they have the advantage that they can be applied, for examples to road surfaces, aircraft wings, or to an agricultural plant ahead of time so that they would interact with ice during formation and, further, they can be applied after the onset of ice formation and serve to prevent continued ice crystal formation. Such upscale uses necessitate a ready source for obtainment of these molecules. In winter flounder, the concentration of AFPs range from 1.0% to 3.0% depending on the species and the season; hence, AFPs are not produced in large enough quantities in arctic fish for the fish to be harvested as a source for an ice prevention agent. There have been some attempts to synthesize AFP using direct chemical processes (Chakrabartty, A. et al. [1989] J. Biol. Chem. 264: 11307-11312); however these processes can be expensive and time consuming.

The advancement of genetic technologies have provided alternate methods for synthesizing AFP's. For example, a semi synthetic winter flounder AFP was produced by Peters et al., ([1989] Protein Engineering 3:145-151) via gene recombination, in Escherichia coli (E. coli). A gene constructed of a fused synthetic deoxyribonucleic acid (DNA) fragment and a DNA fragment derived from a full length winter flounder clone was inserted into a plasmid and the plasmid was placed in the E. coli for production of a fusion protein. The biosynthetic fusion protein produced contained part of a pro-AFP and part of a B-galactosidase peptide and had limited antieeze activity after cleavage from B-galactosidase. Significant advances in cloning recombinant AFPs from selected fish species have generated more fully active recombinant AFPs, and with sufficient yield to allow more rigorous structure/function studies, as well as, potential sources for commercial application. U.S. Pat. No. 5,118,792 to Warren et al., discloses a recombinant DNA approach for generation of AFP fusion proteins derived from winter flounder genes following cloning into E. coli, where then the expressed AFP fusion protein serves as a additive for use to increase storage life of frozen food products and other biologics. U.S. Pat. Nos. 5,932,697 and 5,925,540 to Caceci et al., and U.S. Pat. No. 5,849,537 to Tripp et al., disclose further recombinant DNA strategies for yielding improved or hybrid synthetic winter flounder AFP peptides expressed in E. coli (Caceci et al.,), and winter flounder AFP gene cloned into yeast, S. cerevisiae, to provide a more suitable environment for properly processing (e.g. removal of unwanted presequences) recombinant AFPs and for recovery of significant quantities from fermentation broth (Tripp et al.,).

Many insects and other terrestrial arthropods (including certain spiders, mites, and centipedes) also produce thermal-hysteresis proteins. However, studies regarding insect THPs, including their isolation, structural characteristics and conformation, molecular analyses and protein/ice interactions significantly lag behind the characterization and molecular approaches described for that of the fish antifreeze proteins. Nevertheless, since insects are subjected to colder environments than that encountered by polar marine fishes, it is believed that they have evolved extremely effective and potent THPs. For example, the thermal hysteretic activity present in the hemolymph (circulatory fluid of insects) of the beetle Dendroides canadensis larvae in midwinter averages 3 to 6° C. with some individuals having as much as 8 to 9° C. Considering the maximal activity achievable with very high concentrations of the fish THPs is about 1.7° C., this suggests that insect AFPs potency may be as much as 3 to 6 times greater than those of the fish antifreeze proteins.

Despite the broad phylogenetic range of insects reported to produce THPs, they have only been isolated from four species: the beetles T. molitor and D. canadensis₂ the milkweed bug, Oncopeltus fasciatus, and the spruce budworm, Choristoneura fiuniferina. The molecular masses of these THPs range from approximately 8 to 20 kDa. The amino acid compositions of representative insect THPs are shown in Table 1 below, and can be generally characterized as having higher percentages of hydrophilic amino acids (i.e. Thr, Ser, Asx, Glx, Lys, Arg) than the fish THPs, with approximately 40 to 50 mol % of the residues being capable of forming hydrogen bonds.

TABLE 1
Amino acid compositions (mol %) of representative THPs.

| Amino | 1 | 2 | 2 | 2 | | |
|---|---|---|---|---|---|---|
| acid | (H-1) | YL-1 | T-4 | T-3 | 3 | 4 |
| Asx | 14.3 | 13 | 7.3 | 5.3 | 9.5 | 7.1 |
| Thr | 17.2 | 17 | 6.6 | 2.3 | 6.0 | 2.7 |
| Ser | 10.3 | 7 | 7.4 | 11.1 | 13.0 | 30.5 |
| Glx | 5.2 | 4 | 8.9 | 12.4 | 11.0 | 12.3 |
| Pro | 2.6 | 2 | 5.9 | 0.0 | 5.0 | 0.0 |
| Gly | 6.6 | 8 | 8.3 | 11.4 | 15.0 | 20.0 |
| Ala | 8.4 | 8 | 14.3 | 5.0 | 8.0 | 6.8 |
| ½Cys | 15.9 | 16 | 0.0 | 28.0 | 6.0 | 0.0 |
| Val | 1.7 | 3 | 11.5 | 2.3 | 3.0 | 3.0 |
| Met | 0.2 | 0 | 4.8 | 0.0 | 0.0 | 0.0 |
| Ile | 1.5 | 0 | 7.1 | 1.0 | 1.2 | 1.9 |
| Leu | 1.9 | 0 | 0.0 | 2.2 | 6.5 | 3.1 |
| Lys | 3.4 | 3 | 6.8 | 15.4 | 3.1 | 7.5 |
| Arg | 4.8 | 0 | 2.6 | 0.0 | 8.0 | 0.0 |
| Tyr | 3.9 | 1 | 2.3 | 0.0 | 1.0 | 2.0 |
| Phe | 0.0 | 1 | 3.9 | 0.0 | 2.2 | 1.1 |
| His | 1.9 | 2 | 1.9 | 3.1 | 0.0 | 2.3 |

1 = Dendroides canadensis;
2 = Tenebrio molitor;
3 = Choristoneura fumiferana;
4 = Oncopeltus fasciatus

The insect THPs characterized to date do not have a carbohydrate component typical of fish AFGPs, nor do they have high percentages of alanine residues (e.g. 65%) like the Type I fish (winter flounder) AFPs. Moreover, as seen in Table I, the known insect AFPs appear to fall into one of two categories (Type II or III) based on their amino acid composition. Both Dendroides and Tenebrio possess AFPs consistent with a Type II designation (see Table 1, H-1, YL-1, and T3). As defined here, a Type II classification is derived from Type II AFPs, previously identified from certain fish (e.g., Sea Raven, Atlantic herring, and smelt) that are considered to be rich in cysteine residues, and are homologous to C-type lectins. For example, Type II AFPs from the Sea Raven contain (on a mole basis) 7.6% cysteine, 14.4% alanine, 19% total of aspartic and glutamic acids, and 8% threonine. While the insect Type II AFPs share no homologies to the C-type lectins, their high percentage of cysteine residues (e.g. 15-28%) delineates them as an insect Type II AFP. Similarly, the spruce budworm AFP (Table 1 #3) showing enriched threonine and cysteine is also here designated an insect Type II AFP. In contrast, the Type III designated AFPs (e.g. Table 1, T4 and #4) are devoid of or have only modest cysteine residues and are not especially rich in alanine residues. Since there is no conspicuous dominance of these particular amino acids, as is also in the case of the Type III designation for fish AFPs (e.g., Ocean pout), the non-cysteine rich insect AFP denoted in Table 1 fall into a Type III AFP classification.

Early studies indicate an insect AFP isolated from winter acclimated T. molitor larvae which had different amino acid compositions from any of the other known insect Type II and Type III AFP's (Horwath et al., [1996] Eur. J. Entomol. 93:419-433). Homegeneity of the purified THP was confirmed and its amino acid composition and N-terminal sequence determined. The THP isolated was found to be a 117 residue peptide with mass spectrometry indicating it to have a molecular mass of 12.86 kDa, hence the designation Tm 12.86; (Tm) for T. molitor, and 12.86 for molecular weight. Thermal hysteresis determinations for Tm 12.86 indicate that it is a potent Type III AFP. The invention provides additional Type III insect AFP's, as well as an antisera reactive to Tm 12.86 and an endogenous “activator”, capable of enhancing thermal hysteretic levels of Tm 12.86.

Prior to the present invention, insect AFP genes shown to encode for thermal hysteretically active proteins have been identified and sequenced from only three insect species, and all are genes that encode for Type II insect AFPs. The three insect species are:
1) Choristoneura fumiferana, (spruce budworm) gene sequences for Type II Thr/Cys rich AFP (Tyshenko M. G. et al., [1997] Nature Biotech. 15: 887-890 (TABLE 1 #3).
2) Dendroides canadensis (pyrochoid beetle) gene sequences for Type II Thr/Cys rich AFP (Duman J. G., et al., [1998] J. Comp. Physiol. B: 168: 225-232 (TABLE 1 #1).
3) Tenebrio molitor (tenebrionid beetle) gene sequences for Type II Thr/Cys rich AFP's (Graham L. A, et al., [1998] Nature 388: 727-728 (TABLE 1 #YL1); Liou et al., [1999] Biochemistry 38:11415-24).

The nucleotide sequences and the predicted amino acid sequences for the peptides are consistent with the earlier amino acid composition assessment (Table 1) showing that the Tenebrio Type II AFP (YL1), the spruce budworm AFP and the Dendroides AFP all show enriched amino acid residues for cysteine and threonine, consistent with their being designated as Type II AFPs. Moreover, nucleotide and predicted amino acid sequences of the Type II AFPs from Tenebrio and Dendroides indicate that both sets of proteins are composed predominantly by a series of 12 (Tenebrio) or 13 (Dendroides) amino acid repeats. Additionally, multiple related nucleotide sequences (sharing>80% sequence homology) have been isolated for each of these groups, encoding for numerous isoforms (8 kDa to 20 kDa) from each species. And, these two sets of “repeat sequence” Type II AFPs from Tenebrio and Dendroides were found to share 48-67% identity of residues with one another, corresponding to several conserved regions, which suggest that they are all part of a multigene family encoding these Type II AFPs. In contrast, the sequence analysis of the spruce budworm AFP shows that while being enriched in cysteine and threonine, it bears no similarity to the Type II AFPs from Tenebrio and Dendroides, and also is non-repetitive in sequence. Importantly, however, what the Type II AFPs from all three species do have in common stems from the enriched cysteine and threonine composition of all three. From a comformational perspective, this strongly suggests that these residues are importance in the folded structure and required for the ice binding antifreeze activity. In fact, the disulfide bonded structure is absolutely essential for antifreeze activity in all of these molecules, as disruption of disulfide bridge formation such as treatment with dithiothreitol, results in complete loss of thermal hysteretic activity. The folded structure of the insect Type II Tenebrio and spruce budworm AFPs have recently been reported (iou, Y. C. et al., [2000] Nature 406:322-324; Graether, S. P. et al., [2000] Nature 406:325-328), as being Beta helical with a triangular cross section and rectangular sides that form stacked parellel Beta sheets. This structural arrangement is quite complex, unlike any seen with the fish antifreeze proteins, and may provide for generating greater thermal hysteretic activities of these insect AFPs over that seen from the fish AFPs and AFGPs. U.S. Pat. Nos. 5,627,051 and 5,633,451 to Duman, regarding Dendroides AFPs and U.S. Pat. No. 6,008,016 to Walker, V. K. et al., regarding the spruce budworm AFPs, disclose nucleic acid and amino acid sequences for their respective insect Type II AFPs genes and peptides, and application for exploiting the freezing point depression behavior of these antifreeze proteins.

The unusually complex structural arrangements seen for these insect Type II AFPs clearly require precise folding patterns. This suggests that generating recombinant insect antifreeze proteins that are properly folded to allow for their thermal hysteretic activity, are much more problematic than that experienced with the fish AFPs species. In fact, the recombinant products of the insect AFP genes mentioned above display lower activity (in some cases significantly lower) than that seen from the native endogenous AFPs. Cloning vectors (e.g. E. coli) that may not fully process the translation products, nor contain eukaryotic molecular chaperoning molecules to facilitate proper folding, may make it extremely difficult, even for experts in molecular biology, to recover recombinant products displaying full or even partial thermal hysteretic activity.

The known insect Type III AFPs also have a strong hydrophilic nature (Table 1; T4 and #4), including Tm 12.86 that consists of 57% hydrophilic amino acids. This suggests that they too may have significant globular structures of precise conformational arrangement necessary to impart antifreeze activity. Therefore molecular studies addressed at isolating their genes and expressing active recombinant products may prove challenging. Prior to the present invention, unsuccessful attempts had been made at isolating insect Type III AFPs genes. Tang and Baust made use of an antiserum generated against an antifreeze protein active solution derived from T. molitor, designated AFP-3 (homogeneity of this peptide was not confirmed) to screen a cDNA T. molitor library and isolated a full length clone, the sequence of which was entered into Genbank (NCBI Seq. ID: 785071). This clone was prematurely (or even incorrectly) listed as an antifreeze protein since recombinant products did not display thermal hysteretic activity. Further support that the AFP-3 clone may not be an antifreeze protein comes from extensive studies by P. Davies and coworkers (Rothemund S. et al., [1997] Biochemistry 36:13791-13801]; [1999] Structure 7:1325-1332), molecular biology experts. In numerous attempts they have cloned the insert generated by Tang and expressed in bacteria the encoded peptide they designated as THP-12 (also known as AFP-3). The recombinant product in all attempts did not display any thermal hysteretic activity, and subsequent NMR spectroscopy studies suggest that the protein has a nonbundle helical structure consisting of six alpha helices arranged in a ‘baseball glove’ shape (i.e. with no obvious ice binding motif seen). They have concluded that THP-12 (AFP-3) might be a member of small lipid carrier class of proteins, yet it's biological function is as yet undetermined.

The present invention successfully isolates insect Type III AFP genes. This was accomplished by using the antiserum generated against Tm 12.86 was to screen newly developed cDNA libraries prepared from mRNA populations extracted from fat body and whole larvae of winter acclimated T. molitor. Two full length clones (FW-1 and 2-3) were isolated and sequenced. The first clone was found to encode a predicted 18 residue signal peptide proceeding a 116 residue mature peptide of 13.17 kDa molecular weight, that shared 80% amino acid homology with the N-terminal sequence of the endogenous Tm 12.86. Thus, it appeared that rather than isolating the gene encoding Tm 12.86, a homologue (Tm 13.17) was cloned and sequenced. The search of DNA sequence databases revealed that it was most closely related (57% similarity) to the B1 assessory gland protein of T. molitor, and had only moderate (37%) similarity to AFP-3. The recombinant product of the Tm 13.17 clone recovered from the bacterial expression system did not display thermal hysteretic activity. Similarly, a second clone (2-3) was isolated and sequenced and found to encode for a predicted 18 residue signal peptide preceeding a 115 residue mature peptide of 12.84 kDa molecular weight, having a different overall amino acid composition than the native Tm 12.86, but sharing the same N-terminal sequence as Tm 12.86. This clone shares 52% relateness to Tm 13.17 clone, and more moderate (42%) similarity to either the B1 assessory gland protein or AFP-3. Again, the recombinant product did not display thermal hysteretic activity.

Importantly, thermal hysteresis behavior is the hallmark of AFPs and an essential criteria that must be met before any protein is identified as an antifreeze protein. Thus, these two attempts to isolate an insect Type III AFP gene encoding for thermal hysteretically active proteins did not establish a confirmed success. In fact, the Davies' group working on THP-12(AFP-3) concluded after numerous attempts and then NMR spectroscopy analysis, that at least in that case, it was not an AFP gene sequence that had been isolated and they (personal communication) felt certain that efforts to isolate and clone the Tm 12.86 gene would yield the same unfruitful outcome.

Nevertheless, the present invention discloses successful cloning and expression of thermal hysteretically active insect Type III AFPs from the Tm 12.86 multigene family. However, the challenge of obtaining from the bacterial expression system properly folded recombinant products capable of ice-growth inhibition was substantial, and the procedural methodology not routine or obvious to someone skilled in the art, as evidence by the Davies group conclusion. Since detection of thermal hysteretic activity is a fundamental indicator of antifreeze proteins, numerous procedures have been employed to assess thermal hysteresis, including the microcapillary method, use of the nanoliter osmometer, use of differential scanning calorimetry, and temperature gradient osmometry. The microcapillary and nanoliter osmometer methods are the most common assays used, however, it's not yet possible to directly relate the thermal hysteresis values obtained by one method with that of another method. Additionally, these methods are time consuming, require screening one sample at a time, are subject to experimenter skill, and are ice crystal size dependent. Moreover, thermal hysteresis detection is often limited in sensitivity. Historically, this latter aspect made it quite difficult to establish that several plant species did indeed produce antieeze proteins, given that detectable levels of thermal hysteretic activity in the plant extracts examined were very low, near the limits of detectability for the thermal hysteresis assay.

Another method for assessing AFP efficacy is to monitor the rate of ice crystal growth and morphology microscopically (Raymond, J. et al. [1989], Proc. Nat'l Acad. Sci. USA. 86: 881-885). As noted, one of the most striking and defining physical manifestations of THP presence is the stabilization of seed ice crystals immersed in THP solutions at temperatures maintained within the thermal hysteretic gap. Before stabilization occurs, however, there is evidence to suggest that in certain cases, very limited ice growth does occur initially for crystals. This growth is most evident in THP solutions with low thermal hysteretic activity, and results in ice crystal morphologies unique to the presence of THPs, hexagonal bipyamids that remain stable as long as temperatures are maintained within the hysteretic gap. However, more potent THP, e.g. the Tm 12.86, Type III insect AFP appears to be capable of stopping ice growth completely before bipyramids form. This, and the non-ease of the assay suggest that crystal morphology analysis of ice growth inhibtion behavior of THPs in a “non-frozen” solution maintained within the thermal hysteretic gap is not a means for rapid and routine assessment of antifreeze protein activity.

The ability of THPs to inhibit the recrystallization of ice, usually at very low THP concentrations (Knight, C. et al [1984] Nature 308: 295-296) may provide a more sensitive alternative to determination of thermal hysteresis for assessment of antifreeze protein activity. Recrystallization of any frozen crystalline solid is the process by which a subset of the crystalline grains making up the solid spontaneously grow in size by absorbing adjacent crystal grains. The net result of this process is a rearrangement of the crystal grain distribution such that a fine-grained solid is converted to a coarse-grained solid over time. Also important is the ability of recrystallization to occur under isothermal conditions. In experimental studies of recrystallization, ice samples are usually held at constant, subfreezing temperatures (“annealing” temperatures) while changes in crystal grain size are visually observed over time.

The physical basis for ice recrystallization is a reduction in interfacial free energy. Interfacial water molecules cannot assume the lowest energy hydrogen bonded configuration that exists for molecules within the interior of the crystal lattice, hence the source of interfacial free energy. The reduction in interfacial free energy resulting from recrystallization is explained from two different viewpoints. On a larger scale, recrystallization produces an overall reduction in the total lattice surface area to ice volume ratio for a given sample composed of multiple crystals. A sample composed of many, smaller crystals spontaneously evolves into a sample composed of fewer, larger crystals. The result is a reduction in total interfacial area and a concomitant reduction in interfacial free energy. On a smaller scale, recrystallization involves the movement of boundaries between adjacent crystal grains: some grains grow in size at the expense of neighboring crystals, which are gradually absorbed by the growing crystals. The boundary between an actively growing crystal grain and a neighboring grain is never a straight line but always exhibits some curvature. The tendency of the boundary is to migrate toward its center of curvature such that the degree of curvature is reduced. This is countered by the balance of interfacial tensions at the three grain “Y” junctions to achieve an equilibrium angle. These two antagonistic processes—boundary shortening followed by curvature adjustments at “Y” junctions cause the continual propagation of grain boundaries during the course of recrystallization.

Presumably thermal hysteresis proteins inhibit recrystallization in much the same way as they induce thermal hysteresis, through binding directly to ice crystal surfaces. The propagation of growing crystal boundaries is most likely inhibited by the THP induced Kelvin effect rather than by complete blanketing of ice crystal surfaces with THPs: ice growth between adsorbed THP molecules is inhibited because such growth will increase the overall surface area to volume ratio of a crystal. In terms of adjacent ice crystals, the presence of THPs will cause a “roughening” of boundary surfaces as boundaries attempt to migrate: therefore, interfacial energies will increase and boundary migration becomes energetically unfavorable.

Since recrystallization can significantly degrade the texture and product quality of frozen foods, and is quite detrimental to cell and tissue cryopreservation, chemical agents capable of inhibiting recrystallization have great potential for commercial product development Several issued patents reflect this fact, and include some means for screening for recrystallization inhibition (hereinafter referred to as “RI”) of their AFP products. U.S. Pat. Nos. 5,972,679 and 5,852,172 to Griffith, regarding antifreeze active substances in cold tolerant plants and U.S. Pat. No. 5,118,792 issued to Warren et al., for recombinant winter flounder AFP fusion proteins employ a “splat cooling” method on dry ice, originally devised by Knight, et al., [1988] (Knight, C. A. et al., [1988] Cryobiology 25;55-60) to generate a thin frozen wafer of test solution. U.S. Pat. No. 5,620,732 issued to Clemmings regarding addition of fish AFPs to enhance the storage of ice cream, uses a method of a solution droplet sandwiched between slides/cover slips which is then supercooled to −20 C to freeze the sample. U.S. Pat. Nos. 5,928,877 and 5,849,537 issued to Lusk, Cronan and Tripp et al., respectively, for recombinant expression of winter flounder AFP in yeast, use what they term the Cronan Freeze/Thaw test, basically the same method as that used by Clemmings, yet with the incorporation of a serial dilution component.

All of the above methods require observation over time with a microscope to assess if large ice crystals have replaced smaller ones, and whereby the AFP solutions are expected to produce an outcome of retention of smaller ice crystals than without the AFP additive. For the most part, these test rely solely on visual inspection and rough approximations to assess how large ice crystals have grown, providing no quantitation or, what little quantitative analysis is included in any of these patents is grossly insufficient. Some improved quantitative considerations regarding estimations of activation energies from grain growth dynamics are available (Martino and Zaritzky [1989] Cryobiology 26, 138-148; and Yeh et al., [1994] Biopolymers 34, 1495-150), yet these show large discrepancies in the generated estimates between the two, and are subject to high error rate (15%). More importantly, all of the above examples fail to account for non-specific recrystallization inhibition activity.

It is a common mistake to believe that like thermal hysteresis behavior, the phenomenon of recrystallization inhibition is antifreeze protein specific. THIS IS NOT THE CASE. Non-thermal hysteresis proteins have also been found to induce ice recrystallization inhibition (Knight, C. A. et al., [1995] Cryobiology 32:23-34). Though proteins lacking thermal hysteresis behavior do not adsorb specifically to ice like THPs, the mechanism by which they may induce R.I. presumably involves the same “roughening” of boundaries as occurs when THPs are present. As proposed by Knight, non-THPs may become trapped between boundary interfaces. To advance beyond a non-THP molecule, a migrating boundary will experience a slight increase in surface area produced by the non-THP inclusion. An increase in surface area is energetically unfavorable therefore the boundary migration is hindered. Thus, any RI assessment of antifreeze protein activity, that fails to account for common non-specific effects is problematic, and is an inherent problem in all of these earlier patents.

The invention provides a quantitative assay for RI to monitor the low levels of antifreeze protein activity seen in an in vitro T. molitor fat body cell culture system. The invention assay overcomes the problems of the prior art and early studies which were quite limited in the scope of parameters assessed (now known to be critical for specificity and reproducibility), and appropriate statistical assessment, such that evaluation of the data indicated that they had an exceptionally large error rate (>25%) of misdiagnosis. Included here in the present invention are these improvements and key details that provide for a rigorous, quantitative assessment of “antifreeze protein specific” recrystallization inhibition based on the known sensitivity and concentration dependent behaviors of the highly active purified Type RI THP from T. molitor, Tm 12.86. In addition, specific quantitative guidelines regarding the elimination of non-THP RI effects are also addressed, that will ensure a distinction between non-THP induced RI effects with those specifically attributed to antifreeze proteins. This quantitative and AFP specific RI assay is ideal for specific determination of the presence of antifreeze proteins in unknown solutions or samples, and provides a means to evaluate and rank antifreeze protein activity quantitatively, including the ability to detect and describe THP behavior at concentrations well below those exhibiting measurable thermal hysteretic activity. This increased assay sensitivity and quantitation, under conditions ensuring AFP specificity and reliability, extends the range of solution detection capabilities, that may encompass, but are not limited to evaluation of recombinant AFP products, synthetic AFP analogs, cell culture applications, assessment of activators, etc.

There exists a need for new techniques and compositions suitable for improving the preservation characteristics of organic materials at low temperatures, including storage of frozen foods and the viability of biologics. Ideally these compositions will be inexpensive, yet completely safe and suitable for human consumption or in vivo therapeutic uses. There also exists a need for new techniques and solutions suitable for depressing the freezing point or inhibiting freezing in non-organic systems such as in de-icing treatments. There also exists a need for quantitative evaluations of natural and synthetic compounds inhibiting or restricting ice growth, and new techniques for upscaling these evaluations. The present invention fulfills these and other needs.

### SUMMARY OF THE INVENTION

In the present invention, these purposes, as well as others which will be apparent, are achieved generally by providing nucleic acid sequences encoding proteins having antifreeze properties and compositional characteristics of an insect Type III AFP, wherein the nucleic acid sequences are derived from the Genus Tenebrio, including the species Tenebno molitor (Tm), the yellow mealworm beetle. Six such cDNA clones have been identified and sequenced. These cloned nucleotides have furthermore been attached to a vector and then inserted into the native DNA of a bacterial cell to provide encoding and expression for at least four highly homologous amino acid sequences that are believed to be isoforms of native Tm 12.86, a Type III AFP from T. Molitor. Three of these isoforms display identical N terminal amino acids to that of Tm 12.86. A general process flow diagram for the present invention can be found in FIG. 1.0.

The native Tm 12.86 (for Tenebrio molitor, 12.86 kDa) has previously been isolated and has been shown to be the most potent insect Type III AFP identified from this species to date. Further, following cDNA library preparation from winter acclimated Tenebrio larvae, a single full length cDNA encoding a distinct protein of 13.17 kDa (designated Tm 13.17), related to native Tm 12.86 was successfully isolated and characterized.

The present invention also details five new cDNA clones designated Tm 2-2, Tm 2-3, Tm 7-5, Tm 3-4, and Tm 3-9. The first three encode for a single, predicted 115 residue amino acid sequence, while the latter two encode for 115 residue peptides each with slightly different amino acid variants. Nucleic acid sequences for the five clones are represented in FIG. 4.13. Additionally, consensus sequences are detailed. Amino acid sequences for the three protein variants are represented in FIG. 4.14, with identified consensus sequences.

It was found that Tm 2-2, Tm 2-3, and Tm 7-5 differ in nucleic acid sequencing by just four nucleotides, but not necessarily the same nucleotides differences from one clone to the next. These differences consistently involve 12 of the same nucleotide positions. Yet, Tm 2-2, Tm 2-3, and Tm 7-5 clones encode for the same protein having a molecular weight of 12.84 kDa and an isoelectric point of 7.11. This protein, designated Tm 12.84 is believed to be similar to, but not identical with, native Tm 12.86. Although protein Tm 12.84 has an N-terminus that is identical to native Tm 12.86, Tm 12.86 is composed of 117 amino acids and has a slightly different amino acid composition. Clones Tm 34 and Tm 3-9 also display nucleotide differences associated with the same 12 nucleotide positions. They encode for highly homologous proteins of 12.84 kDa and 12.87 kDa with pI's of 7.11 and 7.14, respectively. Sequence analyses of the five clones have shown that all are highly (97%) homologous genes. Additionally, they share 50% identity (nucleotide sequence) to the Tm 13.17 clone previously identified. The presence of the clones and their strong sequence homology to each other and to the purified native Tm 12.86 indicates that a multigene family of AFPs exists for this Tm 12.86 Family of Type III AFP genes, with some more closely related than others.

The invention details further the relatedness of this Tm 12.86 AFP multigene family to other known genes, through Genbank searches, establishing that the proteins derived from the Tm 12.84 like clones and Tm 13.17 clone are most closely related (nucleic acid similarity, 43% and 57%, respectively) to B1/B2 accessory gland tubular proteins of adult male T. molitor. Also, they are somewhat similar in composition (42% and 37% for Tm 12.84 like and Tm 13.17, respectively) to a lipid carrying protein from Tenebrio designated AFP-3/THP 12 (Tang and Baust, [1995] Genbank NCBI Seq ID: 785071; Rothemund et al., [1999]). Despite the latter protein's suggestive abbreviations, the current assessment of it is not that of an antifreeze protein (Rothemund et al., 1999). Finally, the Tm 12.86 AFP family shows no similarity (20%) to the recently isolated Type II AFPs from T. molitor and D. canadensis.

The invention details through Southern and PCR analyses the arrangement of selected Tm 12.86 homologues in the genome, and the identification of a near 4000 base pair genomic fragment and several larger ones. The 4000 base pair fragment likely contains many AFP genes of approximately the same size, with the larger bands being consistent with several genes amplified in tandem. This is typical of the multigene families of fish AFPs identified.

Another object of this invention includes a series of detailed steps in cloning and procedural methodology that are necessary conditions to establish the characteristic antifreeze protein activity (e.g. thermal hysteresis) of the recombinant proteins from the Tm 12.86 AFP family of clones. The proteins, as initially isolated from the present recombinant DNA process are not themselves active in that they do not exhibit thermal hysteresis activity or the more sensitive inhibition of ice recrystallization (RI). This may be a consequence of initially expressing the recombinant protein with a signal peptide, or the inability of the current bacterial vectors to correctly fold the protein, particularly since 5 conserved cysteine residues, 4 likely involved with disulfide bridge formation, may prove problematic for correct secondary and tertiary conformations occurring within the bacterial host. The invention provides a method of establishing antifreeze protein activity in the proteins isolated from the invention clones.

Establishing activity of the recombinant products involved both a consideration of the presence of the signal peptide, and a means to enrich purification of recombinant product. The signal peptide was deleted and signal minus inserts subcloned in a new expression vector, pET-28a This expression vector allows for rapid purification of the proteins by expressing the AFPs with an N-terminal histidine tag to facilitate affinity chromatography purification (Novagen His-Bind kit) and enrichment of the recombinant AFP. Analyses of transformed clones included restriction enzyme analysis, PCR confirmation with internal and external primers, and DNA sequencing. Following confirmation of successfully generated histidine-tagged AFPs with or without signal peptides, induced protein expression and purification of recombinant AFPs were not entirely satisfactory by themselves, in obtaining antifreeze activity of the recombinant products. This procedure failed to reconstitute activity in both groups, even with numerous and various attempts at additional denaturing and refolding measures.

The initial lack of antifreeze activity by recombinant proteins obtained as described above was indeed puzzling, given the strong hydrophilic and consequently soluble nature of the Tm 12.86 family of proteins. A redirection of subsequent methodological steps was undertaken, given the chemical nature of this family of proteins was not obvious or common procedure. These steps included isolating the bacterial inclusion bodies (an unlikely source for such a soluble protein), followed by denaturation of inclusion body proteins with urea and DTT, His-tag purification and subsequent protein refolding steps (Novagen protein refolding kit). Isolation of recombinant AFPs from the inclusion bodies (and the associated reducing microenvironment and protein compaction there in) is critical for obtainment of antifreeze protein activity by these Tm 12.86 family of Type III AFPs.

The present invention also includes natural or artificial genes that would comprise nucleic acid sequence variation encoding for THP isoforms. Evidence from comparative sequence analyses and Southern analyses (see Example 4) indicate a strong likelihood that representative members of the Tm 12.86 multigene family of AFPs exist within Tenebrionidae (family) and even Tenebrionoidea (superfamily). The super family Tenebrionidea includes both the Tenebrionidae darkling beetles (including Zopheridae) plus the Pyrochroidae family of fire colored beetles including (Dendroides canadensis). Southern analyses with Tm 2.2 probe (FIG. 4.4. 4.5) has indicated a faint level of hybridization to D. canaensis genomic DNA, yet fails to recognize even faintly a band from the lepidopteran DNA (Manduca sexta). Moreover, the DNA sequences encoding Type II AFPs from both Tenebrio and Dendroides show some 48-67% nucleotide sequence similarity. Thus, it is reasonable to expect that members of the Tm 12.86 multigene family of Type III AFPs exist both within the Tenebrionionidae family and even Tenebrionoidea superfamily, and appropriate consensus nucleotide and amino acid sequence are disclosed herein.

Another object of the invention focuses on activating compounds that enhance the thermal hysteresis activity of native Tm 12.86, and the Tm 12.86 family of recombinant proteins. These include some endogenous products of Tenebrio molitor larvae, and specific rabbit immunoglobulins directed against purified Tm 12.86 and its family of recombinant proteins.

Isolation of the nucleic acid sequences encoding this Tm 12.86 family of antifreeze proteins allows the synthesis of the THP proteins in large amounts. Advantageously, these proteins can be used on numerous commercial fronts to enhance the supercooling properties of a fluid to prevent the freezing of fluids at temperatures below their equilibrium melting temperature. The proteins can also be used as de-icing solutions and to prevent or limit ice growth or recrystallization of frozen foods, and provide protection from damage that normally would result from freezing biological materials. Agriculturally, they can be used directly on crops or through transgenic means to prevent or limit frost damage. Other uses include, but are not limited to the cosmetic field, and cryosurgery. Additionally, all these effects can be mediated by adding purified THPs alone, or alternatively the THPs can be combined with various “enhancing activitor” or adjuvant compounds that are known to enhance THP activity.

The invention also details recrystallization inhibition (RI) behavior of thermal hysteresis proteins, in particular how extremely dilute solutions of THPs have been shown previously to inhibit the recrystallization of fine-grained ice samples in a concentration-dependent manner. The high sensitivity of RI to the presence of THPs and the concentration-dependent character of THP induced RI were used to develop a quantitative assay of THP activity. The extent of recrystallization in a fine-grained ice sample was quantified by estimating mean largest cross-sectional area for ice grains in the sample, thus providing the basis for a numerical assessment of RI. A number of different assay characteristics were addressed and detailed, including the specificity of the RI assay with respect to THPs, ice grain size homogeneity within RI ice samples, RI assay sensitivities, applications of the assay, and assay automation.

Critical to assay development is a recent study demonstrating the ability of non-THPs to induce RI. Therefore, the invention details specific conditions allowing non-THP induced R.I. effects, and further documents and establishes appropriate conditions and key parameters, such as, the use of NaCl or PBS (phosphate buffered saline) in solution and higher ice sample annealing temperatures to effectively distinguish between THP induced and non-THP induced RI effects. It confirms these assertions in a quantitative way, while also detailing that elimination of non-THP RI effects using NaCl or PBS or higher sample annealing temperatures is limited by the concentration of non-THPs in solution. Thus, as detailed in the invention, only if certain criteria are met, recrystallization inhibition characteristics can be used to develop a quantitative, specific, and sensitive assay for THPs.

The sensitivity of the RI assay was tested using a series of dilutions of native Tm 12.86, with finding that Tm 12.86 induces RI effects to concentrations as low as 0.5 ug/mil (samples diluted in 0.9% NaCl and annealed at −6 C for 30 minutes). Thus, for Tm 12.86, RI detection of THP activity is at least 200 times more sensitive than detection using standard thermal hysteresis measurements. Also, mean largest grain size (mlgs) measurements for native Tm 12.86 dilutions were used to construct a dilution profile plot of mlgs versus log[dilution], and arcsine(mlgs)^{0.5} which exhibited fairly strong linear character over a wide dilution range. Linear regression was used to derive an “R.I. factor” for the dilution profile, representing a relative measure of RI strength. Dilution profiles were also created for T. molitor and D. canadensis hemolymph samples and compared to the profile for native Tm 12.86. Translational shifts in hemolymph dilution profile plots along the abscissa (log[dilution]) were apparent with changes in hemolymph thermal hysteresis. Moreover, a comparison of regression line slopes between T. molitor hemolymph, D. canadensis hemolymph and purified Tm 12.86 RI dilution profiles revealed that the slopes remain remarkably similar.

The invention details the relationship between T. molitor and D. canadensis hemolymph T.H. values and RI factors, establishes it to be logarithmic, and recommends the RI assay functions best for THP solutions with low T.H. activity.

Several specific examples of application utility of the RI assay are presented whereby detectable thermal hysteretic activity is either limited or absent, such as: detection of possible THP activity in in vitro systems like T. molitor fat body cell cultures; in E. coli lysates containing recombinant THPs, and in the plasma of the cold hardy freeze tolerant frog R. sylvatica. In these examples, significant RI was detectable and quantified from the T. molitor cell culture system and recombinant Tm 12.84 and Tm 13.17 clones, both signal plus and signal minus products, with signal minus AFPs displaying significantly higher RI factors than signal plus AFPs. In contrast, no RI activity was detected in the frog plasma. Thus, the RI assay provides both a very sensitive screening tool for detection of dilute solutions of AFPs, as well as, a quantifiable and statistical means to evaluate AFP concentrations of unknown samples, and evaluative comparisons of relative strengths/potencies of, for example, different AFP types, recombinant mutants, even organically synthesized AFP/AFGP prototypes, and specific contributions of “activating substances”.

The invention then details mathematical modeling of recrystallization and AFP specific RI, and provides some predictive assessments of recrystallization kinetics and characterizations of THP induced recrystallization inhibition effects on slope and y-intercept parameters.

The invention also describes the use of a light scattering approach as an alternative method of quantitative RI assessment, a method that may be most useful with respect to screening larger sample numbers, and in respect to automation of the assay. RI assay automation is also detailed in regards to a “sandwich” method for examining concurrently multiple samples. Also upscale computer assisted image analyses of ice grain fields is discussed where by the image dimensions are specifically calibrated with regard to known parameters and dimensions produced by the serial dilution profiles and RI factor analyses of purified Tm 12.86.

Commercial uses of the AFPs of the present invention can take on many different facets, some of which are currently being pursued by industry, particulary the frozen food industry and those involved in cryopreservation of cells, tissues, organs, even new tissue engineered biologics, and cryomedicine. The noncolligative freezing point depression activity of AFPs has significant advantage over commercial antifreezes and cryoprotectants including, biodegradability, non-toxicity, and environmental safety. Moreover, these insect Type III AFPs display more potent thermal hysteresis activity than that seen with fish AFPs and AFGP, and are further subject to enhancement by activating substances, also a component of the present invention. The freezing point depression activity of the Tm 12.86 family of peptides, their capabilities of masking potential ice nucleators, ability to stabilize supercooled states, and prevent ice recrystallization, coupled with the ability to clone and express these genes in large amounts of recombinant protein make their applicability and availability for commercial use ideal. Moreover, gene transfer technology for use in generating gene modified organisms (GMO) using AFP genes has broad applicability in agriculture/aquaculture for creating cold-protected, transgenic plants, produce, and fish. On another front, there are numerous applications and advantages to using highly effective, non-toxic antifreeze in de-icing solutions (household, road protection, etc.) and with machinery, e.g., freezer coil de-icing and especially aircraft de-icing. This, coupled with a powerful new means to quantitatively assess ice recrystallization rates and comparative potency evaluations for solutions (from natural or synthetic sources) conferring antifreeze protein specific inhibition of recrystallization establishes the advantages, benefits and applications of the present invention.

Other aspects, objects, features and advantages of the present invention will be apparent when the detailed description of the preferred embodiments of the invention are considered with reference to the drawings, which should be construed in an illustrative and not limiting sense as follows:

### BRIEF DESCRIPTION OF THE DRAWINGS

FIG. 1.0 is a general process flow diagram for the present invention.

FIG. 1.1A is an Elution profile obtained when the dialyzed ethanol supernatant was chromatographed on a DEAE-Sepharose C1-6B column using a stepwise increase in NaCl. Ion exchange Peak II (tubes 34-55) was subjected to further purification. FIG. 1.1B is a thermal hysteresis profile of each ion exchange peak at a 50 mg/ml.

FIG. 1.2 is an Elution profile obtained when ion exchange Peak II was chromatographed on a Sephadex G-75 Superfine gel filtration column. Peak 3 (tubes 20-26) was the only fraction exhibiting thermal hysteretic activity.

FIG. 1.3 is a non-denaturing PAGE showing gel filtration Peak 3 as a major band (arrow) with a few lower molecular weight contaminants (25 ug of total protein was loaded). This major band was excised and electro-eluted and found to display significant thermal hysteresis activity.

FIG. 1.4 is a non-denaturing PAGE of ion exchange Peak II gel filtration Peak 3 (narrower selection: tubes 21-24) at 12.5 ug and 25 ug of protein loaded. The gel was stained Coomassie stained, with the arrow labeled Tm 12.86 indicating the major band in gel filtration Peak 3, subsequently shown to have a molecular weight of 12.86 kDa.

FIG. 1.5 is an Elution profile of the Reverse Phase HPLC analysis of gel filtration Peak 3 from ion exchange Peak II. Results indicate that gel filtration Peak 3 elutes as a single species at 30 minutes.

FIG. 1.6 is the results from Mass Spectrometry which indicate that the 30-minute peak off the Reverse Phase HPLC column is indeed one species having a molecular mass of 12,862 Daltons.

FIG. 1.7 is a Tricine SDS polyacrylamide gel electrophoresis of the electro luted band from the non-denaturing PAGE of ion exchange Peak II gel filtration Peak 3. Tm 12.86 was treated with b-mercaptoethanol (w) produces a distinct doublet, which is eliminated if b-mercaptoethanol was left out (w/o), yielding a single band at approximately 12.7-12.9 kDa

FIG. 1.8 is the N-terminal analysis of Tm 12.86 (SEQ. ID No. 1) depicting leucine at the amino terminus.

FIG. 1.9 is a thermal hysteresis activity curve for Tm 12.86 over a concentration range of 0.125 to 25 mg/ml. On average, 2-3 samples for each concentration were tested. Error bars indicate standard error of the mean. Tm 12.86 displays a considerably larger amount when compared to a previously purified T. molitor Type III antifreeze protein.

FIG. 1.10 is a Western blot analysis of a 15% Glycine SDS-PAGE comparing winter-acclimated T. molitor hemolymph (H) to a serial dilution of Tm 12.86. Hemolymph protein (20 ug of total hemolymph protein in 0.5 ul volume) was co-electrophoresed with a serial dilution of Tm 12.86 (in ug). The purified Tm 12.86 served to create a standard curve whereby band intensity could be used to estimate the hemolymph concentration of Tm 12.86. The intensity of the hemolymph band approximates that of 1-1.5 ug of Tm 12.86 in 20 ug of total hemolymph protein or 5-7.5% of the total hemolymph protein in winter-acclimated T. molitor. Furthermore, this 1-1.5 ug from an initial volume of 0.5 ul of hemolymph estimates that the physiological concentration of Tm 12.86 in winter-acclimated T. molitor hemolymph is approximately 2-3 mg/ml.

FIG. 1.11 is an elution profile obtained when Peak IV off the ion exchange column was chromatographed on a Sephadex G-75 Superfine gel filtration column. Thermal hysteretic activity was restricted to Peak 3 (tubes 22-25), while peak 4 (tubes 28-33) was the only peak displaying activator activity.

FIG. 1.12 is thermal hysteresis activity curves showing the enhancement of activity to Tm 12.86 by the addition of an activating factor, Note that the highest degree of enhancement (0.75° C.) occurs around 2.5 mg/ml of antifreeze protein (the physiological range of Tm 12.86 as determined by Western Blot analysis). On average, 2-3 samples for each concentration was used to determine thermal hysteresis. Error bars indicate standard error of the mean.

FIG. 1.13 is an Ultraviolet Absorption spectrum of the gel filtration Peak 4 of ion exchange Peak IV indicating major absorbance peaks at 205, 240, and 275 nm.

FIG. 2.0 is an agarose gel of isolated total RNA; RNA minus mRNA, and mRNA from winter acclimated T. molitor whole larvae. In ordinate: RNA molecular weight scale in kb or position of 18S or 28S, respectively.
Lane 1: the total RNA (4 μg);
Lane 2: RNA (2 μg) after mRNA extracted;
Lane 3: the isolated mRNA (1 μg) from total RNA;
Lane 5: molecular weight marker, LambdaDNA digested by HindIII.
Lane 6: RNA molecular weight marker.

FIG. 2.1 are translation products generated with in vitro translation kit using control mRNA, and isolated mRNA from T. molitor. 5 μl (˜200 cpm) of the translated products were loaded onto a 20% SDS-PAGE gel and electrophoresed and subjected to fluorography. Fluorograph of gel exposed to X-ray film for 2 day at −80° C. In ordinate: protein molecular weight scale in kDa.
Lane 1: translation product (3 μl) in the absence of mRNA (negative control);
Lane 2: translation product using 2 μg of the control mRNA provided by manufacture;
Lane 4 to 9: in vitro translation products (5 μl) directly synthesized by isolated mRNA (2 μg) from winter acclimated (Lane 4; 6 and 8) or unacclimated T. molitor (Lane 5; 7 and 9).

FIG. 2.2 illustrate Coomassie staining of immunoprecipitation samples (lane 1-4) derived from the original in vitro translation products (FIG. 2.1). Immunoprecipitated samples (volume. of 35; 17; 35 and 35 μl for lane 1-4, respectively) and in vitro translation products (1 μl, lane 6-9) were loaded on 17% SDS-PAGE gel and subjected to electrophoresis. In ordinate: protein molecular weight scale in kDa.
Lane 1 to 2: Immunoprecipitation products from samples of T. molitor, Lane 3: Immunoprecipitation products for control from sample of T. molitor;
Lane 4: Immunoprecipitation products for control;
Lane 6 to 7: in vitro translation products directed by isolated mRNA of T. molitor;
Lane 8 to 9: in vitro translation products directed by no mRNA and control mRNA.

FIG. 2.3 is a fluorograph of FIG. 2.2's immunoprecipitation products (lane 1-4) of original in vitro translations, thus identifying peptides incorporating 35S-methionine during in vitro translation which were recognized and immunoprecipitated by Tm 12.86 antiserum. The 17% SDS-PAGE gel and was exposed Biomax MR x-ray film for 27 days at −80° C. In ordinate: protein molecular weight scale in kDa.
Lane 1 and 2: immunoprecipitation (as arrow) of T. molitor samples from in vitro translation products;
Lane 3: immunoprecipitation of control samples
Lane 4: immunoprecipitation from negative control (dH2O replace of in vitro products in immunoprecipitation);
Lane 6 and 7:in vitro products directed by isolated mRNA of T. molitor,
Lane 8: translation product in the absence of mRNA (negative control);
Lane 9: translation product using 1 μl of the control mRNA provided by manufacturer.

FIG. 2.4A is a diagram of the ZAP Express vector and excised pBK-CMV phagemid vector (Stratagene). FIG. 2.4B is a physical map of pBK-CMV phagemid vector (Stratagene). It has a 4518 basepair with multple cloning sites. The portion of the pBK-CMV DNA sequence is shown on the bottom line of the figure. The cDNA of T. molitor was cloned into the two cloning sites EcoR 1 and Xho I (in box). Inserted cDNA can be excised by co-infection with helper phage from the ZAP express vector as a recombinant Bluescript® SK (−) phagemid. T3 and T7 primers (underlined) are used for sequencing the insert DNA from both end. The expression of the cloned gene in the plasmid is controlled by lac promoter.

FIG. 2.5 is an electrophoresis gel of recombinant pBK-cmv plasmid DNA. The pBK-cmv plasmid DNA containing the cDNA insert was isolated from positive colonies and digested with either one [Lane 2 (4 μg digested by Xho 1) and 3 (2 μg DNA digested byEco R I] or two restriction enzymes [Lane 1 (2 μg DNA digested by Xho I andeco R I); or no restriction enzyme (Lane 4 (2 μg DNA) and 5 (2 μg DNA). DNA molecule weight standard (3 μg) is shown in Lane 6. The digested DNA was electrophoresed to seperate the fragments according to sizes. In Lane 1 two different sizes of fragments, the smaller one (˜500 bps) is the expected cDNA insert (pointed by arrow) and the larger one was 4518 bp pBK-cmv plasmid vector, Lane 2 shows partially digested DNA by Xho I and contained 4 fragments, the largest one was bacterial genomic DNA; the second and the smallest bands represent nicked and supercoiled forms of the recombinat plasmid respectively; the third one represents linear form of the recombinant plasmid (<4518 bps) as comparison of bands to non-digested plasmid DNA (lane 4 and 5).

FIG. 2.6 is a complete sequence of the FW1 clone encoding Tm 13.17 (SEQ. ID NO. 2) and its deduced amino acid (SEQ. ID NOs 3 and 4) of the protein of T. molitor. FIG. 2.6A is the full length nucleotide sequence and corresponding deduced amino acid (in single letter nomenclature); The translation start codon, ATG is boxed, and a putative signal peptide sequence are underlined; the stop codon. TGA is in asterisk; polyadenlation signal is in italic and bold, and poly (A) tail is in bold. The arrow indicates the putative cleavage site of the signal peptide. FIG. 2.6B is the signal peptide from deduced amino acid sequence of FW1 cDNA clone. The typical three regions of signal peptide are underlined. The cleavage site is indicated by arrow. FIG. 2.6C is the amino acid sequence and compositional analysis for the predicted mature Tm 13.17.

FIG. 2.7 illustrates the alignment between the nucleotide cDNA sequences of B1 and Tm 13.17 of T. molitor. Identical nucleotide sequence is boxed. The start of the mature protein is marked with an arrow, and the stop codons are shown by a star.

FIG. 2.8 illustrates the sequence alignment between mature Tm 13.17 and AFP-3 of T. molitor. Vertical line indicates identical amino acids; two dots indicate highly conservative replacement, and one dot indicates less conservative replacement.

FIG. 2.9 illustrates the alignment of putative signal peptide sequences of Tm 13.17, AFP-3 and B1 protein of T. molitor. The identical amino acid residues and highly conservative replacement are boxed.

FIG. 2.10 illustrates the alignment of N-terminal amino acid sequences of Tm 13.17 and Tm 12.86. The identical amino acids are boxed, dots indicate conservative replacement amino acids.

FIG. 2.11 illustrates the immunoblot of Tm 13.17 expressed in the XLOLR host with antibody of Tm 12.86. Lane 1: Tm 12.86 from hemolymph; Lane 3 to 5: Tm 13.17 expressed in the XLOLR host cells under induce condition (with IPTG of 1-2 mM), Lane 6 to 7: Tm 13.17 expressed in the XLOLR host cells under non-induced condition (without IPTG). Lane 8 to 9: XLOLR host cells without cDNA insert; Lane 10: pre-stained protein standard and its size was expressed in kDa as labeled. The protein band recognized by the antibody is indicated by the arrow. About 30 μg of total protein for each sample was loaded and electrophoresed, then immunoblotted.

FIG. 2.12 illustrates the alignment of three amino acid sequences for Tm 13.17, B1 and AFP-3. 18 N-terminal amino acid residues of Tm 12.86 is also shown in the alignment. The identical amino acid residues are boxed. Note that the arrangement of the proteins from top to bottom (Tm 12.86, Tm 13.17, B1, and AFP-3) displays first the strong relatedness, and then the falling off identity between the peptides.

FIG. 3.0 illustrates the cDNA nucleotide sequence (SEQ ID NO 5) and amino acid translation of clone 2-2 (SEQ ID NO 7 AND 8). The signal sequence is underlined, and the arrow denotes the predicted beginning of the mature protein. The start codon is boxed, and the stop codon is denoted by a star.

FIG. 3.1 is the cDNA nucleotide sequence (SEQ ID NO 6) and amino acid translation of clone 2-3 (SEQ ID NO 7 AND 8). The signal sequence is underlined, and the arrow denotes the predicted beginning of the mature protein. The start codon is boxed, and the stop codon is denoted by a star.

FIG. 3.2: illustrates comparative nucleotide sequence analysis between clones 2-2& 2-3. Areas of the sequences that are different are boxed.

FIG. 3.3 illustrates predicted amino acid composition and related information for the peptide derived from clones 2-2/2-3.

FIG. 3.4 is a Western blot of SDS-PAGE gel comparing Tm 13.17 and 2-3 recombinant products with Tm 12.86 proteins. Also included is a negative control consisting of proteins from XLOLR E. coli lacking the pBK-CMV phagemid. No significant immunoreactive bands were observed for the XLOLR proteins.
Lane A: T. molitor hemolymph (long day conditions)
Lane B: Prestained standards (Coomassie)
Lane C: Recombinant 2-3
Lane D: Recombinant 2-3
Lane E: Recombinant Tm 13.17
Lane F: XLOLR soluble proteins (negative control)
Lane G: Kaleidoscope prestained standards

FIG. 4.0 is a Southern blot of three cDNA clones, hybridized with the DIG labeled Tm 13.17 cDNA probe at 68° C., and detected chemiluminescently. Film exposure was 1 h at 37° C.
Lane 1: 2-2 cDNA, 180 ng
Lane 2: 2-3 cDNA, 180 ng
Lane 3: Tm 13.17 cDNA, 180 ng
Lane 4: 2-2 and 2-3 cDNAs, 90 ng each
Lane 5: 2-2 and Tm 13.17 cDNAs, 90 ng each
   Arrows:
a: Tm 13.17 probe detecting 2-2 and/or 2-3 cDNA
b: Tm 13.17 probe detecting Tm 13.17 cDNA

FIG. 4.1 is a Southern blot of restriction digested T. molitor genomic DNA. Hybridized overnight with 32P labeled 2-3 probe at 42° C. Film exposure was 48 h at −70° C.
Lane 1: 2-3 cDNA, 20 ng
Lane 2: Molecular weight marker (Hind III
Lane 3: EcoR I cut genomic T. molitor DNA, 20 μg
Lane 4: EcoR I cut genomic T. molitor DNA, 40 μg
Lane 5: BamH I cut genomic T. molitor DNA, 40 μg
Lane 6: BamH I cut genomic T. molitor DNA, 60 μg

FIG. 4.2 illustrates 32P labeled 2-3 probe hybridized to Southern blot at 40° C. overnight Film exposure was for 1.5 hours at −70° C.
Lane 1: 2-3 cDNA, 20 ng
Lane 2: Pst I and Kpn I cut genomic T. molitor DNA, 70 μg
Lane 3: Pvu II cut genomic T. molitor DNA, 70 μg
Lane 4: Hae III cut genomic T. molitor DNA, 70 μg
Lane 5: Hae III and Ban I cut genomic T. molitor DNA, 70 μg
Lane 6: Kpn I cut genomic T. molitor DNA, 70 μg

FIG. 4.3 is a Southern blot of restriction digested T. molitor genomic DNA. Hybridized overnight with 32P labeled Tm 13.17 probe at 42° C. Film exposure was at −70° C. for 48 hours.
A. Lane 1: Tm 13.17 cDNA, 20 ng
Lane 2: Molecular weight marker (Hind III
Lane 3: EcoR I cut genomic T. molitor DNA, 20 μg
Lane 4: EcoR I cut genomic T. molitor DNA, 40 μg
Lane 5: BamH I cut genomic T. molitor DNA, 40 μg
Lane 6: BamH I cut genomic T. molitor DNA, 60 μg
B is identical blot to above, but hybridized with the 32P labeled 2-3 probe.

FIG. 4.4 is a Southern blot hybridized with 32P labeled 2-2 cDNA probe at 42° C. The film was exposed for 16 hours at −70° C.
Lane 1: Molecular weight marker (HindIII (unlabeled)
Lane 2: T. molitor genomic DNA, 30 μg, cut with EcoRI and BanI
Lane 3: T. molitor genomic DNA, 30 μg, cut with EcoRI
Lane 4: T. molitor genomic DNA, 30 μg, cut with HindIII
Lane 5: Dendroides canadensis genomic DNA, 30 μg, cut with EcoRI
Lane 6: T. molitor genomic DNA, 30 μg, cut with PstI

FIG. 4.5 is a Southern blot hybridized with 32P labeled 2-2 cDNA probe at 42° C. The film was exposed for 16 hours at −70° C.
Lane 1: Molecular weight marker (HindIII (unlabeled)
Lane 2: Manduca sexta genomic DNA, 30 μg, cut with EcoRI
Lane 3: T. molitor genomic DNA, 30 μg, cut with Hhal (4 bp cutter)
Lane 4: T. molitor genomic DNA, 30 μg, cut with Rsal (4 bp cutter)
Lane 5 T. molitor genomic DNA, 30 μg, cut with HhaI (4 bp cutter)

FIG. 4.6 illustrate PCR primers used to amplify genomic DNA. FIG. 4.6A illustrates the Tm 13.17 cDNA nucleotide sequence, with the forward and reverse primer sequences boxed. FIG. 4.6B illustrates representative amino acid sequence alignments of 2-2, Tm 13.17, B2, and AFP 3. The primer sequences, which only exactly match Tm 13.17, were taken from the boxed areas. FIG. 4.6C illustrates the percent composition and melting temperatures of the forward and reverse primers shown in FIG. 4.6A.

FIG. 4.7 illustrates PCR products generated with Tm 13.17 forward and reverse primers, and detected with a 32P labeled Tm 13.17 cDNA probe.

FIG. 4.8 is an ethidium bromide stained agarose gel containing T. molitor genomic PCR products in lanes 2, 3, and 4. Lane one contains Lamda HindIII molecular weight markers. The bands seen at the arrow are approximately 3650 base pairs in size. One percent DMSO was added to the reaction.

FIG. 4.9 illustrates PCR products generated with Tm 13.17 forward and reverse primers with 1% DMSO added to the reaction, hybridized with a 32P labeled 2-2 cDNA probe.
Lane 1: Molecular weight marker (HindIII (unlabeled)
Lanes 2-5: Ten μl of a 50 μl total volume PCR.

FIG. 4.10A is the cDNA nucleotide sequence (SEQ. ID NO. 9) and translation of 3-4 (SEQ ID NO. 10 (precursor) and SEQ. ID NO. 11 (mature protein). The signal sequence is underlined, and the arrow denotes the predicted beginning of the mature protein. The start codon is boxed, and the stop codon is denoted with a star. FIG. 4.10B is the amino acid composition and related information of the predicted mature 3-4 protein.

FIG. 4.11A is the cDNA nucleotide sequence (SEQ. ID NO. 12) and translation of 3-9 (SEQ ID NO. 13 (precursor) and SEQ. ID NO. 14 (mature protein). The signal sequence is underlined, and the arrow denotes the predicted beginning of the mature protein. The start codon is boxed, and the stop codon is denoted with a star. FIG. 4.11B is the amino acid composition and related information of the predicted mature 3-9 protein.

FIG. 4.12A is the cDNA nucleotide sequence (SEQ. ID NO. 15) and translation of 7-5 (SEQ ID NO. 7 (precursor) and SEQ. ID NO. 8 (mature protein). The signal sequence is underlined, and the arrow denotes the predicted beginning of the mature protein. The start codon is boxed, and the stop codon is denoted with a star. FIG. 4.12B is the amino acid composition and related information of the predicted mature 7-5 protein.

FIG. 4.13 illustrates the alignment between the cDNA sequences 2-2,2-3, 3-4, 3-9, and 7-5. Nucleotide residues which disagree are boxed. The start of the mature protein is denoted by an arrow, and the stop codon is marked with a star.

FIG. 4.14 illustrates the alignment of the amino acid sequences of 2-2,2-3, 3-4, 3-9, and 7-5, predicted from the nucleotide sequence of the cDNAs. Amino acid residues that differ between sequences are boxed. The arrow denotes the start of the mature protein.

FIG. 4.15 is the Composite of amino acid and predicted amino acid data for Tm 12.86 and its homologous clones (Tm 13.17, 2-2, 2-3, 3-4, 3-9, 7-5).

FIG. 4.16 illustrates the alignment between the amino acid sequences of Tm 12.86, 2-2, 2-3, 3-4,3-9, 7-5, Tm 13.17, B1, B2, and AFP-3. All are sequences obtained from T. molitor. All except Tm 12.86 are amino acid sequences predicted from cDNA nucleotide sequences. The start of the mature protein sequence is at the arrow. Conserved cysteine residues are denoted in yellow. Residues which agree in all nine sequences or ten including the N-terminus of Tm 12.86 are in blue. Residues which agree in at least seven proteins are in orange. An open circle denotes a single amino acid deletion in 2-2, 2-3, 3-4, 3-9 and 7-5.

FIG. 4.17 illustrates the alignment of Tm 13.17, 2-2 (representative of 2-2, 2-3, 3-4, 3-9, and 7-5), B1, B2, and eight pheromone binding proteins from various insects. Arrows above yellow highlighting denotes conserved cysteine residues found in all 12 aligned sequences. Yellow highlighting with no arrow denotes cysteine residues conserved in the insect pheromone binding proteins, but not in the B proteins, 2-2, or Tm 13.17. Red shading shows agreement between one or more of the Tm 13.17, 2-2, or B1/B2 sequences and any of the representative pheromone or odorant binding proteins. (Pbp: pheromone binding protein; Obp: oderant binding proteins, Antpo (Antherea polyphemus); Manse (M. sexta), Drome, Drosophila melanogaster).

FIG. 4.18 illustrates the areas of repeated similarity surrounding the conserved cysteine residues of 2-2, 2-3, 3-4, 3-9, 7-5, Tm 13.17, B1, B2, and AFP-3. Conserved cysteine residues are in yellow. Lysine residues are shown in red, glutamate in green, isoleucine in orange, and valine in blue.

FIG. 4.19 illustrates the percent similarity and percent divergence of the Tm 12.86 homologues, the B proteins, AFP-3 and the Type II AFPs isolated from T. molitor (YL-1) and from D. canadensis (DAFP-1A. The uppermost table compares nucleotide sequences, and the lower table compares amino acid sequences.

FIG. 4.20 illustrates the phylogenetic tree of the same nucleotde sequences displayed in FIG. 4.19.

FIG. 5.0 is the pET-28a expression vector (Novagen Catalogue)

FIG. 5.1 is a schematic illustration of the strategy implemented to generate His-tagged signal plus and signal minus clones and recombinant products.

FIG. 5.2 illustrates the restriction digest screening for pET-2-2 (signal minus insert) in potential clones, demonstrated by the appearance of 350 bp fragment. Also, PBK-CMV double digested to yield a 500 bp fragment served as the positive control. Eighteen potential clones were cultured, mini-preped and restriction digested to screen for incorporation of signal-minus fragment. 10 μl of each mini-prep DNA was digested with BAMHI and XhoI and loaded in lanes lableled 1-18 in a 1% agarose gel. Clones in lanes 2, 4, 6, 7, 8, 9, 11, 12, 13 and 18 show a fragment of 350 bp, as marked by the arrow on the right. A positive control pBK-CMV 2-2 was double digested similarly and the 500 bp AFP fragment is seen and denoted by an arrow to the left. The first lane has 1 μg of 100 bp molecular weight marker.

FIG. 5.3 illustrates further confirmation of cloned signal minus inserts for pET-2-2 and 2-3 (showing 1400 bp fragments) and 13.17 (no 1400 bp fragment) with PvuI restriction enzyme digestion. U: uncut, C: cut. Seen also, pBK-2-2 releasing a 650 bp fragment. 10 μl of miniprep DNA was digested with Pvul and electrophoresed on a 1% agarose gel. First two lanes are 1 kb and 100 bp molecular weight markers. Lane 1 is undigested, self-ligate pET vector and Lane 2 is digested, self-ligated pET vector which was linearized. Lane 3 is undigested pBK 2-2 vector and Lane 4 is digested vector which released a 650 bp fragment. Lane 5 is digested pBK: Tm 13.17 and Lane 6 and 7 are undigested and digested pET-28a respectively. Lane 8, 9, 10 and 11 are few of the selected clones of pET 2-2. Clones in Lane 9 and 11 release a desired fragment of 1400 bp. Similarly, clones in lanes 12, 13, and 14 were analyzed for pET 2-3 and only Lane 12 released the desired fragment of 1400 bp. Finally, one sample of pET-Tm 13.17 was analyzed and the undigested sample was run in Lane 15 and the digested sample was run in Lane 16. The lack of any fragment confined the presence of Tm 13.17.

FIG. 5.4 illustrates confirmation of signal deleted pET clones with PCR. External primers amplifying 500 bp band in pBK-2-2, 2-3, 13.17, but not in pET; Internal primers amplifying 350 bp band in pET-2-2, 2-3, and 13.17. and pBK. The first two lanes from left are molecular weight markers of 1 kb and 100 bp. Positive controls for the PCR reaction are loaded in Lanes 1, 2 and 3 with pBK-2-2, 2-3 and pET without any insert and Lanes 5, 6, 7 and 8 are 2-2 (S−), 2-3 (S−), Tm13.17 (S+) and Tm13.17 (S−) in pET vector, respectively. The absence of any bands postively confirms that there is no contamination from the original vector, pBK-CMV. The second set of samples from Lanes 9 to 16 have been amplified with primers designed to internal sequences of AFP genes. Lanes 9, 10 and 11 are AFP genes in the pBK vector. The amplification of the plasmids confirms the presence of AFP genes. Lane 12 is the pET vector without any insert and the absence of amplified DNA was expected. Lanes 13, 14, 15 and 16 are 2-2 (S−), 2-3 (S−), Tm13.17 (S+) and Tm13.17 (S−) in pET vector, respectively. The presence of a 350 bp fragment confirms the presence of the AFP genes in the pET-28a vector. Lane 15 did not amplify in this gel, but has amplified in other gels (data no shown).

FIG. 5.5 illustrates restriction digest screening for pET-2-2 (signal plus) and pFF-2-3 (signal plus) in potential clones demonstrated by the appearance of 500 bp fragments. Nine potential clones for 2-2 and nine clones for 2-3 were cultured, min-preped and restriction digested to screen for incorporation of signal plus fragment. 10 μl if each mini-prep DNA was digested with BamHI and XhoI and loaded into lanes labelled 1-18 in a 1% agarose gel. Clones in Lanes 3 and 4 of 2-2 and 11 and 18 of 2-3 release the desired fragment of 500 bp. Lanes 6 and 8 failed to produce DNA suggesting that the cultures might be satellite colonies. Molecular weight markers were loaded in the first two lanes with 1 μg of 1 kb marker in the first lane and follwed by 1 μg of 100 bp marker.

FIG. 5.6 illustrates immunoblotting of recombinant proteins of pET: signal plus and signal minus products column purified and thrombin cleaved. Western blot of recombinant products following 15% SDS-PAGE and detection with anti-Tm 12.86 antiserum. A Western blot of recombinant proteins was electrophoresed on a 15% SDS-PAGE and transferred to a PVDF membrane. The membrane was blocked with milk and incubated first with rabbit anti-Tm12.86 and then incubated with horse radish peroxidase conjugated goat anti-rabbit antibody. Lane 1 depects molecular weight markers of 46, 29, 20, 14, 8 and 3.5 kD. Lanes 2 and 3 represent 0.1 μl of T. molitor hemolymph and 1 μg of purified Tm12.86, respectively. Lane 4 is 2 μg of whole bacterial lysate from pET 2-2 (S+) and Lanes 5, 6, 7, 8, 9 and 10 represent 1 μg of column purified, thrombin-cleaved, recombinant proteins of pET 2-2 (S+), 2-2 (S−), 2-3 (S+), 2-3 (S−), Tm13.17 (S+) and Tm13.17 (S−), respectively.

FIG. 5.7 describes the specific cDNA nucleotide sequence (SEQ. ID NO. 16) and translation precursor protein (SEQ ID NO. 17) of His-tagged signal plus 2-2 clone. The signal sequence is underlined, and bold “1” denotes the predicted beginning of the mature protein. The start codon is labeled, and the stop codon is denoted with a star.

FIG. 5.8 describes the specific cDNA nucleotide sequence (SEQ. ID NO. 18) and translation of mature peptide (SEQ ID NO. 19) of His-tagged signal minus 2-2 clone. The His-tag is upstream of the N-terminal of the mature protein. The bold “1” denotes the predicted beginning of the mature protein. The stop codon is denoted with a star.

FIG. 5.9 describes the specific cDNA nucleotide sequence (SEQ. ID NO. 20) and translation precursor protein (SEQ ID NO. 21) of His-tagged signal plus 2-3 clone. The signal sequence is underlined, and bold “1” denotes the predicted beginning of the mature protein. The start codon is labeled, and the stop codon is denoted with a star.

FIG. 5.10 describes the specific cDNA nucleotide sequence (SEQ. ID NO. 22) and translation of mature peptide (SEQ ID NO. 23) of His-tagged signal minus 2-3 clone. The His-tag is upstream of the N-terminal of the mature protein. The bold “1” denotes the predicted beginning of the mature protein. The stop codon is denoted with a star.

FIG. 5.11 describes the specific cDNA nucleotide sequence (SEQ. ID NO. 24) and translation precursor protein (SEQ ID NO. 25) of His-tagged signal plus Tm 13.17 clone. The signal sequence is underlined, and bold “1” denotes the predicted beginning of the mature protein. The start codon is labeled, and the stop codon is denoted with a star.

FIG. 5.12 describes the specific cDNA nucleotide sequence (SEQ. ID NO. 26) and translation of mature peptide (SEQ ID NO. 27) of His-tagged signal minus Tm 13.17 clone. The His-tag is upstream of the N-terminal of the mature protein. The bold “1” denotes the predicted beginning of the mature protein. The stop codon is denoted with a star.

FIG. 6.0 is an SDS-PAGE of Tm 13.17 recombinant protein after His-Tag affinity chromatography. Lane 1, shows the low molecular weight protein standards. Lane 2 shows Tm 13.17 thrombin cleavage.

FIG. 6.1 illustrates the Tm 13.17 recombinant protein evaluated by Western blot screening with anti-Tm 12.86. Lane 1, Tm 13.17 no thrombin cleavage. Lane 2, T. molitor hymolymph. Lane 3, Prestained SDS-PAGE mw standards.

FIG. 6.2 illustrates the recrystallization inhibition of Tm 13.17 Signal plus and Signal minus recombinant proteins. A is the PBS control; B is Bacteria without insert control; C is Tm 13.17 S+ at 1 mg/ml; and D is Tm 13.17 S− at 0.5 mg/ml.

FIG. 6.3 illustrates the R. I. dilution profile for recombinant Tm 13.17 at 10 mg/ml starting concentration. All samples were diluted in PBS, and mean largest grain sizes determined using the random sampling method. The R.I. factor from regression line is 1.93

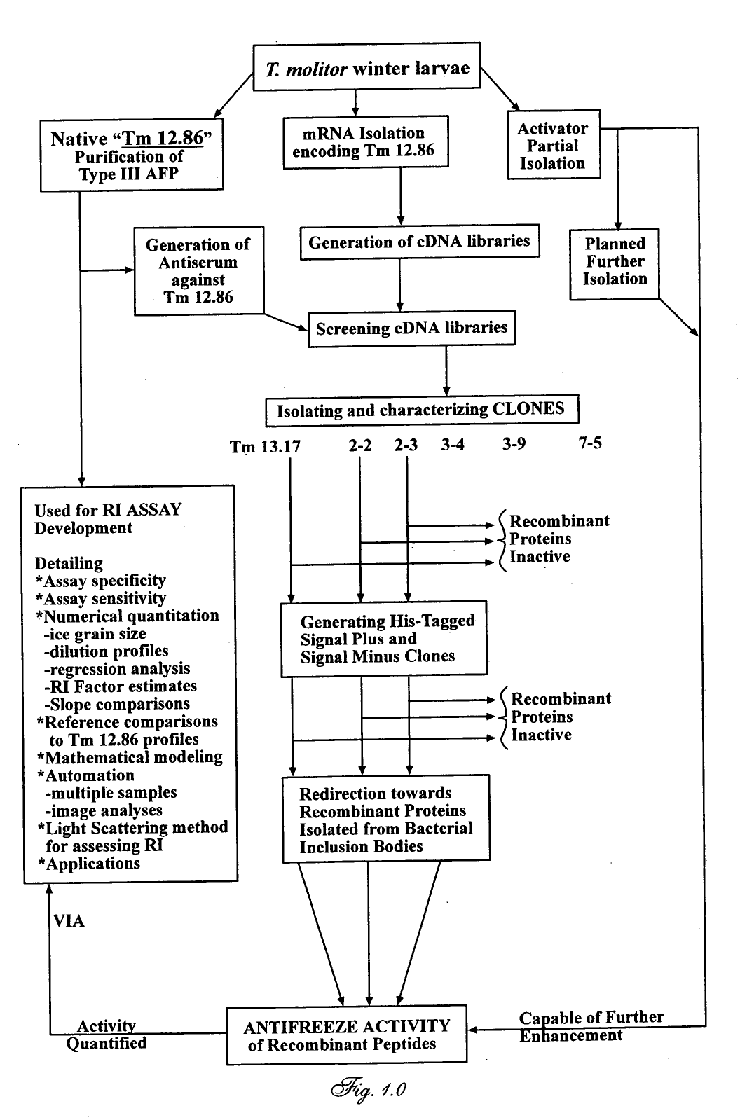

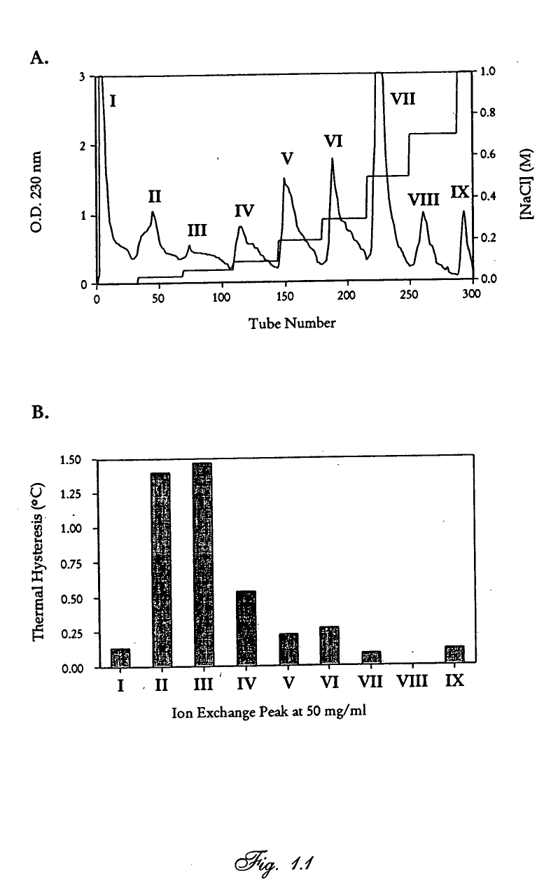

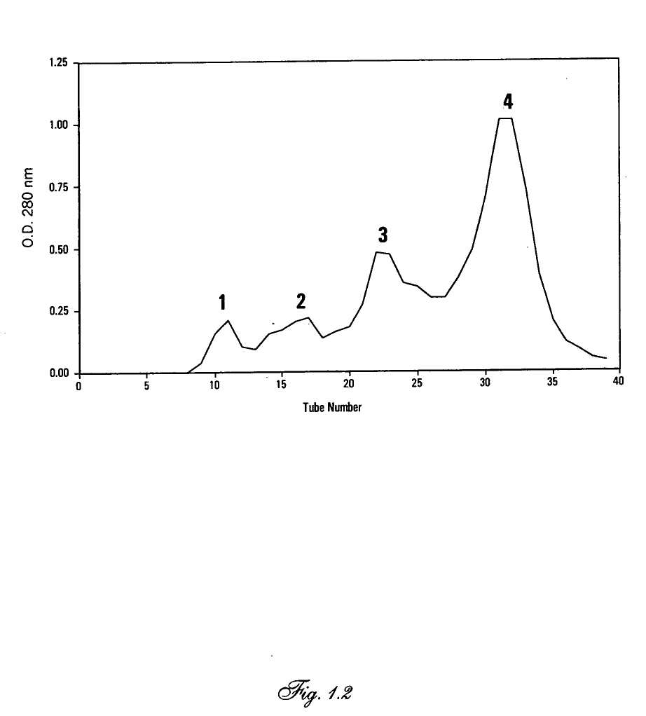

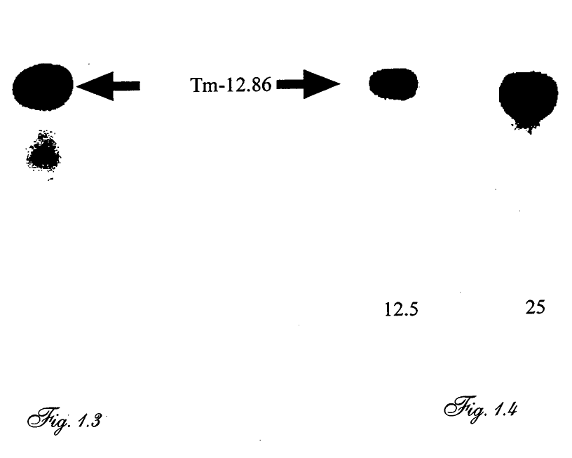

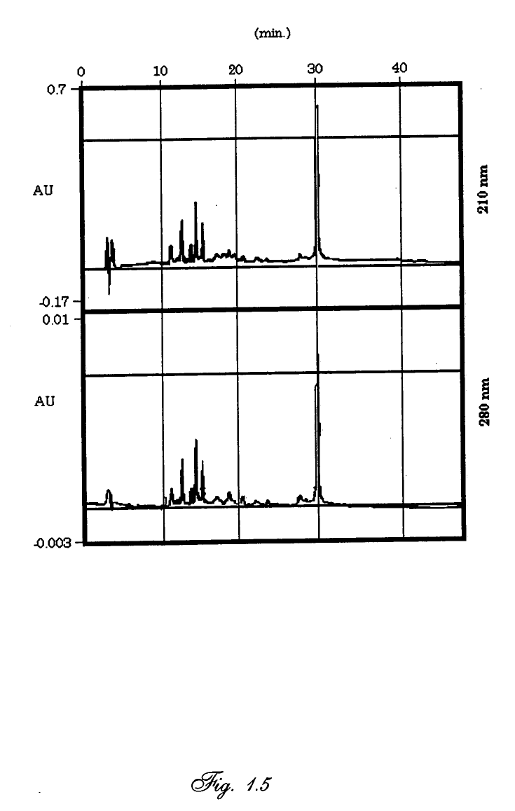

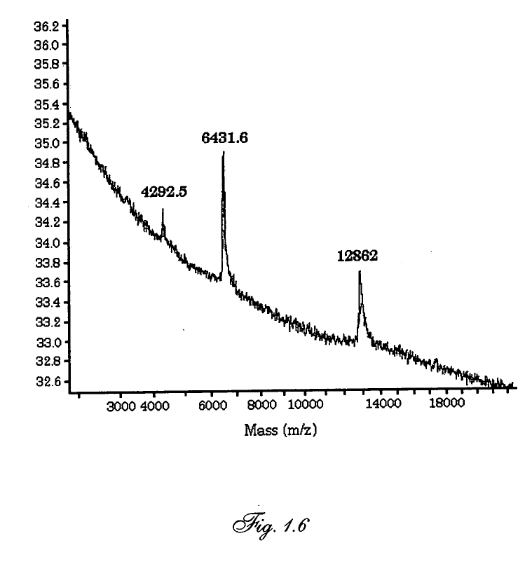

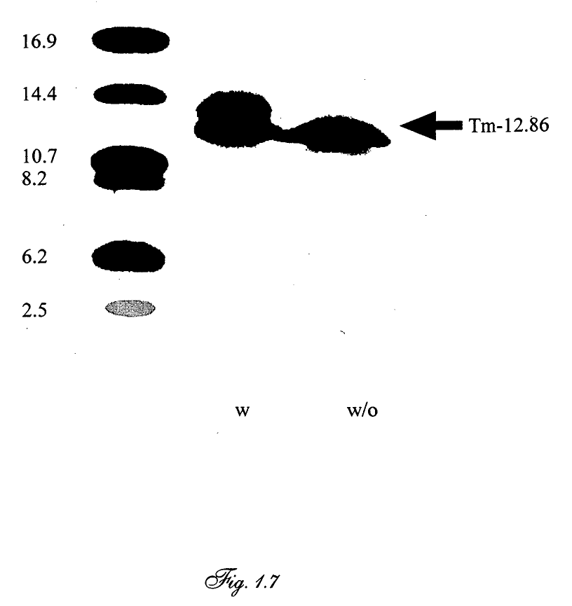

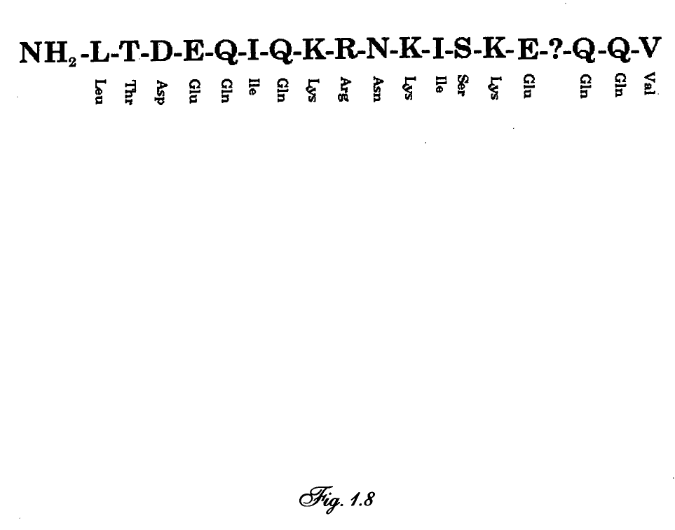

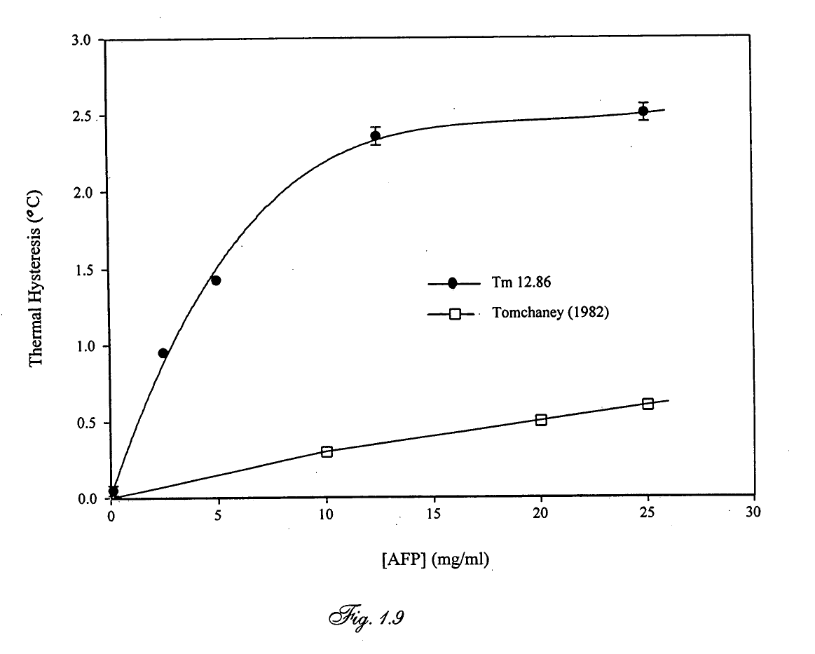

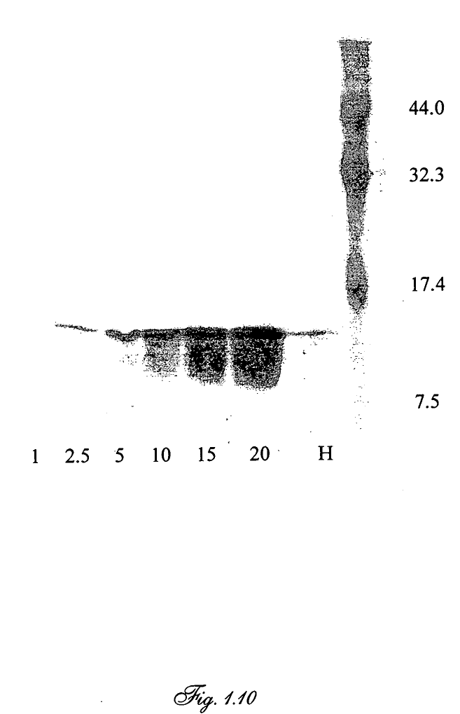

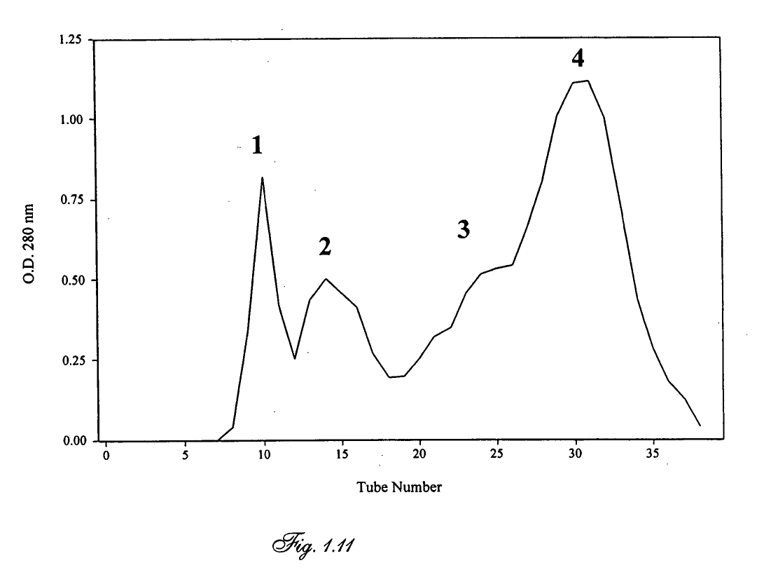

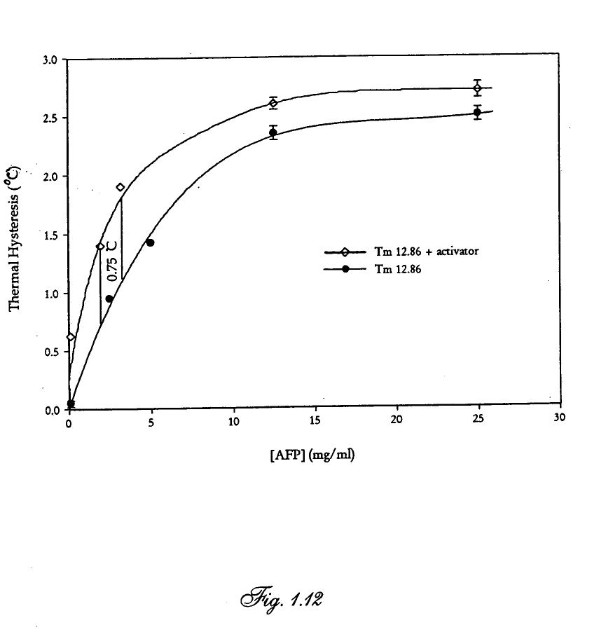

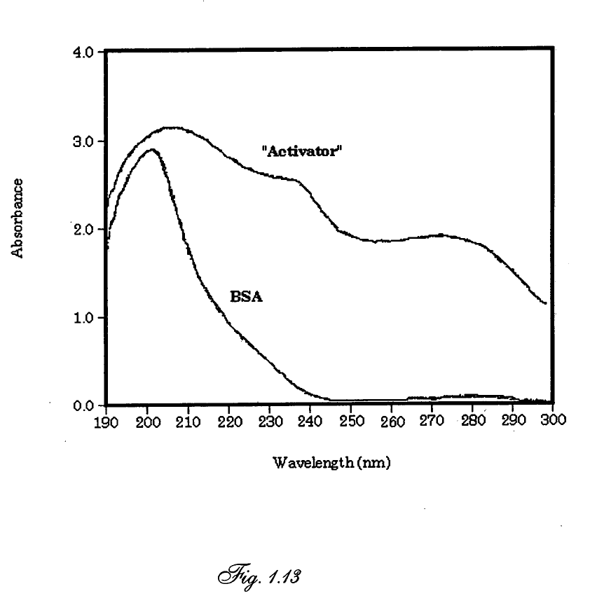

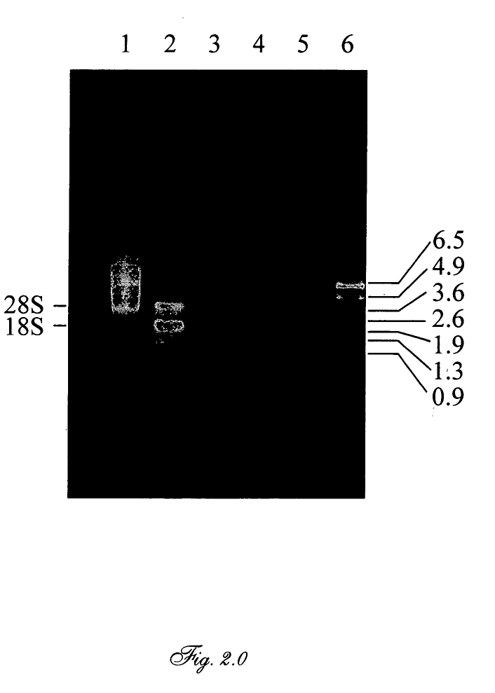

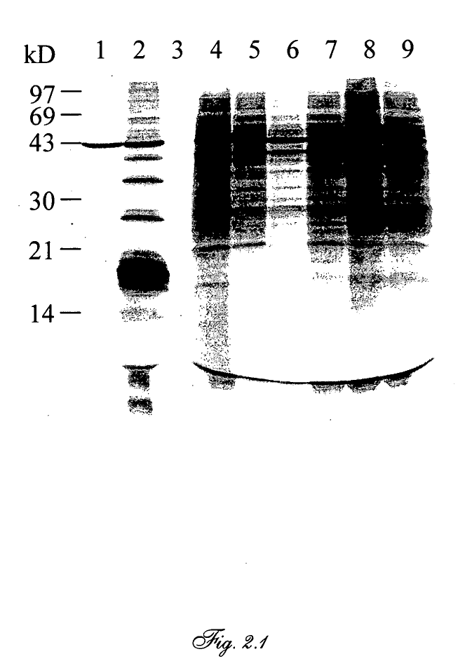

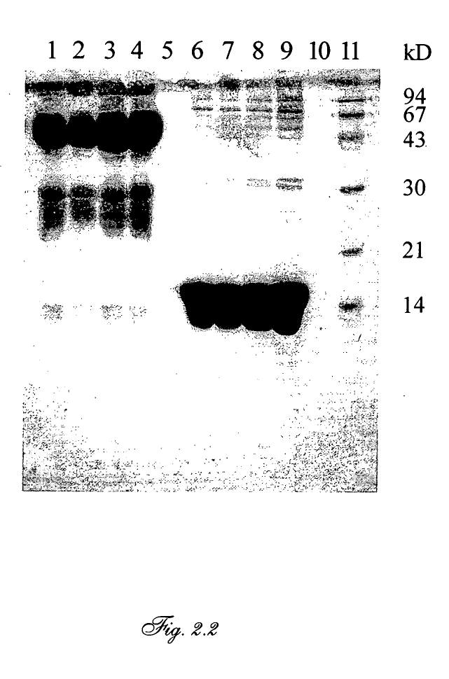

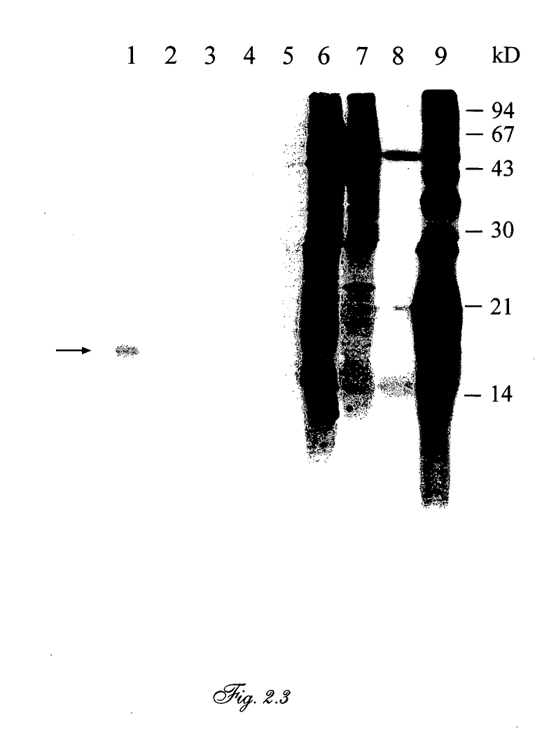

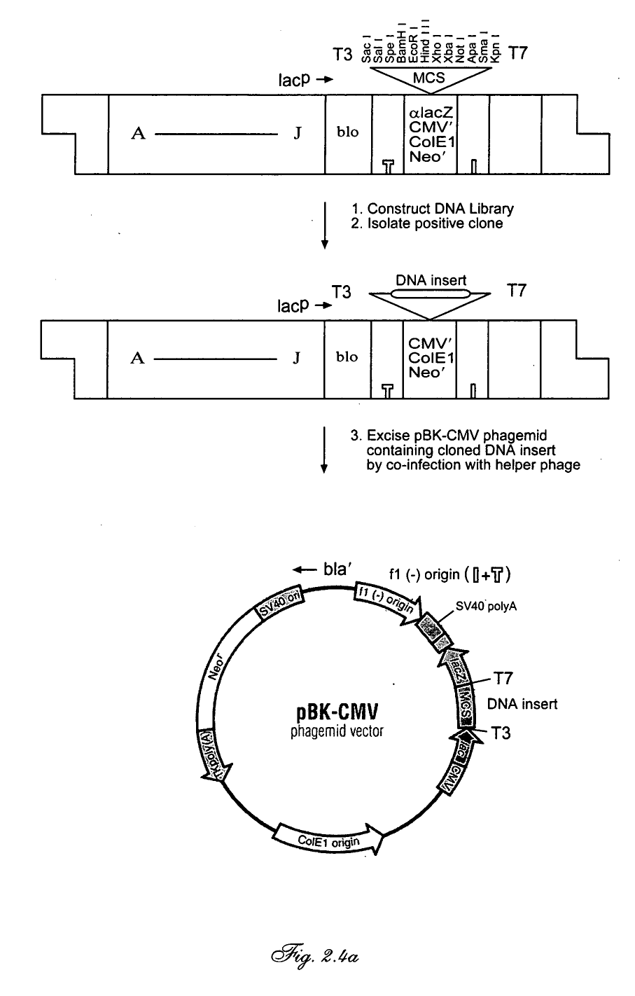

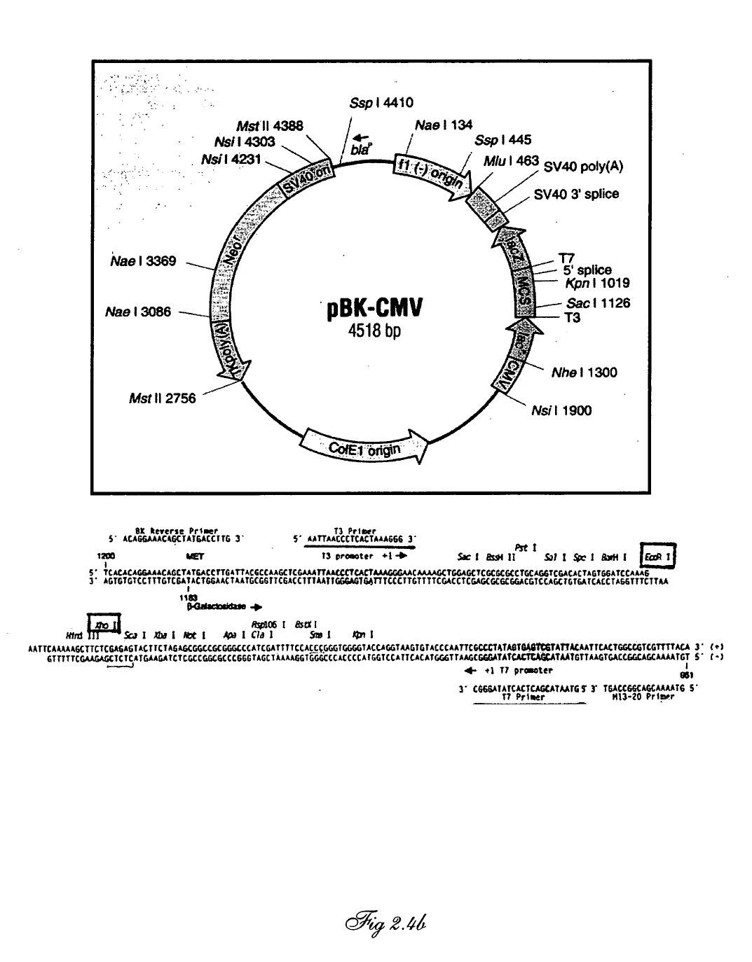

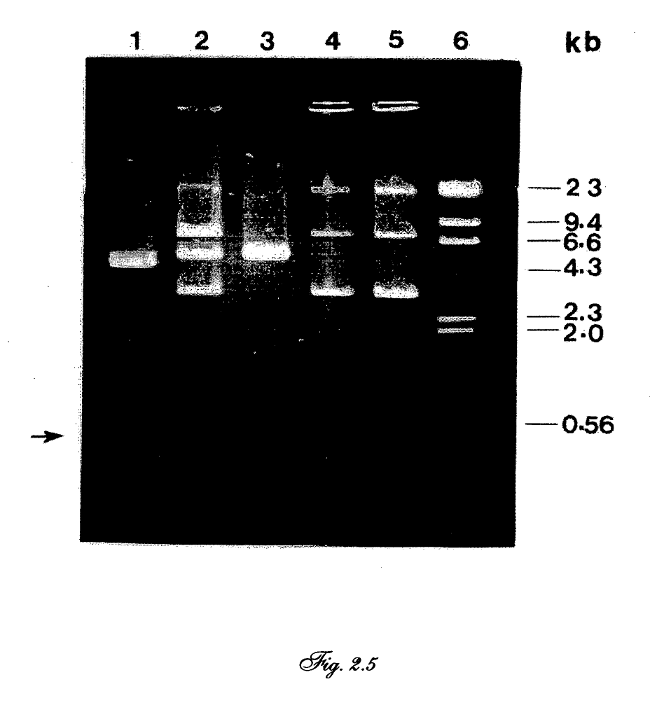

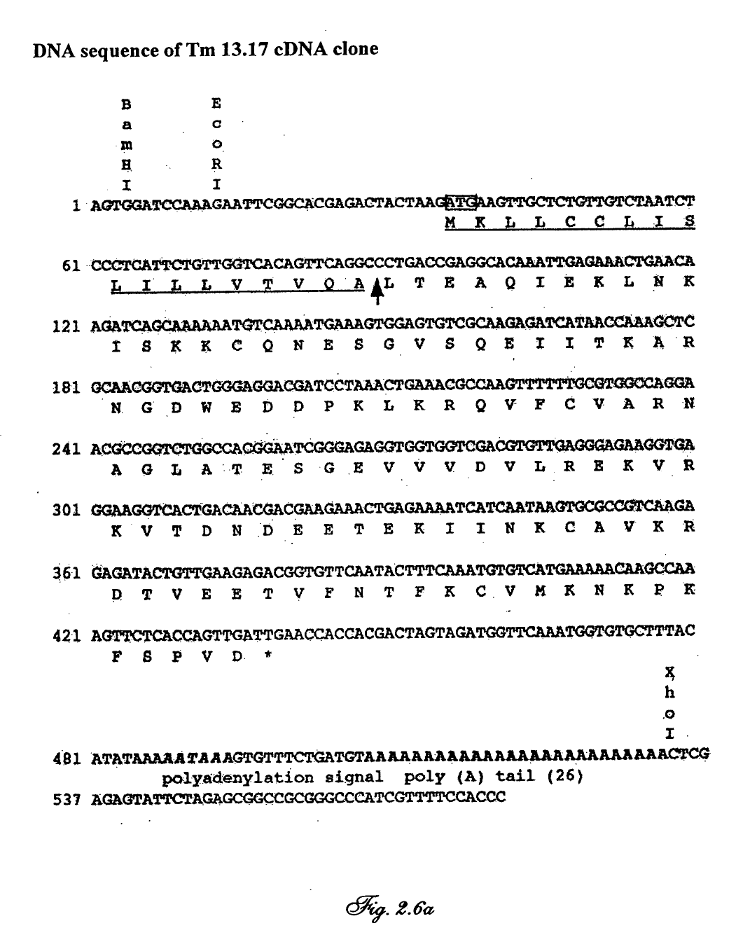

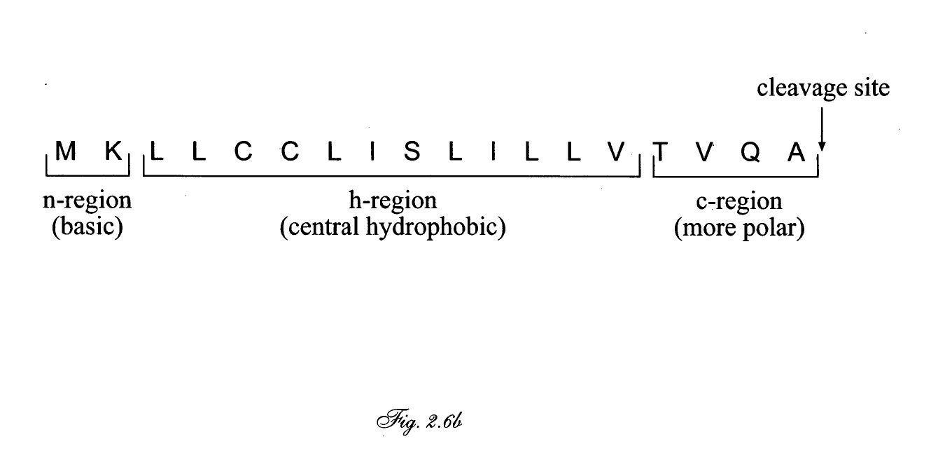

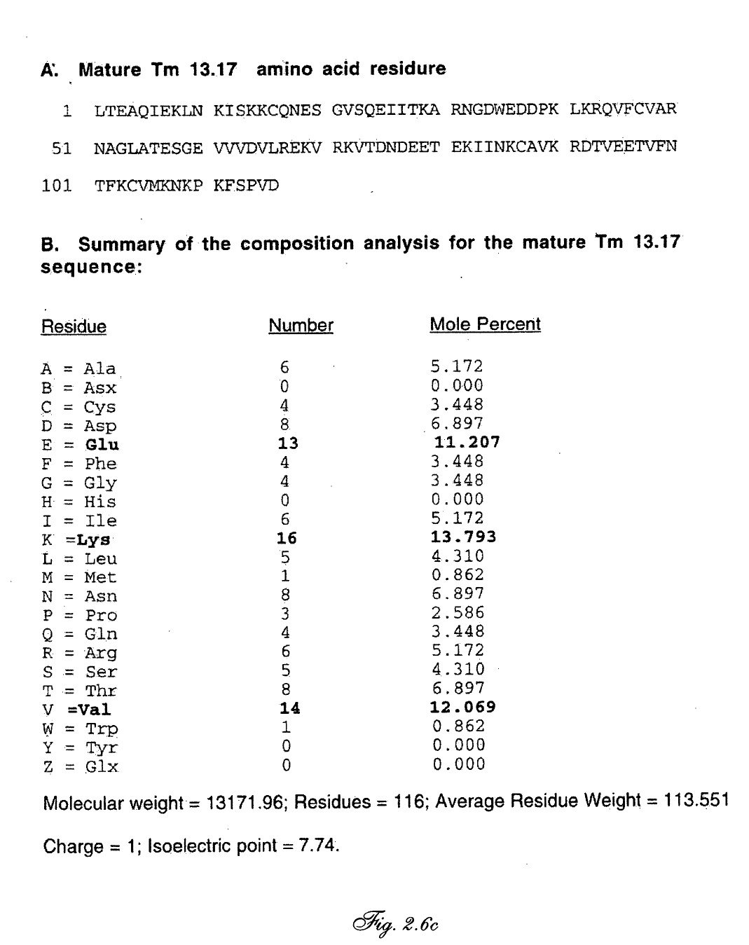

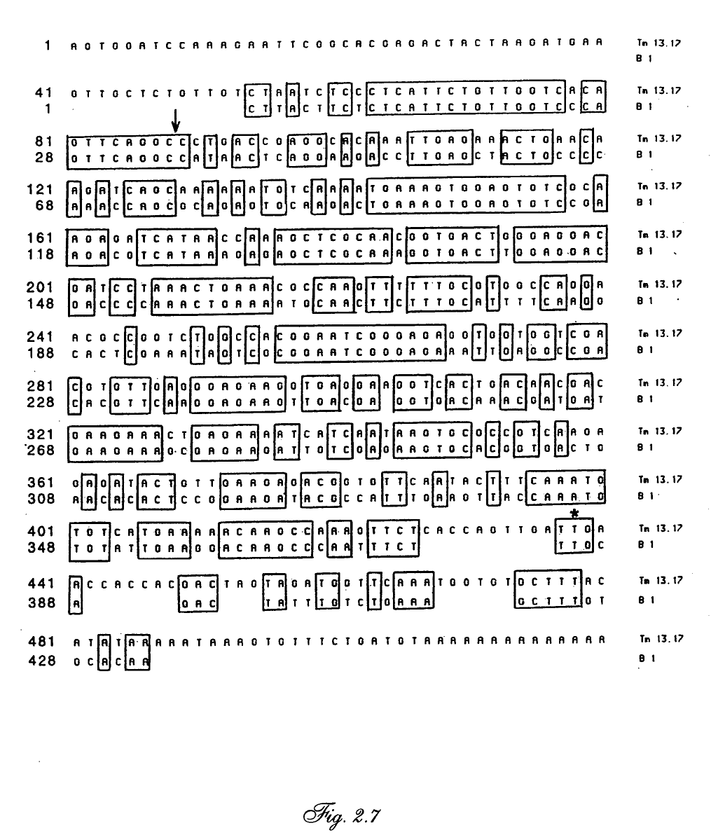

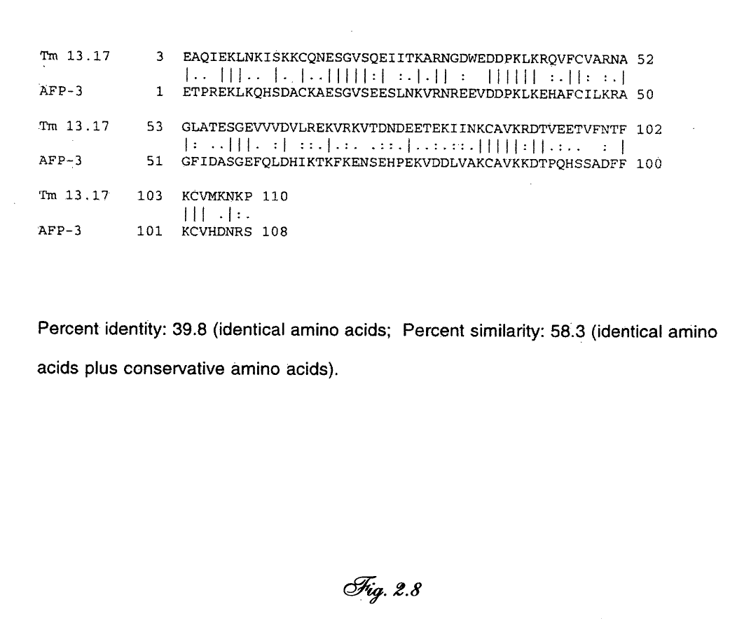

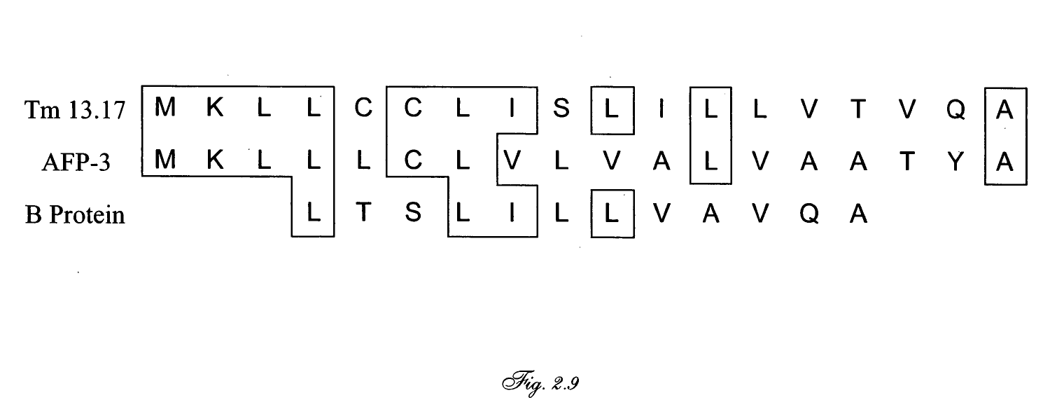

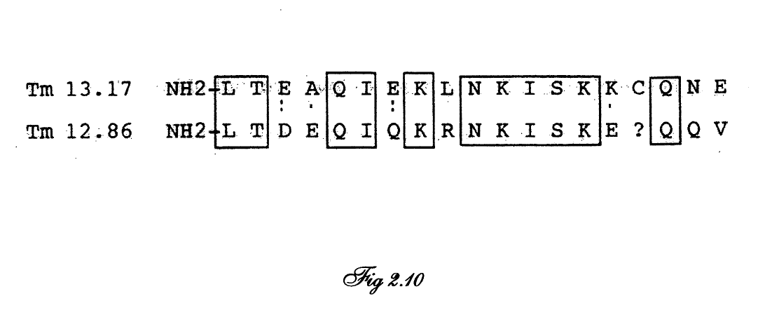

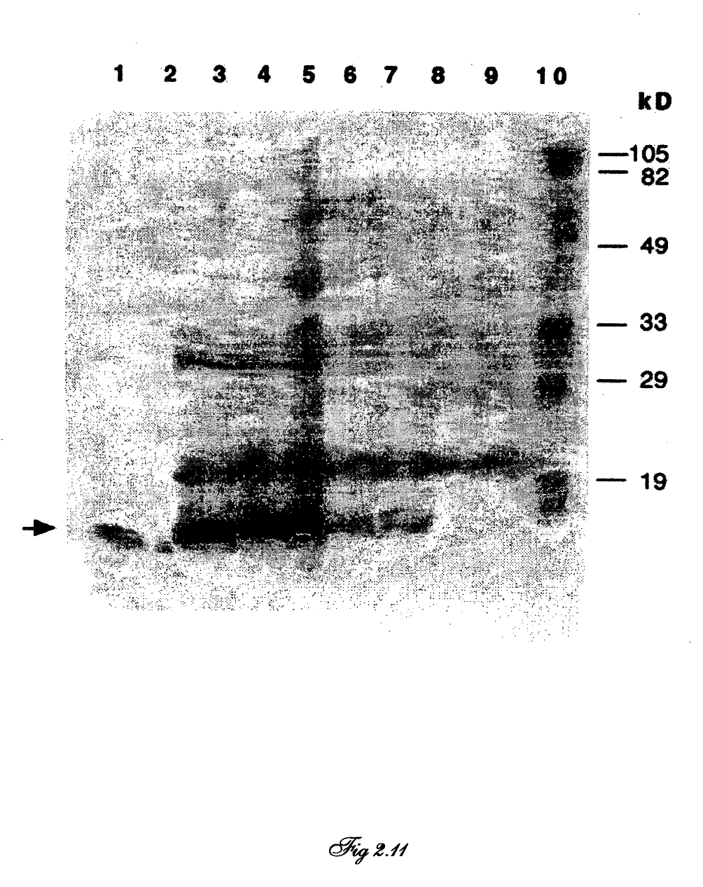

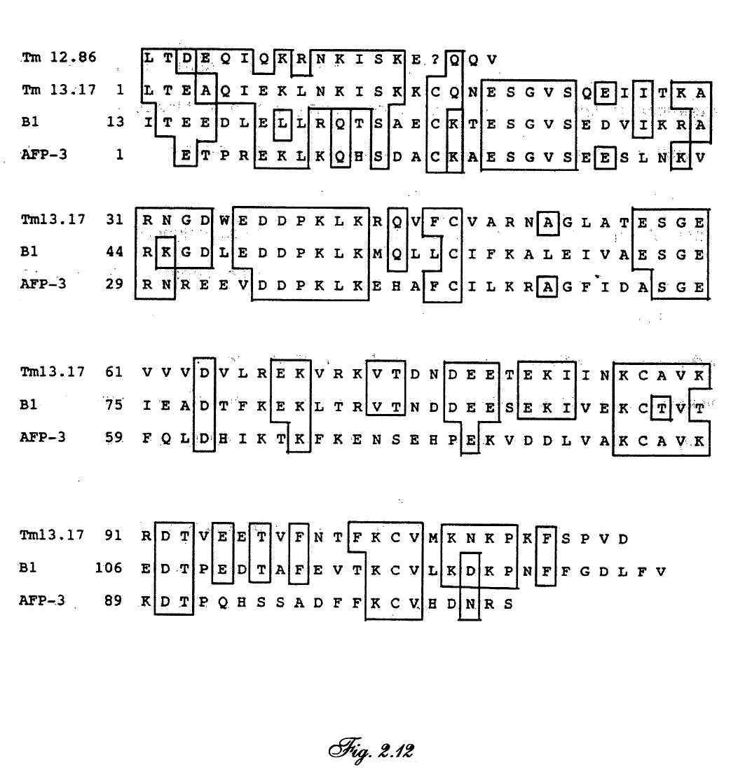

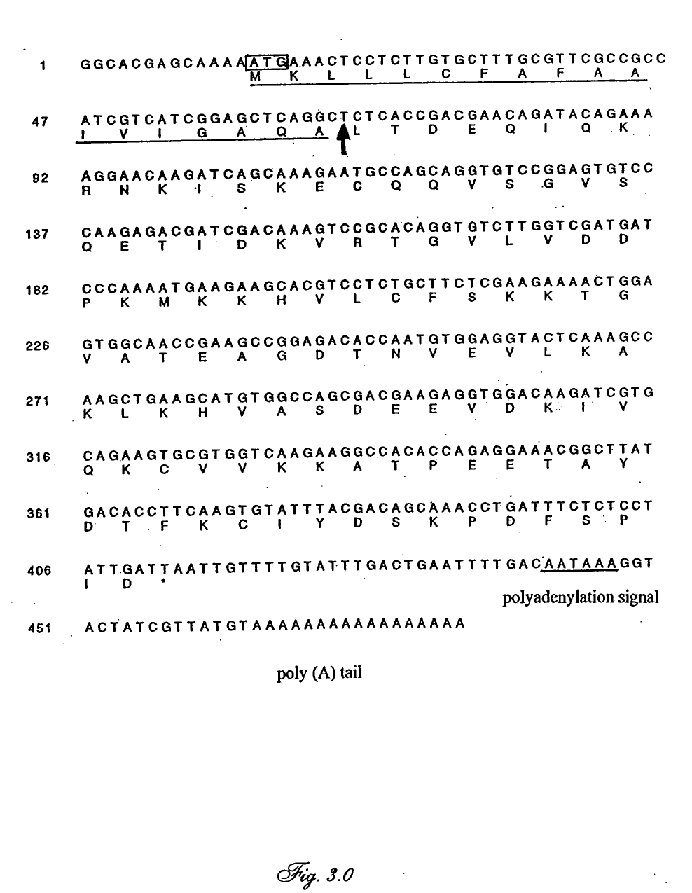

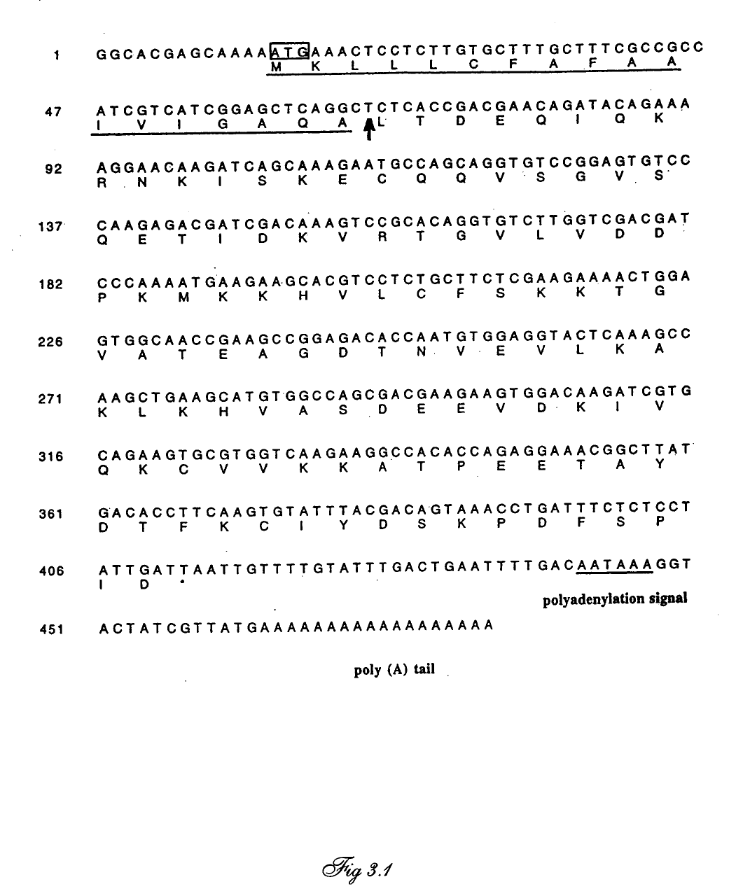

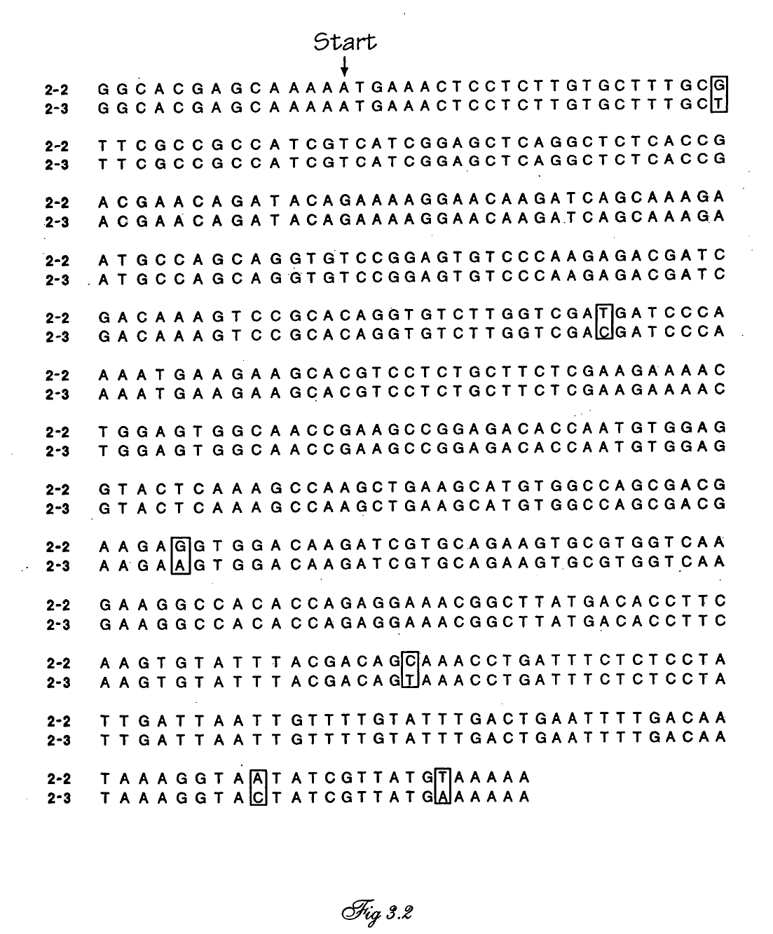

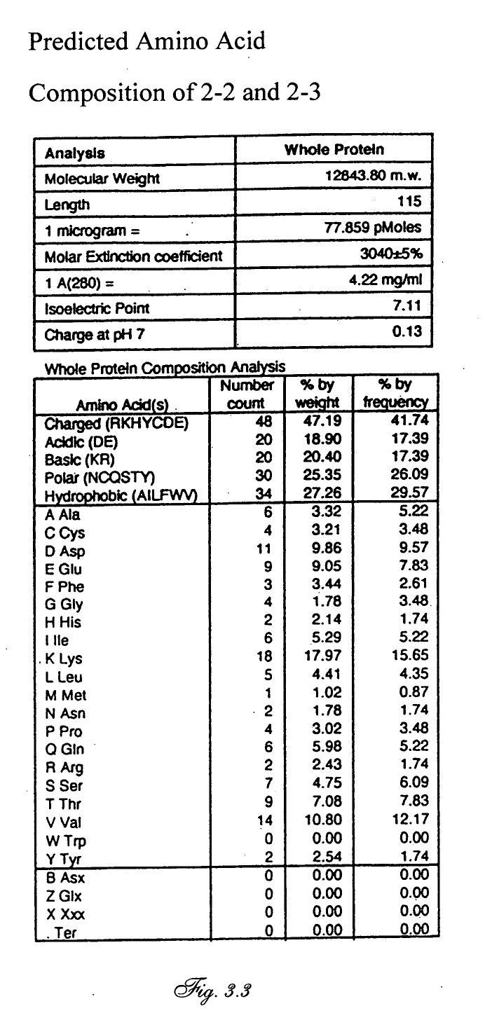

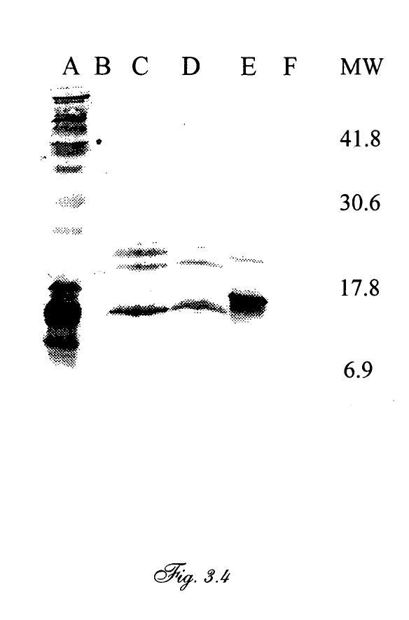

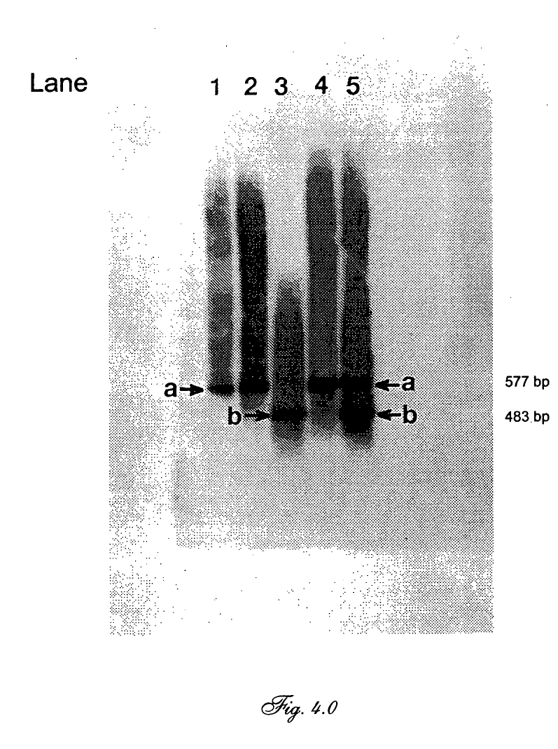

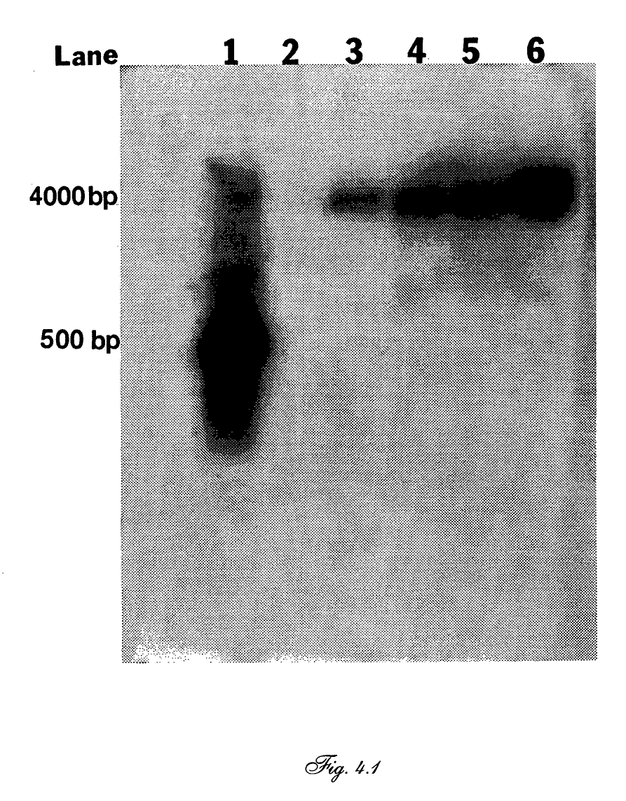

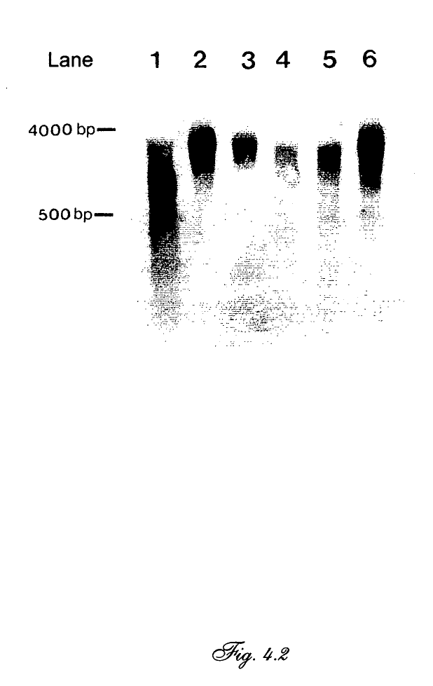

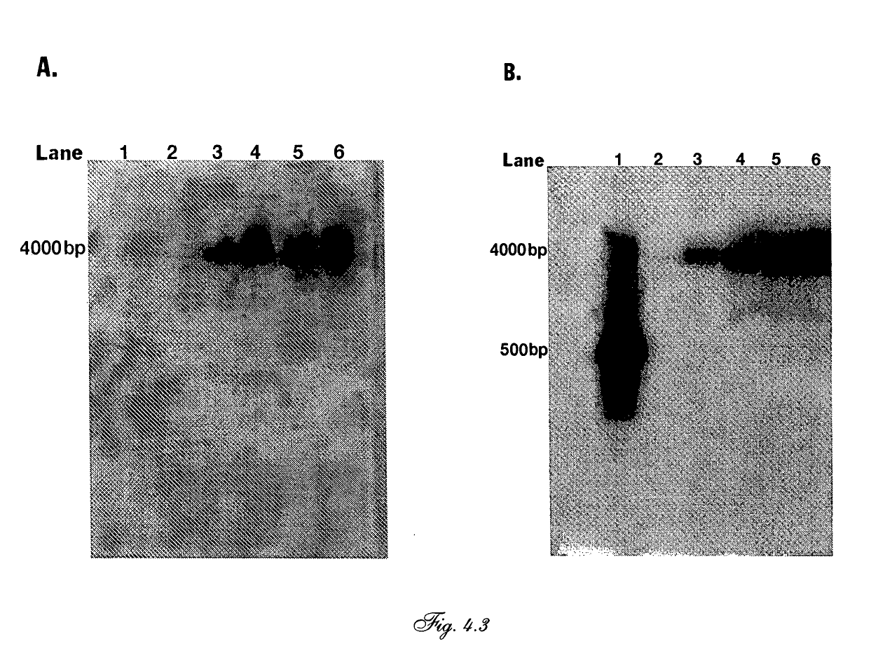

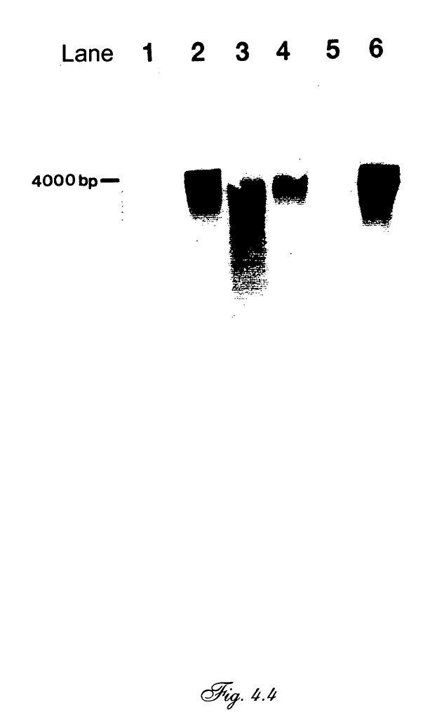

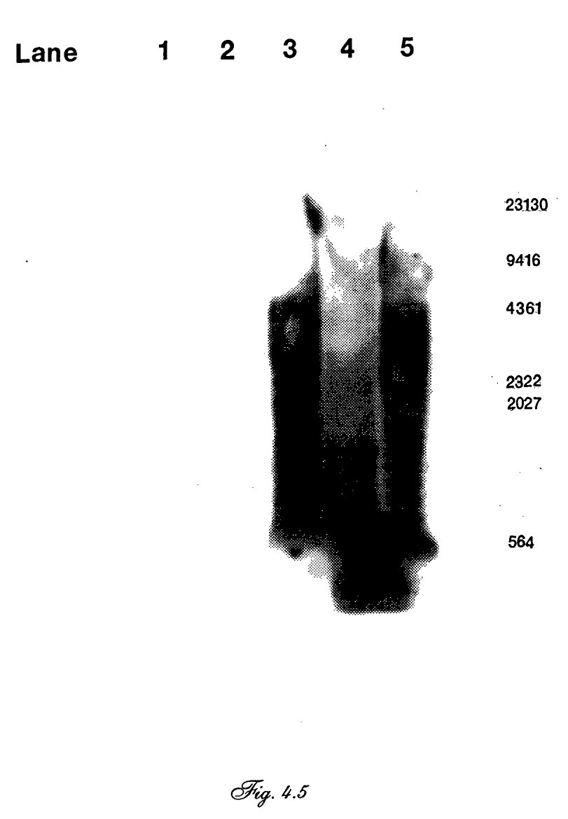

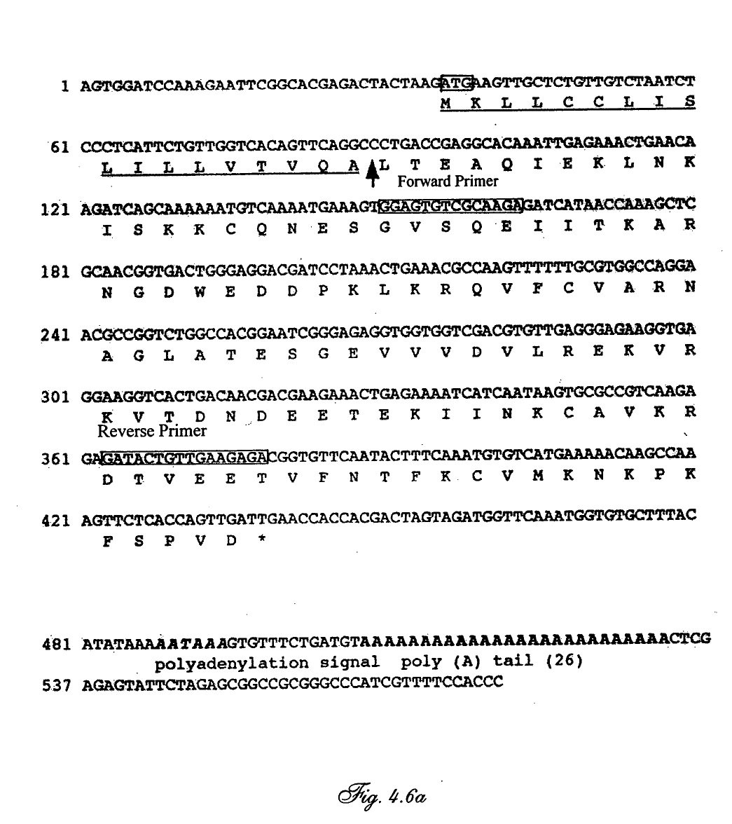

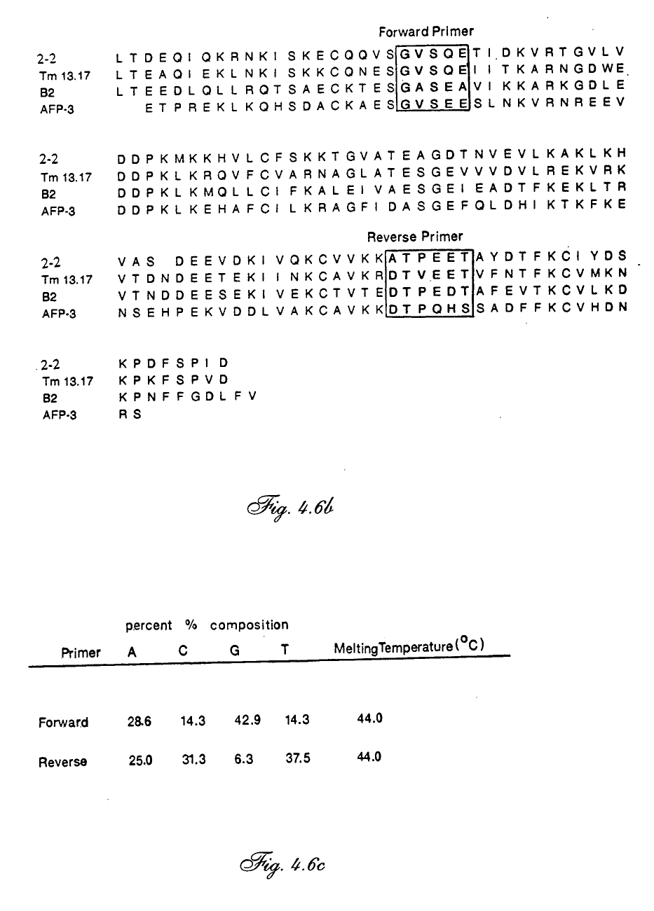

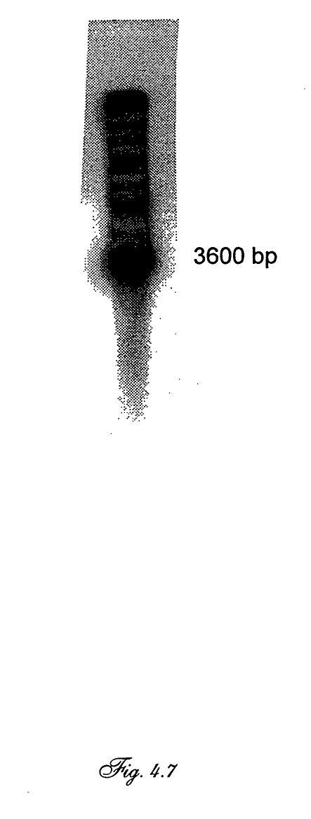

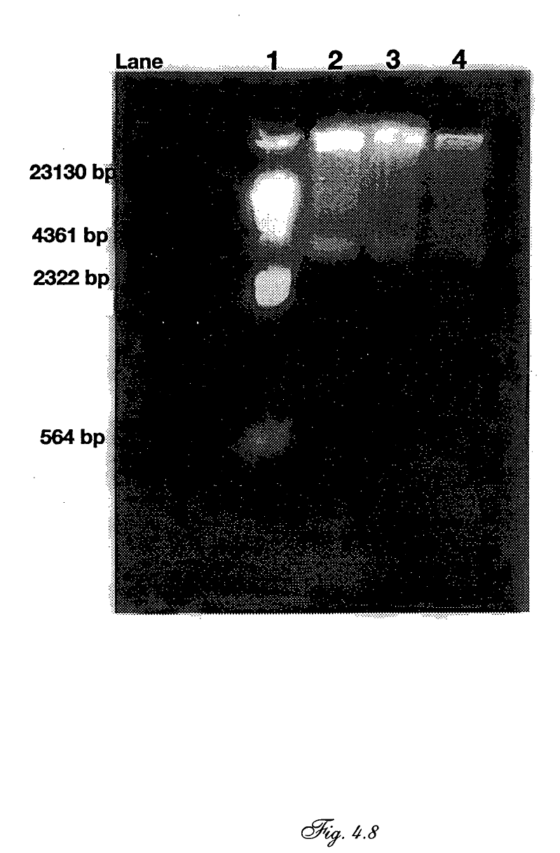

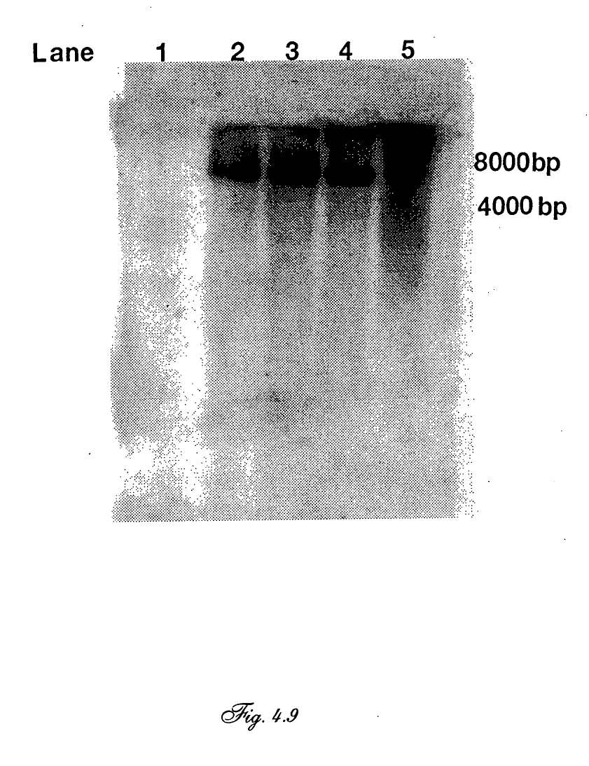

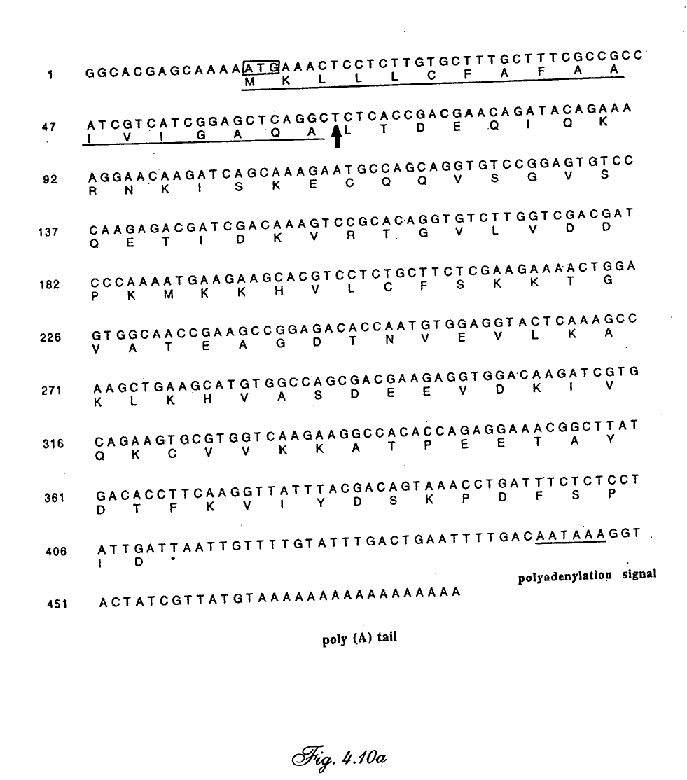

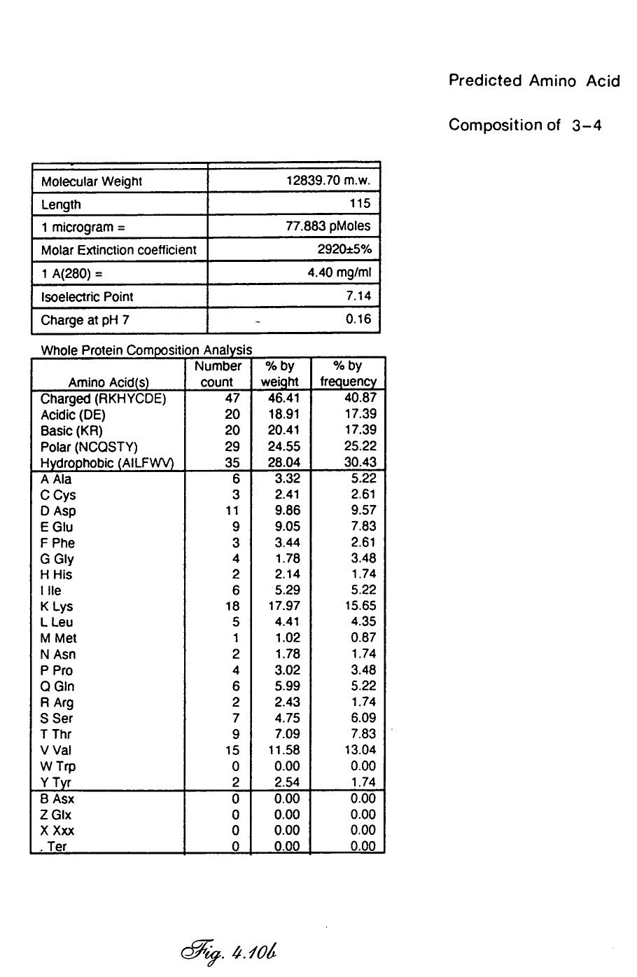

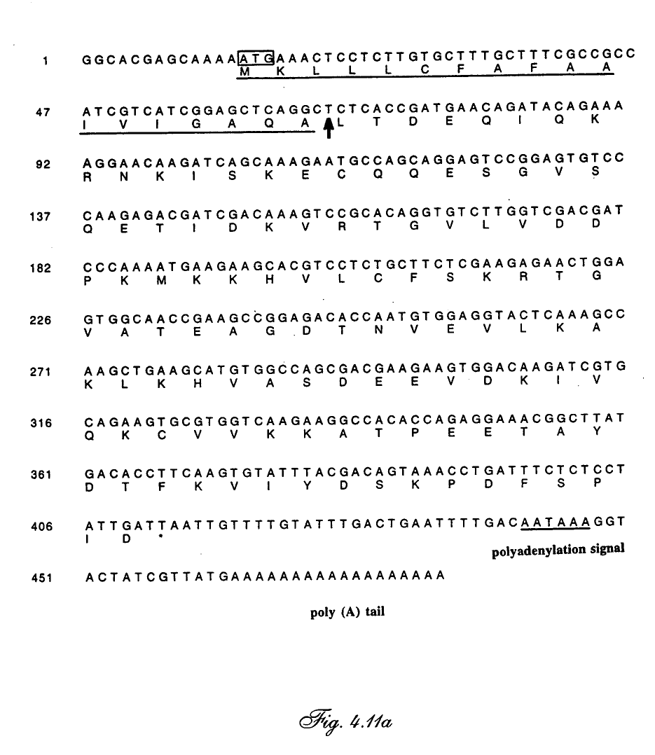

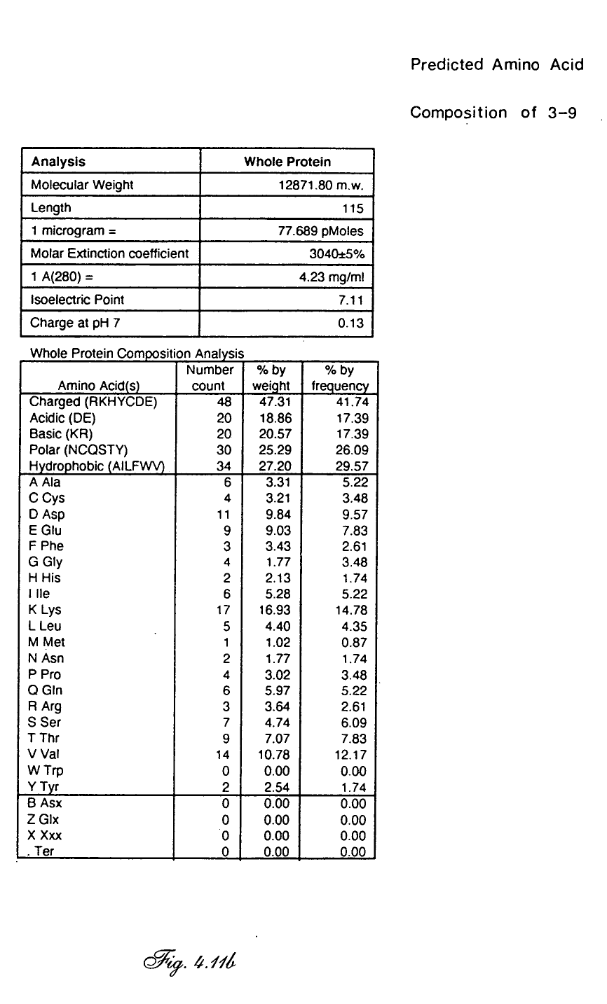

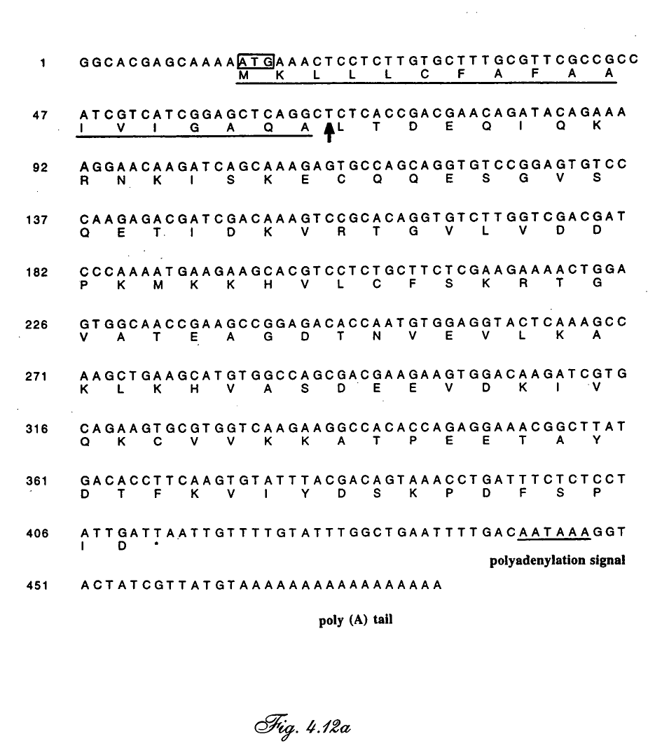

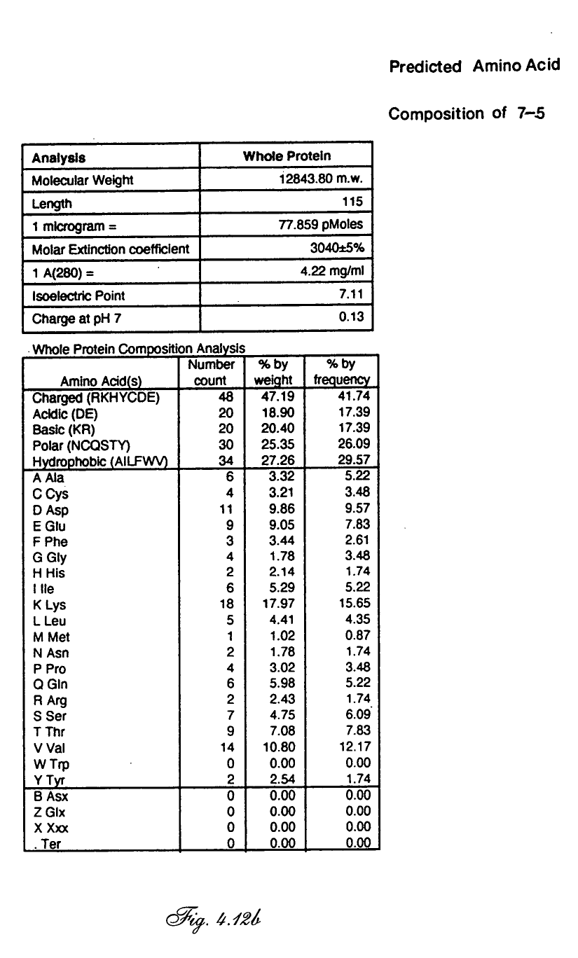

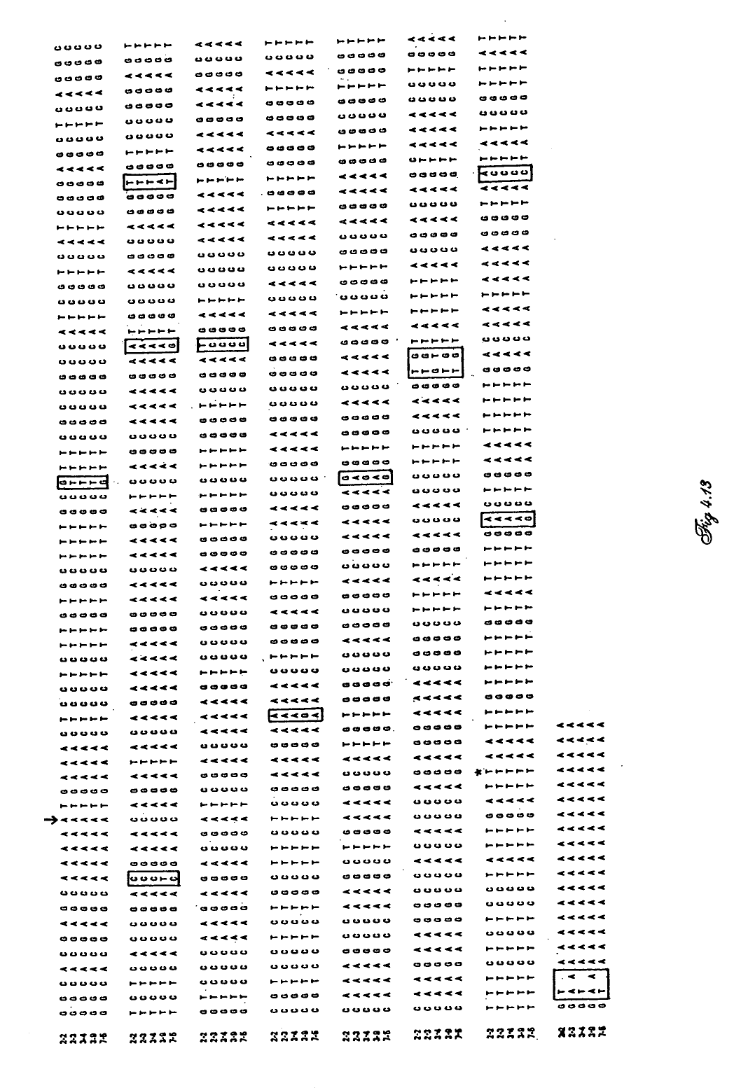

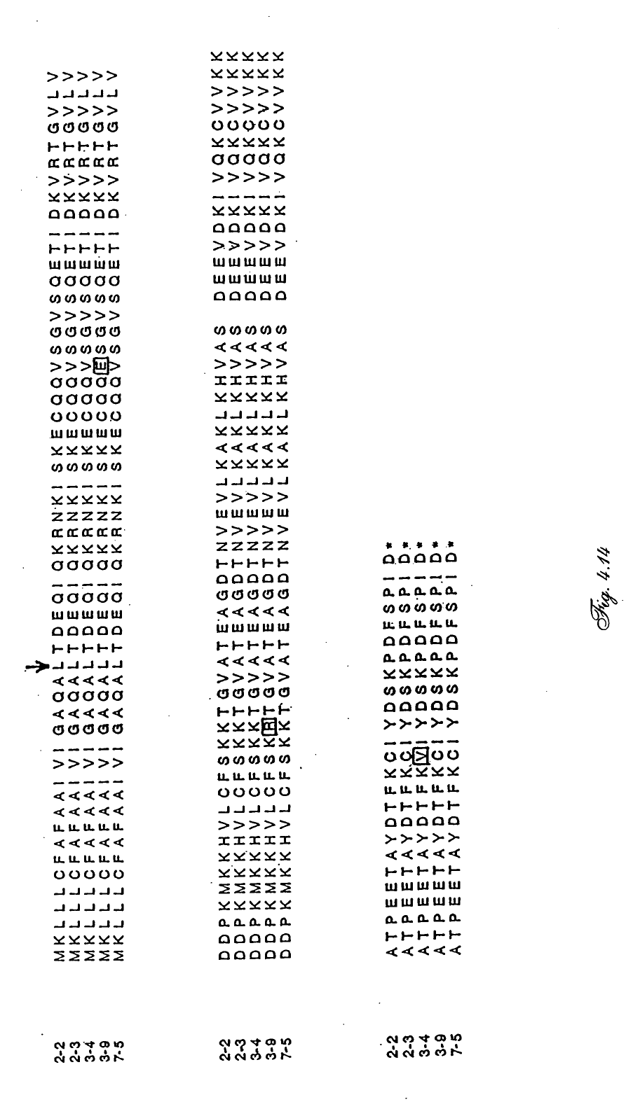

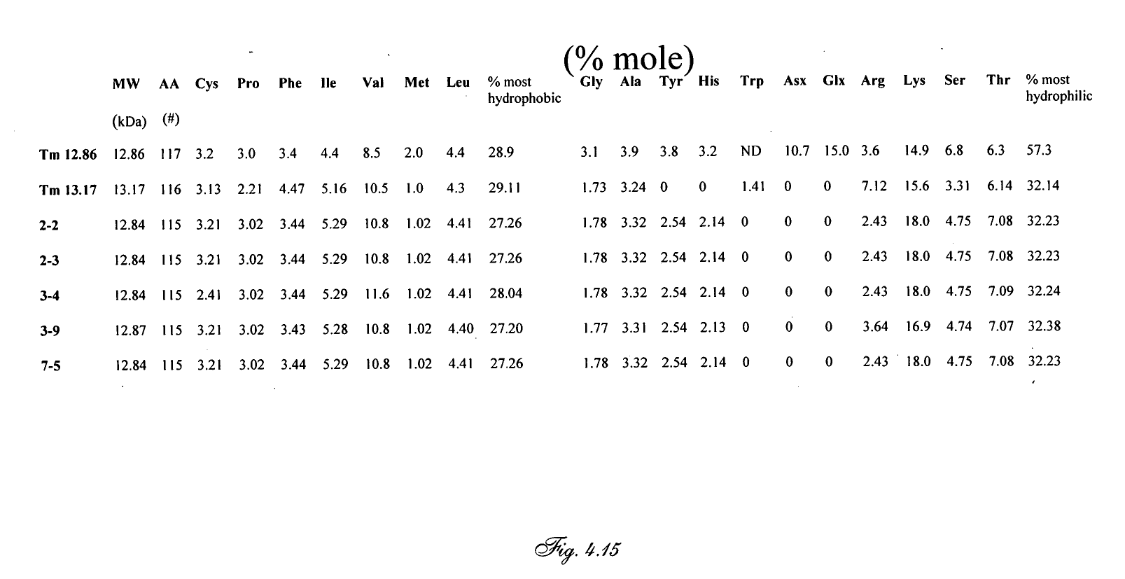

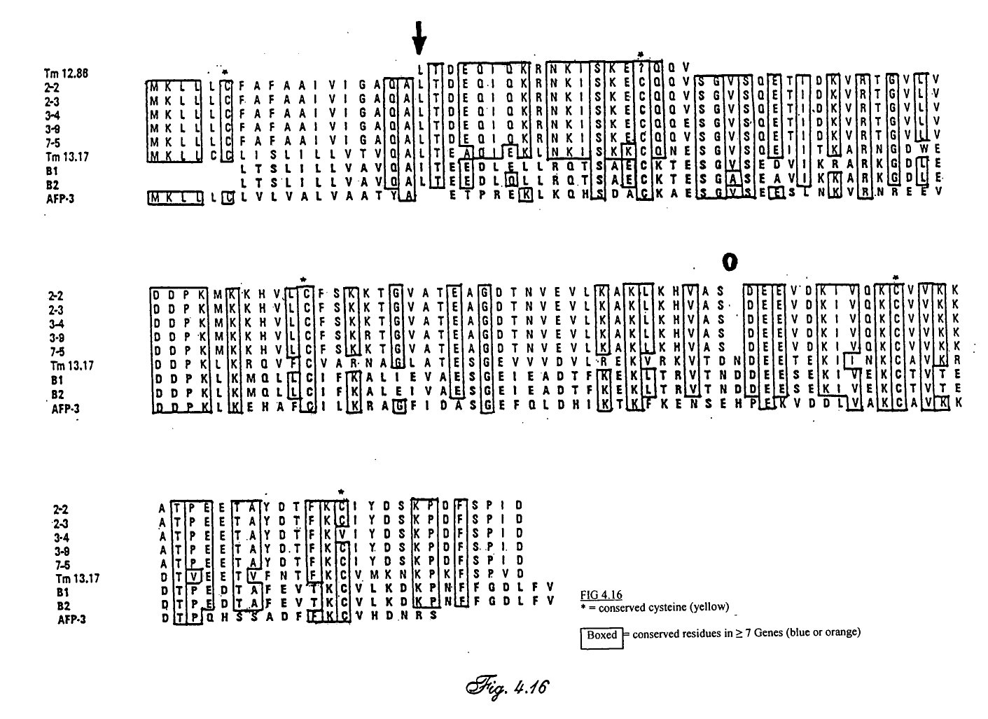

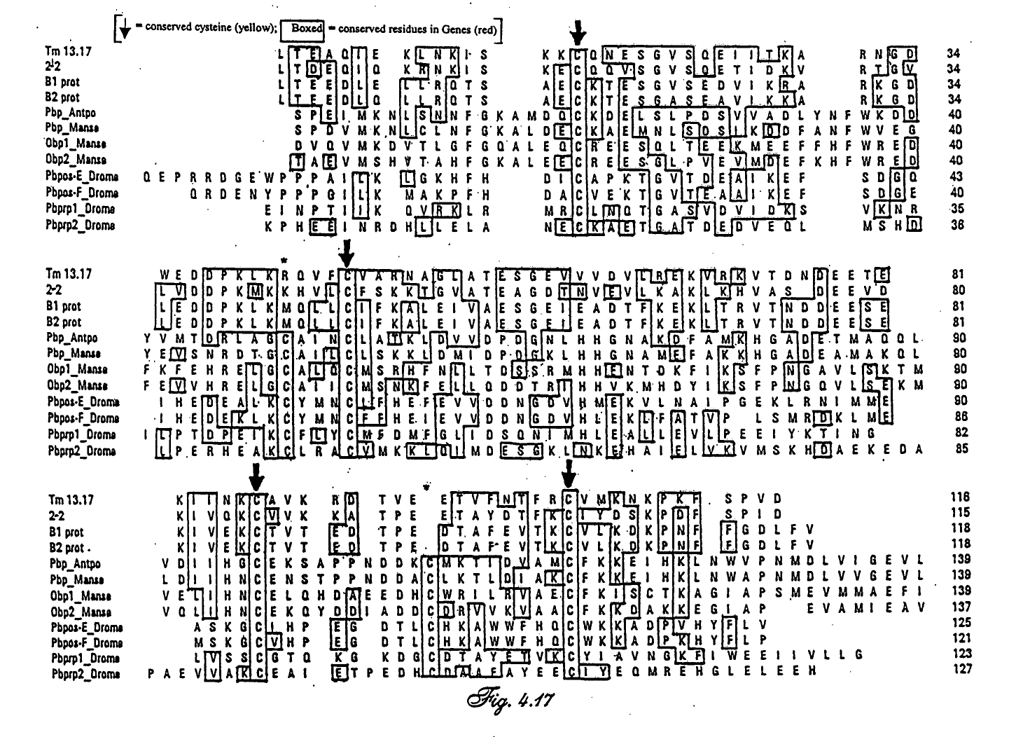

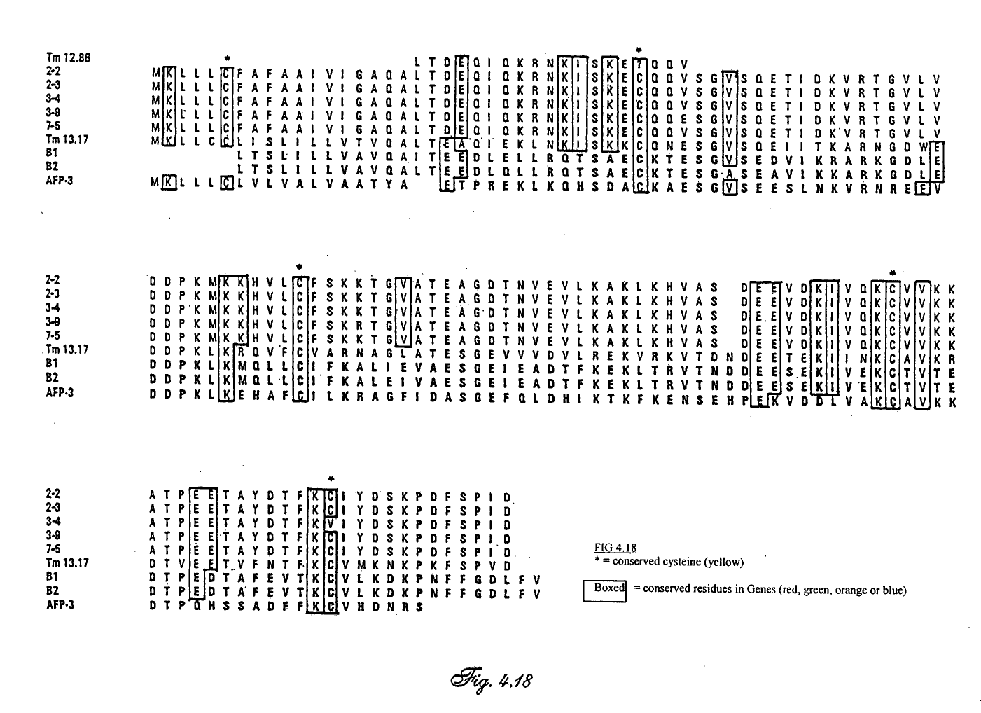

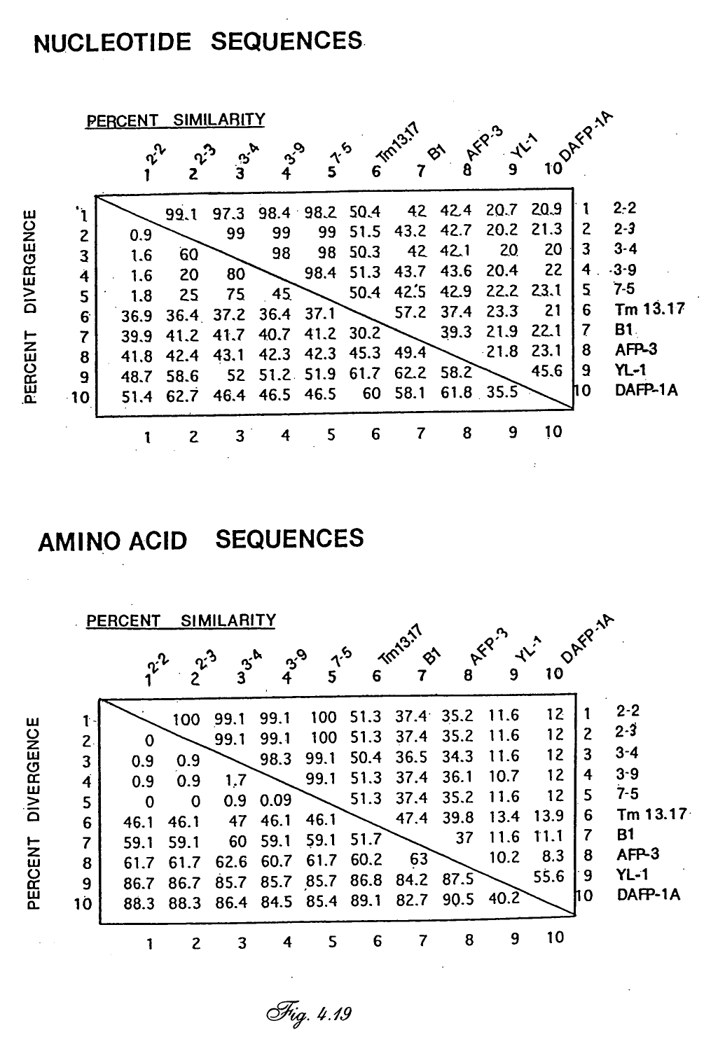

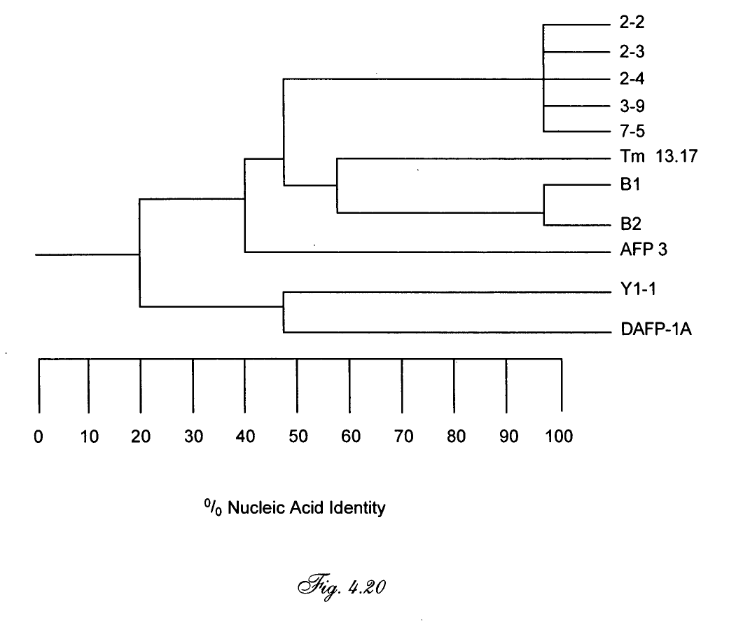

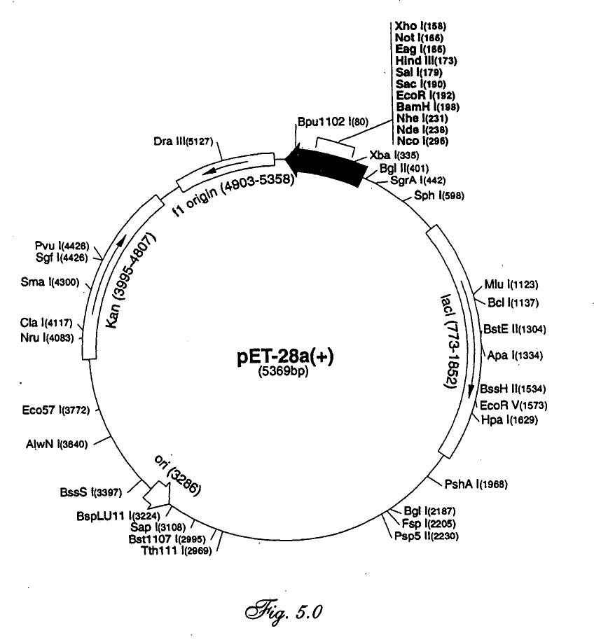

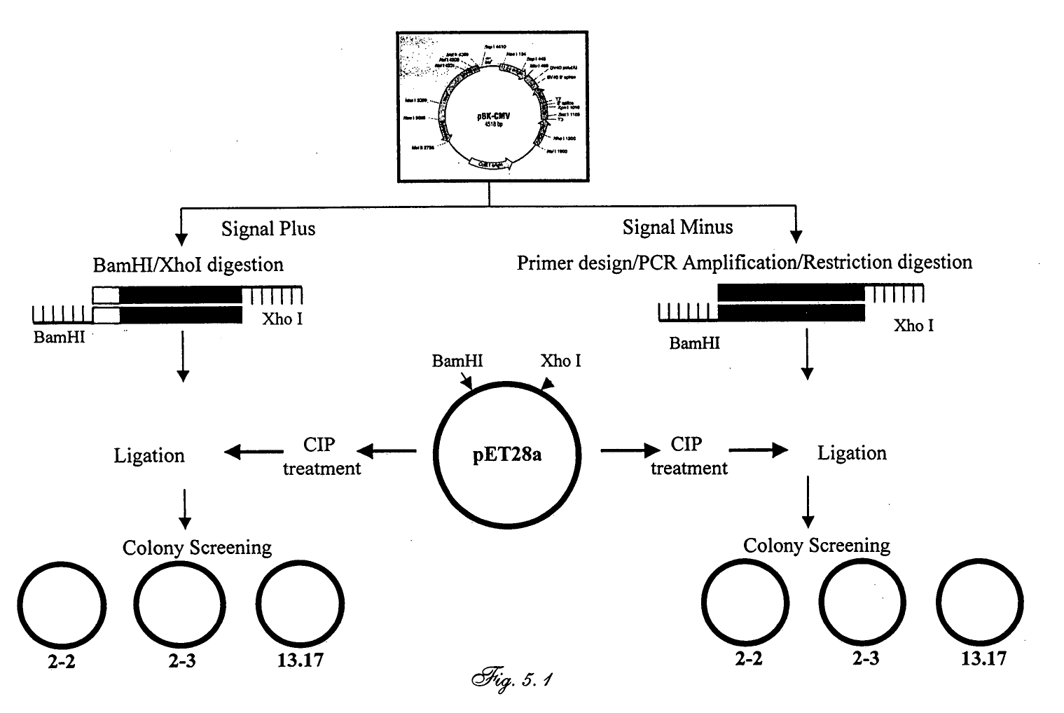

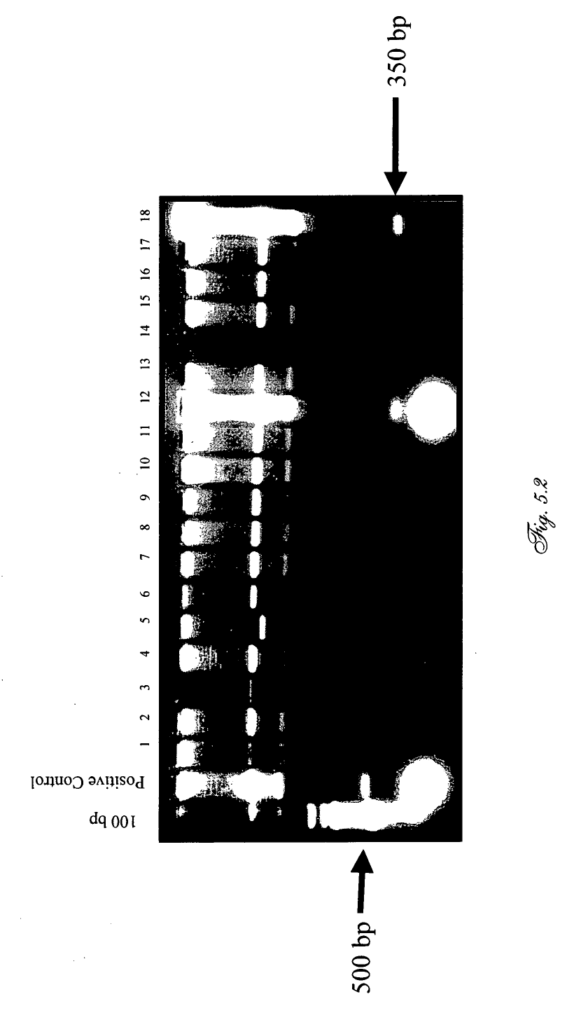

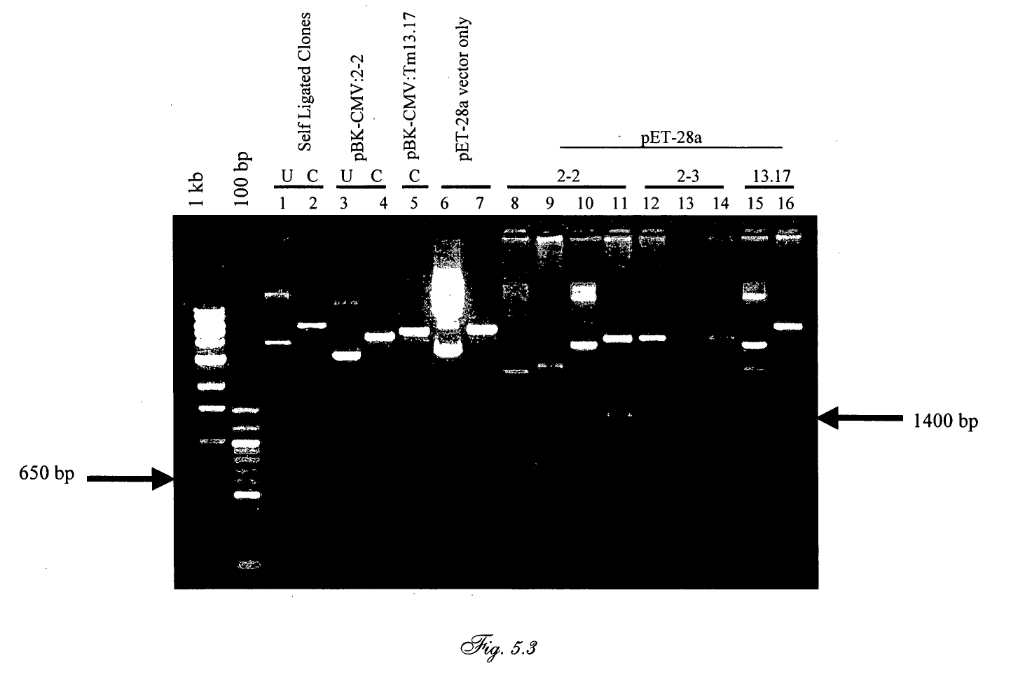

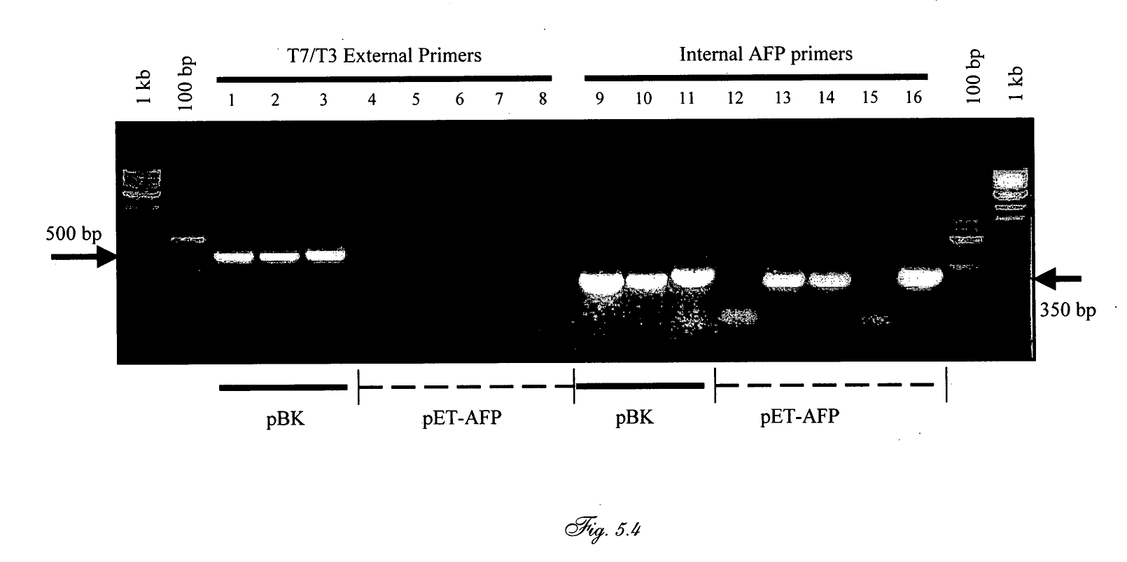

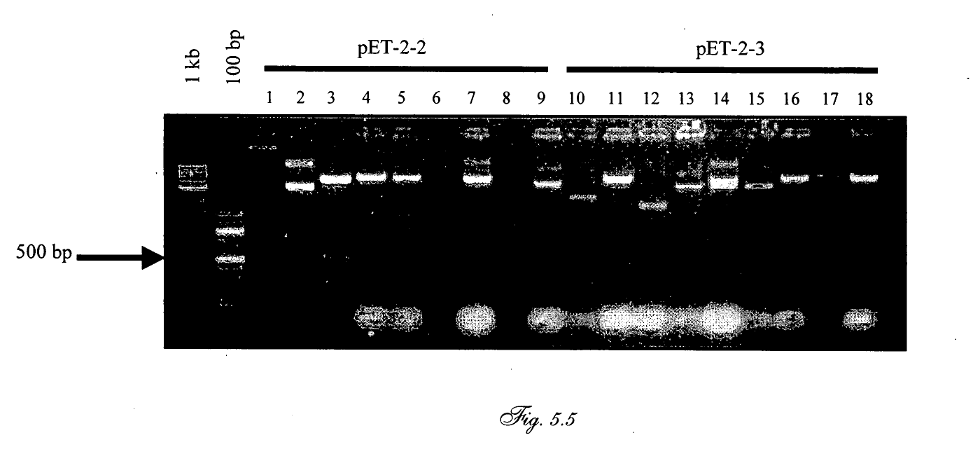

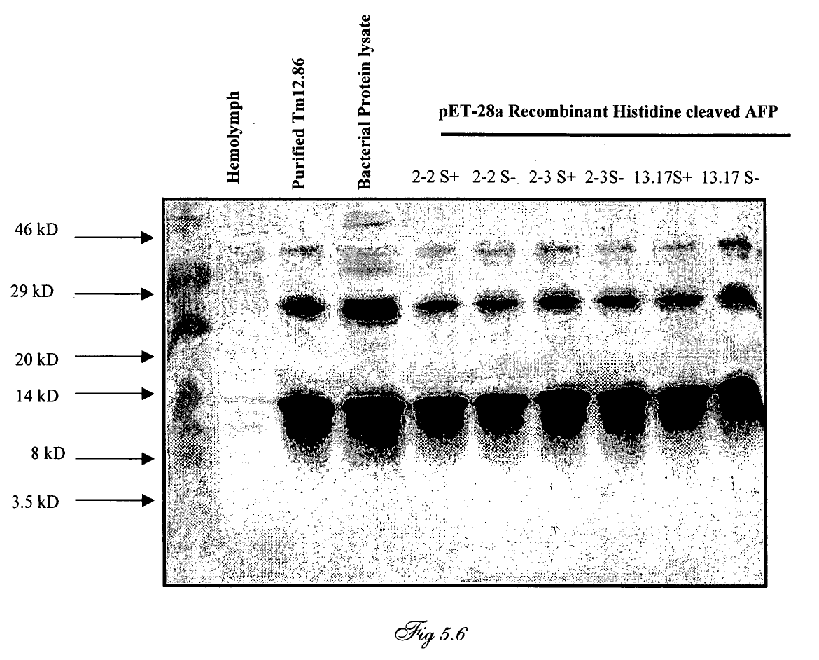

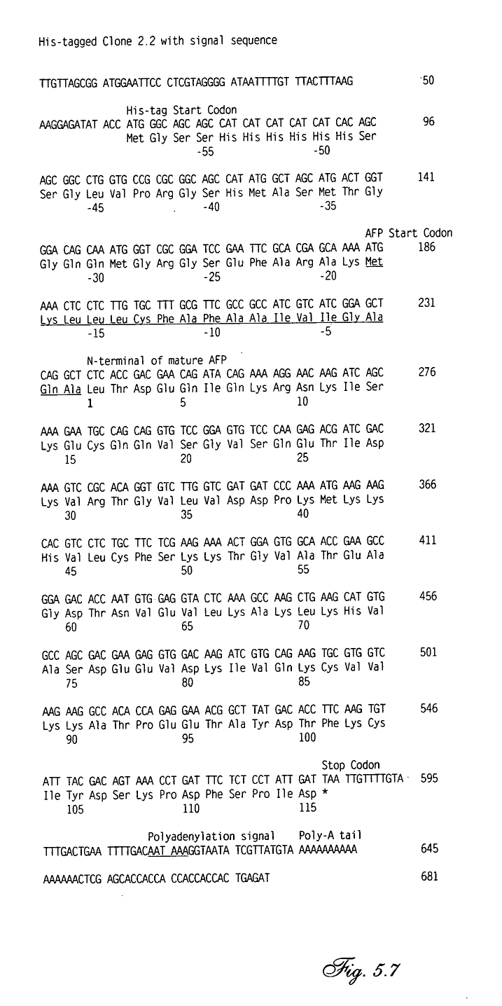

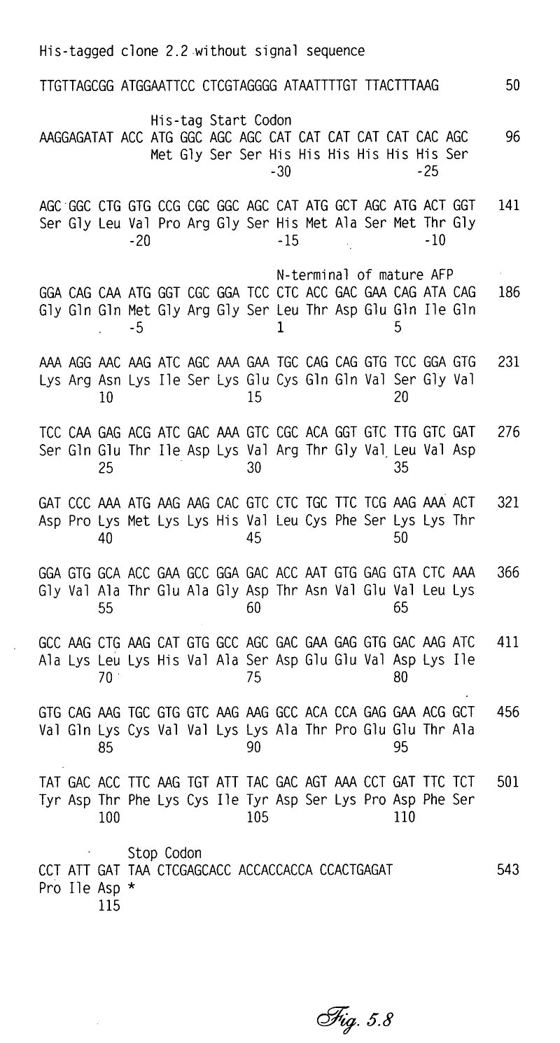

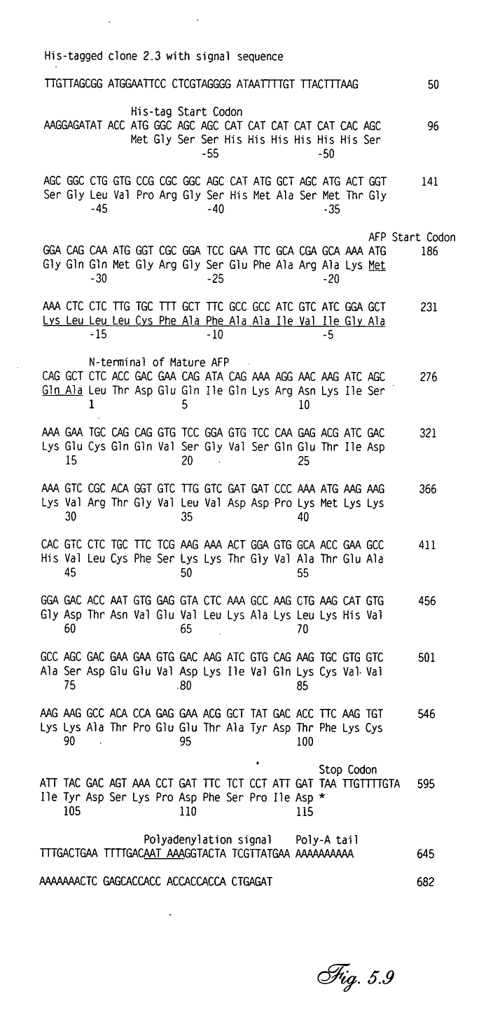

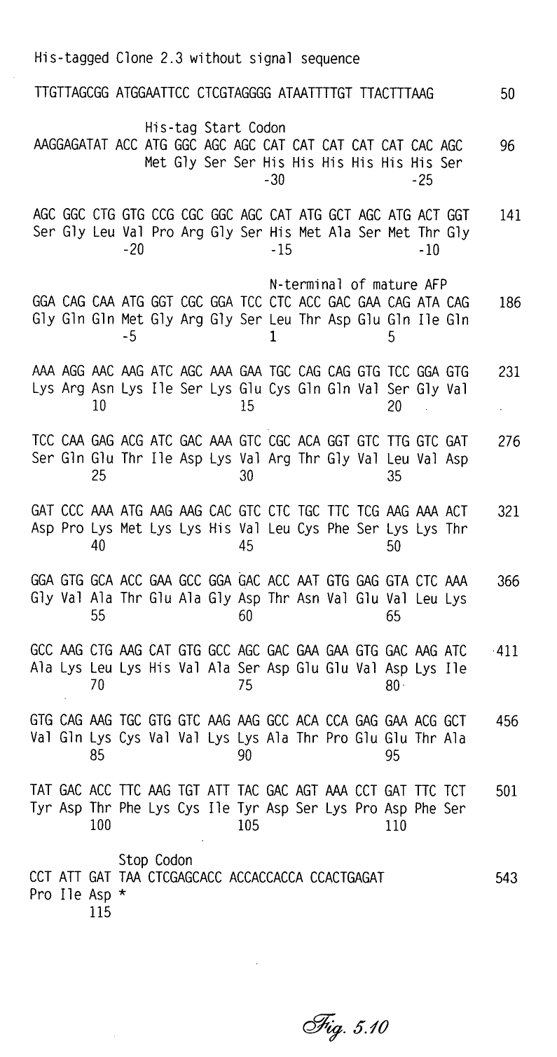

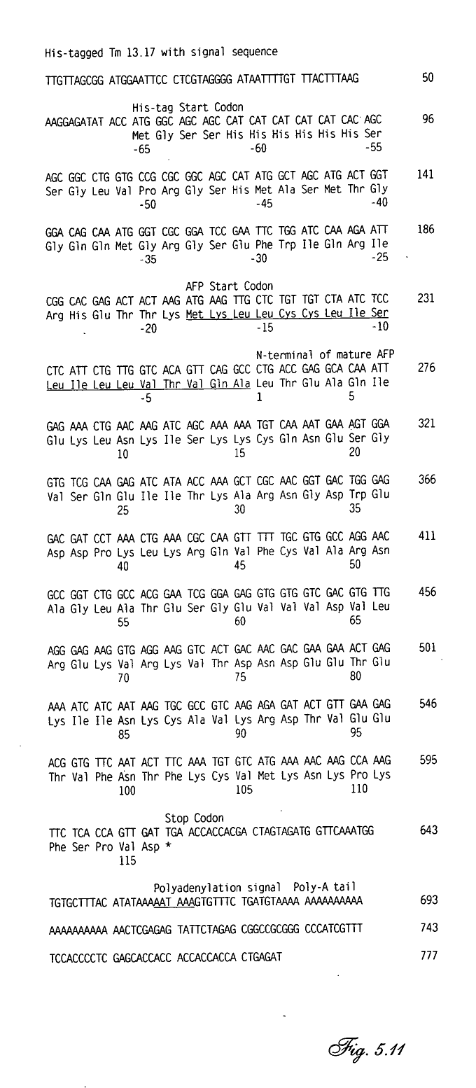

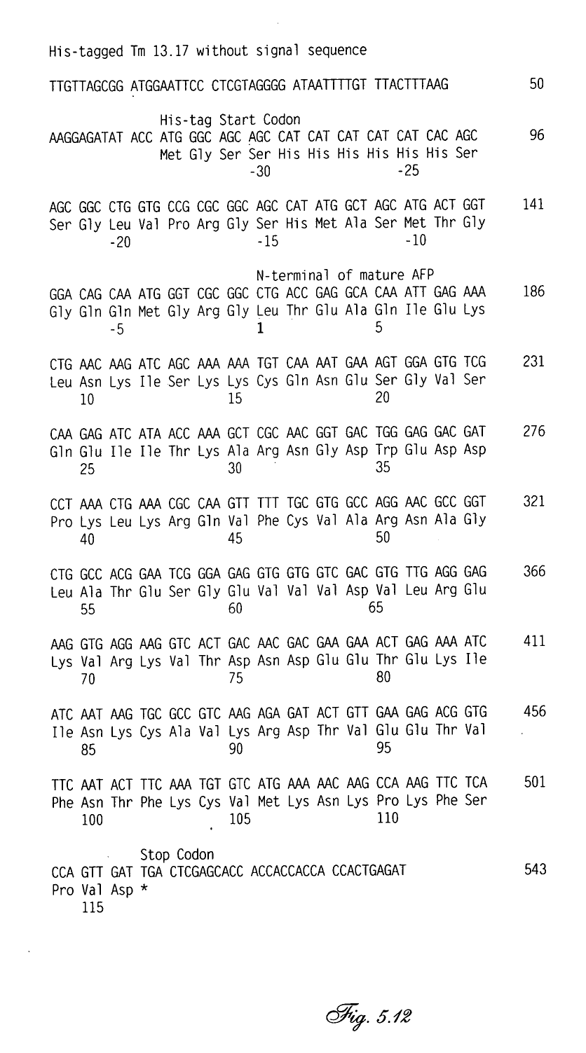

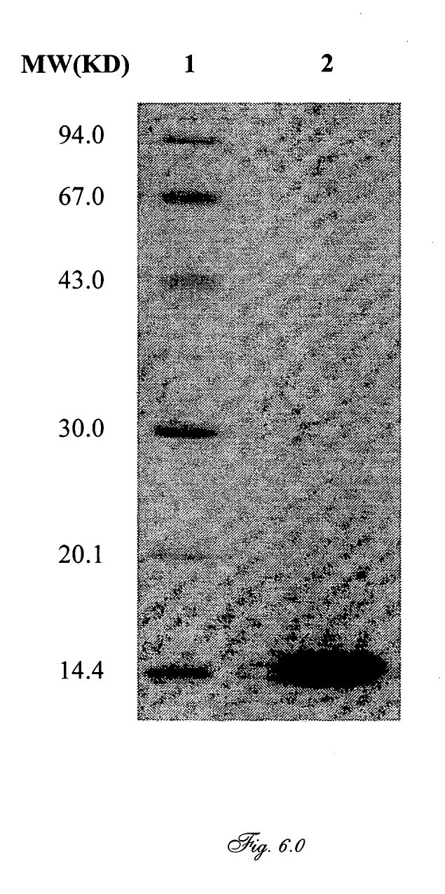

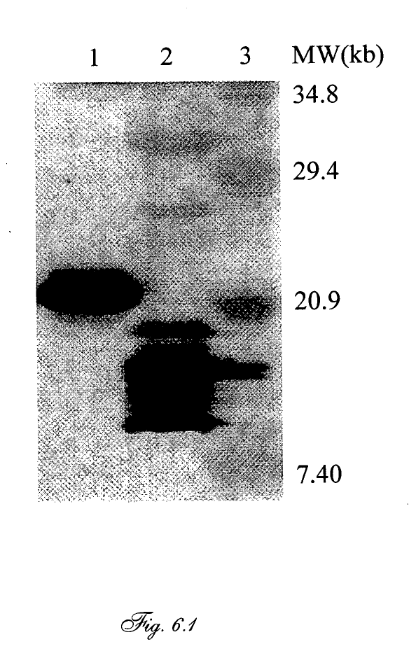

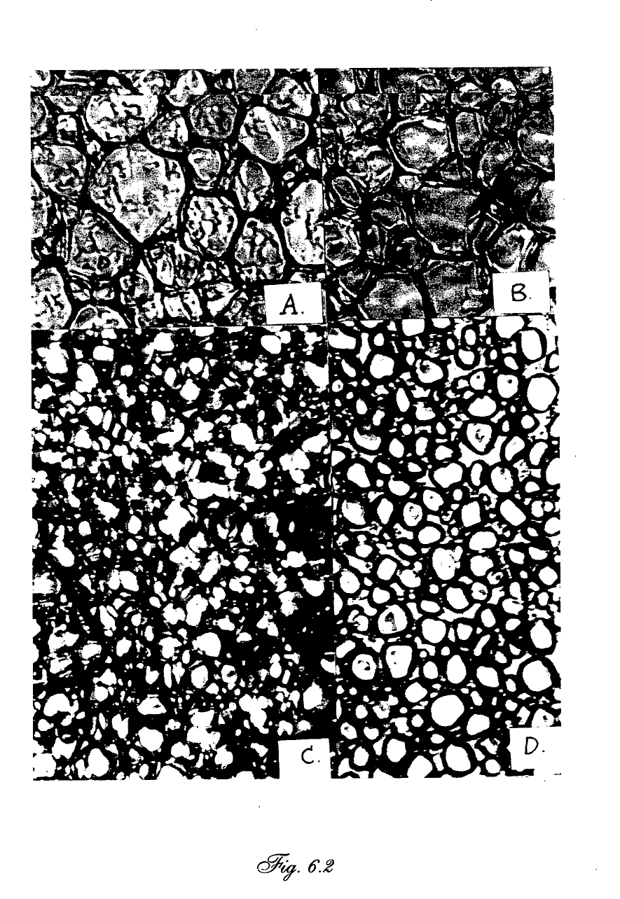

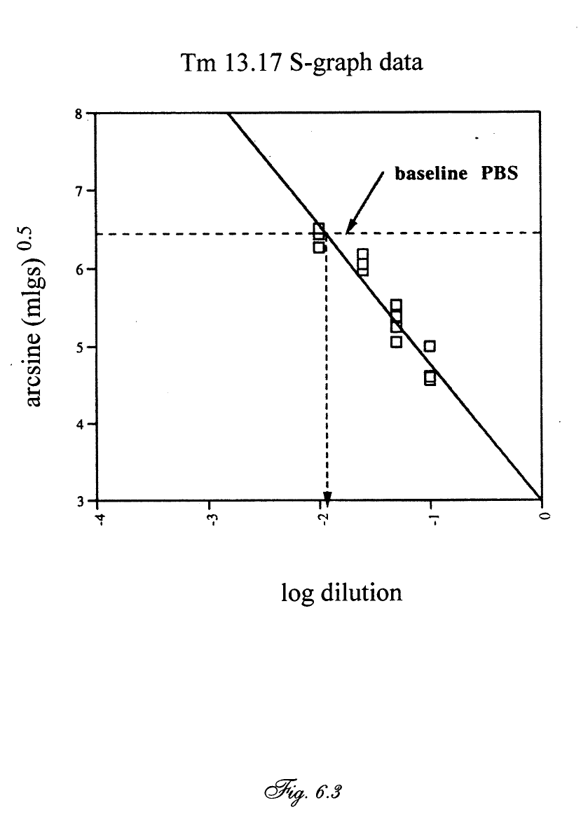

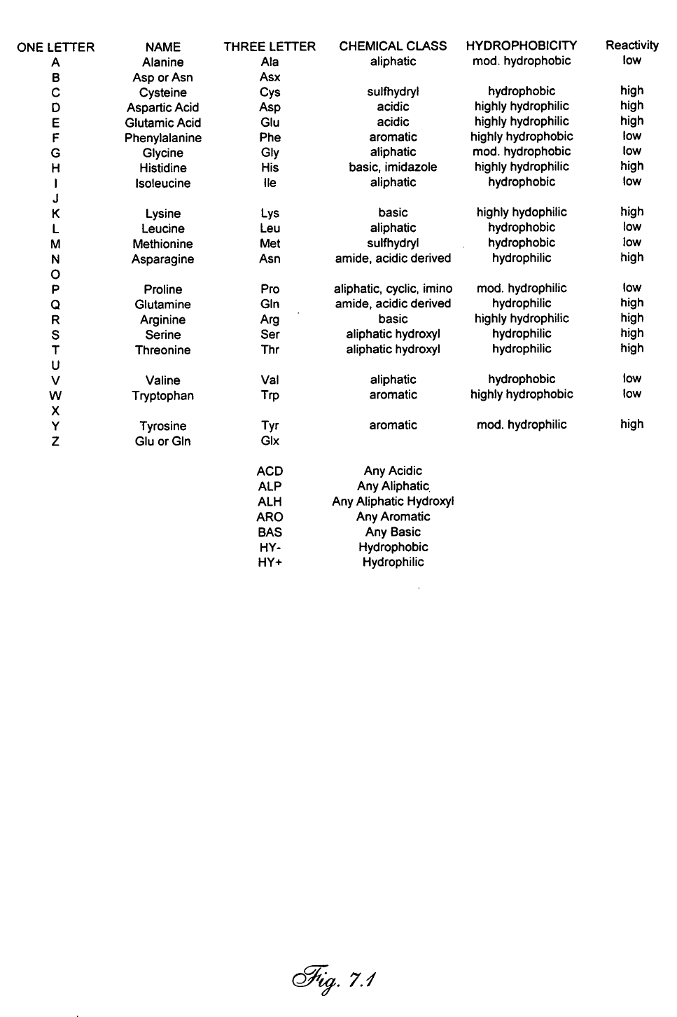

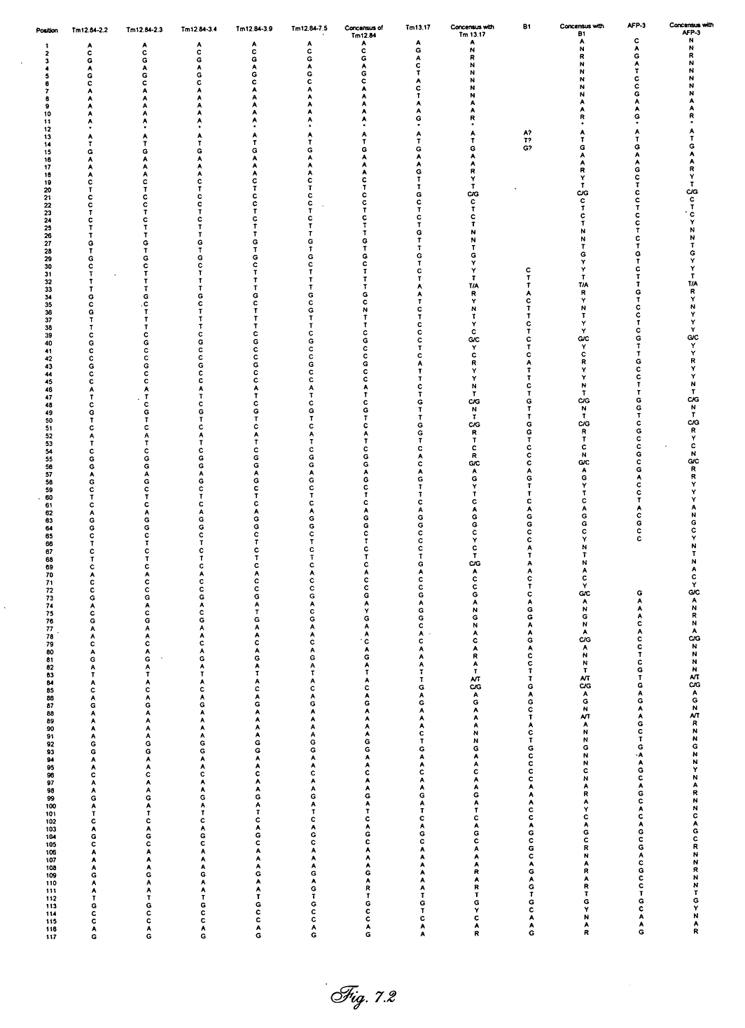

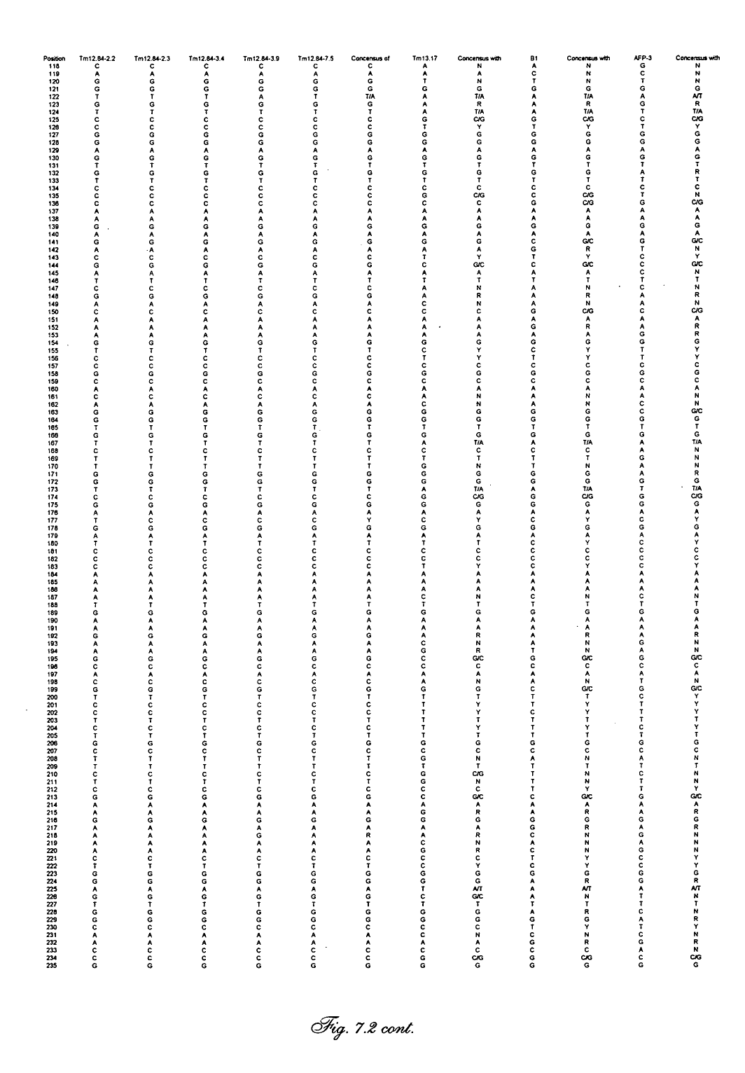

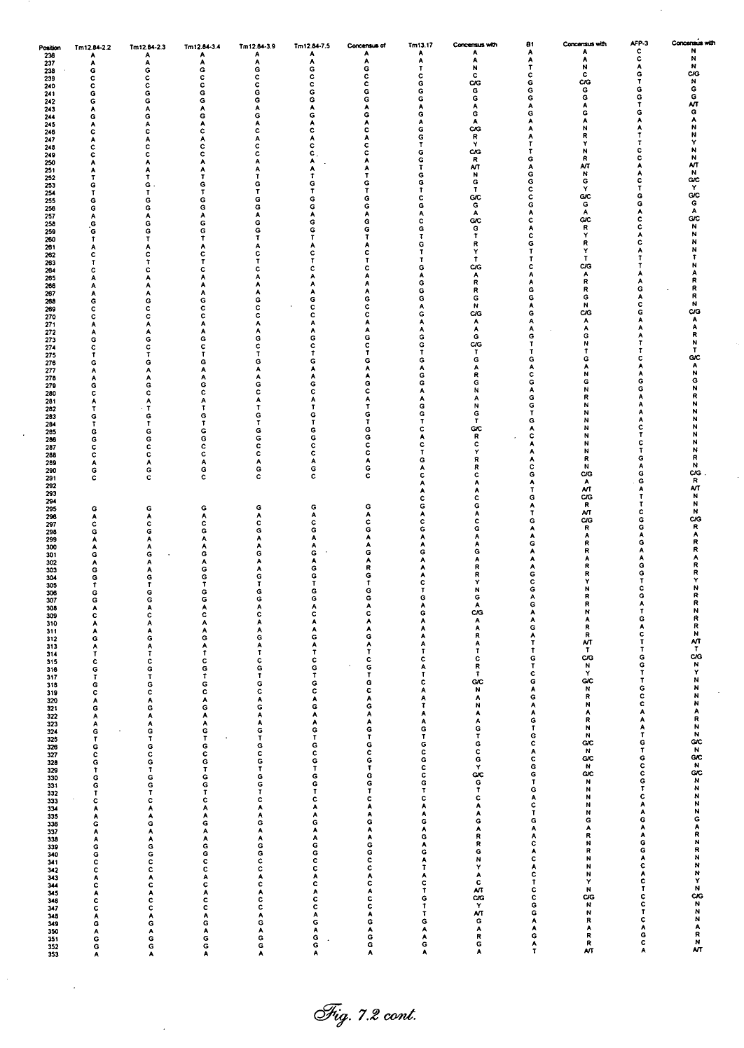

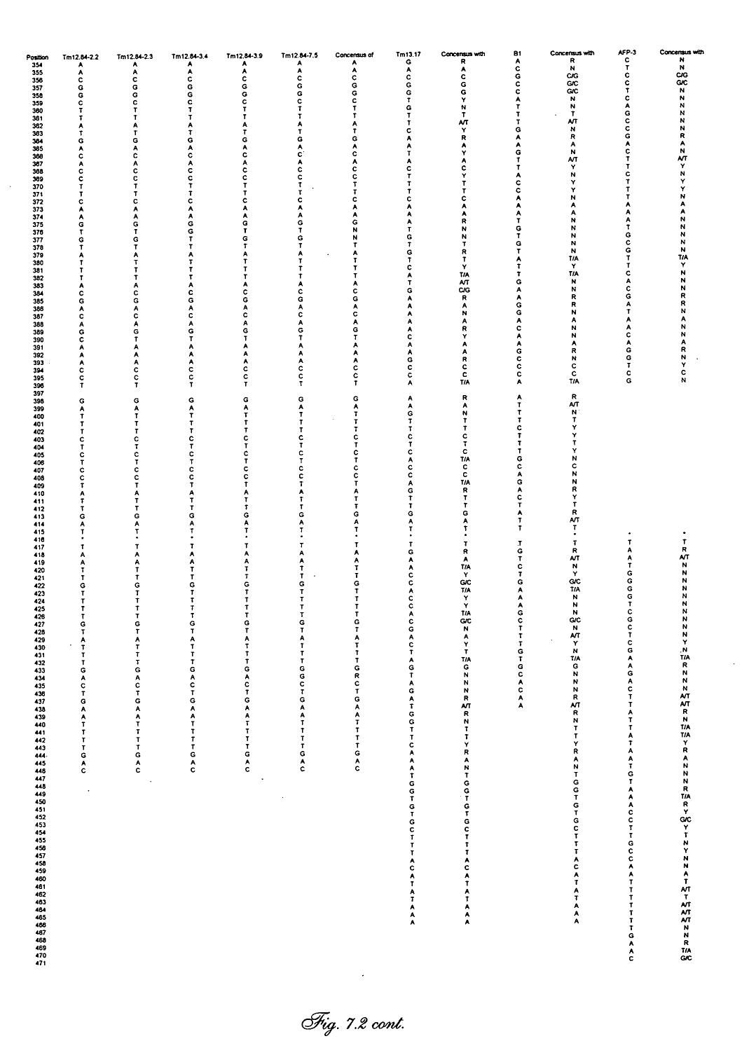

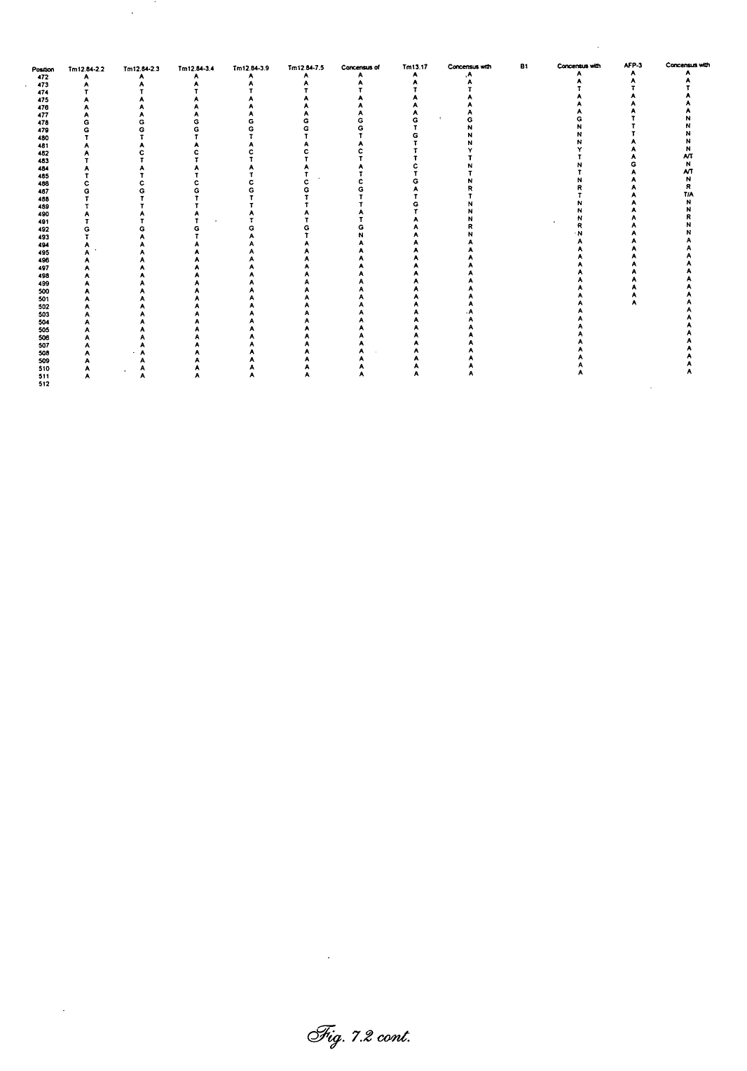

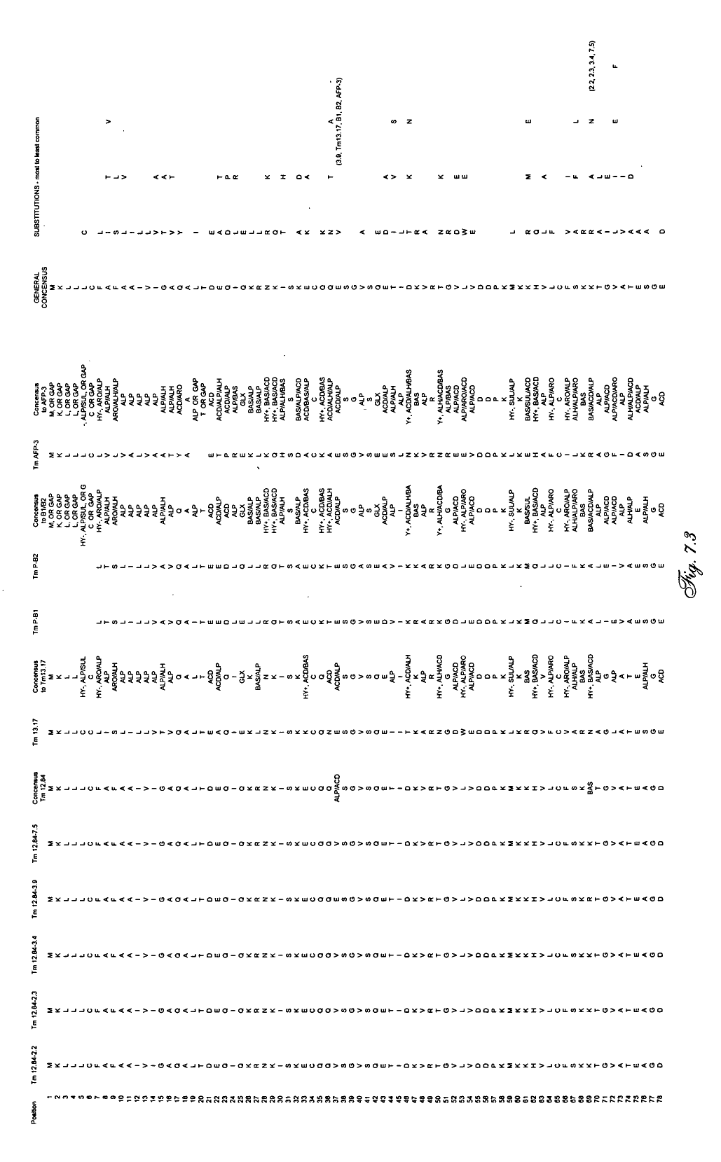

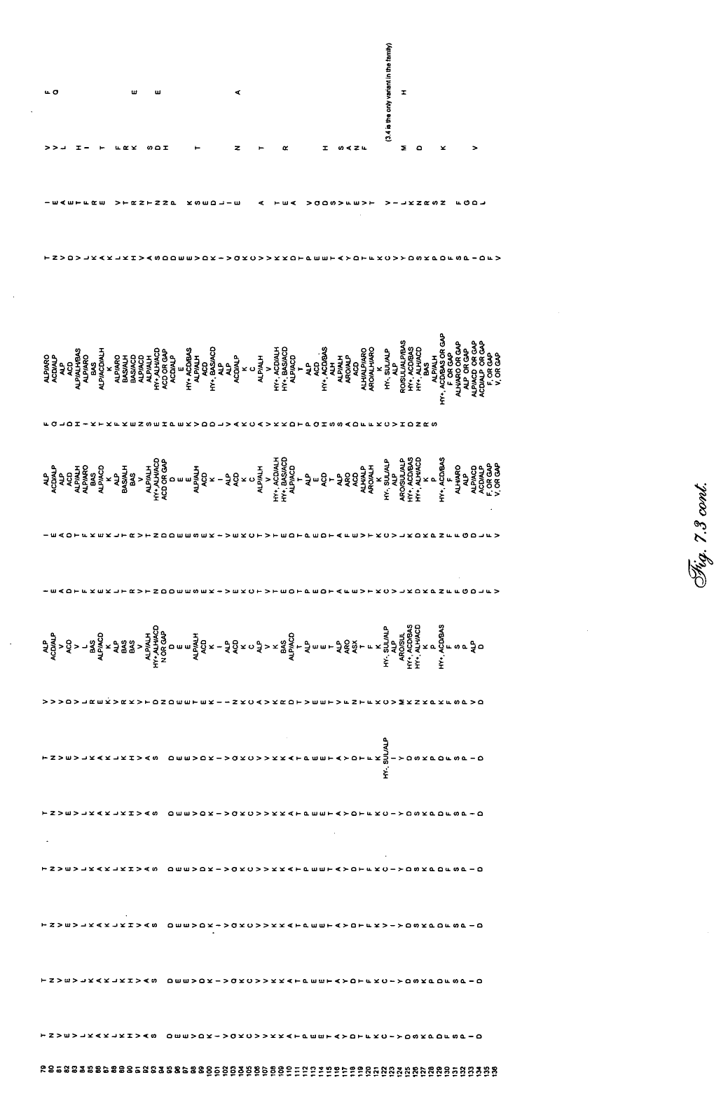

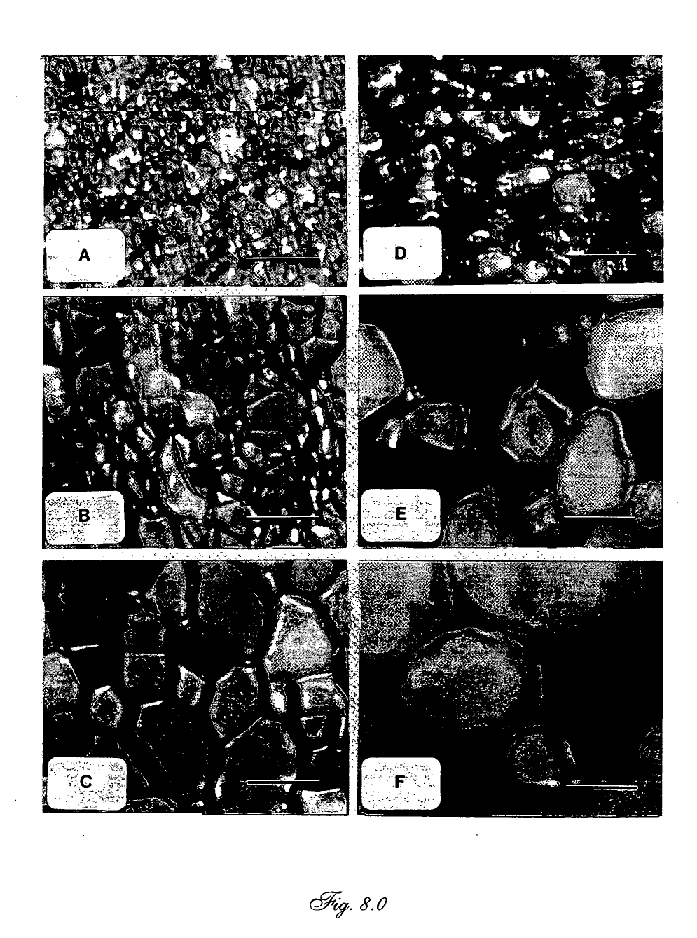

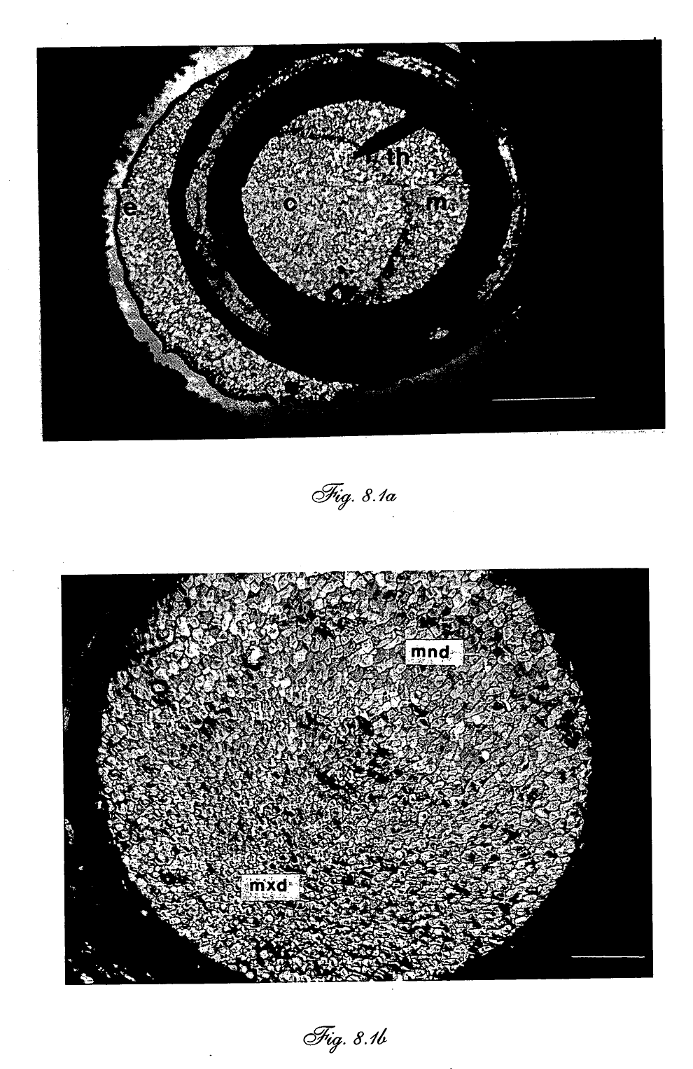

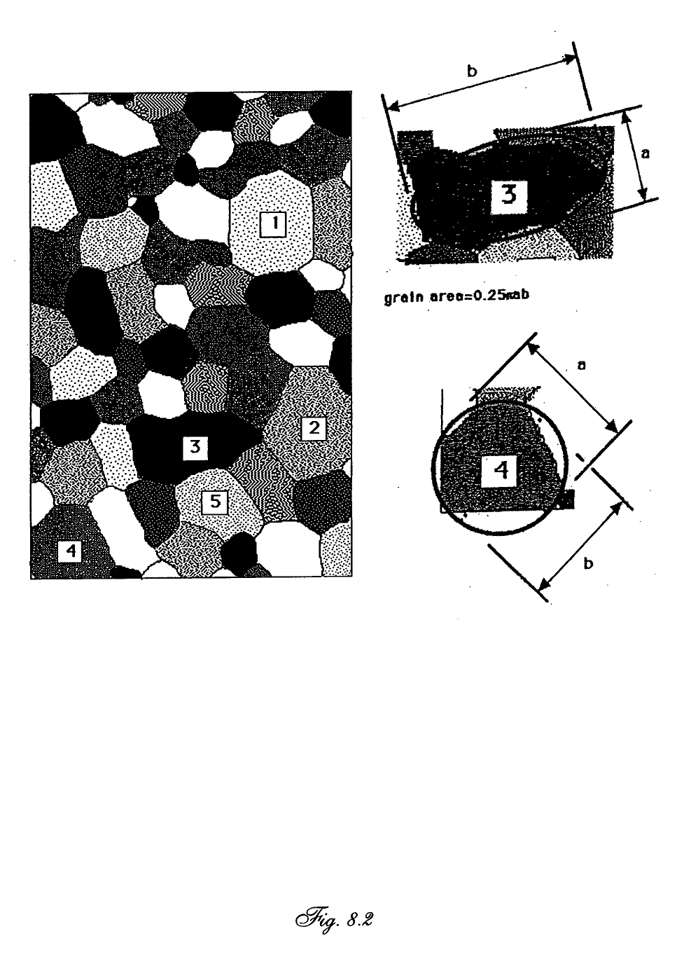

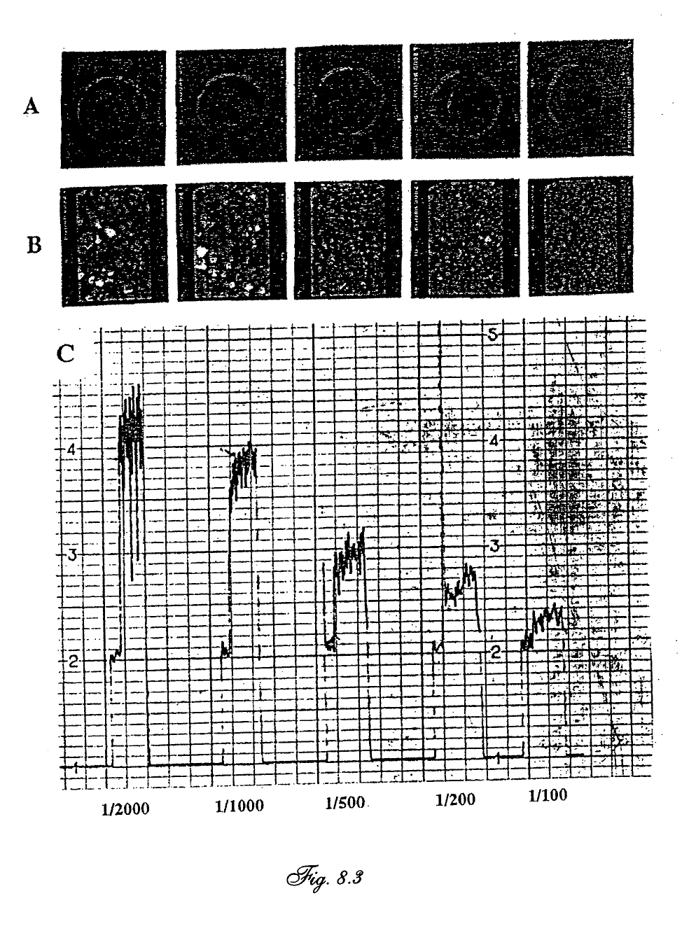

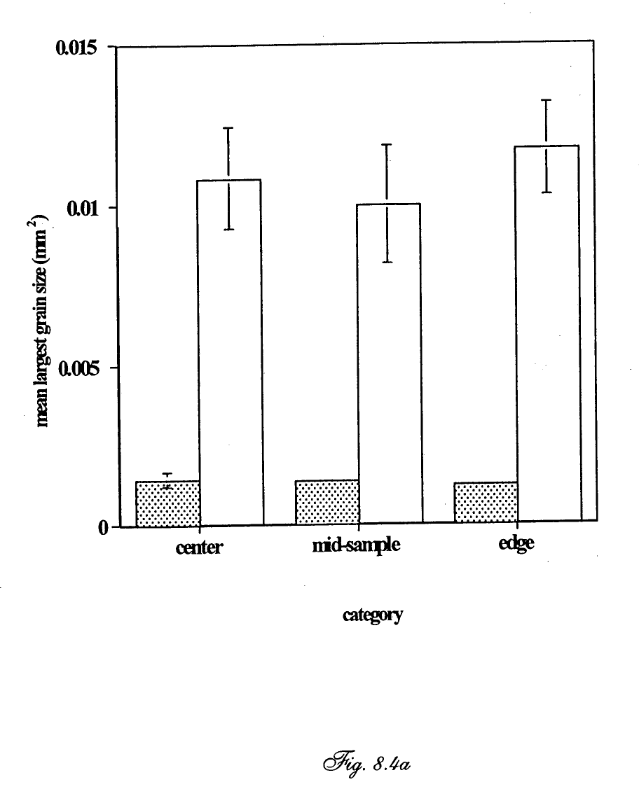

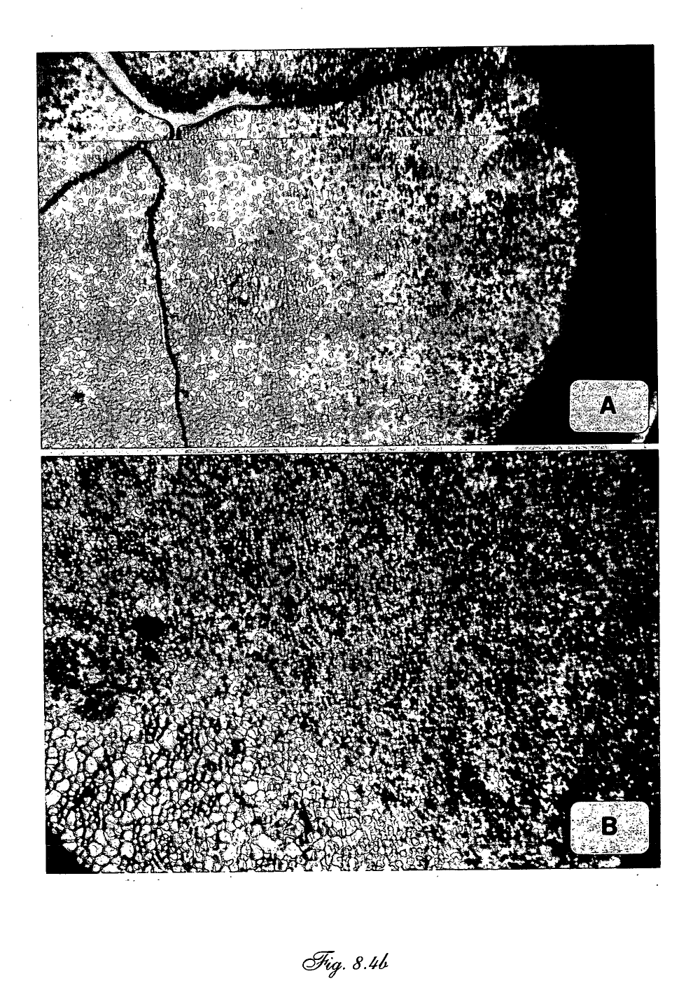

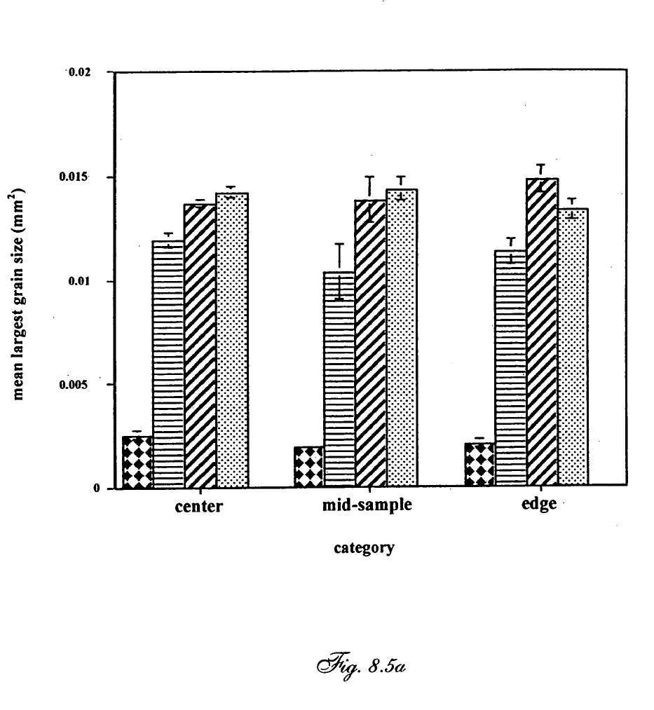

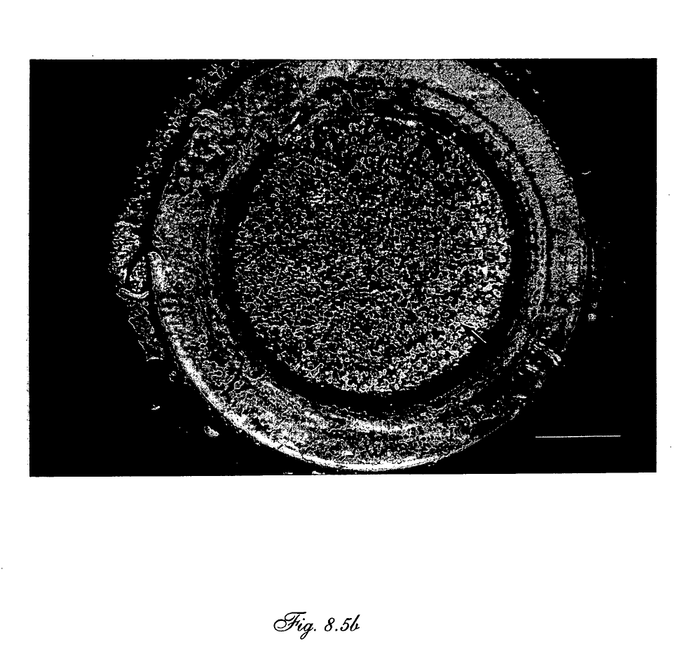

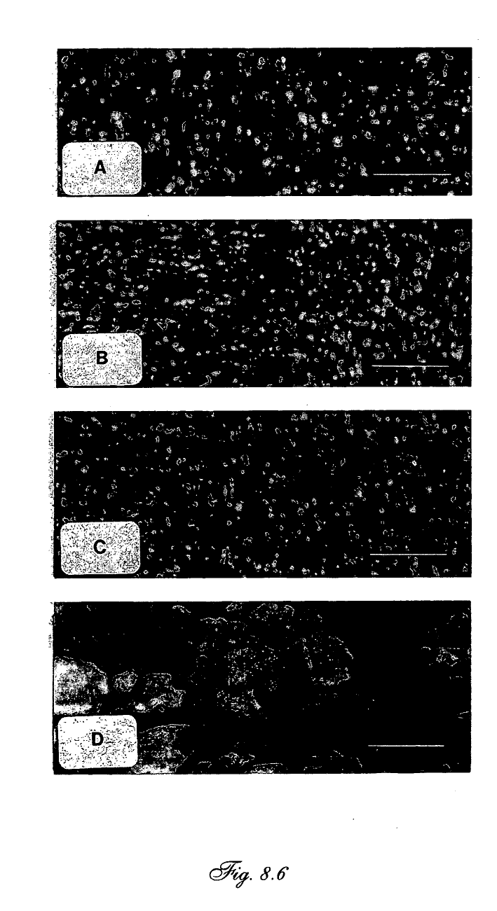

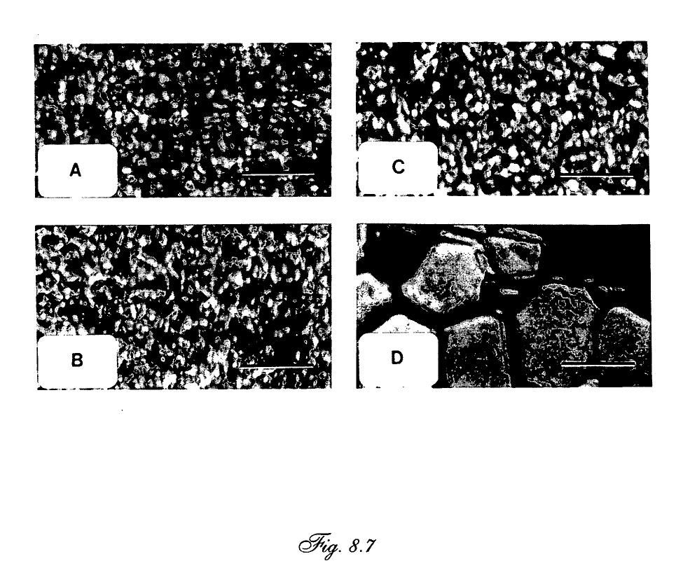

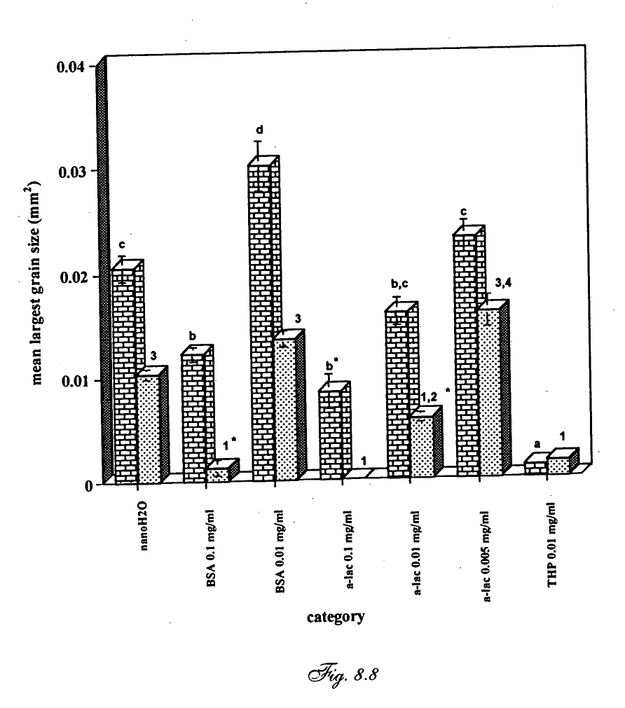

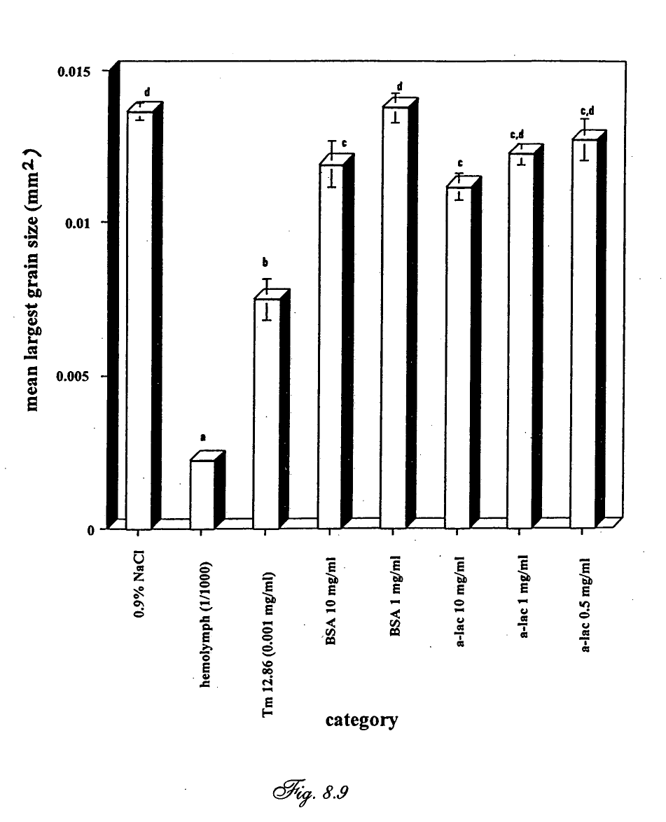

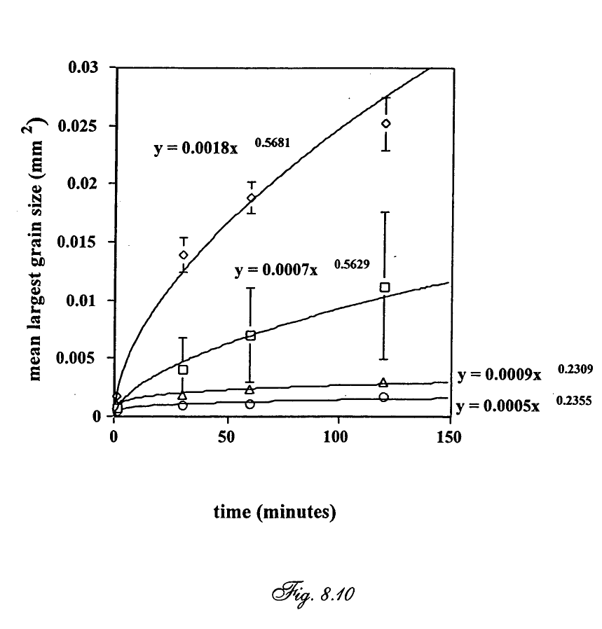

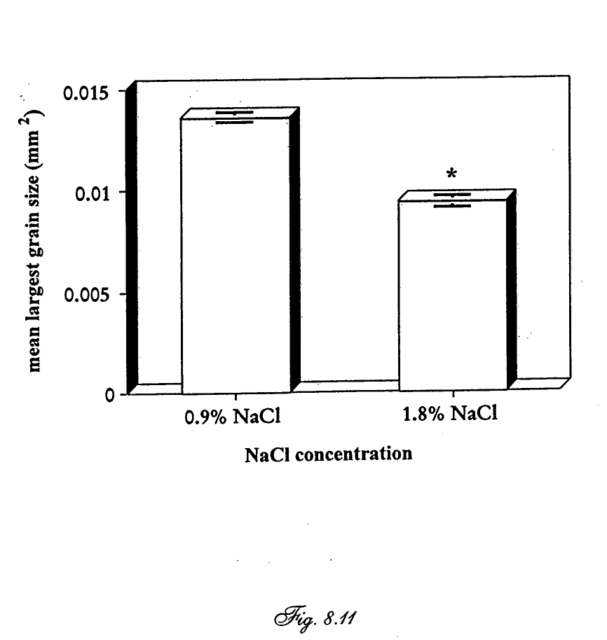

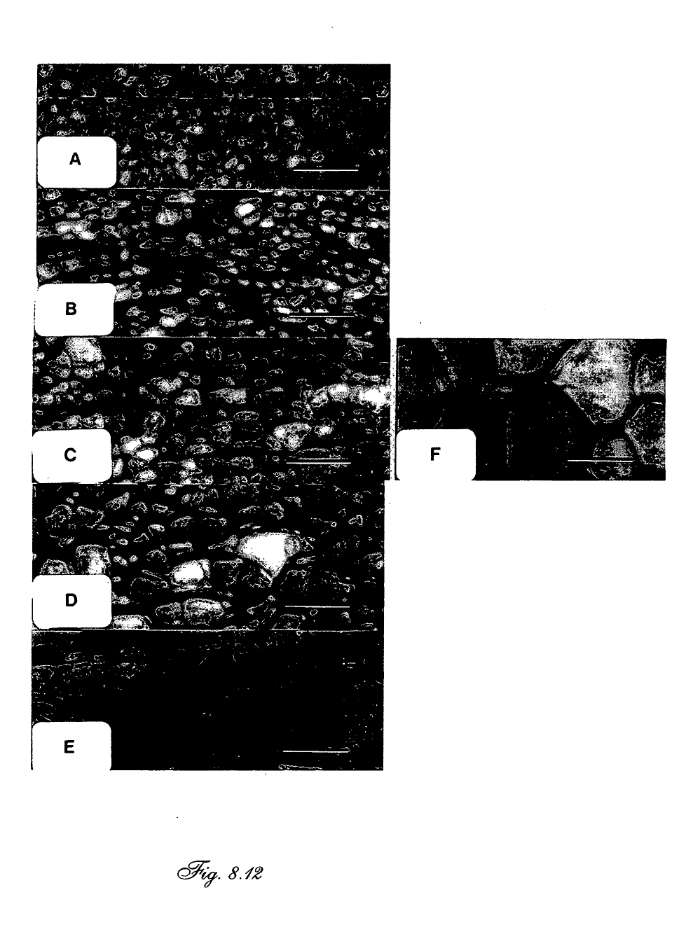

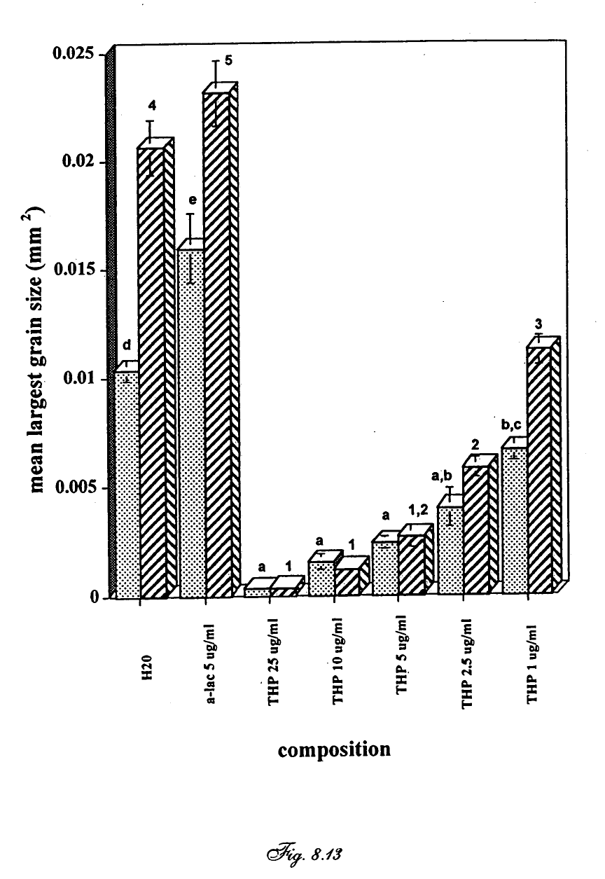

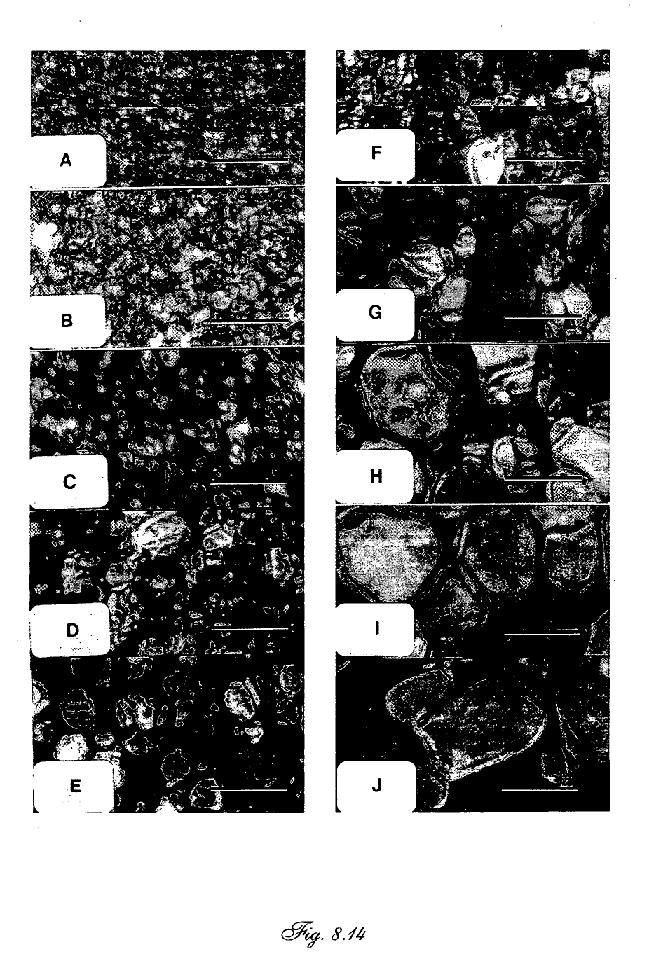

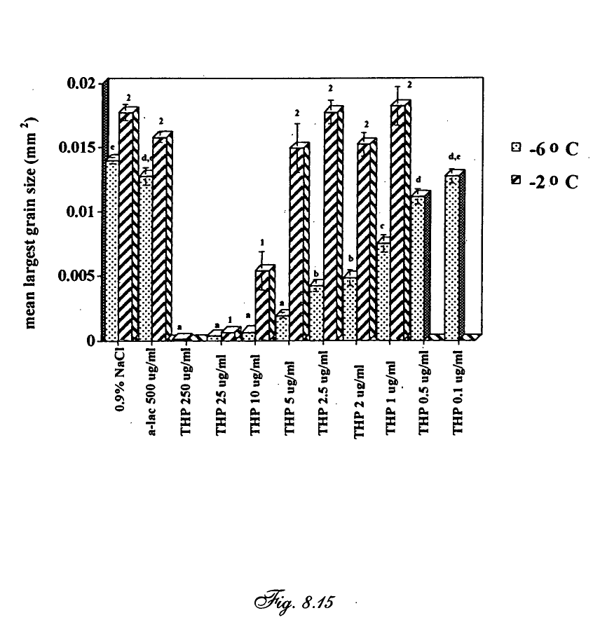

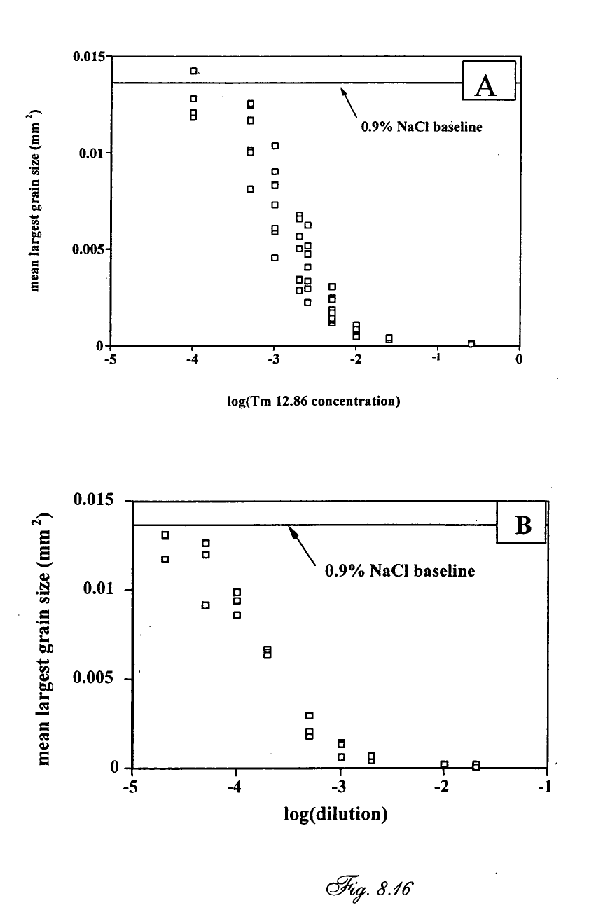

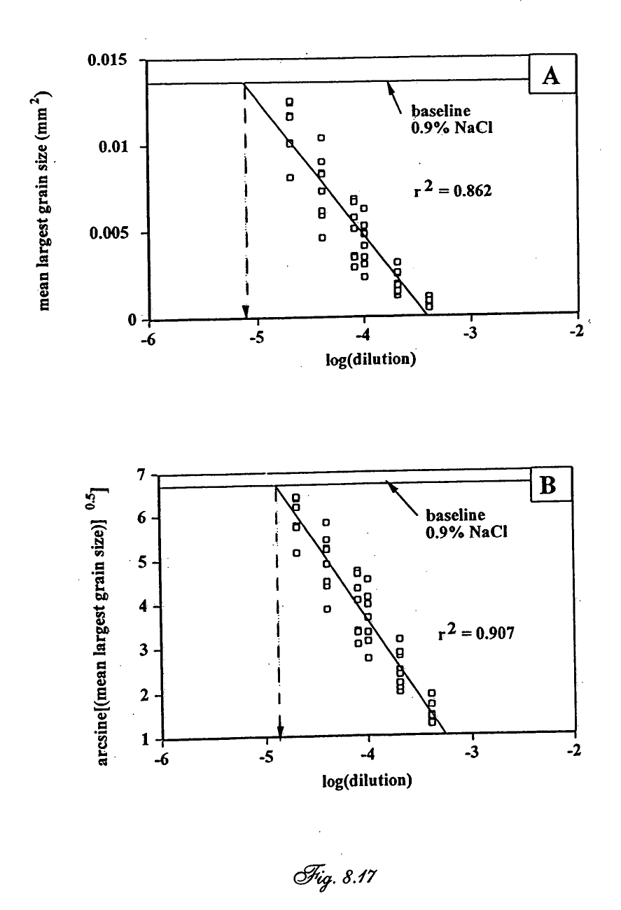

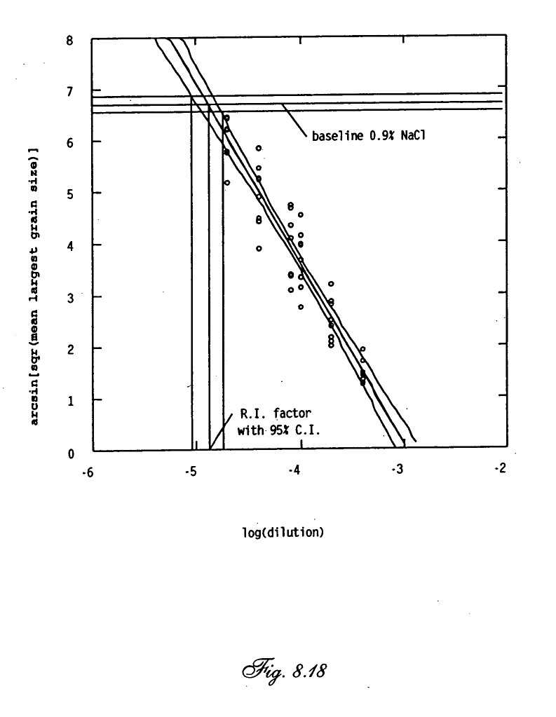

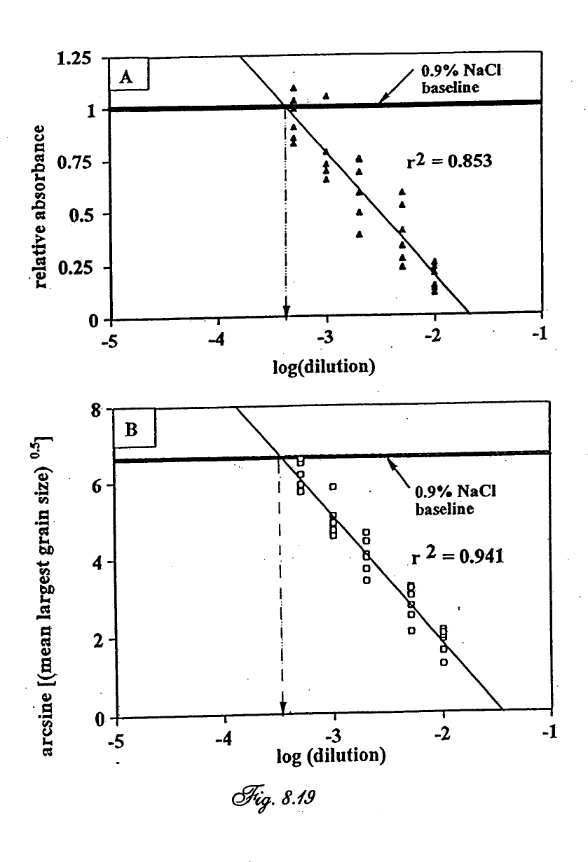

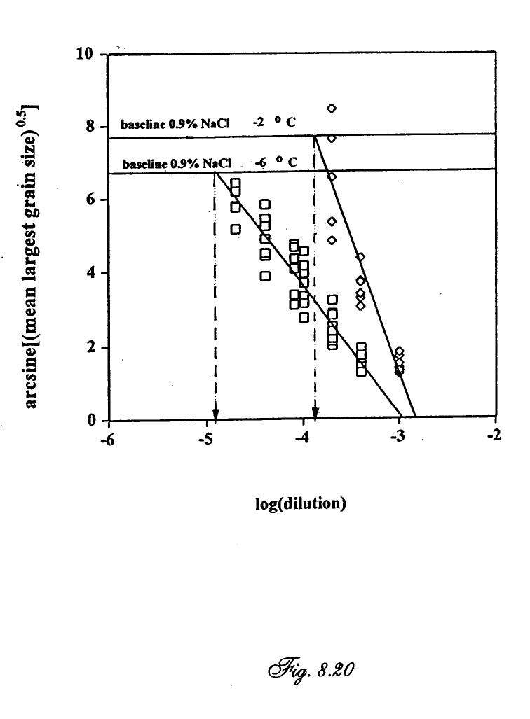

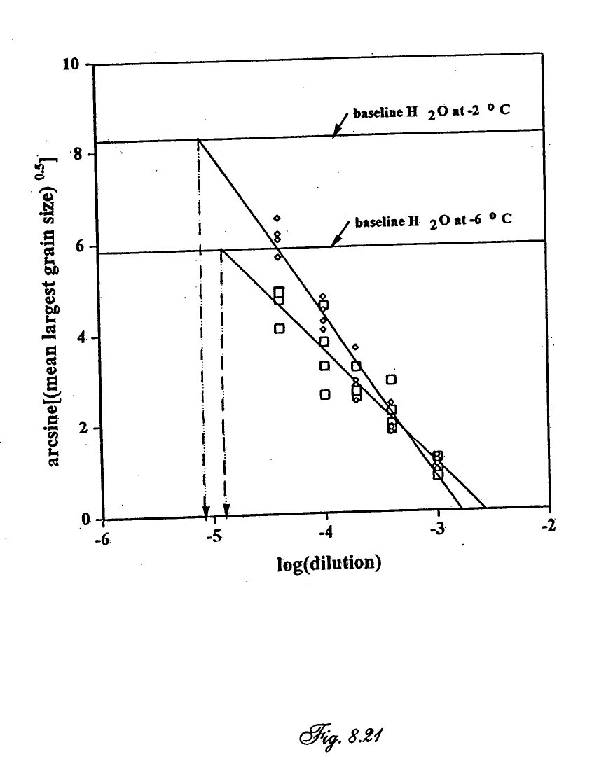

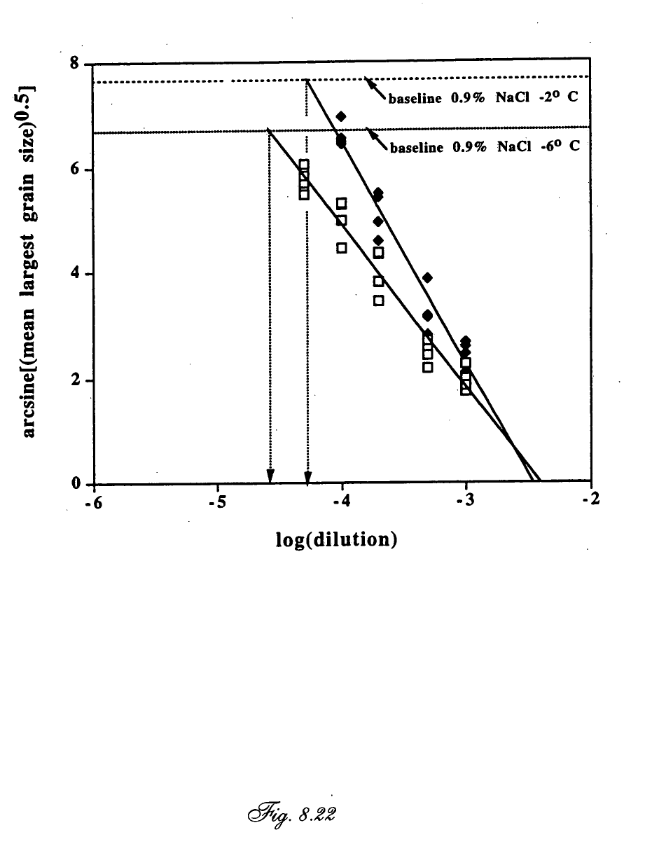

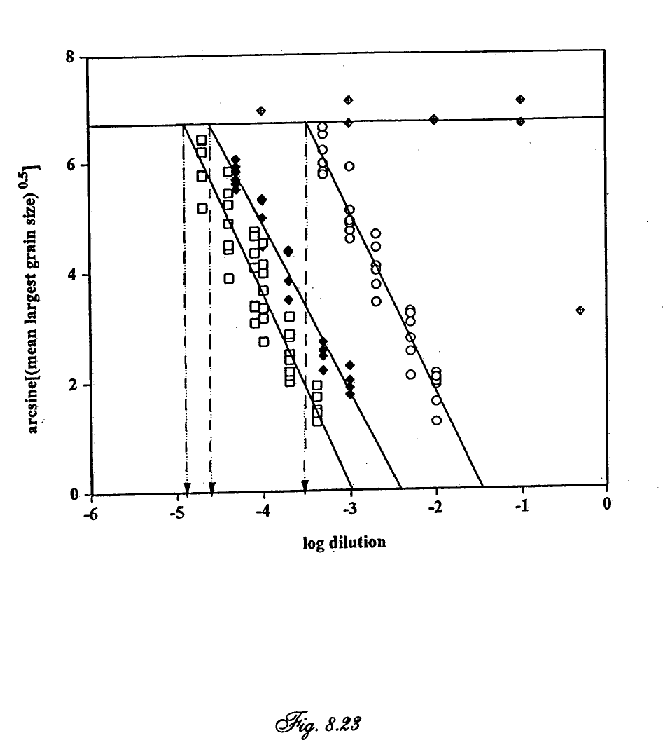

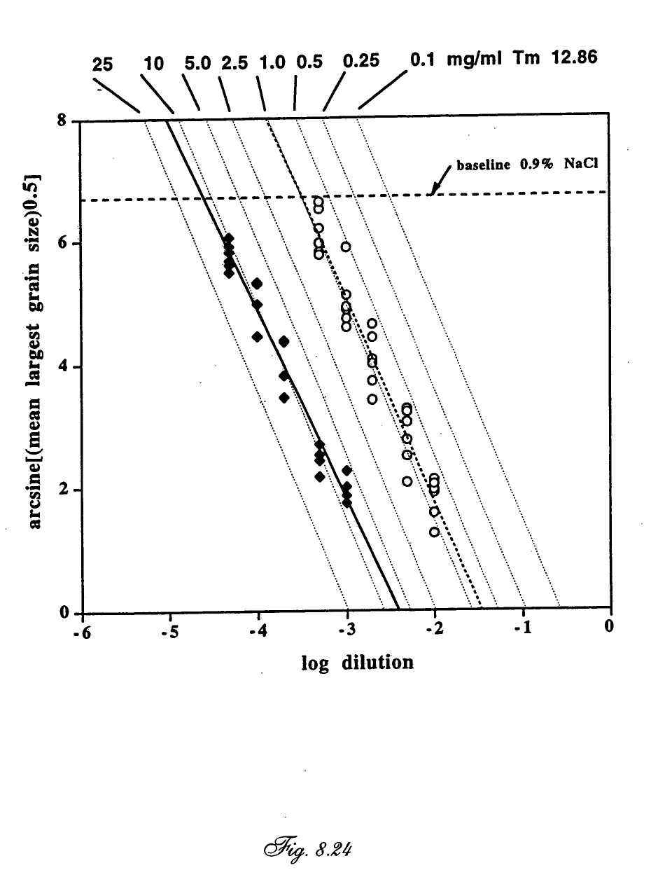

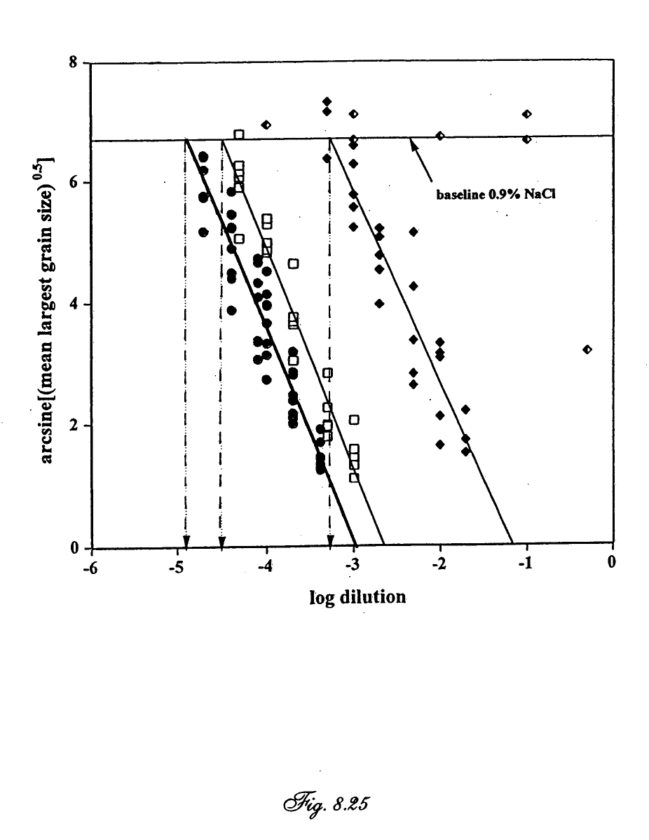

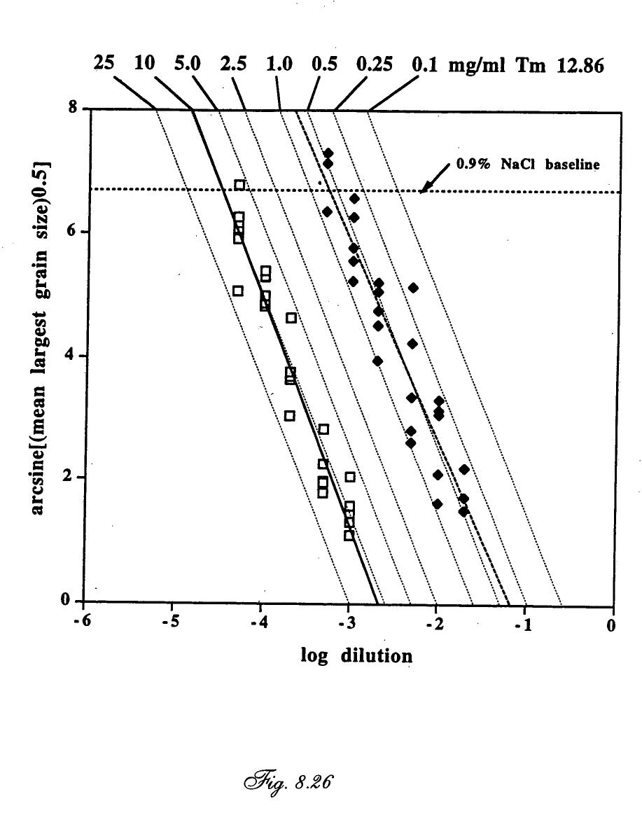

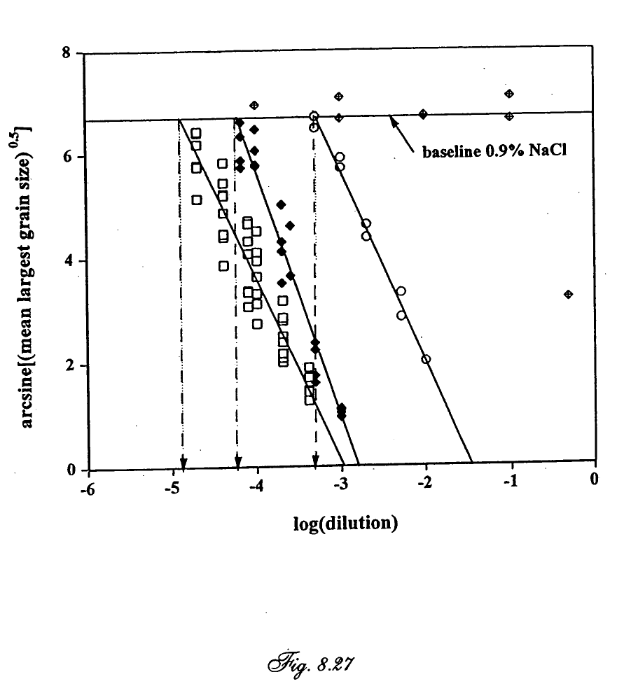

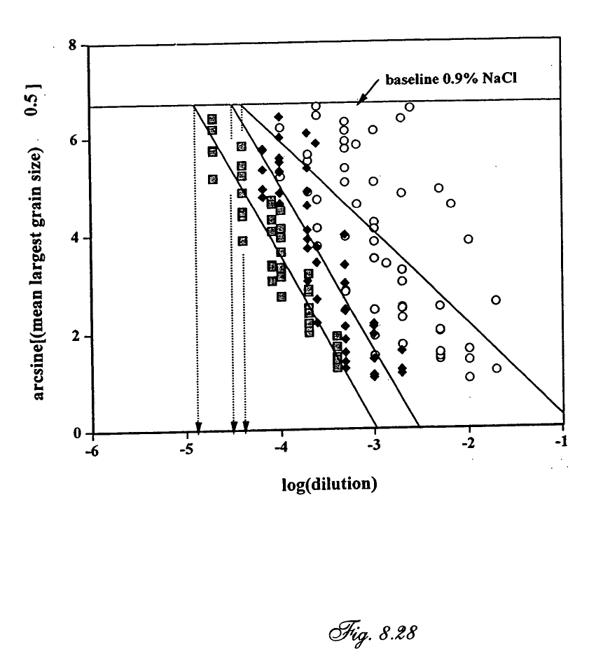

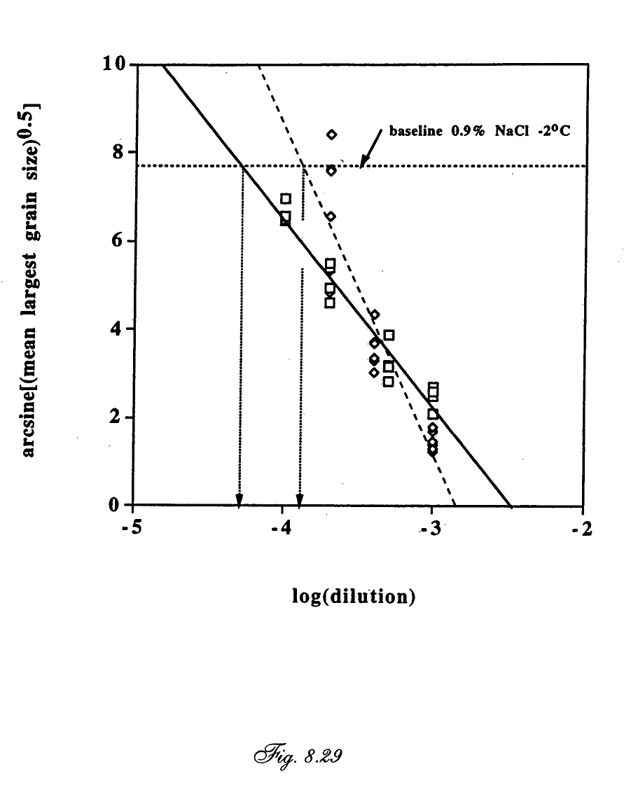

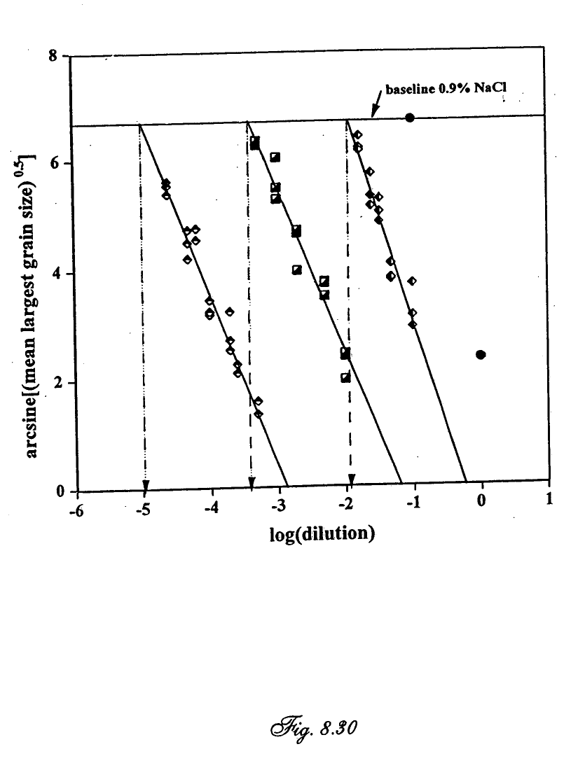

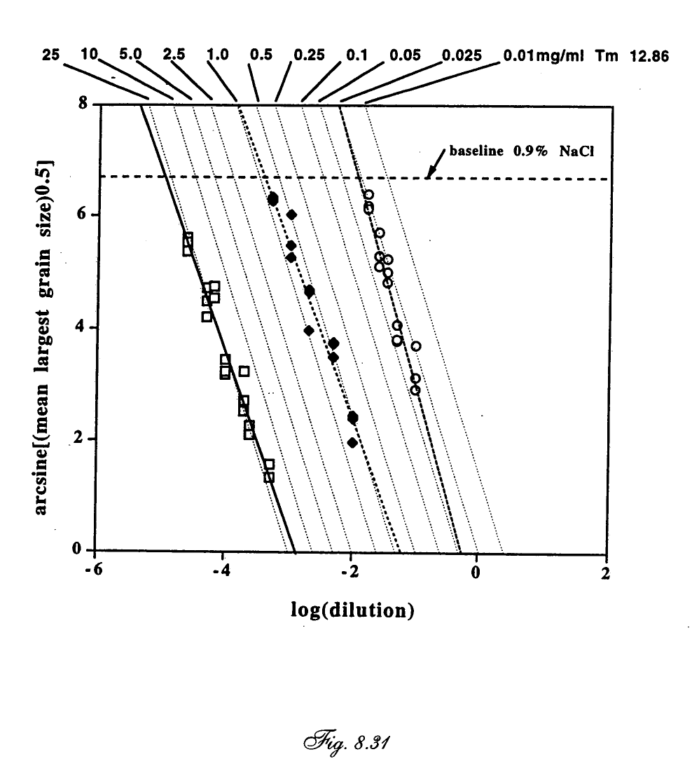

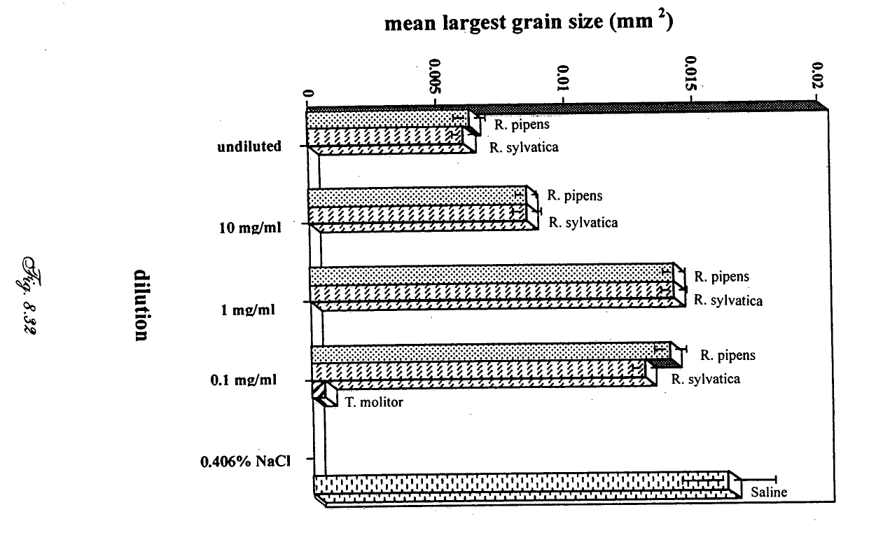

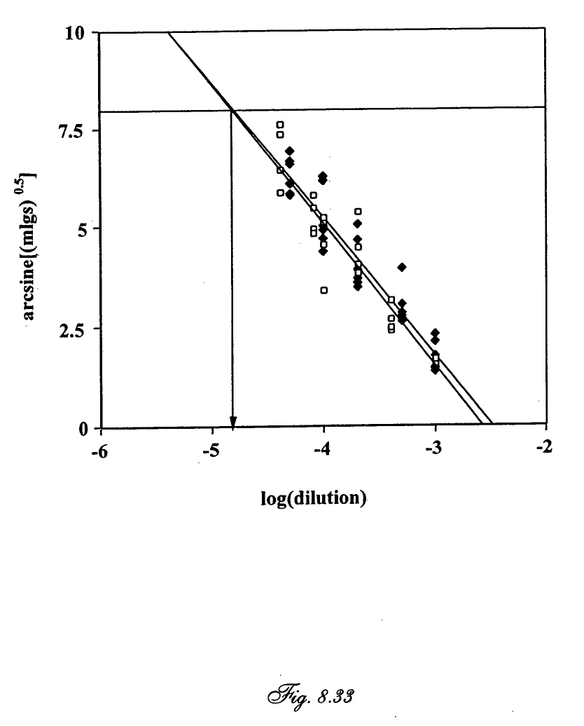

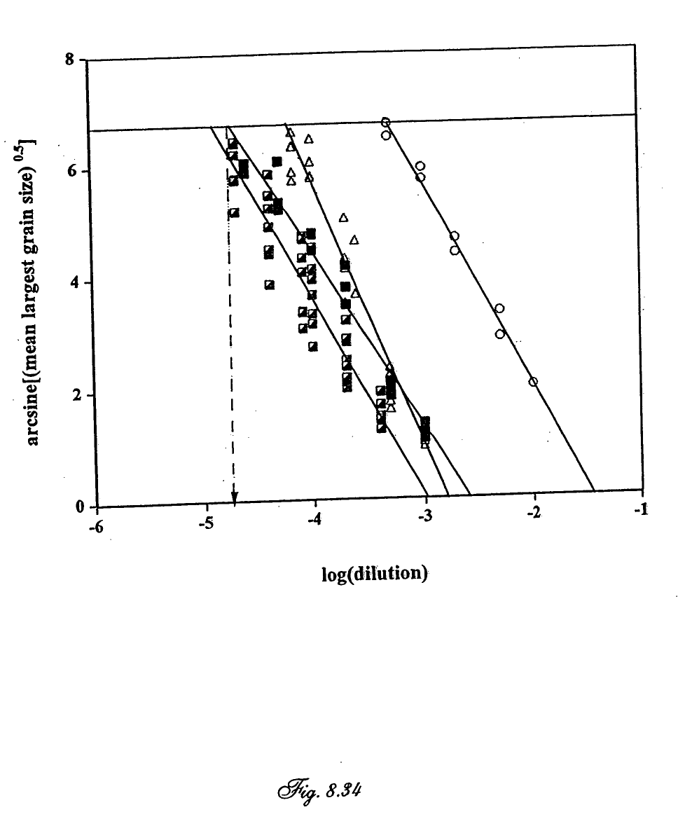

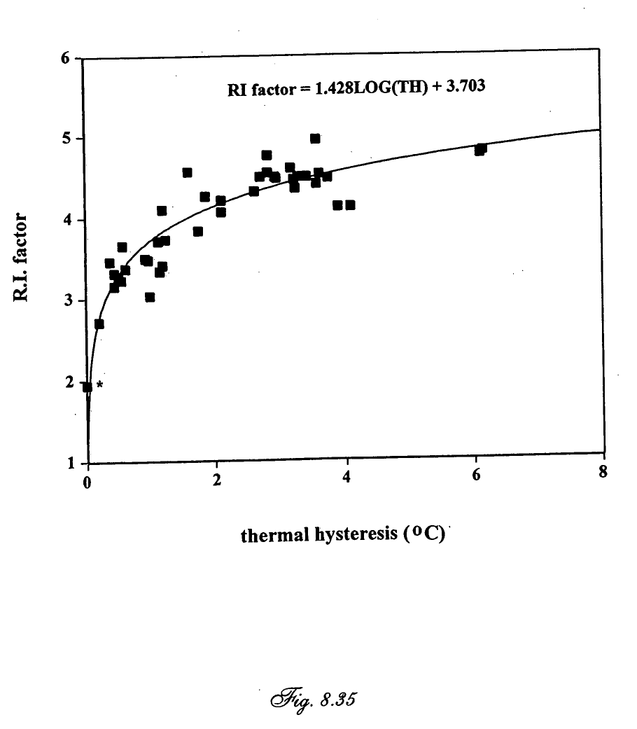

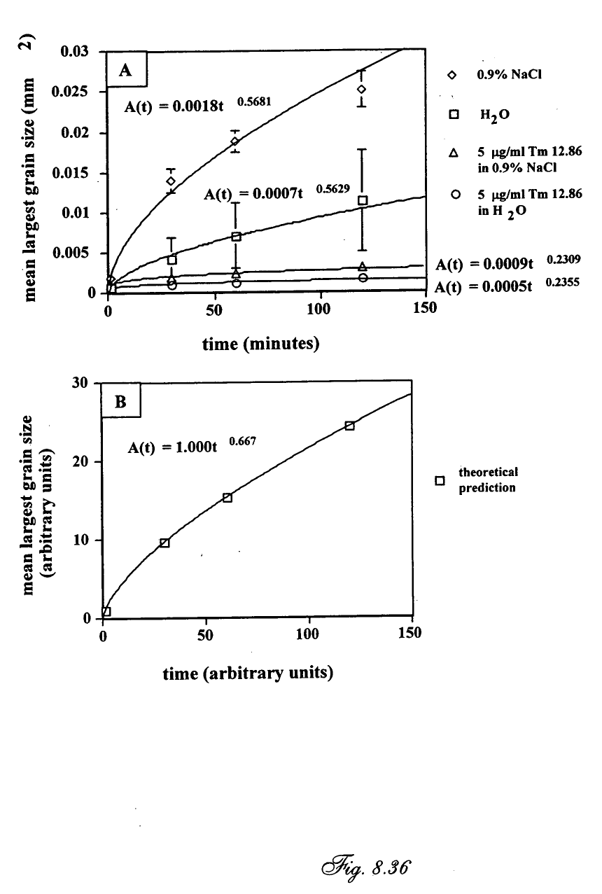

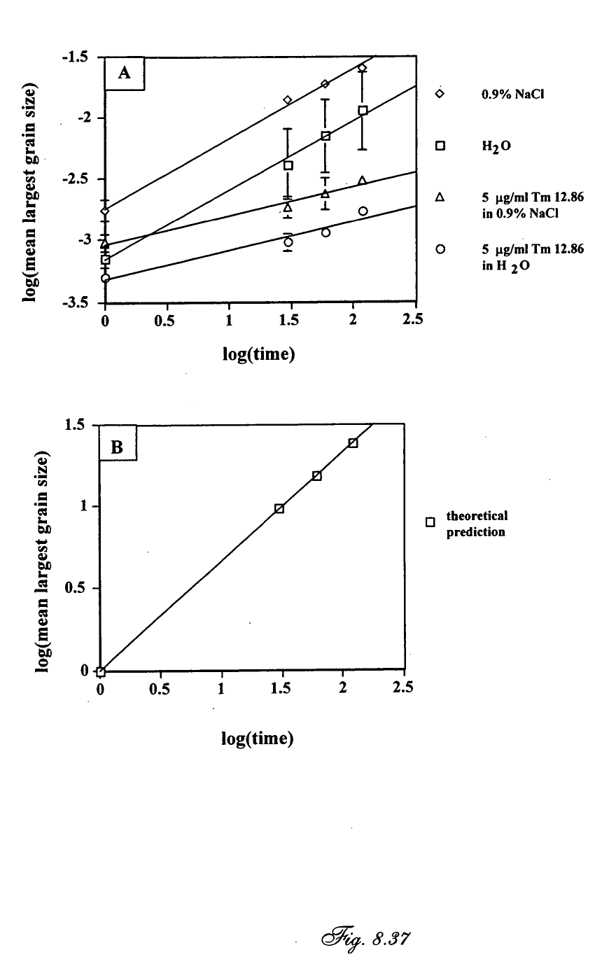

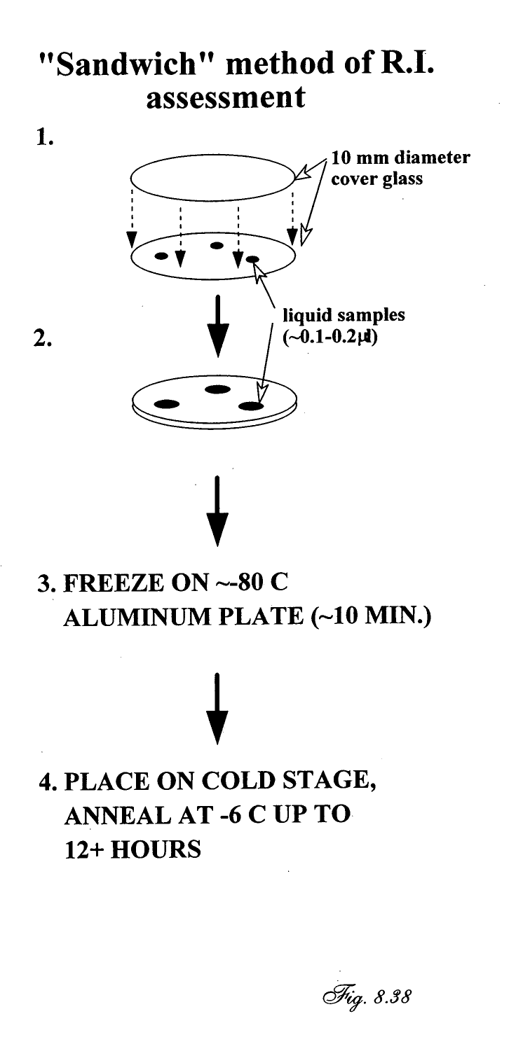

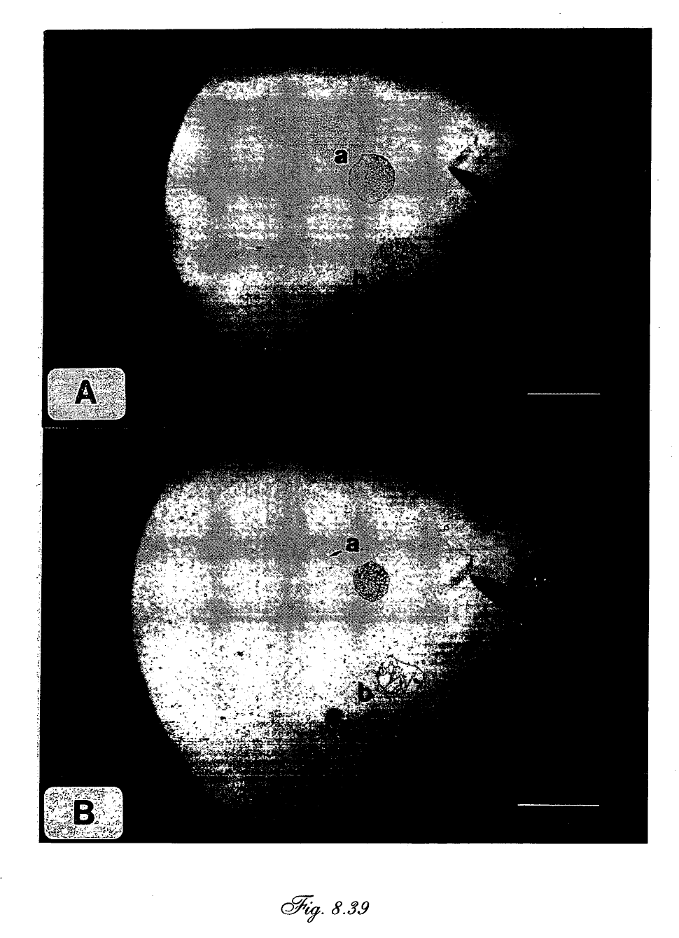

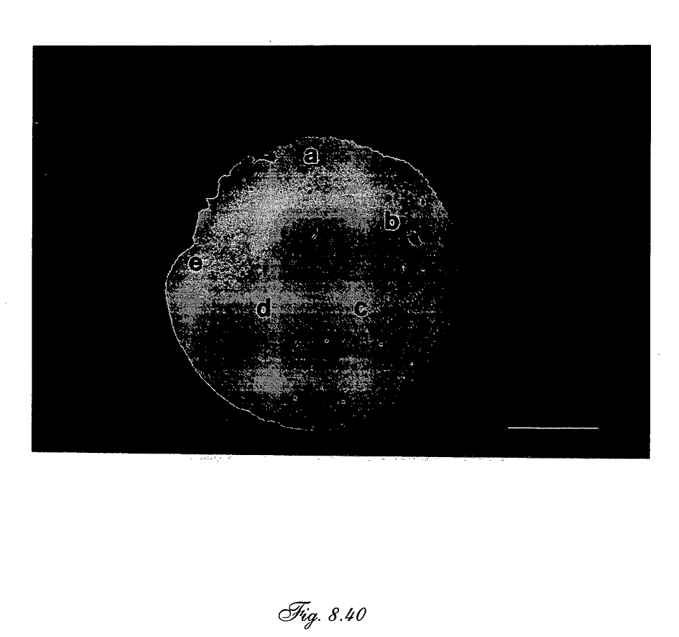

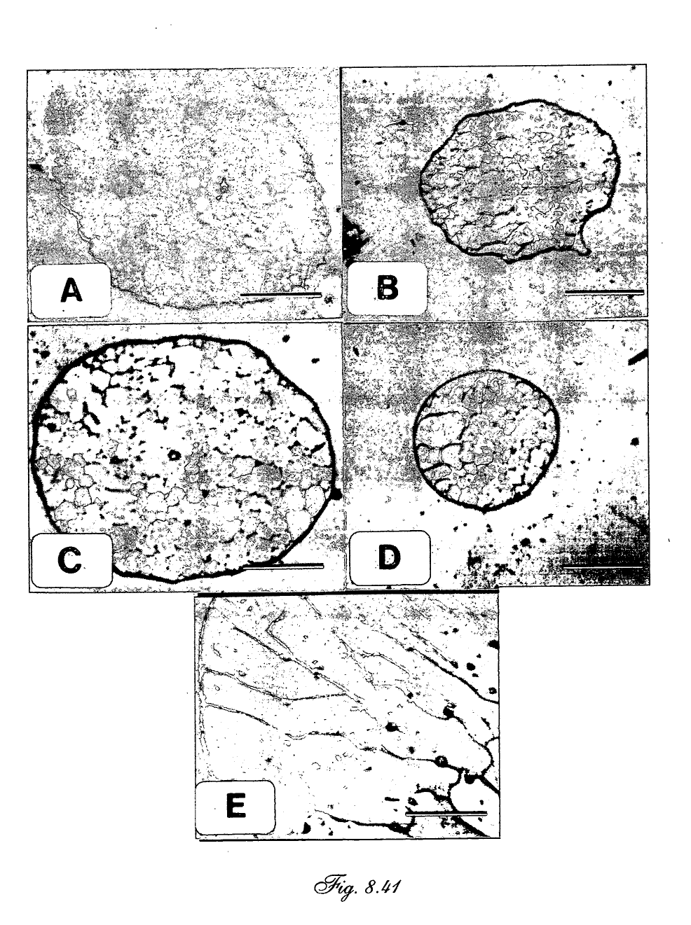

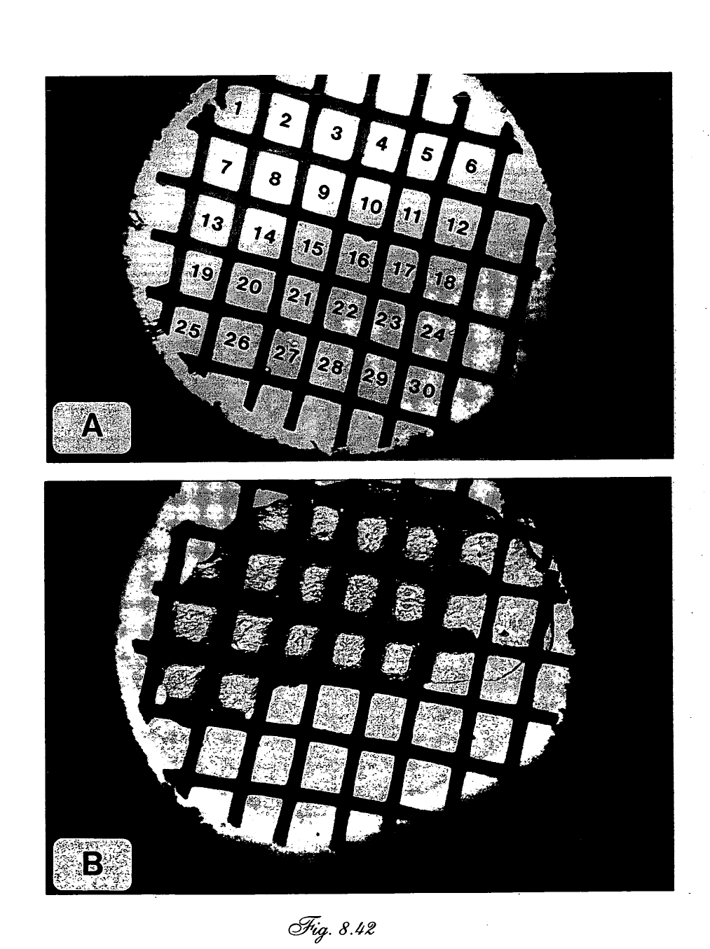

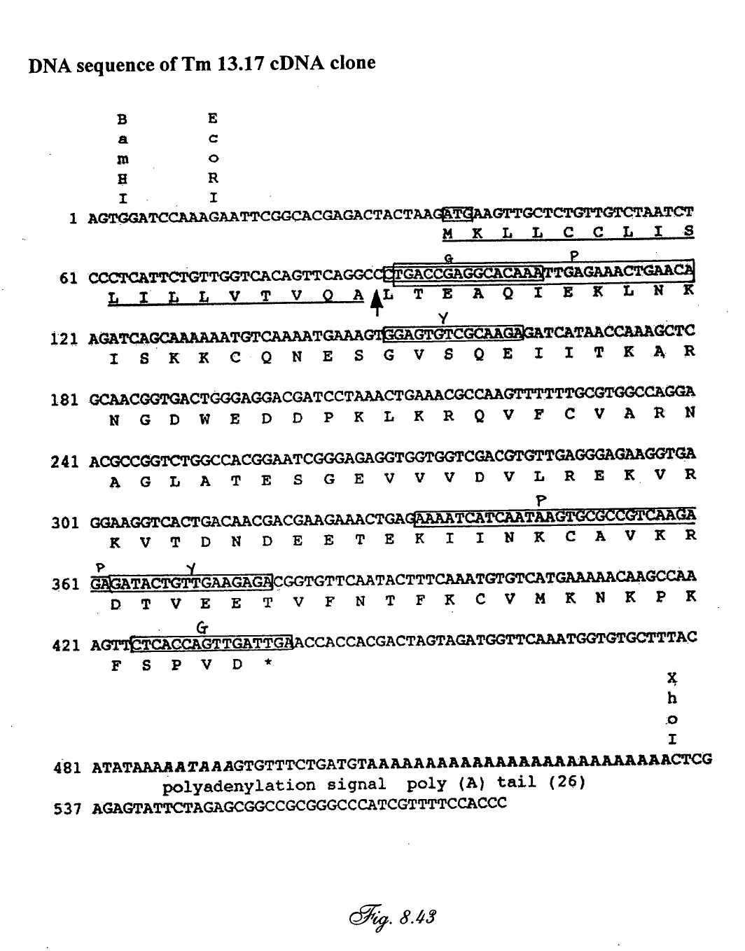

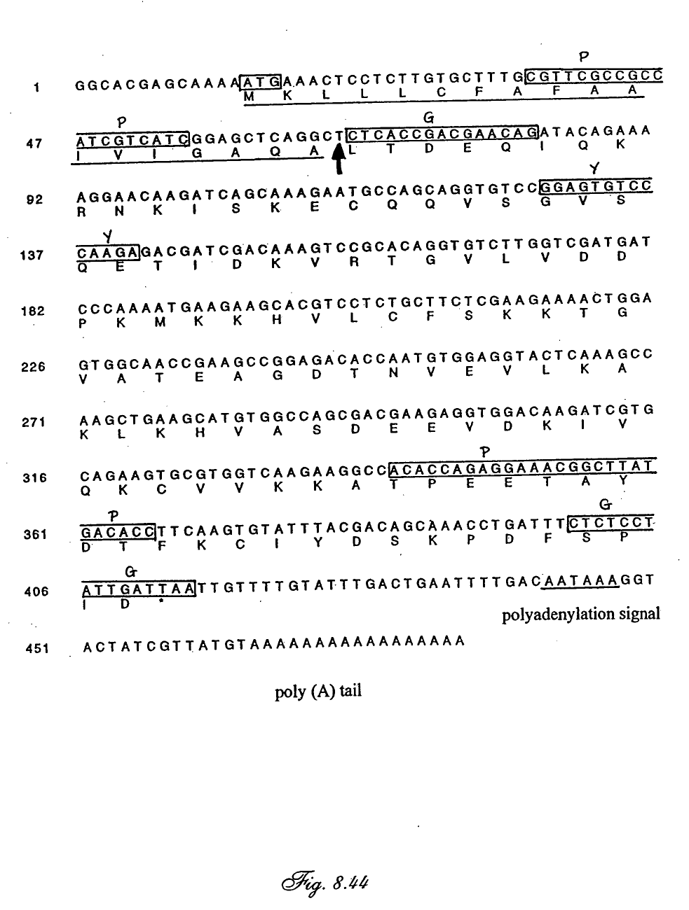

FIG. 7-1 is a table listing of letter designations for amino acids and chemical classifications

FIG. 7-2 describes specific details of the nucleotide concensus sequences developed for the Tm 12.86 family of genes.

FIG. 7-3 describes specific details of the protein concensus sequences encoded by the Tm 12.86 family of genes.

FIG. 8.0 illustrates the recrystallization of H₂O (left) and NaCl (right) occurring after 1 minute, 30 minutes, and 2 hours respectively. All samples were annealed at −6° C. (bars=0.1 mm).

FIG. 8.1 is a low magnification view of a splat-cooled 0.9% NaCl sample annealed at −6° C. for 30 minutes. FIG. 8.1A. Center (c), mid-sample (m), and edge (e) regions are shown. The sample is resting on a support ring (arrow). “th”=thermocouple. (bar at lower right=3.0 mm). FIG. 8.1B. 0.9% NaCl sample annealed at −2° C. for 30 minutes. Putative maximum deformation (mxd) and minimum deformation (mnd) areas are shown. (bar=1.0 mm).

FIG. 8.2 is a schematic representation of a recrystalzed ice sample photograph taken at high magnification (44.5×). The process by which the five largest ice grains per photograph are chosen and grain sizes approximated as elliptical areas is also shown.

FIG. 8.3 are measurements of concentration-dependent RI effects using light scattering. FIG. 8.3A is a low mag. (1.85×) photographs of splat-cooled hemolymph samples diluted in 0.9% NaCl. FIG. 8.3B is a high mag. (44.5×) of samples shown in FIG. 8.3A. FIG. 8.3C shows absorbance traces of photographic negatives corresponding to photographs shown in FIG. 8.3A.

FIG. 8.4A is a comparision of mean largest grain size (mlgs) of H₂O (solid) and Tm 12.86, 2.5 ug/ml (stipled) taken from different sample regions. FIG. 8.4B are ice grain size heterogeneities for a 0.1 mg/ml BSA in at −6° C. for 2 h and FIG. 8.4B) 0.1 mg/ml alpha lac in H₂O at −2° C. for 2 h.

FIG. 8.5A illustrates grain size heterogenity of THPs and non-THPs in 0.9% saline. Histogram grouping (left to right). Tenebrio hemolymph (1/1000 dilution), BSA 10 mg/ml, BSA 1 mg/ml, saline. FIG. 8.5B are low mag (−2.5×) of 0.9% NaCl at −6° C. for 30 min (bar −2 nm).

FIG. 8.6 illustrates that non-THPs cause RI under certain annealing conditions; FIG. 8.6A 0.025 mg/mil Tm 12.86, FIG. 8.6B 0.1 mg/ml BSA, FIG. 8.6C 0.1 mg/ml a-lactalbumin, FIG. 8.6D H₂O control. Samples diluted in H₂O, frozen and annealed at −6° C. for 2 h (bars=0.1 mm).

FIG. 8.7 illustrates higher annealing temperatures can eliminate RI effects of non-THPs (all samples diluted in H₂O). FIG. 8.7A 0.025 mg/ml Tm 12.86, −6° C. for 2 h, FIG. 8.7B 0.1 m.g/ml BSA, −6° C. for 2 h, FIG. 8.7C 0.025 mg/ml Tm 12.86, −2° C. for 2 h, FIG. 8.7D 0.1 mg/ml BSA, −2° C. for 2 h (bars=0.1 mm).

FIG. 8.8 illustrates the effect of higher annealing temperatures on non-THP RI effects in water. Histograms (left-rt) −2° C., −6° C. Letters and numbers reflect statistical relationships within temperatures. * indicate samples with ice grain size heterogeneities.

FIG. 8.9 illustrates effects of non-THPs in saline with respect to mean largest grain size. All samples annealed at −6° C. for 30 min.

FIG. 8.10 illustrates time course for recrystallization comparing saline (diamond), water (square), and 5 ug/ml Tm 12.86 in saline (triangle) or water (circle).

FIG. 8.11 is a comparison of effects of 0.9% and 1.8% NaCl on mlgs, samples annealed at −6° C. for 30 min.

FIG. 8.12 illustrate concentration-dependent effects of Tm 12.86 in H₂O. Samples annealed at −2° C. for 2 h. FIG. 8.12A 25 μg/ml, FIG. 8.12B 10 μg/ml, FIG. 8.12C 5 μg/ml, FIG. 8.12D 2.5 μg/ml, FIG. 8.12E 1.0 μg/ml, FIG. 8.12F H₂O control.

FIG. 8.13 illustrates RI concentration dependent effects of Tm 12.86 in water. Histograms (left to right)−6° C. and −2° C.

FIG. 8.14 illustrate RI concentration-dependent effects of Tm 12.86 in 0.9% NaCl. Samples annealed at −6° C. for 30 min. FIG. 8.14A 250 μg/ml, FIG. 8.14B 25 μg/ml, FIG. 8.14C 10 μg/ml, FIG. 8.14D 5 μg/ml, FIG. 8.14E 2.5 μg/ml, FIG. 8.14F 2.0 μg/ml, FIG. 8.14G 1.0 μg/ml, FIG. 8.14H 0.5 μg/ml, FIG. 8.14I 0.1 μg/ml, FIG. 8.14J 0.9% NaCl.

FIG. 8.15 illustrate RI concentration dependent mlgs values of Tm 12.86 in 0.9% NaCl at −2° C. (solid bars) and −6° C. (stipled bars). Also shown: negative control (0.9% NaCl) and positive control (0.5 mg/ml a-lactalbumin in 0.9% NaCl) mlgs values at −2° C. and −6° C. All samples were annealed for 30 min.

FIG. 8.16 illustrate RI dilution profiles of Tm 12.86 FIG. 8.16A and Tenebrio hemolymph from summer animals FIG. 8.16B. Samples diluted in saline and annealed at −6° C. for 30 min.

FIG. 8.17 illustrate regression line estimates for Tm 12.86 in saline. FIG. 8.17A is untransformed mlgs and FIG. 8.17B is transformed. RI factors (A−5.1, B=4.88) are estimated by the bottom arrows.

FIG. 8.18 illustrates linear regression confidence intervals to estimate variability in RI factor for Tm 12.6 in saline. Sampled annealed at −6° C. C for 30 min.

FIG. 8.19 is a comparison of RI estimates for Tenebno hemolymph in saline FIG. 8.19A light scattering FIG. 8.19B transformed mlgs. Sampled annealed at −6° C. for 30 min.

FIG. 8.20 is a comparison of RI dilution profiles for Tm 12.86 in saline at −6° C. (squares) and −2° C. (diamonds). Samples annealed for 30 min.

FIG. 8.21 is a comparison of RI dilution profiles for Tm 12.86 in water at −6° C. (square) and −2° C. (diamond). Samples annealed for 2 h.

FIG. 8.22 is a comparision of RI dilution profiles for Tenebrio hemolymph diluted in saline at −6° C. (square) and −2° C. (diamond). Samples annealed for 30 min.

FIG. 8.23 illustrates RI dilution profiles for Tm 12.86 (squares), winter Tenebrio hemolymph (diamonds), summer Tenebrio hemolymph (circles), and M. sexta hemolymph control (top data points parallel to baseline 0.9% NaCl (dotted line). Samples diluted in saline and annealed at −6° C. for 30 min.

FIG. 8.24 illustrates estimates of Tm 12.86 starting concentrations which produce RI profiles equivalent to winter acclimated conditions (diamond) and summer (circles) conditions. Samples diluted in saline and annealed at −6° C. for 30 min.

FIG. 8.25. RI dilution profiles for Tm 12.86 (circle), winter Tenebrio hemolymph (square), summer Tenebrio hemolymph (diamonds), and M. sexta hemolymph control (top data points parallel to baseline 0.9% NaCl (dotted line). Samples diluted in saline and annealed at −6 for 30 min.

FIG. 8.26 illustrates estimate of Tm 12.86 starting concentrations which produce RI profiles equivalent to winter acclimated conditions (square) and summer (diamond) conditions. Samples diluted in saline and annealed at −6° C. for 30 min.

FIG. 8.27 illustrates RI dilution profiles for Tm 12.86 (square), winter Dendroides hemolymph (diamond), summer Dendroides hemolymph (circles), and M. sexta hemolymph control (top data points parallel to baseline 0.9% NaCl (dotted line). Samples diluted in saline and annealed at −6° C. for 30 min

FIG. 8.28 RI dilution profiles for Tm 12.86 (square), winter Tenebrio hemolymph (diamond), and summer Tenebrio hemolymph (circle). Samples diluted in saline and annealed at −6° C. for 30 min

FIG. 8.29 is a comparison of RI dilution profile regression lines of Tm 12.86 (diamond) and winter Tenebrio hemolymph (squares). Samples were annealed at −2° C. for 2 h.

FIG. 8.30 is a comparison of regression lines of RI dilution profiles for winter Tenebrio hemolymph (left line), summer Tenebrio hemolymph (middle line) and T. molior fat body cell culture C1 supernatent (right line). Blank culture media (solid circle) is a control for non-THP RI effects. Samples diluted in saline and annealed at −6° C. for 30 min.

FIG. 8.31 illustrates estimates of Tm 12.86 starting concentrations which produce RI profiles equivalent to winter Tenebrio hemolymph acclimated conditions (square), summer (diamond) conditions, and T. molitor C1 cell culture supernatant.

FIG. 8.32 is a comparision of mlgs for R. sylvatica and R. pipens. Samples annealed at −6° C. for 30 min,

FIG. 8.33 illustrates RI dilution profiles of Tm 12.86 (square) and Tenebrio winter hemolymph sample (diamond). Mlgs determined using a random sampling method. Samples annealed at −2° C. for 30 min.

FIG. 8.34 illustrates RI dilution profiles for Tm 12.86 (half filled squares), winter Dendroides hemolymph (solid squares), and additional Dendroides profiles shown previously in FIG. 8.27. Samples diluted in saline and annealed at −6° C. for 30 min.

FIG. 8.35 illustrates the relationship between RI factors and thermal hysteresis.

FIG. 8.36 is a comparision of time course of recrystallization plots for experimental and theoretical prediction.

FIG. 8.37 is a comparision of tme course of recrystallization plots for experimental and theoretical prediction using log/log transformations.

FIG. 8.38 is a schematic diagram outlining an alternate RI procedure.

FIG. 8.39 are photographs of recrystallized samples prepared using the “sandwich” method. FIG. 8.39A is T. molitor hemolymph 1/50 dilution in 0.9% NaCl (identified as “a”) and the 0.9% NaCl control (identified as “b”) after 30 minutes at −6° C. FIG. 8.39B are the same samples after 12 hours at −6° C. (bars=2 mm).

FIG. 8.40 is a low mag. photograph of T. molitor hemolymph in 0.9% NaCl samples and 0.9% NaCl control sample at −6° C. for 12 hours. Sample compositions are indicated above as follows: (a) 1/500 hemolymph (b) 1/1000 hemolymph, (c) 1/2000 hemolymph, (d) 1/5000 hemolymph, and (e) 0.9% NaCl control (bar=4 mm).

FIG. 8.41 are higher mag. photographs of samples seen in FIG. 8.40 (A) 1/500 hemolymph in 0.9% NaCl, (B) 1/1000 hemolymph in 0.9% NaCl, (C) 1/2000 hemolymph in 0.9% NaCl, (D) 1/5000 hemolymph in 0.9% NaCl, (E) 0.9% NaCl control. All samples shown were annealed at −6° C. for 12 hours. All photographs shown are at the same magnification; differences in sample sizes are due to corresponding differences in sample starting volumes (bars=0.4 mm).

FIG. 8.42A is a photograph showing grid placement in the cold stage holding area and assignment of grid square numbers. FIG. 8.42B is a photograph of grid with ice sample fragment.

FIG. 8.43 illustrate regions of Tm 13.17 clone used as DNA probes. Color coded areas denote forward and reverse prmer sequences used for particular experiments with the regions between and including primer sequences denoting the probe. Probe outline by yellow region was used in Example 4, probe from green region used in Example 5, and probe from pink region used for northern analysis.

FIG. 8.44 illustrate regions of Tm 2-2 clone used as DNA probes. Color coded areas denote forward and reverse prner sequences used for particular experiments with the regions between and including primer sequences denoting the probe. Probe usage as in FIG. 8.43.

### DETAILED DESCRIPTION OF THE INVENTION

In accordance with the present invention substantially pure peptides (with encoding nucleotide sequences), that exhibit ice crystal growth suppression activity are provided for use in improving or maintaining various characteristics of frozen or chilled foods and biologics, and as environmentally sound de-icing agents. These antifreeze proteins are of an insect Type III AFP classification and are more potent than any of the known fish antifreeze proteins. These insect Type III AFPs can be derivied from the natural sources through elimination of contaminating insect compounds or through isolating the desired genes, cloning them, expressing them in a suitable host cell, the purifying the expressed protein, all in a fashion that maintains the peptides non-colligative ice growth suppressing behavior. This invention relates to identifying a multigene family of insect Type III AFPs, providing the isolated nucleic acid sequences encoding this novel class of AFPs, and the generation of these peptides in a manner eliciting antifreeze activity. In addition, the invention also provides for antibodies that are reactive to these peptides, and certain novel activating substances capable of enhancing the antifreeze activity of these Type III insect AFPs are described. Further, the invention details a sensitive and quantifiable assay for evaluating recrystallization inhibition (RI) that is capable of eliminating non-specific RI effects thereby allowing for an “antifreeze protein specific” response, and means for upscaling the assay. The following illustrate a general detail of and procedures for obtaining the native proteins, the design, preparation, assembly, cloning and expression of deoxyoligonucleotides for use in the manufacture of thermal hystereticially active recombinant insect Type III antifreeze proteins, the quantitative assay for such, and the use of these antifreeze proteins and related genes and other products.

I. Isolation of Native Tm 12.86

A highly potent insect Type III antifreeze protein, designated Tm 12.86, has been purified from winter acclimated Tenebrio molitor larvae and mass spectrophometry analysis, amino acid composition, N-terminal analysis, gel electrophoresis migration patterns, thermal hysteretic profile and hemolymph physiological concentration been determine accordingly to the procedures of Example 1. Also detailed in Example 1 are procedures taken to generate a polyclonal antiserum against the purified AFP, and an assay protocol for screening for the presence of antifreeze enhancing activators.

Protein Purification. Following homogenization and ethanol extraction of winter-acclimated T. molitor larvae, the resulting supernatant was applied to a DEAE-Sepharose ion exchange column. A total of nine peaks were eluted from the ion exchange column using a stepwise increase in sodium chloride concentration (FIG. 1.1a). Each peak was desalted and then screened for thermal hysteresis at a concentration of 50 mg/ml. Although eight of the nine peaks were found to display some level of thermal hysteretic activity, ion exchange Peaks II and III (eluted by 0.03 M and 0.06 M NaCl, respectively) exhibited the greatest amount of activity (approximately 1.5° C.) (FIG. 1.1b). Peak II was chosen for further purification because more protein was eluted with Peak II (28 mg compared to 8 mg, respectively).

Ion exchange Peak II was found to exhibit multiple bands on non-denaturing PAGE and, therefore, was run on a Sephadex G-75 Superfine column. Four peaks were eluted from the gel filtration column (FIG. 1.2) and screened for thermal hysteresis at a concentration of 25 mg/ml. Only gel filtration Peak 3 displayed thermal hysteretic activity (2.5° C. at 25 mg/ml). Gel filtration Peak 3 (tubes 20-26) was shown to display one major band on non denaturing PAGE with a few minor lower molecular weight contaminants that became visible upon overloading (FIG. 1.3). This major band was excised from the gel and subjected to electro-elution. The electro-eluted excised band displayed 1.95° C. of thermal hysteresis activity. When re-electrophoresed on non-denaturing PAGE, it appeared as a single species having an identical R_{f} value (0.32) to the major band observed before excision (FIG. 1.3). Therefore, electro-elution of the major band on non-denaturing gel electrophoresis of gel filtration Peak 3 provided an effective means for purifying this thermal hysteresis producing fraction from its minor contaminants. Alternatively, more conservative pooling of the gel filtration, Peak 3 (tubes 21-24), produced a purer fraction. Only one band was observed on non-denaturing PAGE when 12.5, and even 25 ug of total protein were loaded (FIG. 1.4). Consequently, all further collections and applications of gel filtration Peak 3 have made use of this method of pooling.

Confirmation of Purity and Molecular Weight Determination. Purity of the thermal hysteresis producing fraction was determined by reverse phase HPLC, mass spectrometry and SDS-polyacrylamide gel electrophoresis. HPLC analysis of gel filtration Peak 3 indicated that gel filtration Peak 3 elutes as a single peak at 30 minutes with some peaking occurring between 12-15 minutes (possibility indicating the presence of minor organic contaminants) (FIG. 1.5). Subjecting the peak at 30-minutes to mass spectrometry confirmed that the profile seen on HPLC is of a single species having a molecular mass of 12,862 Daltons (FIG. 1.6). Note that the peaks at 6431.6 and 4292.5 are consistent with one half and one third the molecular mass of the single species. This purified species will hereafter be identified as Tm 12.86 (for Tenebrio molitor, 12.86 kDa). When gel filtration Peak 3 and the electro-eluted band off non-denaturing PAGE of gel filtration Peak 3 were run on SDS-polyacrylamide gel electrophoresis, Tm 12.86 was found to display an unusual migration behavior under the normal reducing conditions of SDS-PAGE. A distinct doublet was consistently observed between 12.7 kDa and 13.7 kDa when the electro-eluted sample off the non-denaturing gel was treated with sample buffer containing b-mercaptoethanol and run on SDS-PAGE (FIG. 1.7, lane w-w for with b-mercaptoethanol). The same result was seen when gel filtration Peak 3 was run on tricine and glycine SDS-gels. These results would suggest that the thermal hysteresis producing species in gel filtration Peak 3 is a dimer. However, HPLC and mass spectrometry analyses confirm the presence of only one species. Therefore, an alternative explanation for the presence of the doublet, is that it reflects a type of “shadowing” effect (DeVries, personal communication). To explore this possibility, the electro-eluted band off the non-denaturing gel was treated in sample buffer lacking b-mercaptoethanol and run on SDS-PAGE. As seen in FIG. 1.7, Tm 12.86 in lane w/o (w/o—for without b-mercaptoethanol) electrophoresed as a sharp singlet and co-migrated with the lower band of the doublet seen in lane w. The molecular weight of this band (approximately 12.7-12.9 kDa) is consistent with the molecular weight of Tm 12.86 as determined by mass spectrometry (12.86 kDa). Similar results were seen when gel filtration Peak 3 was run on SDS-gels without b-mercaptoethanol.

Compositional and N-Terminal Sequence Analysis. Amino acid composition of the 30-minute HPLC peak revealed that Tm 12.86 is a 117-residue protein that is high in hydrophilic amino acids (57.3 percent mole), especially in Asx (10.7%), Glx (15.0%) and Lys (14.9%), as illustrated in Table 2. Furthermore, Tm 12.86 would not be considered alanine-rich or cysteine-rich (3.9% and 3.2%, respectively).

TABLE 2
Amino Acid Composition and residue numbers for Tm 12.86

| Amino Acid | % mole | # residues | |
|---|---|---|---|
| Cys | 3.2 | 4 | |
| Pro | 3.0 | 4 | |
| Phe | 3.4 | 3 | |
| Ile | 4.4 | 5 | |
| Val | 8.5 | 11 | |
| Met | 2.0 | 2 | |
| Leu | 4.4 | 5 | |
| % Most | 28.9 | 34 | |
| Hydrophobic | | | |
| Gly | 3.1 | 7 | |
| Ala | 3.9 | 7 | |
| Tyr | 3.8 | 3 | |
| His | 3.2 | 3 | |
| Trp | ND | | |
| % Hydrophobic | 14.0 | 20 | |
| Asx | 10.7 | 12 | |
| Glx | 15.0 | 15 | |
| Arg | 3.6 | 3 | |
| Lys | 14.9 | 15 | |
| Ser | 6.8 | 10 | |
| Thr | 6.3 | 8 | |
| % Most | 57.3 | 63 | |
| Hydrophilic | | | |
| | | 117 | total residues |

(ND: not determined)

Amino-terminal sequence analysis for Tm 12.86 revealed the sequence for the first nineteen amino acids from the amino terminus SEQ ID NO 1 and indicated leucine as the amino-terminal amino acid (FIG. 1.8). This result provided added confirmation that Tm 12.86 is a single protein species. To investigate the possibility that a carbohydrate component was associated with Tm 12.86, an additional SDS-PAGE was conducted and stained with PAS.

Tm 12.86 failed to stain with PAS indicating that it lacks a carbohydrate moiety. Manduca sexta hemolymph was used as a positive control.

Thermal Hysteresis Activity Curve. Thermal hysteretic activity of Tm 12.86 was determined for various concentrations of the antifreeze protein (ranging from 0.125 to 25 mg/ml) (FIG. 1.9). Tm 12.86 reaches a saturation point of 2.3-2.5° C. at approximately 12.5 mg/ml (1 mM) and has a lower level of activity at approximately 0.125 mg/ml (10⁻⁵ M). For comparison, also depicted is the activity curve of a previously purified Type III thermal hysteresis protein from Tenebrio molitor (Table 1 T-4). Note that Tm 12.86 is shown to display a considerably higher level of activity than this previously purified antifreeze protein at similar concentrations.

Immunodetection of Tm 12.86 and Determination of Endogenous Hemolymph Concentration. Purified Tm 12.86 was used as an antigen to generate an antibody that has been determined to be very specific and sensitive for the antifreeze protein. FIG. 1.10 shows that Tm 12.86 is immunologically detected down to 1 ug on a Western blot using a 1:5000 serum dilution of this antibody. Moreover, the antiserum was found to react with only one protein species in T. molitor hemolymph (Lane H). Also illustrated, is that increasing amounts of Tm 12.86 are characterized by an increase in band width on a Westem blot.

By displaying winter-acclimated T. molitor hemolymph together with a serial dilution of purified Tm 12.86 (20, 15, 10,5, 2.5, 1 ug) on a Western blot, we have been able to indirectly estimate the endogenous hemolymph concentration of Tm 12.86 (FIG. 1.10). When a comparison of T. molitor winter-acclimated hemolymph was made to the serial dilution of Tm 12.86, a measure of band intensity estimated that 1-1.5 ug in 20 ug of total hemolymph protein loaded, or approximately 5-7.5% of the hemolymph protein, can be considered Tm 12.86. Furthermore, 1-1.5 ug in 0.5 ml of hemolymph estimated that Tm 12.86 exists at a physiological concentration of approximately 2-3 mg/ml (0.15 mM-0.23 mM) in winter-acclimated T. molitor hemolymph. Also noteworthy is that a hemolymph concentration of approximately 2-3 mg/ml for Tm 12.86 would contribute about 1.0° C. of thermal hysteresis to the 2.2° C. observed in winter-acclimated T. molitor hemolymph (FIG. 1.9).

Screening for an “Activator”. To investigate the possibility that other unknown factors may enhance Tm 12.86 activity, the existence of an antifreeze protein activating factor was sought by mixing Tm 12.86 with various peaks from the ion exchange column. Ion exchange Peak IV (FIG. 1.1a) was found to demonstrate an enhancement of activity beyond what would have been expected from the concentration of Tm 12.86 alone. However, this result could have either indicated the presence of an activating factor or may simply have been the additive effect of two different antifreeze proteins in solution (ion exchange Peak IV displayed a sizable amount of thermal hysteretic activity alone—approximately 0.5° C.) (FIG. 1.1b). Thus, ion exchange Peak IV was run on the Sephadex G-75 Superfine gel filtration column. Four peaks were eluted from the gel filtration column and tested for thermal hysteresis (FIG. 1.11). Only gel filtration Peak 3 displayed any antifreeze protein activity. Interestingly, even though gel filtration Peak 4 was not found to display any thermal hysteretic activity, when added to a known concentration of Tm 12.86 (3 mg/ml with 1.1° C. of activity), the level of antifreeze protein activity was nearly doubled (1.85° C. of thermal hysteresis) (FIG. 1.12). These results suggest that gel filtration Peak 4 of ion exchange Peak IV exhibits activator activity.

Investigation of the activation of Tm 12.86 by this activating factor indicated that activation occurs over all antifreeze protein concentrations (FIG. 1.12). At the lower level of antifreeze protein detectability (0.05° C. of thermal hysteresis at 0.01 mM of Tm 12.86) activation is nearly 10 fold (0.6° C.-10⁻⁵ M of Tm 12.8+12.5 mg/ml of activating factor). Furthermore, activation is more pronounced at non-saturated concentrations of antifreeze protein (0.125-12.5 mg/ml of Tm 12.86) and reaches a maximum level around 2-3 mg/ml of antifreeze protein concentration.

The fraction containing activator activity remains relatively impure. Characterization of the activator on gel electrophoresis is difficult because the substance does not seem to pick up Coomassie, Amido Black, and Silver stains. An ultraviolet scan of gel filtration Peak 4 of ion exchange Peak IV shows that the peak absorption for the main components in this fraction are 205, 240, and 275 nm (FIG. 1.13).

The above documents that a highly potent Type III AFP from T. molitor, designated as Tm 12.86 has been purified and characterized. The purity of Tm 12.86 has been confirmed by five separate criteria. On non-denaturing PAGE, Tm 12.86 was observed to run as a single band. Similarly, Tm 12.86 was observed to run as a single band under non-reducing SDS-PAGE conditions. Chromatography of Tm 12.86 on Reverse Phase HPLC yielded only one protein peak. Mass spectrometry of the 30-minute HPLC peak confirmed the presence of only one species at 12,862 Daltons. Finally, amino-terminus analysis of the HPLC peak revealed a single amino-terminus, leucine, defining Tm 12.86 as a single protein species.

Additionally, several lines of evidence, including amino acid composition, molecular weight, migration behavior on SDS-PAGE, and thermal hysteretic activity, indicate that Tm 12.86 is unique among Type III antifreeze proteins previously purified from T. molitor (Table 3). Amino acid analysis of Tm 12.86 indicates that it is characterized as a Type III peptide antifreeze because it lacks a high alanine content (3.9%), contains only a modest cysteine content (3.2%) and maintains a high percentage of hydrophilic amino acids (57.3%). This differs from the high cysteine content found in many previously purified insect Type II thermal hysteresis proteins (Table 1).

TABLE 3
Amino Acid Composition of Type III AFPs from T. molitor

| | | | | | | 2-2/2-3 |
|---|---|---|---|---|---|---|
| AFP's | T-4 | Tm 4 | AFP-3 | Tm12.86 | Tm13.17 | clones |
| MW (kDa) | 17.0 | 9.0 | 11.9 | 12.86 | 13.17 | 12.84 |
| Th C | 0.7 | 0.4* | ND | 25 | ND | ND |
| (30 mg/ml) | | | | | | |
| pl | | | | | 7.4 | 7.11 |
| # residues | | | 116 | 117 | 116 | 115 |

| | |
|---|---|
| Amino Acid | % mole |

| | | | | | | |
|---|---|---|---|---|---|---|
| Cys | 0.0 | 3.0 | 1.4 | 3.2 | 3.4 | 3.5 |
| Pro | 5.9 | 6.0 | 3.3 | 3.0 | 2.6 | 3.5 |
| Phe | 3.9 | 3.0 | 4.8 | 3.4 | 3.4 | 2.6 |
| Ile | 7.1 | 3.0 | 3.5 | 4.4 | 5.2 | 5.2 |
| Val | 11.5 | 7.0 | 5.8 | 8.5 | 12.0 | 12.2 |
| Met | 4.8 | 0.0 | 0.2 | 2.0 | 0.9 | 0.9 |
| Leu | 0.0 | 5.0 | 5.7 | 4.4 | 4.3 | 4.4 |
| % Most | 33.2 | 27.0 | 24.6 | 28.9 | 31.8 | 32.2 |
| Hydrophobic | | | | | | |
| Gly | 8.3 | 9.0 | 7.7 | 3.1 | 3.4 | 3.5 |
| Ala | 14.3 | 7.0 | 6.7 | 3.9 | 5.2 | 5.2 |
| Tyr | 2.3 | 3.0 | 1.6 | 3.8 | 0.0 | 1.7 |
| His | 1.9 | 3.0 | 4.7 | 3.2 | 0.0 | 1.7 |
| Trp | 0.0 | ND | ND | ND | 0.9 | 0.0 |
| % | 26.8 | 22.0 | 20.6 | 14.0 | 9.5 | 12.2 |
| Hydrophobic | | | | | | |
| Asx | 7.3 | 13.0 | 12.1 | 10.7 | 13.8 | 11.3 |
| Glx | 8.9 | 11.0 | 15.9 | 15.0 | 14.6 | 13.9 |
| Arg | 2.6 | 5.0 | 3.9 | 3.6 | 5.2 | 1.7 |
| Lys | 6.8 | 7.0 | 10.7 | 14.9 | 13.8 | 15.6 |
| Ser | 7.4 | 9.0 | 9.0 | 6.8 | 4.3 | 6.1 |
| Thr | 6.6 | 9.0 | 3.2 | 6.3 | 6.9 | 7.8 |
| % Most | 39.6 | 54.0 | 54.8 | 57.3 | 58.6 | 56.5 |
| Hydrophilic | | | | | | |

*Thermal hysteresis was conducted at 50 mg/ml.
ND: not determined.
T-4, from Table 1;
Tm 4: Schneppenheim and Theede, ([1980] Comp. Biochem. Physiol. 67B: 561).

The migration behavior for SDS-PAGE shows that Tm 12.86 runs as a distinct doublet when treated with sample buffer containing b-mercaptoethanol (a disulfide bond reducing agent) and a singlet under non-reducing conditions. This consistently reproducible profile for purified Tm 12.86 has never been described for another insect antifreeze protein and has been suggested to be the result of a “shadowing” effect (DeVries, personal communication). This effect may be the result of incomplete breakdown and/or reformation of disulfide bonds within the single antifreeze protein species. However, increasing the b-mercaptoethanol concentration in the sample buffer does not alter the density of the doublet.

Thermal hysteresis determination for Tm 12.86 indicates that Tm 12.86 is the most potent insect Type III AFP purified from T. molitor to date (Table 3) (FIG. 1.9). This may be attributed to its high percentage of hydrophilic amino acid residues (57.3%). Yet, several other previously purified antifreeze proteins from T. molitor also contain a high percentage of hydrophilic amino acid residues and do not display the same strong thermal hysteresis activity associated with Tm 12.86 (Table 3). Therefore, some structure or sequences specific to Tm 12.86 presumably confer its high level of thermal hysteretic activity. The factors contributing to this should be elucidated more upon determination of the complete amino acid sequence for Tm 12.86.

Surprisingly, the contribution of specific thermal hysteresis proteins to hemolymph antifreeze activity has never been addressed, so the approach used here with purified Tm 12.86 and an antibody generated against it, in Western blot analysis comparing winter-acclimated T. molitor hemolymph to a serial dilution of gel filtration purified Tm 12.86, addressed this void. The outcome determined the physiological concentration of Tm 12.86 as 2-3 mg/ml (0.15 mM-0.23 mM), or a mean of 2.5 mg/ml, for winter-acclimated T molitor hemolymph (FIG. 1.10). A concentration of 2.5 mg/ml contributes an activity of approximately 1.0° C. to a mean thermal hysteretic activity of 2.2° C. observed for winter-acclimated T. molitor hemolymph. This equates to a thermal hysteretic contribution of approximately 45% by Tm 12.86, indicating that it makes a substantial contribution to the antifreeze activity that T. molitor uses in its arsenal against freezing.

An activating component, defined by its own inability to cause thermal hysteresis, yet capable of significantly enhancing the thermal hysteretic activity when mixed with an antifreeze protein, has been identified for Tm 12.86. Thermal hysteretic enhancement of Tm 12.86 by this endogenous activating factor (at 12.5 mg/ml) occurs over all antifreeze protein concentrations and is most pronounced at non-saturated concentrations of the antifreeze protein (0.125-12.5 mg/ml) (FIG. 1.12). Moreover, maximum activation (0.75° C. of thermal hysteresis enhancement) occurs at approximately the physiological hemolymph concentration of Tm 12.86 in winter-acclimated T. molitor, 2-3 mg/ml of Tm 12.86. A mixture of 2.5 mg/ml of Tm 12.86 and 12.5 mg/ml of activator fraction contributes an activity of approximately 1.6° C. to an average thermal hysteretic activity of 2.2° C. observed for winter-acclimated T. molitor hemolymph. This equates to a thermal hysteretic contribution of approximately 72% by Tm 12.86 and its activator, suggesting that T. molitor may be precisely regulating Tm 12.86 and/or the activator for an efficient cold-hardy response.

Only a partially purified activating fraction has been obtained and characterization of the activating factor is limited. It elutes off the gel filtration column in the last peak, therefore appears to be a low molecular mass compound. Upon electrophoresis analysis on non-denaturing and SDS-PAGE the compound is not readily visualized using Coomassie brilliant blue, Amido black, Silver, and Panceau S stains. This may be consistent with the activator being small and, therefore, diffusing from the gel before fixing and staining. It's ultraviolet scan shows peak absorption at 205, 240 and 275 nm deviating only slightly from protein adsorption peaks at 230 and 280 nm, and activation by the activator is lost upon heat treatment, possibly indicating a specific amino acid recognition motif among the antifreeze-activator interaction. This low molecular weight activating substance is in sharp contrast to the 70 kDa activator protein purified from the pryochroid beetle, Dendroides canadensis (U.S. Pat. Nos. 5,627,051 and 5,633,451 to Duman).

The mechanism of action for the antifreeze-activator complex may be one in which the activator(s) flank Tm 12.86 because the greatest amount of thermal hysteretic enhancement occurs over non-saturated antifreeze protein concentrations. Presumably, unprotected ice crystal surfaces occur between neighboring antifreeze proteins at non-saturated antifreeze protein concentrations. Thus, a maximum amount of thermal hysteretic enhancement around 2-3 mg/ml of Tm 12.86 (0.75° C.) may set up the ideal spacing for antifreeze-activator(s) complex to efficiently blanket the surface of an embryo ice crystal.

Interestingly, specific antibodies to the purified D. canadensis THPs (Type II class) have been found to substantially increase thermal hysteretic activity of the THPs (U.S. Pat. No. 5,627,051, and Wu, D. W. et al. ([1991] Biochem. Biophy Acta 1076: 416-420; [1991] Comp. Physiol B 161: 279-283.), presumably by the THP-IgG complex blocking a larger surface area of the seed crystal than would be the case with the THP alone. Thus, it is foreseeable that the polyclonal antibodies included in the present invention directed against Tm 12.86, and functioning through a similar mechanism, could be used as an activating compound of Tm 12.86.

II. Generation of cDNA Libraries; Cloning Tm 13.17

The isolation and characterization of Tm 12.86, and the obtainment of a highly specific and sensitive antibody generated against it, were necessary prerequisites for implementing molecular studies to isolate the gene encoding for this AFP. Steps were taken to construct cDNA libraries from mRNA populations containing the message for Tm 12.86, from whole animal and fat body derived from cold acclimated T. molitor larvae according to the procedures detailed in Example 2. Immuno-screening with Tm 12.86 antibody identified a cDNA clone that was subsequently isolated and characterized (SEQ ID NO. 2) and found to encode for a distinct protein, Tm 13.17 (SEQ ID NO. 3 (precursor peptide) SEQ ID NO. 4 (mature peptide)). The N-terminal sequence of Tm 13.17 shows 61% identity, 83% similarity with that of Tm 12.86 (SEQ ID NO. 1), indicating that this clone is a homologous gene to that of Tm 12.86.

Total RNA and mRNA isolation. Total RNA was isolated from both intact larvae and fat bodies of T. molitor. Approximately 600 μg total RNA were yielded from 1.2 g of whole larvae or tissues. The quality and concentration of total RNA was measured by spectrophotometer, RNA scanning and by the analyses on agarose gel, or denature agarose gels. In general, there was no significant difference in the yield or purity of the total RNA isolated from whole insect and from fat body tissue. Result from spectrophotometer indicated A₂₆₀/A₂₈₀ absorbance ratio from 1.7 to 1.9. Confirmation of the A₂₆₀/A₂₈₀ absorbance ratio as derived from the scan curve of the isolated RNA was 1.8 to 1.9. In the 1% agarose or denature gels, 18S and 28S were clearly visible under UV light. But if the concentration was overloaded, 28S band became less sharp. No DNA or other contamination appeared in the gel. These results indicated the quality of the isolated total RNA is high and therefore was subjected to further experiments for mRNA isolation.

Results from spectrophotometer analysis of mRNA indicted the yield of mRNA was about 1 μg out of 100 μg of total RNA, i.e. within the expected range for the amount of mRNA in general. The A260/A280 absorbance ratio of the purified mRNA was 1.8-2.0, thus higher than that from the total RNA. The measure of quality and quantity indicated that the purity was increased after the process of the mRNA isolation. This is supported further with electrophoretic comparison of total RNA, mRNA and RNA remaining after mRNA removal [poly (A⁻)] (FIG. 2.0). Total RNA before mRNA isolation (lane 1) showed 18S and 28S, which was sharped further in the sample containing RNA minus mRNA (lane 2. In contrast, pure mRNA (lane 3) showed neither 18S or 28S band, but rather a smear bands with several different sizes of mRNA population.

In vitro translation products. mRNA isolated was subject to in vitro translation to identify whether it contains mRNA species encoding for Tm 12.86. Following in vitro translation, products were electrophoresed on 20% SDS-PAGE and visualized by flourography (FIG. 2.1). Many discrete peptides (lane 4 to 9) with apparent molecular weights ranging from more than 97 Kda to less than 14 kDa were synthesized under the direction of exogenous mRNA subject to in vitro translation. In vitro translation products of the isolated mRNA from winter acclimated whole animal (lane 4; 6 and 8) showed no apparent differences from that of unacclimated intact animal (lane 5; 7 and 9). No translation product was detected from the negative control (line 1, in the absence of exogenous mRNA).

To assay for the presence of a labeled Tm 12.86 translated peptide, four samples of the in vitro translation products were subject to immunoprecipitation. FIG. 2.2 presents the Coomassie stained immunoprecipitation samples (lane 1-4) together with orignal in vitro translation products (lane 6-9) from which the immunoprecipitation products were derived. FIG. 2.3 showed the fluorography of FIG. 2.2 displaying bands (lane 1-2) incorporation ³⁵S-methionine during in vitro translation, and that were immunoreactive to anti-Tm12.86. Also for each figure, samples in lane 1 and 2, and 6 and 7 were derived from T. molitor, while lanes 3 and 4, and 8 and 9 represented control samples, either containing all components of translation reaction, but without the addition of T. molitor mRNA (to identify any bands not due to the translation products from the mRNA of T. molitor), or another negative control was created by adding dH2O to mRNA of T. molitor instead of the complete in vitro translation reaction mixture. This control checked for contamination of the translation products from the mRNA solution. In FIG. 2.2, immunoprecipitation and in vitro translation products staining with Coomassie showed totally different patterns, yet no visible difference was seen between immunoprecipitation bands from translation products derived from T. molitor mRNA versus those derived from control, establishing consistency in products between samples and the lack of contamination. However, as seen in FIG. 2.3, only one single band (lane 1 and 2) was specifically detected by Tm 12.86 AFP antibody following immunoprecipitation of the T. molitor in vitro translation samples (lane 6 and 7). In contrast, no immunoprecipitation product was detected (lane 3 and 4) when the two control translation samples (lane 8 and 9) were immunoprecipitated with anti-Tm 12.86. Thus a labeled translation product recognized by the antibody to Tm 12.86 was identified as a product of in vitro translation of mRNA isolated from T. molitor. This established mRNA encoding for the Tm 12.86 AFP are present in the mRNA pool of cold acclimated T. molitor. Interestingly, the apparent molecular weight of the immunoprecipitated peptide is about 17 kDa, slightly higher than the purified protein (12.86 kDa) from intact T. molitor or from hemolymph although it must be remembered that translation product represent unprocessed peptides.

Results from total yield, agarose gel electrophoresis and spectrophotometer analysis all indicted a successful RNA isolation of high quality. Electrophoresis of total RNA also confirmed the presence of ribosomal RNA. Typically, two rRNA (28S and 18S) are abundant in a total RNA pool of eukaryotic organisms. Our results are consistent with this, yet compared to the 18S, the band of 28S RNA was not very sharp when a high concentration of samples were loaded on a 1% agarose gel. Also, some RNA masses larger than 28S were present indicting that there were some high molecular RNA in the isolated sample. These may be primary RNA since RNA molecules can be processed while transcription is still under way (cotranscriptional processing) or after transcription termination (post-transcriptional processing). The resolution patterns of total RNA in both the agarose and denature gels revealed similar patterns with respect to two bands representing ribsomal RNA 18S and 28S. However, the 18S band was stronger in the denature gel than in the agarose gel when the same amount of the sample was applied. The 28S band did not form a sharp band in the nondenatured agarose gel, but the resolution became better when it was separated from other kinds of RNA in the denatured agarose gel. These results suggest that artifacts caused aggregation, and secondary structure was reduced under the denaturing gel in the presence of formamide. Another reason that the 28S band may not have been sharper in the 1% agarose gel was that there was mRNA or some 18S RNA comigrating with the 28S RNA. Support for this is seen in the electrophoresis after mRNA was extracted from the total RNA (FIG. 2.0). This showed that 28S RNA band was much sharper and cleaner than before mRNA was extracted. Overall, the ratio of 28S to 18S RNA seems lower than that reported in the literature. In general, the ratio of 28S:18S is 2:1. However it is difficult to estimate the ratio precisely according to their appearance in a non denature agarose gel. It is also possible that insect RNA may have its own uniform ratio or breaking of 28S RNA during isolation.

Results from in vitro translation of these mRNAs confirmed that the isolated mRNA has a biological function. The rabbit reticulocyte system (Stratagene) with [³⁵S] labeled methionine has every element for protein translation in vitro except for mRNA. When the exogenous mRNA was added, the system underwent the translation of proteins based on the exogenous mRNA templates. These translated profiles obtained represent the protein populations encoded by the foreign mRNA. The results from in vitro translation indicated that many sizes of proteins were translated and that there was a rich profile of physically intact and fully functional mRNA capable of directing the synthesis of proteins in a heterologeous cell-free system.

Furthermore, the results of immunoprecipitation showed that a labeled Tm 12.86 translation peptide was separated from many different proteins of the translation products with apparent molecular weight is about 17 kDa, which is bigger than Tm 12.86. The difference in the molecular weight between the protein recognized by anti-Tm 12.86 from in vitro translation products and the protein purified from the insect suggests that Tm 12.86 is a posttranslation product in the insect. This observation is consistent with the result of N-terminal amino acid sequence analysis of the Tm 12.86, in which the first amino acid residue is leucine instead of methionine indicating that a cleavage process may have taken place. In contrast, in the in vitro system the protein synthesis is directed by foreign mRNA and no posttranslation processing is involved, therefore the protein recognized by anti-Tm 12.86 is slightly larger than the mature Tm 12.86. This documents that 1) the band represented a specific protein which has an epitope recognized by an antibody generated against Tm 12.86; and 2) this antigen expressed and detected during in vitro translation was derived for a mRNA species encoding a Tm 12.86 peptide product.

Therefore, these studies of RNA and mRNA isolation, in vitro translation and immunoprecipitation of a specific peptide detected by Tm 12.86 antibody, consistently suggest that the transcription of Tm 12.86 AFP gene is well expressed and accumulated under the chosen acclimation and experimental conditions. Moreover, we have successfully isolated a mRNA population containing the appropriate information. This provides the foundation for the next goal: cDNA library construction from the mRNA and cloning of the Tm 12.86 antifreeze protein gene.

Construction of cDNA. cDNA library construction involved many steps. Given this, it was important to make sure that each step or phase of the experiment was successfully accomplished, since the latter steps in library formation, and all subsequent experiments using the library are dependent upon the success and quality of the steps proceding them. Therefore, during the whole process of the construction of cDNA libraries, five sequential experiments were carried out to provide information on the quality of each phase of cDNA library preparation. Step 1 involved generation of single strand DNA. First strand cDNA was the product of reverse transcription of isolated mRNA using MMLV-RT and methyl dCTP. The mRNA template was then nicked with RNase H to serve as primers for DNA polymerase I to synthesize the second strand. The syntheses of the first and second strands were tested by electrophoresis of the products in 1% agarose gel and staining by ethidium bromide. Large amounts of DNA were visible in the gel following the synthesis of the first and second strands indicating that both the first and second strands were abundantly synthesized. Double stranded cDNA fragments were then prepared with the proper adaptors and subjected to size fractionation to yield a total of 5 fractions, each with different size cDNA fragments. Two of them were obtained from fat body mRNA as the starting templates [F 1+2 (FB) containing relatively larger cDNA fragments and F3 . . . 6 (FB) containing smaller ones]. Additionally, three different sizes of the fragments were gained when mRNA from whole animal were used as the starting templates [F1+2 (WB); F3+4 (WB) and F5+6 (WB) from large to small fraction size, respectively]. cDNA from each of these 5 fractions were quantified in a simple ethidium bromide plate assay to determine the concentration of cDNA after spinning the columns, and were examined to check sizes of fragments in 1% agarose gel. The results documented that the pool of synthesized cDNA is very diverse in size suggesting that it represented the whole population of mRNA.

Each of the 5 size fractions of cDNA were then ligated into a cloning vector, thus establishing 5 cDNA libraries, 2 for fat body and 3 for whole animal. We examined the ligation efficiency of the synthesized cDNA in the ZAP express vector arms after packaging with Gigapack m packaging Extract. After the packaged DNA was transinfected into XL1-blue MFR′ strain, the clone with cDNA insert could be distinguished with its white color plaque (clones without inserted cDNA show blue color). The plaque formating units (pfu) of the five libraries were determined to be 10⁸-10⁹ pfu/ml after amplification. The efficiency of recombinants in the libraries was 78-98% as indicated by the white plaques suggesting that most of the plaques contained the insert cDNA. There was no significant difference among cDNA libraries made from different mRNA sources or different sizes of fractions in regards to the titer and ratio of white to blue plaques. The above results illustrate that these cDNA libraries have a high titer and high ratio of recombinants. This suggests that these cDNA libraries possess genes that are representative of the tissue.

Screening the libraries. We chose to initially concentrate on the two cDNA libraries [F5+6 (WB) and F3 . . . 6 (FB)] containing the smaller size cDNA libraries because they likely held cDNA fragments less than 1 kb and therefore the appropriate size range for a gene encoding Tm 12.86. The libraries were screened with antiserum raised against Tm 12.86. Twenty plaques were recognized by the antibody during the first plating of phages (5×10⁴ plaques per 150 mm plate). To isolate pure single plaque the twenty plaques were further screened. More than 30 single positive plaques were detected and isolated during the second and third rescreening with the same antibody probe at a low plating density (300-500 plaques per 150 mm plate). The results showed that several plaques from both F5+6 (WB) and F3 . . . 6(FB) cDNA libraries expressed a specific antigen recognized by the antiserium of Tm 12.86.

Excision of cDNA from single phase plaque and analysis of the clones. We selected 7 single positive plaques (same from each of the 2 cDNA libraries) out of more than 30 for excision of the cDNA clone. The cDNA insert within the ZAP express vector was excised in vivo with helper phages and then recircularized to generate subclones in the pBK-cmv phagemid vector. After excision the pBK-cmv phagemid vector (FIG. 2.4a,b) was produced. This was used to infect XLOLR cells (strain of E. coli), and following infection with the pBK-cmv phagemids many colonies appeared on the LB agar plates with kanamycine as selected marker. Since only those colonies with kanamycin resistance containing the pBK-cmv double-stranded phagemid vector with inserted cDNA could replicate themselves in the kanamycin selected plates, these results confirmed that inserts from each of 7 positive plaques expressing antigen against Tm 12.86 antiserum have been obtained.

Following the excision, the plasmid containing cDNA were extracted from the colonies and electrophoresed in 1% agarose gel. The results indicated that all of the seven clones are recombinant plasmids and all were slightly bigger than the vector (4,518 bps). To check the size of the inserted cDNA for each of them, the isolated plasmid DNA were digested with either Xho I or EcoR I or both. FIG. 2.5 shows the restriction enzyme patterns obtained. After digestion with the two enzymes, two bands were seen in the 1% agarose gel (lane 1), one was the insert DNA about 500 base pairs and the other much larger one was pBK-cmv phagemid vector. From either one enzyme digestion (lane 2 and 3), the linear size of fragment was as large as pBK-cmv plasmid vector. The figure also showed the linear, supercoil and nick forms of pBK-cmv plasmid DNA with insert DNA (compare lane 2, with lane 4 and 5). The seven clones have been designated as FB1, FB2, FW1, FBW1,FBW2, FBW3 and FBW4 respectively.

DNA sequence analysis and similarity search. All of the seven clones were initially partially sequenced manually from both strands. All were found to have identical DNA sequence. The FW1 clone was then selected for a complete DNA sequence determination of both strands by automatic sequencing. The nucleotide sequence (SEQ. ID No 2) and deduced amino acid sequence (SEQ. ID Nos 3 and 4) of FW1 is presented in FIG. 2.6a. The full length of the cDNA of the FW1 clone is 577 nucleotides long and contains the cloning site E coR I at position 13 and XhoI at position 530. From the partial sequences of the 6 other clones, no sequence heterogeneity was found from that of the clone of FW1, indicating they all contain the same insert cDNA of T. molitor. There is one open reading frame (ORF) from the 577 base pairs. Its start codon ATG is 35 nucleotides downstream from the 5′-end of the clone and the stop codon TGA is at the position of 438 base pair. The 402 nucleotides within encode a peptide containing 134 amino acid residues with a molecular weight of 15.128 kDa. This also includes a putative signal peptide at the N-terminus with 18 amino acid residues, which shows characteristics typical of other signal peptide sequences, including three distinct regions: a basic positively charged N-terminal region (n-region); a central hydrophobic region (h-region) and a more polar C-terminal region (c-region) (FIG. 2.6b). Thus, the predicted mature protein is of 116 amino acid residues (SEQ ID No. 4), with a molecular weight of 13.17 kDa derived from 348 nucleotides. The mature peptide is designated as Tm13.17 for T. molitor 13.17 kDa molecular weight. The 3′-end untranslated region of 139 nucleotides is A-T rich (A:T:C:G=55:31:27:26) and presents a AATAAA polyadenylation signal which is located 49 nucleotides downstream of the stop codon and 13 nucleotides upstream of the poly (A) tail. The poly (A) tail occurs 26 nucleotides downstream of the polyadenylation signal.

The details and analysis of amino acid composition of the mature Tm 13.17 protein is presented in FIG. 2.6c. The mature peptide is predominantly hydrophilic (Asp, Asn, Glx, Arg, Lys, Ser, Thr, 58.6%) and rich in lysine (13.8%), glutamate (11.2%) and valine (12.1%), but appears to lack histidine and tyrosine.

The search of data bases of the proteins in FASTA and Genetics Computer Group version 7.1 programs reveals that the protein encoded by the clone FW1 is most closely related to the B1 protein of T. molitor (Paesen G. C., and G. M. Happ[1994] Insect Biochem. Molec. Biol. 25: 401-408) and moderately similar to AFP-3 of T. molitor. The B1 protein represents one of the four major protein groups secreted by the tubular accessory glands of adult male T. molitor, and presumably plays a role as a putative receptor of pheromones. AFP3/THP12 is uncertain; once thought to be an antifreeze protein, it's function is now in doubt and appears to be is a small lipid carrying protein (Rothemund et al., [1997] Biochemistry 45: 13791-13801; [1999] Structure 7:1325-1332). FIG. 2.7 shows the sequence alignment between the mature Tm 13.17 and B1 protein. There is a calculated 49% identity or homology between these two proteins, and 73% similarity between conservative replacement amino acids. The relatedness between Tm 13.17 and AFP-3 shows a lesser match, with 39.8% identity and 58.3% conservation replacement (FIG. 2.8). This more moderate homology is also seen between AFP-3 and B1 proteins with 39% identity and 57% similarity. Signal peptides of Tm 3.17, AFP-3 and B1 protein are highly similar (FIG. 2.9). Six out of the first 7 amino acid residues are identical between Tm 13.17 and AFP-3. However, B protein signal peptide is shorter (12 amino acids)) than Tm 13.17 and AFP-3 (18 amino acids).

Similarity of the NH2 terminus between Tm 13.17 and Tm 12.86. A comparison of the N-terminal sequence of Tm 13.17 with that determined from protein analysis of Tm 12.86 (SEQ ID NO. 1) indicates a very strong degree of relatedness (FIG. 2.10). 11 out of 18 N-terminal amino acid residues are identical between Tm 13.17 and Tm 12.86. Moreover, in addition to the identical amino acid residues there are 4 highly conservative replacements. Thus, the N-terminus of these two AFPs shows an identity of 61% and similarity of 83%.

Expression of Tm 13.17 in E. coli. Because the clone was ligated to ZAP expression vector which contain the lac repressor (lac I^{q}) gene, the LacI protein blocks transcription from the lac Z promoter in the absence of the inducer IPTG. This means that a protein can be expressed only when: 1) the cloned gene of the protein is ligated in ZAP express vector and 2) IPTG is presented. Accordingly, an experiment was designed to examine the protein products of five samples of the clone gene with differing amounts (1-5 mM) of IPTG. Two control samples from XLOLR cell with or without IPTG were also tested for protein expression. The results (FIG. 2.11) showed that samples with the clone had positive reaction with antibody of Tm 12.86, while two control samples did not show specific immunoreactive bands. Also, the samples with IPTG had a higher level of expression of Tm 13.17 protein compared to the low level expression of the protein that appeared in the no IPTG samples. These results indicted Tm 13.17 is being synthesized in the induced condition. Also Tm 13.17 has sufficiently close epitopes to Tm 12.86 such that it is being recognized by the polyclonal antiserum generated against purified Tm 12.86.

Thermal hysteresis activity of Tm 13.17. The total protein concentration of the sample was around 2 mg/ml under the inducing condition described Example 2. Tm 13.17 was successfully expressed in the E. coli system based on the result from the western-blot. However, no antifreeze protein activity was detected, either with thermal hysteresis or inhibition of ice recrystallization behavior.

In screening of the two cDNA libraries containing the smaller size cDNA fragments derived from cold acclimated T. molitor we found several cDNA clones containing an insert with molecular weight about 550 base pairs that were recognized by anti-Tm 12.86. The DNA sequence data for the insert of one of these positive clones (FW1) clearly indicated that it is capable of encoding a peptide of 134 amino acid residues with molecular weight 15.128 KDa. The identification of the cloning sites, E coR I at position 13 and Xho I at position 530 indicates that the cDNA clone is the right product from the cloning process. With a characteristic start ATG codon 35 nucleotides downstream from the 5′-end and a stop codon TGA at the position 440 followed by a 3′-end untranslated region of 139 A-T rich (A:T:C:G=55:31:27:26) nucleotides containing AATAAA polyadenylation signal and the poly (A) tail, these indicate that the cloned cDNA carries complete genetic information of the coding region of the gene. The existence of a 18 amino acid putative signal peptide proceeding of the mature protein shows a common feature among fish AFPs.

The signal peptide identified in the deduced amino acid sequence of the cDNA clone (FW1) shown in FIG. 2.6b, possesses characteristics of a peptide destined for vesicle transport, and are consistent with AFPs being secreted. Most of the characterized AFPs secreted across the endoplasmic reticulum contain the characteristic signal peptide composed of three structurally and functionally distinct regions as the basic building blocks of a signal sequence. A basic positively charged N-terminal region (n-region) is required for efficient translocation, a central hydrophobic region (h-region) is critical for translocation and a more polar C-terminal region (c-region) specifies the signal peptidase cleavage site. Because the c-region is particularly important for specifying the site of cleavage, only certain amino acids are fitted at position −3 and −1 of the region. The residue in position −1 must be small, i.e. either Ala, Ser, Gly, Cys, Thr, or Gln; and the residue in position −3 must not be aromatic (Phe, His, Tyr, Try), charged (Asp, Glu, Lys, Arg) or large and polar (Asn, Gln). The putative signal peptide of 18 amino acids of Tm 13.17 perfectly fits the characteristics mentioned above. It can be divided into three regions, n, h, and c regions. The cleavage site is at position between residues 18 (Ala) and 19 (Leu) of precursor. The amino acids of Ala and Val at precursor position 16 and 18 (or −3 and −1) fit the “(−3, −1)-rule” required for the amino acid in these positions. Thus, the structures of the three regions match well to the classic signal peptide.

The signal peptides of the Tm 13.17, AFP-3 and B1 proteins show high similarity. However, it seems that the signal peptides of Tm 13.17 and AFP-3 are more closely related. Both of them contain 18 amino acid residues and 6 out of the first 7 amino acids are identical. They end with alanine as the last residue of the signal peptide for the putative cleavage during the process of the protein secretion. In contrast, the signal peptide of B1 protein only contains 12 amino acids with less relatedness to that of Tm 13.17 and AFP-3. However, it is unknown whether this difference plays any significant role in secretion of the B1 protein from Tm 13.17 and AFP-3.

The composition of amino acids indicates that Tm 13.17 should be classified as a Type III AFP. Table 3 presents a comparison of molecular weight, thermal hysteresis activity, and amino acid compositions of previously identified Type 3 THPs from T. molitor, together with the information of Tm 13.17. In the most hydrophobic group amino acids, Tm 13.17 is most similar to Tm 12.86. Tm 13.17 and Tm 12.86 also show a high percentage of hydrophilic group amino acid residues. More than 13 percent of lysine residues are found in these proteins, however, they have a lower percentage of serine residues compared to the other Type III AFPs of T. molitor. Therefore, according to the pattern of amino acid composition and richness of particular amino acids, it appears that there are at least two distinct groups among all of the known T. molitor Type III proteins. Tm 13.17 and Tm 12.86 form one group with very high hydrophilic residues (>57%), valine-rich in hydrophobic groups, and a modest percent of cysteine residues. Note that AFP-3 does not share these features and so appears closer to the remaining other Type III AFPs. Since Tm 13.17 shares a similar pattern in amino acid composition with Tm 12.86, even though no thermal hysteretic activity has been detected for Tm 13.17, its strong relatedness to the other protein supports that Tm 13.17 could have a similar function to Tm 12.86.

Although Tm 13.17 has similar characteristics as other AFPs of T. molitor as discussed above, it also shows its own distinct features regarding amino acid composition. Compared to other AFPs identified in T. molitor, the peptide of Tm 13.17 has the highest percentage of the most hydrophilic amino acid group (58.6%) and lowest percentage of the middle hydrophobic group amino acids (9.5%) (Table 3). The latter may be due to the fact that Tm 13.17 has no histidine and tyrosine residues. Furthermore, Tm 13.17 displays the highest percentage of valine residues (12%). These distinctions may provide an explanation of why Tm 13.17 shows more relatedness to the accessory gland protein B1, than to AFP-3. In fact, the most homology among these AFPs is found between Tm 13.17 and Tm 12.86. They share identical or highly conserved replacement amino acid at their NH₂ terminus, yielding an 83% similarlity (FIG. 2.10, Table 3). Also, Tm 13.17 possesses sufficiently close epitopes to be recognized by anti-Tm 12.86.

Interestingly, an analysis of the sequence alignment of Tm 13.17, AFP-3 and B1 proteins show many highly conserved regions (FIG. 2.12). Significantly, 4 cysteine residues located in 4 different places are completely conserved among the three proteins. It has been reported that the cysteine residues in fish Type II AFPs are also completely conserved. Cysteine is reported to be involved in formation of disulphide bonds of AFPs in fish, however, whether this feature has any significant biological meaning for the present study is unknown. The exact function of B1 protein is unknown although it is proposed to be a putative receptor of pheromones of T. molitor. The similarity between B1 and Tm 13.17 raises a question of whether Tm 13.17 or other AFPs may play other significant roles in T. molitor physiology in addition to their antifreeze activity and importance to winter survival.

To show antifreeze activity of Tm 13.17 encoded by the cDNA clone, the protein expressed from the clone FW1 were extracted and tested for thermal hysteretic activity. The recombinant product did not display any antifreeze activity. The failure to detect antifreeze activity for the protein expressed from bacterium is not unusual. Actually, it is very common that a eukaryotic protein can be well expressed in prokaryotic system but with no original biological activity due to several reasons including a proper folding and posttranslation modification. Recombinant protein expressed in prokaryotics is not always able to fold into their native three-dimensional conformation. Another possible factor is that the degradation together with inefficient translation may cause low recombinant protein accumulation in bacteria, however the western analyses do not support this.

It is quite surprising that a corresponding cDNA clone for Tm 12.86 was not found from the screening of cDNA libraries. The cloning of the AFP gene in this study is based on the premise that any antigen produced as a result of a cDNA clone and recognized by Tm 12.86 antibody should identify clones containing a cDNA encoding Tm 12.86. However, none of the deduced amino acid sequences from seven positive cDNA clones examined in this study matched the N-terminus sequence of Tm 12.86 AFP although a substantial similarity was observed. There are several possible reasons to explain the unexpected results. First, the strong homology between Tm 12.86 and Tm 13.17, including sufficient similarity of epitope has allowed both molecules to be recognized by Tm 12.86 antibody. The degree of recognition may depend upon the immunological assessment conditions, i.e. the sensitivity of the system, the amount of antigen and the concentration of the antiserum. It is possible that the conditions designed for the cloning in this study was favorable to detection of 13.17 AFP. We noticed that no strong positive cDNA clones were found when a 1:2000 dilution of the primary antibody was used for the cDNA library screening. However, the same dilution of primary antibody reacts very well with Tm 12.86 in the westem-blot. In order to have a positive screening of the cDNA libraries a higher concentration of Tm 12.86 antibody was used (1:1000), which resulted in several strong positive clones. These positives would represent a combined profile of both Tm 12.86 and Tm 13.17. Thus, it may have been random chance that the seven positive clones examined in this study encoded for Tm 13.17. Alternately, perhaps expression of Tm 13.17 in cDNA clones is better than that of Tm 12.86, thus, the strong positive clones screened were exclusively Tm 13.17. It is of interest therefore to see if the polyclonal antibody to Tm 12.86 contains monospecific IgG's for Tm 12.86, and monospecific IgG's for Tm 13.17.

Equally possible, the transcription of Tm 12.86 mRNA may be not have been as abundant as that of Tm 13.17 under the conditions studied (3 week short photoperiod, cold acclimation). However, since the Tm 12.86 has been successfully isolated from the insect under these conditions, in theory, the level of transcription of Tm 12.86 should be abundant at this time, thus, this may not be the major factor of why the cDNA clone for Tm 12.86 was not found in the cDNA libraries. However, since we have no information on the time course of appearance of AFP, mRNA versus AFPS, this cannot be rule out as a factor. Also, at this moment it is not clear how abundant expression of the Tm 13.17 is in the insect. During the purification of Tm 12.86 several other fractions displayed AFP activity, consistent with the fact that the insect produces several different AFPs. Each protein purification procedure designed has its own desire to purify a particular protein. Even slight difference in features of two proteins will result in successfully purifying one protein but not the other. At present our only identification of Tm 13.17 is via a reaction to anti-Tm 12.86, and since these protein likely co-migrated on 1 dimension western analysis, we may never have detected the presence of an additional protein. Northern analysis with the Tm 13.17 cDNA clone, or 2 dimensional gel electrophoresis analysis is expected to facilitate clarification of these issues regarding the production of Tm 13.17 in the insect both at the transcriptional and translational levels.

Screening of the cDNA libraries to isolate the cDNA clone of Tm 12.86 requires either manipulation of the immunological screening conditions, or colony hybridization screening by using degenerated oligonucleotide probes derived from amino acid sequence of Tm 12.86 or DNA fragment directly from Tm 13.17 cDNA clone under different stringency conditions. Another possible reason that we did not find a Tm 12.86 clone may be because Tm 12.86 might be a posttranslation product of a larger gene in T. molitor. Recent studies have found that protein granules from the fat body (a site for THP storage) show several larger molecular weight immunoreactive bands, in addition to Tm 12.86/Tm 13.17. Since the present study only examined the two cDNA libraries [F5+6 (WB) and F3 . . . 6(FB,)] which had small sizes of cDNA fractions (smaller than 1 kB), it would be very unlikely to have cDNA clones in these two libraries capable of encoding a relatively larger protein (more than 30 kD). However, havinbg a cDNA sequence for Tm 13.17, it is possible to conduct northern analysis to determine the number and size of transcrits of Tm 12.86 and Tm 13.17 separately.

The cloning of a putative antifreeze protein gene for T. molitor was found to be a homologous gene to that encoding for Tm 12.86. The clone encodes for a precursor of 15.128 kDa and a mature peptide of a 13.17 kDa, with sufficiently close epitopes to be recognized by anti-Tm 12.86. The discovery of a homologue to Tm 12.86 and comparative sequence analysis between the N-terminal of Tm 13.17 and Tm 12.86 suggests the presence of multigene family of Tm 12.86 like genes in T. molitor. The presence of multigene families for antifreeze proteins have been described previously for fish antifreeze proteins. Interestingly, the lesser relatedness of Tm 13.17 to AFP-3 suggests that the latter represents a more distantly related member of this multigene family from T. molitor. This might also imply that the AFP-3 genes diverged from the Tm 13.17/B1 group at an earlier evolutionary stage, than did the Tm 13.17 gene and its homologous gene for Tm 12.86 separate from the B1 gene. While divergence of Tm 13.17 from Tm 12.86 genes would have occurred the most recently. It is possible that Tm 13.17 and its homologue Tm 12.86 may display differential patterns of biosynthesis; intracellular localization; and secreted levels; differences in environmental controls for these cellular responses; and of potency in antifreeze activity. Any or all of these may have significant impact on the overwintering response of the species.

III. Isolation of Clones 2-2 and 2-3

The invention includes several clones of the Tm 12.86 family of genes that have been isolated and characterized from the cDNA libraries generated as initially detailed in Example 2. Description of two of these clones (2-2 and 2-3) are detailed below, based on the procedures presented in Example 3. These clones have been isolated from two of the cDNA libraries not originally screened in Example 2. Primary immunoscreening of the F₁₊₂ phage cDNA library (at 50,000 pfulml phage density) resulted in the identification of ˜22 immunopositive plaques. Two of these plaques, designated 2-2 and 2-3, were removed for further immunoscreening of phages to ensure purity. Phagemids (pBK-CMV) were excised from 2-2 and 2-3 phages and ultimately transferred to XLOLR E. coli, also designated as 2-2 and 2-3 clones.

EcoRI and XhoI restriction endonuclease double digests of the 2-2 and 2-3 pBK-CMV phagemids revealed the presence of cDNA inserts of very similar or identical sizes for both 2-2 and 2-3. A comparison to the HindIII digested lambda DNA markers showed that the 2-2 and 2-3 cDNA inserts are somewhat less than 564 bp. in size. No internal EcoRI or XhoI locations were apparent for either 2-2 or 2-3 based upon gel electrophoresis results. The smaller sizes of the 2-2 and 2-3 cDNA inserts was rather unexpected, since the larger cDNA fraction of F₁₊₂ was used for screening in this case. However, there is likely to be considerable overlap between the various cDNA library fractions. A second EcoRI and XhoI double digest comparing the 2-2 cDNA size with the Tm 13.17 cDNA size revealed a visible difference in the gel electrophoresis results. The Tm 13.17 cDNA and 2-2 cDNA appear to be approximately 500-510 bp. and 470-480 bp. respectively when comparing R.E. digest fragment bands to low molecular weight DNA standards.

Nucleotide sequencing for clones 2-2 (SEQ. ID NO. 5) and clone 2-3 (SEQ. ID NO. 6) and predicted amino-acid residues (SEQ. ID NO. 7 and 8) for clones 2-2 and 2-3 are shown in FIG. 3.0 for clone 2-2 and FIG. 3.1 for clone 2-3. The 2-2 cDNA insert consists of a sequence 482 bp. in length, while the 2-3 full cDNA sequence is 483 bp. in length. An evaluation of amino acid translation of the 2-2 cDNA sequence using all six possible reading frames revealed only one likely open reading frame (ORF) consisting of 133 amino acids. An identical amino acid sequence was deduced for 2-3. Toward the start of the ORF for 2-2 and 2-3, a sequence of 18 amino acids corresponds exactly with the amino terminus sequences of Tm 12.86 (SEQ. ID NO. 1, FIG. 1.8). Preceding this 18 amino acid sequence within 2-2 and 2-3 is another 18 amino acids (FIGS. 3.0 and 3.1) that constitute a putative signal peptide characteristic of proteins synthesized for export.

The size of the mature 2-2/2-3 protein after signal peptide cleavage is 115 amino acids constituting a molecular weight of 12,843 Daltons. Amino acid composition of the peptide from clone 2-2/2-3 is presented in FIG. 3.3. The protein has a predicted isoelectric point of 7.11. A comparison of amino acid compositions for 2-2/2-3, Tm 13.17, and Tm 12.86 is shown in Table 3. The compositions appear to differ slightly for all three cases, though an accurate comparison may be difficult to ascertain since the amino acid compositions for Tm 12.86 was obtained using chemical methods. Regardless, there are no major differences between the proteins. All appear to have a large proportion of hydrophilic residues (>55%), and are relatively cysteine-poor, as compared to the cysteine rich YL1, a Type II AFP from T. molitor (Table 1). In addition, no repeating structure in amino acid structure as encountered for YL14 is immediately apparent for 2-2/2-3.

An interesting characteristic of the 2-2 and 2-3 cDNA nucleotide sequences is the apparent existence of four nucleotide differences occurring within the ORF for 2-2 and 2-3 (FIG. 3.2). As might be expected since both the 2-2 and 2-3 sequences encode identical proteins, these nucleotide differences all occur at the third position of their respective ORF codons. Errors occurring during the reverse transcription process or subsequent replication processes cannot be dismissed entirely, however, though the likelihood of such errors seems remote. Errors in sequencing would appear to be more likely, yet in the case of 2-2 and 2-3, multiple sequencing reactions from two independent excisions were performed and nucleotide differences confirmed by consulting the raw data with nucleotide peaks.

Recombinant 2-2/2-3 protein characterized via Western blot shows that the protein is immunoreactive with the anti-Tm 12.86 anti-serum (FIG. 3.4). Negative controls consisting of bacterial proteins derived from XLOLR E. coli lacking any pBK-CMV phagemid did not show significant immunoreactivity (LANE F). Another interesting characteristic of the recombinant protein is the appearance of a doublet (two closely spaced bands) rather than a single band on the Western blots, a unique feature also occurring for purified Tm 12.86 (FIG. 1.7).

The 2-2 and 2-3 recombinant proteins are also observed to comigrate with purified Tm 12.86 and Tm 12.86 in hemolymph based on the results of the Western blots. This is a rather unexpected result since the recombinant protein is synthesized as a lacZ-2-2 (or 2-3) fusion protein (the cDNA is inserted within a lacZ gene on the pBK-CMV vector). Since the mature 2-2/2-3 protein in vivo is putatively 12.84 kD, very similar to the 12.86 kD of the purified THP, it is possible that the amino terminus of the lacZ-2-2/2-3 protein (including signal peptide) was cleaved by an E. coli peptidase. The 2-2/2-3 protein with signal peptide has a molecular weight of about 14.7 kD, which would be expected to migrate at a noticeably slower rate than the 12.86 kD protein. This is not observed on the Western blots, although a set of fainter bands is evident above the 17.8 kD marker indicating the possible presence of some lacZ-2-2/2-3 fusion product (FIG. 3.4). In addition, recombinant Tm 13.17 is also present on the blots as a comparison to 2-2/2-3 and Tm 12.86. The recombinant Tm 13.17 migrates at a discernably slower rate than 2-2/2-3 or Tm 12.86, with a major band appearing on the Western just below the 17.8 kD marker. Whether or not any post-translational modification of Tm 13.17 in E. coli occurs is difficult to ascertain. Whether or not 2-2 and 2-3 correspond to Tm 12.86 is still uncertain as well. The molecular weight of the putative 2-2/2-3 peptide (“Tm 12.84”) is very close to that of Tm 12.86. In addition, amino acid compositions between 2-2/2-3 and Tm 12.86 vary somewhat (Table 3). However, the total number of amino acid residues in the mature 2-2/2-3 peptide is 115, while the total number of residues for Tm 12.86 is 117.

The results with 2-2/2-3 indicate that they, together with Tm 12.86, and Tm 13.17 constitute homologous but distinct proteins derived from a family of closely related genes. Evidence of multigene THP families has been found recently for Type II insect THPs from T. molitor and D. canadensis and also for certain cold water fishes. The Western blots of other fractions eluted from the anion exchange column show the existence of additional strongly immunoreactive bands apparently distinct from Tm 12.86.

IV. Isolation of Clones 3-4,3-9, and 7-5

Sections I to III illustrate that Tm 12.86 AFP is a member of a multigene family, and the presence of additional homologous genes further support this. Addressing how many gene homologues Tm 12.86 has, and how these homologous genes may be arranged in the genome of T. molitor, for example whether they are in tandemly linked, or scattered throughout the genome is described here through Southern analyses and PCR of genomic DNA, using procedures detailed in Example 4. Additionally, further screening of the cDNA library discloses three new homologous genes. The nature and degree of relatedness of these genes will shed light on the character of the gene family and how it may have evolved.

A. Southern Analyses

Comparison of Probe Labeling Methods. Chemiluminescent detection in Southern analyses was unsuccessful at detecting hydribization profiles of the T. molitor Tm 12.86 family of genes. Psoralen Biotin labeling and detection methods proved exceptionally poor. Only nanogram or greater amounts of unlabeled cDNA could be detected on dot blots, not the picogram quantities needed to detect single gene copies on a Southern blot. Moreover, Southern analysis failed to detect bands in the genomic DNA. Only signals from the positive controls were observed. Ethidium bromide staining of restriction enzyme digested DNA show that it has been effectively cut. DIG labeling and detection system gave somewhat better results yet it also was found not to be sensitive enough for use on the T. molitor Southern blots. While it could easily detect one picogram of the Tm 13.17 cDNA on a dot blot, it could not easily detect the Tm 13.17 gene on the Southern blot. Ethidium bromide staining of the restriction enzyme digested genomic DNA on a gel showed that it had been effectively cut. Even at the lowest stringency, the homologues which are known to exist could never be detected. After much trial and error in the conditions of hybridization and washing, a single band could be seen. This band could only be seen when very large quantities of digested genomic DNA were loaded on the gel (50 micrograms or more), and under conditions of relatively low stringency (hybridization at less than 50C). In a digest with PvuII, a band appears at approximately 4000 base pairs. In a BamHI digest, a band also appears at a slightly higher molecular weight. With an EcoRI digest, a single band can be seen also at approximately 4000 base pairs. Even when stringency conditions were dramatically lowered, as in where hybridization took place at room temperature in a 50% formamide buffer, only one band could be seen in each lane, although one might have expected that several bands would be visible. At hybridization temperatures below approximately 30C in the Standard buffer, non-specific binding of the probe was observed in all the lanes of the blot. This was determined to be non-specific binding because the probe bound to the molecular weight markers as well as to most of the DNA in the lanes of restriction enzyme digests, making it impossible to distinguish any individual bands. Note, under the conditions used in these studies, the cDNAs 13.17, 2-2, and 2-3 cross hybridize very easily with one another, even under conditions of high stringency (FIG. 4.0). This should mean that all three would be visualized on a Southern blot under conditions of low stringency using any one of the probes. However since only one faint band could be seen using this labeling and detection system even under extreme conditions (i.e. very large digests and low hybridization conditions) another strategy of labeling and detection had to be utilized.

The outcome of Southern blotting with 32P labeled probes proved successful and showed a single, dense band at a molecular weight much higher than that of the control (a lane of the cDNA alone, around 400 base pairs) (FIG. 4.1). A relationship can be shown between the amount of DNA in the original digest, and the intensity of the band detected by the probe. Lanes with more DNA produce darker bands. These bands appear to be about the same size as the much fainter bands previously seen with the DIG labeled probes, around 4000 base pairs. The size differences of bands produced by cutting with different restriction enzymes and detected by the probed was often discernible and shown to be significantly different (FIG. 4.2). Since the molecular weight markers run on these gels were not detected by the probe, is not likely that the high molecular weight bands observed are due to non-specific binding of the probe to large amounts of DNA. Also, when the images are compared to the DNA on the original gel, the binding of the probe does not correlate with the areas of highest DNA concentration, making non-specific binding of the probe to the DNA unlikely. Ethidium bromide staining of the gel shows that the DNA has been effectively cut by the restriction enzymes. Unlike what is seen with non-specific binding, the highest concentrations of DNA do not grow progressively darker with increased film-exposure time. The bands seen on all Southerns were always close to the same size with some variation, with one exception. On one particular blot, several barely distinguishable bands were detected in a single lane. This was a lane cut with the restriction enzyme Hae III, known to cut the cDNA Tm 13.17. Although these bands could not be properly analyzed due to their ambiguity, they likely correspond to the same restriction fragments that would be seen in the cDNA digested with this enzyme. The smaller bands from the HaeIII digest are not seen on other blots (FIG. 4.2, lane 4), probably due to their faintness, or they may have run off the end of the gel.

The same single, large band was detected repeatedly on many Southerns (FIG. 4.2). No other bands were detected on these blots, so it stands to reason that both the Tm 13.17 and the 2-2 and 2-3 genes may be contained within the single band seen on the Southems. Identical blots were hybridized under the same conditions, one with the 32P labeled Tm 13.17 probe, the other with the 32P labeled 2-3 probe. Both blots showed identical bands of about 4000 base pairs (FIG. 4.3). Blot B shows a positive control of Tm 13.17 cDNA in lane one, while blot A does not have a visible positive control (the DNA presumably escaped from the well). The blots are otherwise exactly alike. The same band was hybridized no matter which probe was used, strongly suggesting that the genes corresponding to the probes are all contained within the one band. If there were a gene located in another area, the ease with which the probes cross-hybridize suggests it would have been detected at lower stringencies, but no other band was ever detected. Hybridization of the probe was shown to be specific by very faint hybridization to Dendroides canadensis, a coleopteran like T. molitor (FIG. 4.4, lane 5) and no hybridization at all to Manduca sexta (FIG. 4.5, lane 2) DNA, which is a lepodopteran (common name, tobacco hornworm). All probes consistently bound to the same area of the blots when any of the previously cited six base-pair recognition restriction enzymes were used to digest genomic DNA samples.

Since all restriction enzymes used that had six base-pair recognition sites failed to produce more than one band on a Southern, certain enzymes with four base-pair recognition sites were used in an attempt to cut apart the larger hybridizing fragment. The enzymes employed were HhaI and RsaI, and results showed that these enzymes do cut the hybridizing fragments into much smaller pieces, resulting in a smear-like band which contains fragments down to less than 50 base pairs in size (FIG. 4.5). Lane five, cut with HhaI, shows approximately seven distinct bands underlying the smear of fragments. The hybridization of the probe is specific by the complete absence of hybridization to a lane of EcoRI digested Manduca sexta DNA (FIG. 4.5, lane 2). All lanes contained identical amount of digested DNA, approximately 30 μg.

Synopsis of Southern Blotting. Overall, the results of the Southern blotting procedures show single bands of sizes approximately 4000 base pairs, varying only slightly according to which restriction enzyme was used. These high molecular weight bands were seen only with the more sensitive labeling and detection methods and were the only bands noted. Intensity of the bands increased under conditions of lower stringency, but no other bands ever appeared no matter how low conditions of stringency were. No bands were ever seen in the genomic DNA lanes with the Psoralen Biotin labeling and detection system. With the DIG PCR labeling system and chemiluminescent detection, only faint bands could be seen, and only one band per lane. These bands were only detectable when each lane of the gel contained about 50 micrograms or more of cut genomic DNA, and when stringency was very low. With the more sensitive 32P labeling and detection system, high molecular weight bands were clearly and easily seen. These bands are quite dense and wide compared to those obtained with the DIG labeled probes, although they appear to be about the same size. The intensity of the bands increased with the amount of genomic DNA in the lane, and the size varied slightly with the restriction enzyme used. These same bands hybridized to any of the probes, suggesting that all three corresponding genes are contained in this one fragment. When an enzyme that cut within the cDNA sequence was used, more than one band of smaller sizes could be produced, but these bands were much fainter and in fact almost indistinguishable. When enzymes that recognized four base-pair sites were used to digest the DNA for Southern blotting, the bands shift to much smaller molecular weights. This is due to the fact that the four base-pair cutting enzymes cut more often, resulting in smaller fragment sizes.

There was much difficulty in trying to produce Southern blot results with the Tm 13.17, 2-2 or 2-3 probes. The problems begin at DNA isolation. The T. molitor larvae from which DNA was isolated have dense, almost crystalline protein storage granules. These granules are difficult to break down with Proteinase K, and can easily lead to protein contamination in the DNA sample. Also, the genome of T. molitor is composed of nearly 50% non-coding satellite DNA, which means that DNA samples must be doubled in order to have the expected number of gene copies present. The DNA was also difficult to cut with restriction enzymes, at least in part because so much was needed of each digest in order to see a band after detection. This problem was solved by dividing up the digests into smaller amounts, and then recombining them, and by digesting with many times the amount of enzyme theoretically necessary, for long periods of time (24 to 48 hours).

Although the DIG labeled probe was able to detect its counterpart cDNA on a dot blot down to levels less than one picogram with chemiluminescent detection methods, it was very difficult to see even one faint band on a Southern blot with the same probe. Since the problem was not with the hybridization of the probe to its homologous sequence, or in the subsequent detection of the probe, the trouble seems to lie with the genomic DNA on the blot Either the DNA is blocked somehow from hybridizing efficiently with the probe, or the target gene is in very small copy number, or a combination of both difficulties.

Evidence from the Southern blot suggest that the putative gene family, or at least the three related genes Tm 13.17, 2-2 and 2-3 which are shown to cross-hybridize easily with one another under the conditions used in this study, are close together on the same chromosome. Even at the lowest stringencies of hybridization and washing, there is only one band visible when the restriction enzyme does not cut within the cDNA sequence itself. This band, when visualized with the 32P labeling of the probe, is always of a molecular weight of about 4000 base pairs, and is quite thick and dense. In these studies, the band hybridizes equally well with the Tm 13.17 or 2-3 probe. This evidence suggests that either all of the gene fragments presumably being detected are either very close in size, or that they are linked together in tandem, and the restriction enzyme used has not cut between them. The likelihood of genes that are spread throughout the genome being of such similar size as to not be distinguishable on a Southern blot is quite low. Many different restriction enzymes were used, and with genes that are located far apart from one another, the restriction sites for a particular enzyme are likely to be at varying distances with respect to each gene, thereby producing fragments of a different size for each gene. When genes are close together on the same chromosome, it is more likely that they will be together on the same restriction fragment, and thus be detected as one large band on a Southern blot. If one or more of the genes were on a different chromosome, it would be likely that these two copies or two genes would be seen as two separate bands, because the restriction sites on either side would be different distances from the gene, and the resulting fragments would be of various sizes. Ethidium bromide staining of restriction digests show that the genomic DNA is completely cut.

The fact that all three cDNA homologues cross hybridize with one another under high stringency conditions (i.e. 68 C) suggests that all of the corresponding genomic copies are being visualized on low stringency Southern blots. These three or more genes, however, are indiscernible as individual bands. It can be shown that the single band contains at least the genes for Tm 13.17, 2-2 and/or 2-3, because no other bands appear no matter which of the probes is used. The same single band is detected after hybridization with any of the three probes. Two identical blots, one hybridized with the Tm 13.17 probe, and the other with the 2-3 probe, both showed the same bands at around 4000 base pairs. Because of the apparent size of the fragments, it can be hypothesized that there are either approximately eight genes linked together with spaces of 100 base pairs or less between them, or there are fewer genes that contain introns, or are separated by larger gaps. This does not rule out the possibility of more homologues elsewhere in the genome or also linked with the known genes. There may be other homologues that are too different to hybridize efficiently with the available probes under the conditions used.

Specificity of the probes has been demonstrated by the fact that the 2-2 cDNA probe does not bind at all to large amounts (30 μg) of Manduca sexta genomic DNA, while it hybridizes heavily to the same amount of T. molitor DNA in three other lanes. The probe binding non-specifically to large amounts of DNA would have resulted in some detection in the M sexta lane. The 2-2 cDNA probe does show faint hybridization to 30 μg of Dendroides canadensis DNA, at about the same molecular weight as it hybridizes to T. molitor DNA. The hybridization conditions of the blots containing the M. sexta, and the T. molitor DNA were the same, suggesting that the slight binding to the D. canadensis DNA is specific to a homologous ortholog of the T. molitor gene(s). This is not entirely surprising, since D. canadensis, like T. molitor is a coleopteran, and thus they are more closely related than M. sexta is to either of them. The band seen in D. canadensis' lane may be faint because the DNA the probe has hybridized is more different from the probe sequence than it is in T. molitor. It may also be fainter because there are fewer genes located in the band. The duplication events that created the Tm 12.86 homologues in T. molitor may have happened after the phylogenetic split between the two insects, or there may be so much sequence divergence that the T. molitor probe does not recognize the D. canadensis gene(s) very well.

The restriction enzymes that recognize sequences of six or more base pairs failed to cut apart the Tm 12.86 family of genes, but it can be expected that if the genes themselves were cut up, the bands would move farther down the blot. Employing four base-pair cutting restriction enzymes such as HhaI and RsaI decreases the size of the hybridizing fragments on a Southern. Because the enzymes cut more often, and also cut several times within the known cDNA sequences, the genomic DNA is cut into smaller pieces, resulting in a smear with fragment sizes down to less than 50 base pairs. This was shown to be true in FIG. 4.5. HhaI cuts Tm 13.17, but not 2-2 or 2-3. RsaI cuts 2-2 and 2-3, but not Tm 13.17. The larger bands on the blot (i.e. 4000 bp) may be one or the other of these genes that is not cut by that particular enzyme. Since the blot of the DNA cut with these enzymes was hybridized under low stringency conditions, cross hybridization of the probes can be expected. It is not known whether these enzymes cut between the genes analogous to these cDNAs, or whether the smaller fragments result entirely from cuts within the known cDNA sequences.

Because so much genomic DNA is necessary to visualize probe hybridization to a Southern blot, it is likely that either this family of AFPs is in low copy number, or properties of the genomic DNA of T. molitor make it difficult for the probes to hybridize efficiently. The difficulty in obtaining Southern blot data for these genes, and the relative ambiguity of Southern results in this study made it necessary to use other means of characterization for this family of genes.

B. PCR Amplification of Genomic Fragments

PCR Amplification of Genomic DNA. With the first two PCR methods, several fragments were amplified initially using primers from the termini of Tm 13.17. Although these PCR products were not visible on an ethidium bromide stained gel, hybridization at 42 C with 32P labeled Tm 13.17 cDNA probe reveals a major band at about 3500-4000 base pairs (FIG. 4.7). Several larger, though fainter bands are visible above the major band. Various possibilities exist for what may be contained within these bands. The major band may contain only the Tm 13.17 cDNA, hybridized at low stringency to the Tm 13.17 probe, or it may contain many genes of approximately the same size, which were amplified simultaneously in part to a low (30C) primer annealing temperature during PCR. Because the PCR products could not be visualized with ethidium bromide staining, effects of primer annealing temperature on amplification could not be assessed. The larger bands seen on the Southern may be several genes amplified in tandem, as Southern blot evidence suggests is their configuration in the genome. In order to begin to differentiate these possibilities, several more experiments with the hybridization of PCR products to 32P labeled probes were conducted.

PCR products obtained when 1% DMSO was added to the reaction mixture were clearly visible on an ethidium bromide stained agarose gel (FIG. 4.8). This larger amount of product is probably due to the ability of DMSO to denature DNA, aiding in strand separation between elongation cycles. DMSO may also affect the melting temperature of the primers, but since PCR was attempted using many different primer annealing temperatures without DMSO to no avail, this is less likely to be the benefit of the added DMSO. The activity of Taq polymerase is inhibited by DMSO, but clearly the benefits this solvent confers to the amplification process in this case outweighs its negative effects.

The PCR products obtained by adding DMSO to the reaction mixture, from reactions using Tm 13.17 primers at low (35 C) primer annealing temperature, were run on a gel and Southern blotted. This time, hybridization was at medium stringency (50C), and blots were probed with the 32P labeled 2-2 cDNA probe. Results show hybridization to the PCR product, apparently at a slightly higher molecular weight than the bands hybridized by the Tm 13.17 probe (FIG. 4.9). These results suggest that the genes for both 2-2 and Tm 13.17 were amplified in the PCR, but Southern analysis suggests they are found at different molecular weights when the product is run on a gel. The 2-2 clone is already known to have several other nearly identical counterparts. If these genes are linked and are being amplified together, perhaps this explains the higher molecular weight of the PCR fragment they are found in.

Cloning of the PCR Products. The initial attempt to clone the PCR generated fragment of the Tm 12.86 gene family using the Prime PCR Cloner Cloning System from 5 Prime−→3 Prime, Inc was unsuccessful. This method failed to yield a significant number of recombinant bacteria. Transformed bacteria were plated on ampicillin, since the pNoTA vector they received conferred ampicillin resistance. Blue/white selection was the technique used to differentiate recombinant plasmids, which would appear as white colonies on a plate of transformed bacteria. In this case, through two separate attempts, all of the colonies remained blue meaning the LacZ gene located on the pNoTA plasmid they had been transformed with had not been interrupted by an inserted DNA fragment.

A second method used to try to clone the PCR fragments was by direct ligation into the p-Gem vector, which was supplied with the Perkin Elmer Terminator sequencing kit. PCR products were digested with Eco RI and ligated directly int the vector. This method resulted in many recombinant plasmids. However, upon sequencing some of these inserts, most were found to be T. molitor satellite DNA sequences, by BLAST comparison at GenBank. This is not surprising since more than 50% of the Tenebrio genome is comprised of satellite DNA, and all of this satellite DNA was in the 1 μg sample of genomic DNA added to the PCR. Additionally, the p-Gem vector is not specifically designed to clone larger fragments of DNA such as the 3500-4000 base pair fragments generated in these reactions, therefore, it probably favored the much smaller (300-500 base pairs) satellite DNA fragments. It is also possible that there were no EcoRI sites in the PCR generated fragments. Because of these difficulties, it was necessary to use a third method to try to clone and sequence the PCR products.

The TOPO™ XL PCR Cloning Kit (Stratagene) is designed to clone long (3-10 kb) PCR products. It uses the linearized and topoisomerase-activated 3.5 kb vector pCR(-XL-TOPO. Positive selection is with disruption of the ccdB (control of cell death) gene. This gene encodes the CcdB protein, which knocks out bacterial DNA gyrase, an essential enzyme that catalyzes the ATP-dependent negative supercoiling of DNA. Any bacterial cell that contains a plasmid without an insert to disrupt the ccdb gene will not survive, ensuring the selection of insert-containing colonies.

In this study, cloning large (4000 bp) genomic PCR products from T. molitor has been unsuccessful with TOPO™ XL PCR Cloning Kit and other previously mentioned methods. No colonies were obtained that contained insects of more than 50-100 bps. Although these results do not imply that cloning these fragments is unfeasible, the large size of the fragments and their relatively low concentration in the background of a microgram of genomic DNA template from the PCR adds to the difficulty.

C. Cloning Additional Homologues

A total of five new immunopositive clones were sequenced. Many more positive clones were observed (on average seven per plate in the primary screening), but due to the inherent difficulty in separating the positives from the background plaques, and the need for secondary and tertiary screenings, only five were eventually isolated. Out of the five, two of these clones appear to be false positives, since their sequences are unrelated to Tm 13.17 or 2-2 and 2-3. These may be due to endogenous peroxidases that were not completely knocked out by the peroxide treatment. The remaining three clones were nearly identical in nucleotide sequence to the existing 2-2 and 2-3 clones, and were designated 3-4,3-9, and 7-5 (FIGS. 4.10, 4.11, and 4.12) having SEQ ID NO's 9, 12, and 15 respectively, and encoding for peptides (precursor and mature) having SEQ ID NO's 10-11, 13-14, and 7-8, respectively for each clone.

The signal peptide of 3-4 is identical to that of 2-2 and 2-3, and the mature polypeptide predicted from the full length 3-4 cDNA (FIG. 4.10) is 115 amino acid residues in length. The 3-4 clone differs from the other Tm 12.86 homologue proteins only by one amino acid residue: the substitution of a valine for the cysteine residue 13 residues upstream from the stop codon. The molecular weight of 34 is approximately the same as 2-2 and 2-3, at 12.84 kDa.

The full length 3-9 cDNA (FIG. 4.11) predicts a mature protein of 115 amino acid residues, again with a signal peptide identical to 2-2 and 2-3. The 3-9 peptide differs from 2-2 and 2-3 at two residues (FIG. 4.14). There is a substitution of a glutamine for a valine 19 residues from the start of the mature protein, and a conservative substitution of an arginine for a lysine about midway in the protein sequence. These substitutions give 3-9 a predicted molecular weight of 12.871 kDa, larger than 2-2, 2-3, 3-4, and 7-5.

The full length 7-5 cDNA (FIG. 4.12) has an identical predicted mature protein to 2-2 and 2-3, and differs from both only at two nucleotide residues, which do not change any amino acid residues. Consequently, 7-5 has a molecular weight identical to 2-2 and 2-3, at 12.842 kDa.

It is likely that clones nearly identical to one another and to 2-2 and 2-3 make up the largest component of the cDNA library size division 1&2. Positive clones were selected randomly from the cDNA library, yet five out of six of these clones are nearly identical to one another. This suggests that the majority of the clones in this library are very similar to one another. This could be perhaps explained through a repetitive gene duplication event, or 2-2, 2-3, 3-4, 3-9, and 7-5 may be different alleles of the same or similar gens, resulting from the polymorphic population used to create the cDNA library.

D. Sequence Comparison for Relationship within the Tm 12.86 Multigene Family

As detailed, the homoloque Tm 13.17 was the first full length cDNA insert identified and characterized in the Tm 12.86 gene family. Although the predicted amino acid sequence at the N-terminal of Tm 13.17 is similar to that of Tm 12.86 (FIG. 2.10), the two are not identical, nor are their molecular weights. The NH2 termini of Tm 12.86 and Tm 13.17 have 11 out of 18 identical amino acid residues, with four highly conservative replacements, giving them a similarity of 83%. The Tm 13.17 cDNA clone (FIG. 2.6) is 577 nucleotides long, with the start codon (ATG) 35 nucleotides downstream from the 5′ end. The stop codon is at the 438 base pair position, with 402 nucleotides encoding a 134 amino acid peptide of 15.128 kDa, including the putative signal peptide of 18 amino acid residues. The signal peptide shows typical characteristics, including a basic (+) charged N-terminal region, a central hydrophobic region, and a more polar C-terminal region. The predicted molecular weight of the 116 amino acid protein is 13.17 kDa, and it is followed by an AATAAA polyadenylation signal 49 nucleotides downstream of the stop codon, and 13 nucleotides upstream of the poly (A) tail.

Also detailed, a BLAST search of GenBank has revealed that Tm 13.17 shows the most relatedness to the B proteins of the tubular accessory sex glands of the male T. molitor, and FIG. 2.7 displays the nucleotide sequence alignment between Tm 13.17 and B1. Tm 13.17 and B1 share 41% identity, and 73% similarity between conservative amino acid replacements. The B proteins, (B1 and B2), are one of four major protein groups secreted by the tubular accessory glands, and have a deduced molecular mass of around 13.3 kDa. The B proteins appear at about day eight of adult development, when they account for 42% of new protein synthesis in the tubular accessory glands. At other stages of development they are barely detectable. The B proteins are in turn significantly related to certain moth pheromone binding proteins in nucleotide and amino acid sequence. The function of the B proteins is still not known, but because of this similarity to pheromone binding proteins, and their presence in the tubular accessory glands of the male T. molitor where such binding proteins are likely to be found, it is likely that the B proteins are also pheromone or lipid binding proteins.

The similarity of the B proteins to Tm 13.17 is such that it is entirely possible that either the Tm 12.86 family of homologues are pheromone binding proteins themselves, certain of which are also able to act as AFPs by binding ice, or that these AFP genes are derived from pheromone binding proteins, changing their function from pheromone binding to ice binding. Thus, it is conceivable that the Tm 12.86 family of AFPs have two functions in T. molitor, or have evolved from a gene encoding a similar type of binding protein.

The 2-2 and 2-3 cDNAs, also identified by the antibody to Tm 12.86, share approximately 53% identical amino acids with Tm 13.17, and only differ from each other at six nucleotide residue, four in the open reading frame (FIG. 3.2). These nucleotide differences do not however alter amino acid sequence, therefore 2-2 and 2-3 both code for the same protein of 115 amino acids with a predicted molecular weight of 12.843 kDa (FIG. 3.3). Moreover, this protein has an identical N-terminal sequence to Tm 12.86. The rest of the Tm 12.86 amino acid sequence is unknown, but there are slightly different molecular weight and protein composition between the predicted proteins of 2-2 and 2-3, and Tm 12.86 (Table 3) Tm 12.86, at 117 amino acids in length, has two more residues than 2-2 and 2-3, at 115 amino acids.

Here three new cDNA clones were identified, also with the antibody to Tm 12.86. These clones, called 34, 3-9, and 7-5, are all very similar to each other and to 2-2 and 2-3. They differ at boxed nucleotide positions (FIG. 4.13), resulting in two distinct amino acid position changes in the predicted mature proteins for 3-4 and 3-9, while 7-5 is identical in amino acid sequence to 2-2 and 2-3 (FIG. 4.14). The nucleotide sequences of 3-4, 3-9, and 7-5 are 98-99% identical to those of 2-2 and 2-3. While 3-4 and 7-5 have predicted molecular weights of 12.839 kDa and 12.843 kDa, respectively, 3-9 has a predicted molecular weight of 12.871 kDa. Thus the predicted molecular weight of 3-9 is slightly closer to the measured molecular weight of Tm 12.86 than any of the other clones isolated so far. The amino acid compositions and other details of 3-4, 3-9, and 7-5 are found in FIGS. 4.10, 4.11, and 4.12, while a comparison of the amino acid compositions of all the Tm 12.86 clones to Tm 12.86 is seen in FIG. 4.15.

AFP-3 is another cDNA isolated from T. molitor (FIG. 2.12), and shown to encode for a small lipid binding protein, but still unresolved as to whether it is also an antifreeze protein gene (Rothemund S. et al., [1997] Biochemistry 45: 13791-13801]; [1999] Structure 7: 1325-1332). It is related to the Tm 12.86 homologues, having 39.8% identity with Tm 13.17 (57% similarity with conservative amino acid residue replacements), and consequently is more distantly related than 13.17 is to 2-2 and 2-3. Nevertheless, even this distant relatedness suggests AFP-3 may belong to the same multigene family. AFP-3 is 39% identical to the B proteins, sharing 57% similarity.

There are highly conserved regions of amino acids between Tm 13.17, 2-2,2-3, 3-4, 3-9, 7-5, B1, B2, and AFP-3, which may be important to their function (FIG. 4.16). Most notably, four cysteine residues are conserved in the protein coding sequence, and one in the signal peptide (FIG. 4.16) (B1 and B2 lacking the cysteine in the signal). The same cysteine residues are conserved when Tm 13.17, B1, B2, and 2-2, 2-3, 3-4, 3-9 and 7-5 are lined up with pheromone binding proteins from various insects (FIG. 4.17). Also, when the amino acid sequences of 2-2 (representative of 2-2, 2-3,3-4,3-9, and 7-5) Tm 13.17, B1, and AFP-3 are aligned, it can be seen that a mutation has occurred in one of the sequences which causes a shift in amino acid sequence by the addition or deletion of a single amino acid, or three nucleotides (FIG. 4.16, marked by an open circle). If it is assumed that the B proteins are ancestral to Tm 13.17, Tm 12.86, and the other Tm 12.86 clones, then the 2-2, 2-3, 3-4, 3-9 and 7-5 proteins must have evolved in part with a deletion of one amino acid at this position. Since these latter proteins are apparently the most similar to the antifreeze protein Tm 12.86, this deletion could be important to the function of Tm 12.86 as an antifreeze. Also, Tm 12.86 is apparently two amino acids larger than the predicted 2-2, 2-3, 3-4, 3-9, and 7-5 mature proteins. Since the full amino acid sequence of Tm 12.86 is not known, it is also not known where this two amino acid discrepancy is, or whether it is relevant to the function of the protein. It may be relevant that the 2-2, 2-3, 3-4, 3-9, and 7-5 proteins lack a significant hydrophobic domain beginning near residue 42 in Tm 13.17, B1, and AFP-3, as well as in certain insect pheromone binding proteins. Recently, comparative sequence analyses for insect Type II, high cysteine AFPs have been published. Type II, high cysteine AFPs from Dendroides canadensis [DAFPs] (Duman, J. G. et al., [1998] J. Comp. Physiol. B 168:: 225-232) show a high degree of similarity to the Type II Tenebrio AFPs YL-1-YL4 (Graham, L. A. et al., [1998] Nature 388: 727-728; Liou et al., [1999] Biochemistry 38: 11415-24)). The similarities are sufficiently high (48-67%) as to suggest that the same homologous gene family is present in the two different species of insects. For this to occur, these genes must have been in place before the divergence of the two species. If this is so, they should be found in all insect species diverging at the same time or after D. canadensis and T. molitor. In these Type II AFPs a pattern of cysteine repeats every six residues is conserved, and it is important to the function of the antifreeze protein in forming disulfide bridges, allowing for repeated units to be stacked side by side in a Beta helical structure (Liou, Y. C et al.,[2000] Nature 406: 322-324)). In the YL AFPs, it is postulated from Southern blotting data that there are 30-50 tightly linked copies of the AFP genes, differing in the number of repeated units. This pattern of gene duplication and tandem linkage is also seen in the unrelated fish AFP gene families. Between the cysteine residues, other patterns of amino acids are repeated as well, forming repeat units of 12 or 13 residues. In both fish and insects, AFP gene families tend to contain repeated units of a certain number of amino acid residues. These repeat units are most often originally taken from segments of existing DNA, coding or non-coding, and then amplified many times to create a novel gene. Often in Type I and Type II antifreeze proteins, the repeat unit is also the smallest unit necessary to bind an ice crystal and cause thermal hysteresis. After the first repeat unit has bound to the surface of the ice, other repeat units may follow in sequence. Homologous genes may simply be made up of different numbers of these repeated units.

The present invention details Tm 12.86 homologues that are similar to one another, and code for identical or similar proteins, but there are no obvious discernible repeat units in these genes. There is a possibility that the areas surrounding the conserved cysteine residues suggest ancient duplication, but this could also be expained by their importance in the functional mechanism of the protein. The cysteine residues are associated with one or more lysine and/or isoleucine residues on either side, as well as valine residues appearing somewhere after the cysteine residues (FIG. 4.18). The Tm 12.86 gene family is not closely related in nucleotide or amino acid sequence to any other known AFP families. There exists the possibility that the Tm 12.86 AFP homologues are actually serving a different purpose in the organism, and may serve in addition as antifreeze proteins, making them dual function proteins. Alternately, because the Tm 12.86 homologues are closely related to a pheromone binding protein, it can be hypothesized that this may have at one time been their primary function, and that their ability to bind pheromones, after a few key mutations, may have become secondary while a primary function became translated into their ability to bind ice, making some or all of them into thermal hysteresis proteins. In families of functional AFPs that have been amplified for the purpose of producing a greater amount of a certain protein, the genes should be nearly identical in order to conserve function. This is the case with most of the fish AFPs, as well as with the Type II insect AFPs. This also seems to be the case with the Tm 13.17, 2-2, 2-3, 3-4, 3-9, and 7-5 cDNA clones. All of these clones are very similar, or nearly identical to one another, in both nucleotide and amino acid sequence. This suggests that these genes have been duplicated by a mechanism such as unequal crossing over, resulting in several copies. One can form a hypothesis with regard to the nature of the Tm 12.86 homologues, based on evidence gathered from Southern blots, PCR amplification of genomic DNA, and sequence alignments. It is clear that the Tm 12.86 homologues are members of a multigene family, the members of which are located near one another on the same chromosome. The evidence for this statement is a) the consistent high molecular weight bands on the Southern blots, b) the fact that these same bands hybridize equally to all three cDNA probes, while no other bands are detected on the blot, and c) only the use of restriction enzymes which cut within the known cDNA sequences results in smaller band sizes.

There are several members in the Tm 12.86 family, based on the comparison of Southern blot and PCR data, as well as cDNA library screening. Six distinct clones have been isolated with strong relativeness to Tm 12.86. The size of the PCR product and the hybridizing band on the Southerms (about 4000 base pairs) allows for the presence of approximately six genes of around 500 base pairs in size, or less than six genes which contain introns or significant sequence between the genes. There may also be more than one 4000 base pair fragments present that can not be separated adequetely by the gel electrophoresis described here. Since the 2-2, 2-3, 3-4, 3-9, 7-5 and Tm 13.17 predicted proteins are significantly similar to the B1 proteins and insect pheromone binding proteins, they likely were derived from and/or shared a common ancestral gene with pheromone binding proteins. Given this, they may serve more than one function in the insect. The ability of Tm 12.86 to bind ice requires the acquisition of ice-binding domains, which somehow allow the protein to adsorb onto the surface of an ice crystal, perhaps by hydrophilic/hydrophobic interactions. The mechanism by which Tm 12.86 may bind ice is not known, but since it does not have a repeat unit structure like AFP Types I and II, its mechanism may be closer to those of the Type III or IV fish AFPs, or it may have a different, as yet undescribed mode of action. The transition of Tm 12.86 into an ice-binding protein could have been facilitated by its ability to bind something else, such as pheromones or other lipid molecules. It is possible that some of these Tm 12.86 homologues or additional ones being isolated may not prove to be antifreeze proteins. This could be due to a problem such as incorrect protein folding in the bacterial host, or, if this gene family does serve more than one function, perhaps only one or a few of the homologues have evolved the ability to bind ice through certain mutations in the gene sequence. Also, it is likely that the Tm 12.86 gene family probably evolved from an entire gene, and not from de novo synthesis from part of a gene or a region of non-coding DNA. The relationship of the Tm 12.86 gene family to the B proteins, and insect pheromone binding proteins in general, suggests that this family arose from duplication and alteration of a pre-existing gene.

FIGS. 4.19 and 4.20 illustrate the known relationship between the Tm 12.86 homologues, the B proteins, AFP-3, and the Type II insect antifreeze proteins from T. molitor (YL-1) and D. canadensis (DAFP-1A). FIG. 4.19 shows two tables, the top one comparing nucleotide sequence identity, and the lower comparing amino acid sequence identity. In both nucleotide and amino acid sequence, 2-2, 2-3, 3-4, 3-9, and 7-5 are more than 98% identical to one another. Tm 13.17 is about 50% related to the other Tm 12.86 homologues in nucleotide sequence, and about 51% related in amino acid sequence. B1 is also closely related to Tm 13.17, with 57.2% identical nucleotides, but only 47.4% identical amino acids. AFP-3 is the least related in this family, with about 42% relatedness to the 2-2, 2-3, 3-4, 3-9, and 7-5 clones in nucleotide sequence (about 35% identical amino acids), 39.3% relatedness to B1 (37% amino acid identity), and sharing only 37.4% nucleic acid identity with Tm 13.17 (39.8% amino acid identity). The Type II AFPs, YL1 from T. molitor and DAFP-1A from D. canadensis, are very similar to each other (45.6 nucleic acid residue identity and 55.6 amino acid identity), but not significantly related to the Tm 12.86 homologues, or AFP-3 and B1. FIG. 4.20 simplifies the comparative tables with a phylogenetic tree, based on percent nucleic acid identity between the sequences. This tree shows that YL-1 and DAFP-1A are on an entirely different branch from the Tm 12.86 homologues. Among the other sequences depicted by the tree, Tm 13.17 is the most closely related to the nearly identical 2-2,2-3, 3-4, 3-9, and 7-5 clones. B1 and B2, however, are 5more closely related to Tm 13.17 than Tm 13.17 is to the other Tm 12.86 homologues. AFP-3 is the most distant relative, shown to branch off before any of the others.

V. Generation of Signal Plus and Signal Minus His-Tagged Clones

Several members of the Tm 12.86 Family of genes have been cloned. However, the recombinant products generated in each failed to demonstrate significant antifreeze protein activity, either recrystalliation inhibition (RI) or thermal hysteresis. In fact, efforts to establish antifreeze activity of these recombinant peptides have proven to be non-trivial, and not routine or obvious to someone skilled in the art. Establishing antifreeze activity of these recombinant products has required two phases of cloning modifications. The first phase, i.e. that of generating signal plus and signal minus His-tagged clones, is detailed here following procedures described in Example 5.

Three hypotheses were advanced for the lack of activity: 1. Since functional assay of the recombinant protein was carried out in the bacterial lysate, it may be that endogenous E. coli proteins may be non-covalently binding to the antifreeze protein and thus masking its function. 2. The presence of the uncleaved signal peptide may prevent the protein from folding in a proper three-dimensional state. 3. The lack of eukaryotic machinery that precisely controls the micro-environment and presence of chaperone proteins and other post-translational modifiers may be crucial for correct folding and functionality of the protein

A. Effects of Bacterial Proteins.

The effect of bacterial proteins on antifreeze activity was evaluated by testing different concentrations of purified antifreeze protein on it's ability to inhibit recrystallization (RI) and the impact (if any) that the presence of bacterial proteins have in this regard (See Example 8 detailing the RI assay). The results were evaluated by visual inspection of photomicrographs. Control sample with no antifreeze protein show large crystals that grew at the expense of smaller sized crystals. When Tm12.86, the positive control was tested in protein extraction buffer its recrystallization inhibiton activity was preserved at both concentrations i.e. 0.025 mg/ml and 0.0025 mg/ml. Moreover, the average crystal size of 0.025 mg/ml sample was smaller than that of the 0.0025 mg/ml sample. Inhibition of recrystallization was also clearly observed in samples with Tm12.86 in XLOLR lysate. Moreover, the average crystal size with 0.025 mg/ml samples were smaller than that of the more dilute AFP solution. As expected, the negative control with only bacterial lysate did not exhibit any recrystallization inhibtion as displayed in a pattern similar to that of protein extraction buffer control. The data resulting from these experiments strongly suggested that bacterial proteins do not hinder the activity of antifreeze proteins. These data do not support the first hypothesis that proposes that bacterial proteins may specifically or non-specifically inhibit antifreeze activity.

B. Generation of Signal Minus His-Tagged Clones

To explore whether the presence of an uncleaved signal peptide may be preventing antifreeze activity of the recombinant products, we deleted the region of the cDNA that encodes for the signal peptide and the remaining insert was expressed in E. coli to generate signal-minus recombinant proteins. Further, we made use of a new expression vector pET 28a which is capable of attaching a histidine tag to the protein of interest to facilitate enriched purification of the recombinant products.

Analysis of Purified pBK-CMV [2-2, 2-3 and Tm 13.17]. The first step in sub-cloning putative AFP genes in pET-28a vector involved the generation of large amounts of plasmid in which these genes were originally cloned. In this process, pBK-CMV 2-2, 2-3 and Tm 13.17 were transformed in DH5a cells, and individual colonies were cultured and scaled to a larger volume. Transformation of the colonies for the specified period of time resulted in small “glassy” colonies. It was observed that by adding the entire 0.5 ml of the bacterial media on a single LB-Agar plate resulted in a “lawn” where individual colonies were difficult to isolate. To avoid this, 50 ul of the media on each plate resulted in a good number of distinct colonies. Purification of plasmid DNA from a large culture of these colonies resulted in relatively uncontaminated DNA, as measured by the ratio of OD at 260 nm and 280 nm. The ratio of all purified samples ranged from 1.5-1.7, with a value of 1.8 reflecting highly pure DNA. The yield of the plasmid DNA ranged from 50-70 ug. Two micrograms of pET-28a and each pBK-AFP samples were restriction digested and electrophoresed on a 1% agarose gel. Uncut pET-28a was found to have two distinct bands at approximately, 17 kb and 12 kb, while uncut pBK-AFP plasmids were found to have three distinct bands (from top to down), nicked, normal and supercoiled, migrating at 20 kb, 8 kb and 4 kb, respectively. When pBK-AFP samples were double-digested with BamHI and XhoI, two different bands were displayed, a larger band of 4.7 kb, and a smaller and faint band of 500 bp. The 500 bp fragment is the expected size of the AFP genes. The double-digestion of pET-28a released a 60 bp fragment that was too small to visualize. These steps confirm the successful purification of plasmid DNA containing the AFP clones and thus facilitated their usage for sub-cloning purposes.

Generation of Signal Peptide Deleted Fragment(s). Signal peptide deleted fragments were generated by PCR with primers designed to sequences downstream of the signal peptide and upstream of the stop codon. Additionally, two artificial restriction sites, BamHI and XhoI, were designed in the primers in order to incorporate these sites in the fragments (SEQ ID NO's 40-43). The plasmid DNA isolated in the previous step was used as a template in the PCR reaction. Following PCR, the entire reaction product was then electrophoresed on a 1.5% agarose gel, and a distinct and strong band was observed at 350 bp. Since this is the expected size of the AFP clones when the signal peptide, poly-A tail and other non-coding regions are removed, this result suggests that the primers and the PCR reaction successfully yielded a signal deleted cDNA fragment.

Restriction Digestion of pET-28a and Signal-Peptide Deleted Fragments. The PCR amplified fragments were cut from the gel and purified. Since PCR amplification of DNA resulted in blunt-ended fragments, it was necessary that these fragments be digested with the appropriate enzymes before proceeding with the ligation. Accordingly, the gel purified fragments were double digested with BamHI and XhoI in order to generate “sticky” ends. Similarly, the sub-cloning vector, pET-28a was also double-digested. Following this step, 1/10 th of the total reaction volume of both pET-28a and signal-deleted fragments were electrophoresed on a 1% agarose gel to confirm that gel purification and double-digestion were indeed achieved. As expected, a single band at 350 bp for PCR generated fragments, and a single band at 5.5 kb for pET-28a was seen. This sets the stage for sub-cloning.

Screening for pET-28a-AFP (signal minus). Ugation of the vector and the insert was catalyzed by T4 DNA Ligase. The ligase was then heat inactivated and the reaction product was transformed in bacteria The bacteria were plated on LB-Agar plates containing kanamycin resistance. These bacteria would then harbor one of two types of plasmid populations, 1) self ligated vector and 2) vector with the insert. To differentiate between these, individual clones were cultured and plasmid DNA was extracted and analyzed by double-digesting with BamHI and XhoI. The screening of several potential signal-minus (S−) clones containing 2-2 S−, 2-3 S− and Tm 13.17 S− are depicted in FIG. 5.2 (for 2-2 S−). This shows the screening of 18 clones for pET-2-2 (S−) by double-digesting with BamHI and XhoI. Eleven out of 18 clones were found to release a 350 bp fragment, which corresponds to the expected size of signal deleted AFP fragments. Similarly, screening of eighteen clones (nine each) for pET-2-3 (S−) and pET-Tm13.17 (S−) by double-digesting with BamHI and XhoI, resulted in seven of nine clones of 2-3S− and four of nine clones of Tm 13.17 S− found to release a 350 bp fragment. This suggests that these clones have successfully incorporated the PCR amplified fragment in the pET-28a vector. In addition, we sought to further confirm this result by employing two additional methods, 1) by digesting with PvuI enzyme, and 2) amplifying by PCR using internal and external primers. The danger of accumulating random mutations is higher when employing PCR in the cloning strategy since Taq (Thermo aquaticus) DNA polymerase has a low proof-reading efficiency of 10⁻⁶. While the fidelity of small fragments may be high, the chance of random mutations for larger fragments increases. Therefore, the accuracy of a few potential clones of pET-2-2 (S−), pET-2-3 (S−) and pET-Tm13.17 (S−) were determined by digesting with PvuI restriction enzyme.

Confirmation of Potential Clones with PvuI Restriction Enzyme. A PvuI site is found outside the multiple cloning sites (MCS) of the pET-28a vector. In addition, clones 2-2 and 2-3 have an internal PvuI site, while Tm 13.17 does not have any Pvul site. Thus, restriction digestion of pET-28a and pET-Tm13.17 (S−) linearizes the vector while digestion of pET-2-2 (S−) and pET-2-3 (S−) should release a fragment of 1400 bp. As a control, the pBK-2-2 and Tm 13.17 was digested with PvuI. The pBK vector has two PvuI sites, one inside the multiple cloning sites (MCS) and another outside. Cutting the pBK-CMV vector will yield a fragment of 1800 bp. However, the original cloning step in generating the cDNA library (Example 2) resulted in loss of one of the PvuI sites found in the multiple cloning site. As a result, cutting the pBK-2-2 is expected to release a 650 bp fragment, while pBK-Tm 13.17 will not yield any fragment. In addition, clones from the self-ligated colony of pET-28a were also digested, which should resemble purified pET-28a. The self-ligated colony may result from a complex recombination event and therefore may not look identical to the original. In fact, self-ligated colonies may sometimes contain only the antibiotic gene and other sequences may be lost. The outcome of Pvul restriction digests is shown in FIG. 5.3, with PvuI digestion of self-ligated pET-28, pBK-2-2, pBK-Tm 13.17, purified pFF-28a and potential pET-AFP clones. As predicted, PvuI digestion of self-ligated pET and purified pET28a resulted in linearization of the plasmid, and digestion of pBK-2-2 resulted in a 650 bp fragment. Similarly, digestion of pBK-Tm 13.17 resulted in linearizing the plasmid without dropping any fragments. Furthermore, when selected clones of pET-AFP were digested and examined, pET-2-2 and pET-2-3 releaed, as predicted, a band of 1400 bp, while pET-Tm 13.17 did not release any bands. Since samples in lane 8, 10, 13 and 14 failed to yield the expected fragment, they proved to be false positives and were subsequently discarded. In sum, these results have provided an additional confirmation that PCR amplified, signal-deleted AFP fragments have been successfully sub-cloned in the pET-28a vector.

Further Confirmation of PET-AFP Vectors with PCR. For additional confirmation that we have successfully sub-cloned the signal-deleted fragments in pET vector, further testing was performed using PCR. In this regard, the signal-deleted fragments were amplified by using two sets of primers 1) T7 and T3 external primers that are found only in pBK-CMV vectors and 2) internal primers with sequences directed to the AFP genes. Amplification of the pBK-cDNA vectors with external primers would be expected to yield 500 bp bands, while pET-cDNA vectors would not be expected to show such bands. In contrast, use of internal primers to amplify pBK-cDNA and pET-AFPs should result in bands of 350 bp. Additionally, use of internal primers with pET-28a (no inserts) should not result in any bands. These results shown in FIG. 5.4, do indeed demonstrate each of these expected results, and as such strongly confirm the successful sub-cloning of signal-deleted cDNA in pET vectors.

Cloning and Screening Signal Preserved AFP Genes in pET-28a. Fragments of AFP with an intact signal peptide were generated by double-digesting pBK-AFP vector with Baml and XhoI. The 500 bp fragments released in FIG. 5.2 was gel purified along with the digested pET vector. Following ligation, bacteria was transformed and plated in LB-kanamycin plates. Plasmid DNA extracted from bacteria was then analyzed with BamHI and XhoI enzyme for the presence of the cloned insert FIG. 5.5 shows and example of screening of pET signal-plus AFP clones. Lanes 3 and 4 (2-2S+) and 11 and 18 (2-3S+) resulted in the release of the desired 500 bp fragment. The remaining clones were negative and subsequently discarded. Clones in lane 6 and 8 failed to produce any plasmid and suggests that the culture may have originated from a satellite colony. Similarly, four out of eighteen clones of pET-Tm 13.17 S+ released the desired fragment of 500 bp. These results confirm that the signal preserved AFP fragments have been successfully incorporated in pET-28a. Further confirmation with other enzymes or PCR was not performed since this strategy did not involve the use of PCR amplified inserts.

Sequencing of pET-AFP vectors. For final confirmation that signal-plus and signal-deleted inserts were successfully subcloned into the pET vector without accruing mutations, the sequence analyses of plasmids were performed. Plasmids from bacterial stocks of pET-AFP clones were extracted using procedures detailed in Example 5. The plasmids were amplified by using the 17 promoter sequence found in the upstream region of the multiple cloning site. Following this, sequence analysis of the clones was conducted on a ABI Prism Sequencer. The positive control was pET vector without any insert. The results were compared with the original sequences and were found to have no error. Some sequences were unrecognized by the software and manually read and verified for accuracy. In addition, the sequences encoding for the histidine tag, the thrombin cleavage site and the T7 tag were preserved in all the clones. The sequencing results of pET-[2-2S+, 2-2S−, 2-3S+, 2-3S−, Tm 13.17S+ and Tm 13.17S-] are presented in FIGS. 5.7-5.12 (SEQ ID NO's 16-27) FOR NUCLEOTIDE AND PEPTIDE SEQS.

Determination of Parameters for Optimal Yield of Recombinant pET-AFP Protein(s). Expression of the recombinant proteins was performed in BL21, a strain of bacteria suited for protein expression. pET-AFP plasmids were transformed in this strain of bacteria and colonies were cultured. The protein expression of pET-2-2 (S+) was induced with IPTG and small aliquots of the culture were removed every hour for up to five hours and twenty four hours since induction. Finally, the LB-media was analyzed for secreted proteins and all the experimental samples were analyzed by SDS-PAGE. The results showed that the bacterial culture did indeed express recombinant protein following IPTG induction. A single band between 14 and 20 kDa was seen to appear from 2-5 hours post induction and continued to express proteins up to 24 hours. Band intensity appears to have continued to increase during this time and 24 hours after IPTG induction. The induction of pET (no insert) serves as a negative control as seen with the lack of bands in the 14-20 kDa region. A positive control for the experiment was pBK-CMV-Tm 13.17 expressed in XLOIR strain of bacteria, as this is seen in a strong band in this region. These results suggest that recombinant protein expression is optimal at OD600 0.5-0.6 for 5 hours after inducing the culture with 1 mM of IPTG.

Determination of Optimal Conditions for Thrombin Cleavage. To establish optimal conditions for thrombin mediated proteolytic digestion of the histidine tag, the duration of digestion and concentration of thrombin was varied in batch purified recombinant pET-2-2 (S+). A positive control provided by the company was also digested. Proteolytic digestion of recombinant proteins was marked by a reduction in molecular weight. The positive control shows two bands with molecular weights predicted in the company literature. The results suggest that the histidine-tag was effectively cleaved from 10 ug of recombinant protein when digested with 0.001 units of thrombin for 4 hours at 20C.

Purification of His-tagged Recombinant Proteins. After establishing the optimal conditions for protein expression, the pET-AFP cultures were scaled to a larger volume and recombinant histidine-tagged protein was purified through column chromatography. The yield of the purified proteins resulted in about 3.0 mg from a 100 ml culture. The purified histidine-tagged recombinant proteins were then subjected to proteolytic digestion by thrombin to remove the histidine tag. Samples were then evaluated electrophoretically. With each cloned insert (2-2S+, 2-2S−, 2-3S+, 2-3S−, Tm 13.17S+ and Tm 13.17S−), a major band was detected near 14 kDa, which appears to co-migrate with purified, native Tm 12.86. This demonstrates first that an expected size-specific, recombinant protein is expressed in the case of each cloned insert. Secondly, because the appropriate size of the recombinant protein appears similar to that of native Tm 12.86, it appears that the histidine tag was successfully cleaved from the recombinant protein during subsequent thrombin cleavage step. Interestingly, differences in migration pattern between signal preserved and signal deleted proteins were not observed. However, one might observe this if the percentage of the acrylamide was increased in the gel.

Immunodetection of Recombinant Proteins. A western blot analysis of the recombinant proteins was performed which could indirectly test the efficacy of a bacterial system to express properly folded fusion proteins. The reason for this is that successful detection with a western, may suggest that the appropriate epitopes are being displayed by the recombinant protein. FIG. 5.6 shows the results of the western blot analysis. For each of the cloned inserts, pFT: 2-2 (S+), 2-2 (S−), 2-3 (S+), 2-3 (S−), Tm 13.17 (S+) and Tm 13.17 (S−), respectively, a broad band between 20 and 14 kDa is seen, indicating that the recombinant proteins were immunodetected by the antibody specific to Tm12.86.

Thermal Hysteresis Activity of pET-Recombinant Proteins. The histidine-tag cleaved recombinant proteins were tested for functional activity by employing both capillary tube thermal hysteresis detection and a recrystallization inhibition (RI) method. Proteins were tested at concentrations of 50 mg/ml, 20 mg/ml, 5 mg/ml, 1 mg/ml and 0.5 mg/ml by employing the capillary tube method. Similarly, RI was employed to test proteins at concentrations of 1 ug/ml, 0.5 ug/ml, 100 ng/ml and 10 ng/ml. All recombinant proteins failed to exhibit antifreeze activity at any concentrations. Following this, the proteins were denatured with 6 M urea and refolded in serial dilutions of urea (5 M, 4 M, 3 M, 2 M, 1 M, 0.5 M and 0 M). Samples were lyophilized and resuspended in water. The refolded proteins were tested again for functional activity at similar concentrations, but no antifreeze activity was detected.

The first efforts to isolate the gene for Tm 12.86 did not yield that particular gene, but resulted in the serendipitous discovery of other homologous antifreeze protein genes. To date, we have isolated several homologous cDNAs which have N-terminal sequences that are identical or similar to that of Tm 12.86 and cross-react with its antibody. Expression of these clones in a bacterial system resulted in a strong band at the appropriate molecular weight and cross-reactivity with Tm 12.86 antibody. However, functional analysis of the whole bacterial lysate failed to exhibit antifreeze activity. This describes some of the efforts made to reconstitute antifreeze activity in these recombinant proteins.

Specific or Non-Specific Inhibition by Endogenous Bacterial Proteins. It was first considered that the lack of antifreeze activity was attributed to the specific or non-specific inhibition of antifreeze proteins by endogenous bacterial proteins. In this scenario, bacterial proteins were envisioned to bind to the antifreeze proteins themselves and cause steric hindrance. To test this theory, the recrystallization inhibition (RI) activity of purified Tm12.86 was tested under two conditions, 1) in protein lysis buffer and 2) in endogenous bacterial proteins devoid of recombinant AFP proteins. When Tm 12.86 was diluted in protein lysis buffer and tested for RI activity, it inhibited recrystallization of ice-crystal. In contrast, control reactions testing RI activity of the protein lysis buffer or bacterial lysate resulted in relatively larger ice crystals. This demonstrated that ingredients in the lysis buffer do not inhibit recrystallization. Additionally, when Tm 12.86 was diluted in bacterial lysate devoid of recombinant antifreeze proteins and tested for RI activity at two different concentrations, the functional antieeze activity of Tm 12.86 was seen in both samples. In contrast, bacterial proteins devoid of Tm 12.86 failed to inhibit recrystallization, and thus exhibited no antifreeze activity. This experiment conclusively demonstrated that endogenous bacterial proteins do not inhibit, specifically or non-specifically, the antifreeze activity of our native antifreeze protein Tm 12.86. Therefore, we would conclude that steric hindrance by bacterial proteins is not a likely explanation for lack of antifreeze activity of these recombinant proteins.

Inhibition of Antifreeze Activity Due to Improper Folding. The alternate explanation for the lack of antifreeze activity focuses on the lack of a proper folding given the presence of an uncleaved signal peptide. Eucaryotic systems employ signal peptides to direct the transportation of proteins to different compartments and organelles. Upon reaching the appropriate destination, the signal peptide is cleaved and the remaining protein undergoes refolding to attain its proper conformation. The absence of such a mechanism in bacteria prevents the cleavage of signal peptides that may result in improper folding and thus inhibit the functionality of the protein. To address this problem, the signal peptides of complimentary DNA (cDNA) of AFP genes 2-2, 2-3 and Tm 13.17 were deleted. This was achieved by employing polymerase chain reaction (PCR) with primers designed downstream of the signal peptide and upstream of the stop codon. In addition, the primers were tagged with BamHI and XhoI restriction sites that were convenient for sub-cloning purposes. The PCR reaction resulted in a single strong band with a reduced molecular weight that reflected the loss of the signal peptide, poly-A tail and other non-coding regions of the gene. The PCR amplified gene product was digested with BamHI and XhoI to yield sticky ends to enable the sub-cloning in a new expression plasmid, pET-28a.

The pET expression system enables the purification of recombinant proteins by co-expressing an N-terminal histidine tag of six amino acids. During purification, the histidine tag binds to an immobile nickel resin and subsequent washings effectively isolate the recombinant protein from the bacterial proteins to yield a highly pure sample of the desired recombinant protein. If desired, the histidine tag can be cleaved by proteolytic digestion of thrombin leaving only a small number of non-polar residues remaining attached to the N-terminal.

The results from our study have established that we succeeded in subloning signal-deleted and signal preserved AFP genes in the new pET-28a vector. This was confirmed when the signal-deleted and signal preserved plasmids were double-digested with BamHI and XhoI and a fragment of 350 bp and 500 bp was released respectively. Additionally, restriction digestion of the plasmids with PvuI resulted in the release of appropriately sized fragments. Their authenticity was further confirmed by employing PCR with primers designed to the internal regions of the AFP genes. The amplification of a 350 bp fragment confirmed the presence of signal-peptide deleted AFP genes in the new expression vector. Lastly, the sequencing of the pET vectors confirmed the deletion of the signal peptide in 2-2 (S−), 2-3 (S−) and Tm 13.17 (S−) and the lack of frame shift or other mutations. Similarly, the sequencing of signal preserved AFP homologs confirmed the presence of the AFP genes and the absence of frame shift or mutations.

The successful cloning of AFP homologs in the new expression system paved the path to express and rapidly purify the recombinant proteins with an N-terminal histidine tag. We also determined parameters suited for maximum expression of the recombinant protein, and concluded that the optimal conditions for protein expression was achieved by inducing a bacterial culture at OD600 of 0.5-0.6 with 1 mM of IPTG (final concentration) for 5 hours. Under these conditions, a 100 ml culture resulted in a yield of approximately 3 mg of purified protein.

Despite the fact that a better system for generating an enriched amount of recombinant proteins at a fraction of time and cost associated with the traditional method was developed, these proteins did not exhibit antifreeze activity in either the thermal hysteresis capillary method or the recrystallization inhibition assay. Removal of the histidine tag from the proteins followed by denaturation with urea and renaturation in serial dilutions also failed to reconstitute activity. In all cases, the recombinant proteins failed to exhibit antifreeze activity. The lack of activity suggests that the proteins may be incorrectly folded regardless of the presence or absence of signal peptides.

VI. Recombinant Proteins Isolated from Inclusion Bodies Displays Antifreeze Activity

Establishing antifreeze activity of the recombinant products of the Tm 12.86 gene family has required two phases of modifications from the original clones. Phase 1 detailed in Example 5 produced inserts that either retained or eliminated the N-terminal signal peptide. Furthermore, purification and enrichment of the recombinant proteins was enhanced through the addition of a His-tag. Nevertheless, these improvements were by themselves insufficient to establish antifreeze activity of the recombinant products, even when numerous and various attempts at additional denaturing and refolding procedures were employed. Phase 2 (detailed in Example 6) required a direction of focus that concentrated on proteins directed to the bacterial inclusion bodies. This was an unusual direction to pursue given the strong hydrophilic nature of the Tm 12.86 like peptides, and that a related recombinant protein, AFP-3/THP12 isolated from the bacterial supematent was found to be properly folded as a small lipid binding protein (Rothemund S. et al., [1999] Structure 7: 1325-1332). In fact, unexpectedly, the inclusion bodies turned out to be a critical step for the obtainment of antifreeze protein activity by the Tm 12.86 family of Type III AFPs.

His-tag recombinant protein Tm 13.17. The Tm 13.17 mature protein (signal minus) was subcloned into pEF-28a expression vector, which was capable of linking 6 histidine amino acids with a single thrombin cleavage site at the N-terminus of the recombinant protein Tm 13.17. During affinity chromatography, the histidine-tagged protein was bound to Ni2+ resin, and then eluted by elution buffer (FIGS. 6.0, and 6.1). The purified his-tagged product can then be cleaved with thrombin proteinase. Originally, all recombinant products were processed through the proteolytic removal of the His-tag, since it was hypothesized that the N-linked His-tagged may also interfere with antifreeze activity. However, as determined later, the thrombin cleavage step is not essential for activity, since the presence of the His-tag does not interfere with antifreeze activity.

The activity of recombinant protein Tm 13.17 and 2-2. The yield of recombinant protein isolated from the inclusion bodies was substantially lower than that obtained from the supematent fraction (Example 5). However, when the recombinant product was tested for RI activity (see Example 8 for details), as shown in FIG. 6.2, the recrystalization inhibition assay demonstrated that the size of ice crystal mf 1 mg/ml recombinant protein sample was significantly smaller than the PBS control as well as, the sample of proteins from bacterial without the insert. An RI dilution profile for recombinant Tm 13.17 at 10 mg/ml starting concentration is shown in FIG. 6.3 with a calculated “RI factor” from the regression line determined to be 1.93. (See Example 8 for discussion of RI factors).

Additionally, thermal hysteretic activity was found for the recombinant Tm 13.17 peptide, and the recombinant Tm 2-2 product, which at a concentration of 0.5 milligrams per milliliter depressed the freezing point by 0.2 C, while at 1 mg/ml had thermal hysteresis (TH) of 0.35° C. and at 5 mg/ml had TH of 1° C. These levels of activity for the recombinant AFP are about 60% that of the native Tm 12.86. Even at this level, the clones display a considerably higher level of activity than fishes antifreeze protein at similar concentration. Given the strong relatedness of the Tm 12.86—like clones, one might expect each of their corresponding proteins to display antifreeze activity.

Importance of Inclusion Body Isolation. When the denaturing and refolding procedures followed in Example 6 are employ on recombinant proteins obtained from the supematent (as in Example 5), the recombinant proteins still fail to display antifreeze activity. Thus, something associated with the packaging into, and/or the microenvironment of, the inclusion bodies is essential for establishing antifreeze activity of the Tm 12.86 family of Type III AFPs.

The development of molecular biology techniques to express a gene or cDNA in a suitable host heralded the promise of mass producing beneficial proteins at a fraction of time and cost and has been accomplished in basic research, clinical settings and industrial applications. Despite the overwhelming success, some rare proteins have resisted conventional attempts to be produced in a foreign host. These proteins have unique three-dimensional structures that are difficult to achieve in a foreign host. As a result, these misfolded (and inactive) proteins become insoluble and aggregate into dense, non-membrane bound structures called inclusion bodies that appear in the periplasmic space. In fact, overexpressing native or foreign proteins results in the accumulation of some fractions in inclusion bodies due to the inability of the host to fold/modify the proteins at the same rate at which it is synthesized. Intensive research on the nature of inclusion bodies have led to some useful procedures to reactivate misfolded proteins. The first step is bacterial lysis by repeated freeze-thaw, lysozyme and/or sonication, and followed by high-speed centrifugation at 15,000-30,000 g to isolate the inclusion bodies. This is followed by, 1) solubilizing the aggregated proteins with a denaturant such as SDS and/or urea, 2) removal of contaminants, and 3) denaturation and renaturation.

Given these findings, it is perplexing from our observations that recombinant AFP proteins are predominantly expressed as soluble fractions in the cytosol, and poorly expressed in inclusion bodies. In fact, soluble cytoplasmic fractions (3 mg/100 ml culture) of AFP are produced 150 times in excess of insoluble fractions from inclusion bodies (50 ug/100 ml culture). Based on reported literature, one would expect to find activity in soluble fractions since related molecules have been shown to be correctly folded (Rothemund S. et al., [1999] Structure 7: 1325-1332). Moreover, it would be unlikely, and one would not expect to detect antifreeze activity in the misfolded and insoluble inclusion bodies.

This confusing observation may be attributed to the presence of cysteine residues in the AFP homologs in this invention. The thiol (—SH) side chains of cysteine residues can form covalent disulfide bonds. Whether AFP homologs form disulfide bonds is not yet known, but experiments with native Tm 12.86 has shed some light on this matter. We have observed different electrophoretic patterns. of Tm12.86 treated with or without (-mercaptoethanol, a powerful reducer of disulfide bonds). Untreated proteins had a single band, while treated samples had two bands that migrated in the same molecular weight range as the untreated protein. This observation has not yet been properly explained, but one can infer that this could be attributed to the presence of complex disulfide bond(s). The Tm 12.86 family of AFP homologs, for the most part share similar disulfide bonds, i.e. the predicted amino acid sequence of the mature peptides encoded by our AFP clones indicate four cysteine residues (excluding 3-4) and thus a properly folded protein may have up to two disulfide bonds.

Disulfide bonded proteins produced in the bacterial cytosol aggregate into inclusion bodies due to improper folding as a result of the reducing environment in the cytosol. However, the periplasmic space provides the ideal oxidizing conditions for disulfide bond formation. In our situation, the AFPs may not have formed the disulfide bonds, but yet predominantly remain soluble due to a unique, albeit misfolded, structure that prevents aggregation. The overexpression of AFPs, like any other protein, will result in the production of inclusion bodies. The small fraction of AFPs that form inclusion bodies become exposed to oxidizing conditions which favor disulfide bond formations. It is important to note that denaturation by urea results in loss of hydrogen bonds, but preserves disulfide bonds. Thus, the disulfide bonds formed in the inclusion bodies are preserved during the subsequent denaturation/renaturation steps, which may be unnecessary but this needs to be verified. In addition, dithiotireitol (DTT) prevents cysteine oxidation and new disulfide bonds formation in subsequent purification steps.

In hindsight, finding functionally active AFPs in inclusion bodies may have some analogies to the native situation. We have shown in vivo that after expression, Tm 12.86 makes it's way to crystalline structures called protein granules, which are subsequently broken down to meet physiological demands. The internal environment of protein granules have not been studied, but it would not be surprising to find that it provides an essential oxidizing environment (similar to inclusion bodies) for AFPs to “age” and become functionally active.

Another explanation that may be attributed to inactivity in soluble cytosolic proteins involves the empirical observation that renaturation of proteins is optimal at 10-100 ug/ml since higher concentrations could lead to aggregation. In this light, the soluble cytoplasmic proteins were folded in the laboratory at much higher concentrations than suitable.

Attaining Functional AFPs in E. coli. The amount of functional AFPs in inclusion bodies is low and thus employing this route of purification becomes fairly expensive. However, there are several methods to increase the production of inclusion bodies. These are, 1) incubating the culture at 42° C. as opposed to 37° C., 2) varying the amount of dissolved oxygen in the media, and, 3) addition of ethanol to the media to a final concentration of 3% (w/v).

Other methods that may be employed to increase the amount of functional AFPs in the soluble fraction is to decrease the rate of protein synthesis by, 1) reducing the incubating temperature from 37° C. to 30° C., and 2) adding non-metabolizable carbon sources such as deoxyglucose at the time of induction. Alternatively, a complicated approach may be to co-express molecular chaperones such Hsp 60, Hsp 70, GroES, GroEL, DnaK/DnaJ/GrpE and/or ClpA/X. To address disulfide bonded proteins, overexpressing DsbC protein, a disulfide isomerase, along with the AFPs may enhance correction of incorrect disulfide bonds. Additionally, supplementing the renaturation buffer with glutathione, cysteine and cysteamine may allow for appropriate bond formation. Importantly, new expression strains developed at Novagen with mutations in the either thioredoxin (trx B) or glutathione (gor 522) or both (Origami™) pathways is a quick and convenient system to explore.

Eucaryotic Expression Systems. Given the challenge of obtaining the properly folded Tm 12.86 like AFPs, with the necessary disulfide bridge linkages etc, one may have improvement of both yield and folding characteristics in Eukaryotic vectors such as yeast and baculovirus systems.

VII. Concensus Sequences for the Tm 12.86 Gene and Protein Family

Concensus sequences for the genes and proteins of the Tm 12.86 family (cladistic tree shown in FIG. 4.20) were identified as detailed in Example 7 paying careful attention to the types of substitutions and chemistry involved. Both a full general concensus sequence was described for the entire Tm 12.86 gene family encoded proteins, and consensus sequences for the nested genes within the family are also described (i.e. concensus sequence for Tm 12.84-6 like, consensus sequence expanded to include Tm 13.17 like, concensus sequence expanded to include B1/B2 like, and concensus sequence expanded to include AFP-3 like, genes and their encoded proteins (SEQ ID NO's 44-48). Detailed in FIGS. 7.2 and 7.3 are the full breath of the concensus sequences for nucleotides and amino acids, respectively, and for each grouping the most representative concensus sequence, and also positions and types of substitutions either occurring or deemed acceptable. See FIG. 7.1 for reference to amino acid letter designations and chemical classifications.

Protein Sequences, Starting with Tm 12.84: (Refer to FIG. 7.3)

The 5 clones in this series are highly conserved. At the protein level, one (3-9) shows a substitution at position 37 (from the initial methionine) of an amino acid with an acidic side chain (glutaic acid) for one with an aliphatic side chain (valine). Since valine is the most common, it is placed in the concensus sequence, with the understanding that glutamic acid is a recognized substitution for this gene family. Clone 3-9 also shows a substitution at position 69 of an amino acid with a basic side chain (arginine) for another with a basic side chain (lysine). Again, since lysine is most common, it is included in the concensus, with arginine a recognized and expected substitution. Another clone (34) shows a substitution at position 122** of an amino acid with a hydrophobic sulphydryl group (cysteine) with another having a hydrophobic, aliphatic side chain (valine). Since cysteine is most common it is included in the concensus with valine noted as a potential substitution. For alignment purposes in FIG. 7.3, a gap is present at position 94 in the sequence for ALL Tm 12.84 clones, since they share the smaller, 115 residue number. Thus, as will be the case for all Tm 12.84 clones, residue position numbers in FIG. 7.3, listed after 94 will reflect this extra number assignment. Therefore, as in the example above, clone 3-4 has the valine substitution actually at position 121 from the initial methionine, as seen in SEQ ID NO. 10).

As more distant relatives of the gene family are considered, it is important to note the strongly conserved features of the group as these are most probably responsible for their common functions (i.e. antifreeze activity) and certainly provide clues as to their evolutionary origins. In developing the concensus sequences, we have included the furthest members of the family (refer to FIG. 4.19 and 4.20); the assessory gland proteins B-1 and B-2 from T. molitor, putatively thought to be pheromone binding proteins; and AFP-3(THP-12), also from T. molitor and demonstated to be a small lipid carrier, but whose status as an AFP is in doubt. Additionally, note that B-1 and B-2 lack a complete open reading frame, missing both the N-terminal methionine, and a suitable, “in frame” stop codon at the C-terminus (as determined from their first translated amino acid). Nor do they have a poly adenylation signal and poly A tail. Since the comparisons are based only on partial sequences, we can expect the concensus to change as their complete sequences are revealed. Therefore, further comparison has focused on full length members of the family.

Every cysteine residue save the last is completely conserved in every member of the family. They are found at positions (from the initial methionine) 6, 34, 65, 105, and 122 from the initial methionine (FIG. 7.3). Regions around these cysteine residues are also conserved with particular conservation of lysine, glutamine, glutamic acid, isoleucine, and valine. When these residues are substituted in any of the family members the replacement is typically a substitution of kind, with one aliphatic amino acid replacing another, or a basic replacing a basic, and so forth. Even when the substitutions are not in kind, other aspects of the side chain chemistry are similar. For example, in AFP-3, the concensus glutamic acid is occasionally replaced by either arginine or lysine. Although these would appear to be opposites (basic groups for an acidic one), both groups are polar, hydrophilic, and reactive.

Another area that is remarkably conserved are the proline residues at positions 57, 112, 128, and 132 (FIG. 7.3). Indeed, positions 55 to 59 are conserved in every member of the family and consist of acidic side chains on one side of the proline and basic side chains on the other. This suggests the potential to form a stabilized hinge on which these proteins would readily fold and interact with water. There are also several other completely conserved residues, including the aliphatic hydroxyl, serine, at positions 31, 38, and 41; lysine, at positions 58, 61, 87, 104, and 121; and the aliphatics, alanine, glycine, and valine at positions 18, 40, 77, and 107. Even the residues that are not completely conserved are often similar in side chain chemistry, hydrophobicity, reactivity, or size.

Together, the conserved residues and similar substitutions form a general pattern that contributes to the special chemistry of this family of proteins, including their ability to bind to ice and prevent crystal growth. SEQ ID NO. 48 presents a full general consensus peptide sequence for the entire Tm 12.86 gene family. With this in mind, although never tested, the close similarity of the B1 and B2 T. molitor proteins (indeed more so than AFP-3) suggest that these will likely exhibit antifreeze activity.

At the Gene Level. The nucleotide sequence shows similar conservation of sequence (FIG. 7.2). Where amino acid residues are conserved, so to are the codons for them (allowing for third position “wobble”). Only in positions where many amino acid substitutions have occurred within the family do we find potential for any nucleotide substitution to occur within the DNA sequence. This suggests rather strongly that these genes arose by divergence from a common “ancestral” gene, rather than by convergence to a common chemistry from disparate genes. This is important as it further establishes the relatedness of these genes and justifies their inclusion in a single gene family.

Additionally, the evidence obtained both from comparative sequence analyses and Southern analyses (see details from Example 4) indicate a strong likelihood that representative members of the Tm 12.86 multigene family of AFPs exist within Tenebrionidae (family) and even Tenebrionoidea (superfamily). The superfamily Tenebrionoidea includes both the Tenebrionidae family of darkling beetles (including Zopheridae) plus the Pyrochroidae family of fire colored beetles (including D. canadensis). Southern analyses with Tm 2-2 probe (FIGS. 4.4 and 4.5) has indicated a faint level of hybridization to D. canadensis genomic DNA, yet fails to recognize even faintly a band from lepidopteran DNA (Manduca sexta). Moreover, recall the DNA sequences encoding Type II AFPs from both Tenebrio and Dendroides show some 46% nucleotide sequence similarity. Thus, it's reasonable to expect that members of the Tm 12.86 multigene family of Type III AFPs exist both within the Tenebrionidae family and even Tenebrionoidea superfamily.

VIII. Quantification of Recrystallization Inhibition (RI).

Recrystallization occurs in any frozen crystalline solid, whereby large ice crystals spontaneously grow over time replacing smaller adjacent crystals, and it can significantly degrade the texture and product quality of frozen foods, and is quite detrimental to cell and tissue cryopreservtion. Therefore, there is great commercial potential for products that can limit or prevent this process. The ability of THPs to inhibit recrystallization, referred to as RI has now been well documented. Thus, an embodiment of the present invention is the applicability of the Tm 12.86 gene family and their encoded Type III AFPs for such ventures.

Also, given that RI effects can occur at titers of AFPs/AFGPs that are too low to generate a thermal hysteresis, and this RI behavior appears to be THP concentration dependent, a strong potential exists for generating and using an “RI assay”, that is more sensitive than the alternative determination of thermal hysteresis for assessment of antifreeze protein activity, and one capable of being upscaled and automated. However, two significant problems have impeded the development of such an assay: 1) A means to establish a rigorous, quantitative assessment of RI behavior based on the documented sensitivity and concentration-dependent behavior of a highly pure solution of a known AFP; and 2) the means to eliminate the confounding effects of RI-like behavior generated by non-THPs, thereby ensuring an “antifreeze protein specific” response. An embodiment of the present invention includes the establishment of a rigorous, quantitative assay of RI behavior based on the documented profile of purified Tm 12.86, a highly active Type III AFP from T. molitor, that includes specific quantitative guidelines and measures that allow for the elimination of non-THP RI-like effects. Moreover, another embodiment of the invention describes the feasibility of this quantitative RI assay to determine the presence of antifreeze proteins in unknown solutions or samples, and to provide a framework in which to evaluate and rank antifreeze protein activities and potency. Thus, the present invention provides for RI assay sensitivity and quantitation, under conditions ensuring AFP specificity and reliability, that extends the range of solution detection capabilities, encompassing, but not limited to evaluation of recombinant AFP products, synthetic AFP analogs, cell culture applications, assessment of activators, etc. Also, the invention includes mathematical modeling of the AFP induced RI effects and some aspects toward upscaling and automation.

Sample ice grain size distributions and the quantification of RI effects. The splat cooling technique was used to generate flash frozen samples (small wafers<1 cm diameter) that were maintained on a refrigerated cooling stage and viewed microscopically (procedures detailed in Example 8). The splat cooling technique typically yields a frozen wafer composed of fine-grained crystals (FIG. 8.0). Some ice grain size heterogeneities in splat cooled samples occur that are not considered significant from mere qualitative observations, yet become more problematic regarding any quantitative assessment. We hypothesized that factors such as uneven distribution of solutes and variations in ice sample thickness might influence average ice grain sizes at different sample locations. We first conducted a more systematic study of ice grain size homogeneity, applying a quantitative method of measuring mean largest grain size (mlgs) (detailed in Example 8, also see FIG. 8.2) with statistical evaluation to compare ice grain sizes at different ice wafer locations. The outcome of these experiments was then used as a guide for the development of a single, composite mean largest grain size measurement for each ice sample. For example, FIG. 8.1a shows the existence of an apparent “boundary line” separating two sample locations, here designated as sample “center” and “mid-sample” respectively. This boundary was visible for a majority of splat-cooled samples, and appeared to be related to a slight heterogeneity in sample thickness at this location. The first test of ice grain size heterogeneity was performed for H₂O samples (without solutes) annealed at −6° C. for two hours. Each sample was placed on the cold stage support in a manner similar to that shown in FIG. 8.1 and, after annealing, high magnification photographs of the sample center and mid-sample areas were obtained. A comparison of milgs values between the two areas revealed no significant differences (FIG. 8.4a) (p=0.15, n=5 samples). Similar analyses were performed for samples containing 0.01 mg/ml alpha -lactalbumin (α-lac), (chosen because it's molecular weight is closer to that of Tm 12.86 than BSA) and 0.001 mg/ml Tm 12.86. For 0.001 mg/ml Tm 12.86, no significant differences in sample center and mid-sample areas were detected at the α=0.05 level for samples annealed at −6° C. for 2 hours (FIG. 8.4a (p=0.069, n=4). However, for lactalbumin a difference in mlgs for center and mid-sample regions was evident for the 0.01 mg/ml α-lac samples (p=0.02, n=3). For higher concentrations of non-THPs in H₂O such as 0.1 mg/ml bovine serum albumin (BSA) annealed at −6° C., heterogeneities become much more profound as shown in FIG. 8.4b. These particular heterogeneities do not appear to be location-dependent within the samples, although they are most certainly related to the ability of non-THPs to induce RI effects (this phenomenon is presented in greater detail below).

In addition to the H₂O samples, 0.9% NaCl samples annealed at −6° C. for 30 minutes were tested for mean largest grain size heterogeneities by making the same comparison of sample center and mid-sample areas. Again, results indicated no detectable difference in mlgs between the two areas (p=0.195, n=16 samples) FIG. 8.5a. A third region near the sample edge was also included as a comparison to the center and mid-sample areas for the 0.9% NaCl samples. Still, no significant differences were detected among the three sample regions for 0.9% NaCl (p=0.21, n=16 each for sample center and mid-sample regions; n=8 for sample edge regions). Similar comparisons of sample center, mid-sample, and edge regions for 0.9% NaCl samples containing T. molitor hemolymph (1/1000 dilution), 10 mg/ml BSA, and 1 mg/ml BSA also failed to reveal any significant differences among the three sample areas within each category (p>0.45, n=6 for both BSA/0.9% NaCl categories, and p=0.08, n=7 for hemolymph/0.9% NaCl samples; see FIG. 8.5a). Thus, grain sizes appear to remain homogeneous between the sample center, mid-sample, and sample edge regions for 0.9% NaCl samples regardless of non-THP or THP content at −6° C. annealing temperatures.

An ice grain size heterogeneity, however, was consistently detected for 0.9% NaCl samples annealed at a temperature of −2° C. rather than −6° C. Ice grain sizes appeared to decrease significantly in areas of greatest sample (gravity-induced) deformation. The same pattern of ice grain heterogeneity with respect to the sample support ring (FIG. 8.1b) was consistently observed for each succeeding sample. To test this assertion, mean largest grain sizes were compared for sample areas located with respect to the sample support, here designated as “maximum deformation” and “minimum deformation” areas. The results indicated that a significant position-related effect occurs for ice grain size with respect to the sample support (p=0.0006, n=7 samples). Therefore, with regard to 0.9% NaCl samples at −2° C. annealing temperatures, mean largest grain sizes are significantly smaller at sample locations associated with the greatest apparent sample deformation. In contrast, an identical analysis performed for 0.9% NaCl samples annealed at −6° C. for 30 minutes revealed no detectable difference in mean largest grain size between sample “maximum deformation” and “minimum deformation” locations (FIG. 8.5b) (p=0.123, n=7). Similarly, no significant differences in mean largest grain size comparing “maximum deformation” and “minimum deformation” locations were obtained for H₂O samples annealed at −2° C. for two hours (p=0.72, n=5).

The results of the ice grain size homogeneity studies involving both H₂O and 0.9% NaCl samples with and without various THP and non-THP solutes were used as a guide to assess a single representative mean largest ice grain size for each ice sample in subsequent RI studies. Although only one significant difference between sample center and mid-sample regions (0.01 mg/ml α-lac in H₂O annealed at −6° C. for 2 hours) was noted out of several cases tested, we concluded that a composite milgs for most samples (with the exception of samples containing 0.9% NaCl and annealed at −2° C.) should still be computed as the mean of individual sample center and mid-sample area mean largest grain sizes. This method was applied to samples containing 0.9% NaCl and annealed at −6° C. for 30 minutes, and also to H₂O solution samples annealed at either −2° C. or −6° C. for two hours.

The ice grain size heterogeneity apparent for 0.9% NaCl samples annealed at −2° C. created a much greater difficulty with respect to composite mlgs determinations. For most of the experiments involving samples diluted in 0.9% NaCl and annealed at −2° C., the composite mean largest grain size was computed as the mean of maximum and minimum deformation mean largest grain sizes as defined in FIG. 8.1b. Another approach that minimized the effect of ice grain size heterogeneity for 0.09% NaCl samples annealed at −2° C. involved incorporating a random sampling technique to define mlgs (detailed in Example 8 Section F).

Non-thermal hysteresis proteins and recrystallization inhibition. In addition to ice grain size heterogeneity, yet another concern with respect to the development of a quantitative recrystallization inhibition assay of THP activity is the ability of non-THPs to induce RI under certain conditions. Therefore, we sought to quantitatively evaluate and detail specific sample treatment criteria that would eliminate non-specific RI effects.

a). The use of higher annealing temperatures to eliminate the RI effects of non-THPs in H₂O. We first examined ice samples consisting of 0.1 mg/ml (10⁻⁶ M) bovine serum albumin (m.w.=68 kDa) and 0.1 mg/ml (10⁻⁴ M) alpha-lactalbumin (m.w.=14.4 kDa), both in H₂O solution. When these samples were annealed at −6° C. for 2 hours, a strong R.I. effect was clearly evident when comparing high magnification photographs (195×) of the non-THP samples to a photograph of a negative control sample consisting only of H₂O (FIG. 8.6). In fact, ice grain sizes for the 0.1 mg(ml BSA and 0.1 mg/ml α-lac samples for the most part appear indistinguishable from those of a positive control containing 0.025 mg/ml Tm 12.86 in H₂O.

FIG. 8.7 compares the effects of −2° C. and −6° C. annealing temperatures on recrystallization using photographs of samples containing 0.025 mg/ml THP and 0.1 mg/ml BSA in H₂O. At −6° C. and two hours annealing time, grain sizes for the THP and BSA samples appear similar—both exhibit RI effects. However, at −2° C. and two hours annealing time, the inhibitory effect of the BSA sample appears to have been eliminated to a great extent (though not completely), while that of the THP remains. The same effect was also observed for 0.1 mg/ml α-lac samples at −2° C., though here significant grain size heterogeneities were apparent. Therefore, these results indicated that higher annealing temperatures might be used to help eliminate non-THP induced R.I. while maintaining THP-specific R.I. effects. However, the higher annealing temperature introduces more within sample heterogeneity.

To quantitatively evaluate the effectiveness of higher annealing temperatures with respect to the elimination of non-THP R.I. effects, composite mean largest grain size values were determined and statistically compared for the following solutions of non-THPs in H₂O: 0.1 mg/ml BSA, 0.01 mg/ml BSA, 0.1 mg/ml α-lactalbumin, 0.01 mg/ml α-lactalbumin, 0.005 mg/ml α-lactalbumin, 0.01 mg/ml Tm 12.86, and pure H₂O control samples all annealed at either −2° C. or −6° C. for two hours. As seen FIG. 8.8 detailing mlgs, there is a low threshold level of non-THP concentration in which RI effects can no longer be detected for both −6° C. and −2° C. annealing temperatures. For BSA in H₂O, RI effects were still evident at 0.1 mg/ml (p=0.0002, n=4 for 0.1 mg/ml BSA/H₂O, n=5 for H₂O), but were eliminated at 0.01 mg/ml (p=0.172, n=4 for 0.01 mg/ml BSA/H₂O, n=5 for H₂O) at −6° C. annealing temperature, two hours annealing time. With respect to the BSA samples annealed at −2° C., RI effects were again evident for BSA concentrations at 0.1 mg/ml (p=0.004, n=4 for 0.1 mg/ml BSA/H₂O, n=5 for H₂O), but eliminated at 0.01 mg/ml, though in this case mean largest grain sizes for the 0.01 mg/ml level were significantly larger than those observed for the H₂O controls (p=0.0012, n=4 for 0.01 mg/ml BSA/H₂O, n=5 for H₂O). For α-lac in H₂O, threshold concentrations for the elimination of RI appeared (statistically) similar to those of BSA, though slightly lower for an annealing temperature of −6° C. R.I. effects were still evident at 0.01 mg/ml (p=0.021, n=4 for 0.01 mg/ml α-lac/H₂O, n=5 for H₂O) but eliminated at 0.005 mg/ml for an annealing temperature of −6° C.; however, mean largest grain sizes for 0.005 mg/ml α-lac samples were significantly larger than the corresponding H₂O controls at −6° C. (p=0.0016, n=4 for 0.005 mg/ml α-lac/H₂O, n=5 for H₂O). At −2° C. annealing temperature, the α-lac RI effect was significant at 0.1 mg/ml (p=0.0003, n=4 for 0.1 mg/ml a-lac/H₂O, n=5 for H₂O), but not detected at 0.01 mg/ml (p=0.246, n=4 for 0.01 mg/ml a-lac/H₂O, n=5 for H₂O).

In summary, RI effects are clearly evident for non-THP proteins and at relatively low dilutions of these molecules (e.g. 0.01 mg/ml). RI effects for both BSA and α-lac in H₂O were generally eliminated between 0.1 mg/ml and 0.01 mg/ml (0.005 mg/ml for α-lac at −6° C.) for both −2° C. and −6° C. annealing temperatures. Higher annealing temperatures (i.e. −2° C.) do help to eliminate some of the non-THP R.I. effect as can be seen readily in FIG. 8.7, though curiously this RI elimination effect was less evident when making statistical comparisons using mlgs values. Based on experimental evidence obtained here, the RI—eliminating effects of −2° C. annealing temperatures appear to be most significant for the higher BSA or α-lac concentration solutions in H₂O (i.e. 0.1 mg/ml or higher) but does not completely eliminate the RI effect at these concentration levels, and may introduce greater sample heterogeneity.

b.) The use of NaCl solutions to eliminate non-THP R.I. effects. The ability of NaCl solutions to eliminate non-THP induced recrystallization inhibition was also assessed quantitatively. BSA (10 mg/ml and 1 mg/ml) and α-lactalbumin (10 mg/ml, 1 mg/ml, and 0.5 mg/ml) dissolved in 0.9% NaCl solution were used again as representative non-THPs. The addition of NaCl substantially accelerated recrystaUization: ice grain sizes for 0.9% NaCl samples annealed at −6° C. for 30 minutes were observed to correspond roughly with grain sizes of H₂O samples annealed at −6° C. for 2 hours, thus providing the means to more rapidly assess recrystallization for multiple samples. Therefore, all samples containing 0.9% NaCl were splat cooled and annealed for 30 minutes rather than two hours. In addition, all samples were annealed at −6° C. (since intrasample heterogeneity is quite significant with 0.9% NaCl annealed at −2.0° C. for 30 min).

After annealing, composite mean largest grain sizes for the α-lac and BSA samples were assessed and compared to mean largest grain sizes for both positive (0.001 mg/ml Tm 12.86 in 0.9% NaCl) and negative (0.9% NaCl) control samples. A graphical presentation of the resultant mlgs data is provided in FIG. 8.9. For both the α-lac and BSA categories, the presence of NaCl provided a much more potent means of eliminating non-THP RI effects (while retaining strong THP activity) as compared to the use of higher annealing temperatures for non-THPs in H₂O. For this reason, the concentrations of α-lac and BSA used in this evaluation were considerably higher (0.5 mg/ml to 10 mg/ml) than those used for non-THPs in H₂O (0.005 mg/ml to 0.1 mg/ml). In the case of BSA/0.9% NaCl samples, RI activity was eliminated for 1 mg/ml concentrations (p=0.999, n=6 for 1 mg/ml BSA/0.9% NaCl, n=16 for 0.9% NaCl), but was still detectable for 10 mg/ml concentrations (p=0.0011, n=6 for 1 mg/ml BSA/0.9% NaCl, n=16 for 0.9% NaCl). For α-lac/0.9% NaCl, the threshold concentration for elimination of RI was also 1 mg/ml (p=0.085, n=7 for 1 mg/ml α-lac/0.9% NaCl, n=16 for 0.9% NaCl), while statistically significant RI effects were detected at the 10 mg/ml level (p=0.0005, n=7 for 10 mg/ml cα-lac/0.9% NaCl, n=16 for 0.9% NaCl).

In summary, RI effects for both BSA and α-lac in 0.9% NaCl were eliminated at concentrations between 10 and 1 mg/ml. Thus, a roughly 10-fold increase in efficacy of non-THP RI elimination was observed with the addition of NaCl over the use of higher annealing temperatures with the BSA/H₂O or α-lac/H₂O solutions. Yet relatively strong RI characteristics were retained for dilute samples of both Tm 12.86 (0.001 mg/ml) and T. molitor hemolymph (1/1000 dilution) in 0.9% NaCl (p<0.0002 for comparisons of Tm 12.86 (n=8) and T. molitor hemolymph (n=7) with all other categories of non-THPs/0.9% NaCl and 0.9% NaCl controls).

In addition, the unusual situation in which more dilute samples of BSA and α-lac in H₂O exhibited composite mlgs values larger than the corresponding H₂O controls did not occur for the BSA and α-lac samples in 0.9% NaCl.

We also examined and quantified the acceleration effect of recrystallization behavior attributed to the use of NaCl, and whether this compromised the RI behavior of the THP solutions. To quantify the acceleration effect, composite mean largest grain sizes for 0.9% NaCl, pure H₂O, 5 μg/ml THP in 0.9% NaCl, and 5 μg/ml THP in H₂O were assessed at 1 minute, 30 minutes, and two hours. The results are presented in FIG. 8.10. Acceleration of recrystallization is apparent for both 0.9% NaCl and 5 μg/ml THP in 0.9% NaCl samples relative to their respective controls; however, the inhibitory effect of Tm 12.86 in both H₂O and 0.9% NaCl is still evident throughout these time frames. Thus, since the use of saline solutions of THPs and THP-containing hemolymph samples allowed for a decrease in ice sample annealing time from 2 hours to 30 minutes without compromise to their RI behavior, most of the remaining studies were conducted with the use of 0.9% NaCl and a 30 minute annealing time.

Finally, we explored the influence of salt concentration on ice grain size during recrystallization. A simple comparison between 1.8% NaCl and 0.9% NaCl ice samples annealed at −6° C. for 30 minutes reveals a statistically significant difference in composite mean largest grain sizes (p<0.001, n=5 samples for 1.8% NaCl, n=7 samples for 0.9% NaCl (FIG. 8.14)) where, perhaps surprisingly, the more concentrated salt solution attenuated the acceleration of recrystallization. Like high concentrations of non-THPs, this effect may become important in RI studies involving pure or concentrated insect hemolymph samples: differences in salt content are likely to occur between different individuals and different insect species. For studies of RI in T. molitor and D. canadensis hemolymph presented here, the effect is probably not significant due to the relatively dilute samples used (usually 1/100 or greater dilutions in 0.9% NaCl). However, this “solute load” effect capable of attenuating recrystallization may have significant consequences for experimental systems generating hyperosmotic conditions, particulary those attempting to evaluate the effectiveness of AFPs on RI.

Based on our results concerning the recrystallization accelerating effects of 0.9% NaCl, the relative homogeneity of ice grain sizes within 0.9% NaCl solutions at −6° C. annealing temperatures, and the potency of 0.9% NaCl solutions with respect to the elimination of non-THP R.I. effects while maintaining strong THP RI activity, we used 0.9% NaCl solutions annealed at −6° C. for 30 minutes for most subsequent applications of the RI assay presented here.

Concentration-dependent RI effects of THPs: the development of a quantitative RI assay. Once parameters for eliminating non-THP induced RI effects and establishing a sample composite mean largest grain size (based on ice grain size homogeneity) were detailed, the RI concentration-dependent effects of THPs were characterized and quantified. For the first analysis splat cooled ice samples consisting of 25, 10, 5, 2.5, and 1 μg/ml dilutions of purified Tm 12.86 in H₂O were annealed at both −6° C. and −2° C. for two hours, then photographed and analyzed to determine composite mean largest grain sizes. For each annealing temperature, the results indicated that Tm 12.86 inhibits recrystallization in a concentration-dependent fashion, with decreasing inhibition as the THP concentration was decreased from 25 to 1 μg/ml. Photographs showing the concentration-dependence of R.I. for Tm 12.86 at an annealing temperature −2° C. are presented in FIG. 8.12. Mean largest grain sizes for these Tm 12.86/H₂O samples at both −2° C. and −6° C. are given in FIG. 8.16. For both annealing temperatures, mlgs values for the Tm 12.86/H₂O solutions remained significantly smaller than those observed for the H₂O and 5 μg/ml α-lactalbumin controls down to and including the 1 μg/ml level (p<0.02 for both −2° C. and −6° C., n=4 for all categories except H₂O (n=5); the 1 μg/ml level represents the most dilute Tm 12.86/H₂O concentration tested). Therefore the lower limits of detectable RI effects for Tm 12.86 in H₂O are at least 1 μg/ml.

The concentration dependent RI effects of THPs in 0.9% NaCl were also evaluated Based on the results obtained for Tm 12.86 in H₂O, a broader range of Tm 12.86 concentrations were tested in 0.9% NaCl. Mean largest grain size evaluations were conducted for 250, 25, 10, 5, 2.5, 2, 1, 0.5, and 0.1 μg/ml Tm 12.86 in 0.9% NaCl samples annealed at −6° C. for 30 minutes. Photographs of Tm 12.86/0.9% NaCl samples annealed at −6° C. are presented in FIG. 8.14. Evaluations were also conducted for 25, 10,5, 2.5, 2, and 11 g/ml Tm 12.86 in 0.9% NaCl samples annealed at −2° C. for 30 minutes; in the case of −2° C. samples composite mean largest ice grain sizes were determined using the maximum/minimum deformation sampling technique described previously.

Like Tm 12.86 in H₂O, composite mean largest grain sizes for the Tm 12.86/0.9% NaCl solutions again indicated a concentration-dependent effect with respect to RI for both −2° C. and −6° C. annealing temperatures; unlike the Tm 12.86/H₂O solutions, however, an abrupt decrease in the limit of detectable RI was apparent at −2° C. in comparison to −6° C. (as seen in the mlgs graph of FIG. 8.15). At −2° C., significant RI effects were lost between 10 μg/ml Tm 12.86 and 5 μg/ml Tm 12.86 (p≦0.00013 for Tm 12.86 concentrations≧10 μg/ml, n=7 or 8 for all Tm 12.86 categories, n=16 for 0.9% NaCl controls; p=0.726, n=8 for 5 μg/ml Tm 12.86 and n=16 for 0.9% NaCl controls). At −6° C., the limit of detectable RI was more similar to that of Tm 12.86 in H₂O: significant RI effects were lost between 0.5 μg/ml and 0.1 μg/ml (p<0.0002 for Tm 12.86 concentrations≧0.5 μg/ml, n=4 for 250 and 25 μg/mlTm 12.86, n=7 or 8 for 10, 5, 2.5, 2, and 1 μg/ml Tm 12.86, n=16 for 0.9% NaCl controls; p=0.783, n=4 for 0.1 μg/ml Tm 12.86 and n=16 for 0.9% NaCl controls).

A plot of mean largest grain sizes as a function of the logarithm of Tm 12.86 concentration is given in FIG. 8.16a for samples annealed at −6° C. for 30 minutes. The resultant curve exhibits linearity within the THP midrange concentration region (˜10 μg/ml to 0.5 μg/ml). Mean largest grain sizes tend to level off for both the more dilute (less than 0.5 μg/ml) and more concentrated (greater than 10 μg/ml) THP concentrations. For concentrated Tm 12.86 solutions, ice grains are extremely small and difficult to measure, thus mean largest grain sizes may be overestimated. For dilute Tm 12.86 dilutions (less than 0.5 μg/ml), mean largest grain sizes can no longer be distinguished from those derived from 0.9% NaCl control samples. In addition to purified Tm 12.86 RI dilution profiles, hemolymph samples from T. molitor_were also evaluated for RI behavior. A single T. molitor larva with hemolymph thermal hysteresis of 2.6° C. was diluted to 1/50, 1/100, 1/500, 1/1000, 1/2000, 1/5000, 1/10000, 1/20000, and 1/50000 concentrations in 0.9% NaCl. The samples were splat-cooled, annealed at −6° C. for 30 minutes and evaluated for composite mean largest grain size. The mean largest grain size data is plotted as a function of log(dilution) in FIG. 8.16b. The resultant curve, similar to the profile derived for the Tm 12.86 dilution series, also exhibits linearity within the midrange region, with mean largest grain sizes leveling off for both the more dilute (less than 1/20,000 dilution) and more concentrated (greater than 1/1000 dilution) hemolymph samples.

Linear regression was used to characterize the approximately linear portion of the Tm 12.86 mean largest grain size dilution profile (10 μg/ml to 0.5 μg/ml) for samples diluted in 0.9% NaCl and annealed at −6° C. A coefficient of determination (R²) of 0.862 revealed a fairly strong linear relationship between mean largest grain size and the logarithm of Tm 12.86 concentration within this region (FIG. 8.17a).

The association of THP concentration with ice grain size through linear regression provides a basis for the development of a numerical factor that, in a manner analogous to thermal hysteresis measurements, describes the potency of a THP solution with respect to RI capability. This factor, designated here as the “RI factor”, is equal to the absolute value of the logarithm of the minimum THP dilution required to eliminate RI activity. To calculate the RI factor, regression analysis is first performed to provide an approximation of the relationship between mean largest grain size and THP concentration. The -log(dilution) corresponding to the intersection of the regression line with the baseline 0.9% NaCl or H₂O mean largest grain size then defines the R.I. factor.

FIG. 8.17a illustrates the RI factor computed graphically for the dilution profile of purified Tm 12.86, here estimated at 5.1. Since an RI factor of 5.1 describes the “R.I. sensitivity” of this reference Tm 12.86 THP solution (i.e. a 25 mg/ml starting solution of Tm 12.86 must be diluted ˜10^{5.1} times before losing RI activity), one can then proceed with the development of a quantitative RI assay based upon the estimation and comparison of RI factors for various THP solutions.

Since the linearity of mlgs with respect to THP concentration is the basis for development of the RI factor, one difficulty with this method arises due to the inherent curvature of dilution plots caused by the “leveling off” of mlgs values for both very dilute and very concentrated THP samples (see FIG. 8.16a). However, when a transforming function is applied to mean largest grain size (mlgs) values this serves to improve linearity within the approximately linear area of the mlgs plot. Using the arcsine(mlgs)^{0.5} transformation, the coefficient of determination (R²) for the Tm 12.86 profile is increased from 0.862 to 0.907 (˜5% increase). FIG. 8.17b shows the results of the plot transformation for Tm 12.86 in 0.9% NaCl. The RI factor estimate for the transformed Tm 12.86 dilution profile is now 4.88. The arcsine(mlgs)^{0.5} has improved linearity in all other profiles (14) presented in this study by an average of at least ˜7% (based on R² determinations), and thus has been incorporated into all further RI factor calculations.

We next examined variability of dilution profile data through the use of ninety five percent confidence interval bands surrounding the Tm 12.86 dilution profile regression line. Intersection of the bands with standard error of the mean (SEM) lines of the baseline 0.9% NaCl mean largest grain size provided a conservative estimate of RI factor variation. The use of 95% confidence interval bands is demonstrated for the Tm 12.86 dilution profile in FIG. 8.21. The resultant RI factor for the Tm 12.86 profile with 95% confidence bands is 4.88±0.147.

Further confirmation of the mean largest grain size method of RI factor determination was provided by ice sample “light scattering” behavior, an alternate method we have explored for quantifying recrystallization (detailed in Example 8 Section F2). To accomplish this task, replicate dilutions (in 0.9% NaCl) of hemolymph from a single T. molitor larva with a thermal hysteretic activity of 0.97° C. were subjected to splat cooling followed by annealing at −6° C. for 30 minutes. For each sample, two photographs were obtained: the first consisted of a single, whole sample photograph at 1/2000 second exposure to determine light scattering characteristics; the second consisted of a high magnification (44.5×) photograph used to determine mean largest grain size. Scanning densitometry plots of whole sample photograph negatives showed a general increase in absorbance with increasing hemolymph dilution (FIG. 8.3). Also, a general increase in ice grain size with increasing hemolymph dilution was apparent. Densitometry peak amplitudes were graphed as a function of the logarithm of hemolymph dilution, and a linear regression analysis performed. Corresponding mean largest grain sizes for all samples were also graphed and subjected to a linear regression estimate. The results are presented in FIG. 8.19a and b using the RI factor as a means of comparing the two methods of recrystallization inhibition measurement. Regression of sample absorbance data results in an RI factor of 3.40±0.22, while the regression of mean largest grain size data produces an RI factor of 3.50±0.13.

Although both the light scattering and mean largest grain size measurement methods result in similar RI factors, variation in the light scattering data is greater than that seen from mgs determination: coefficients of determination are R²=0.853 for the light scattering data regression and R²=0.941 for the mlgs data regression. Thus, composite mlgs assessment is both an accurate and reliable parameter to quantitatively assess recrystallization and the ability of AFPs to inhibit or retard this event. Moreover, it allows for the determination of an RI factor that indicates the efficacy of AFP induced RI, and a means for comparing potency of AFP solutions. While the light scattering assay is capable of effectively evaluating the extent of recrystallization occurring over time, it is not as accurate or sensitive of a method. However, it nevertheless holds great potential as more of a “screening tool” for large numbers of samples.

Given the above, showing that the use of linear regression with RI dilution profiles provided the best way to characterize the RI behavior of THP solutions not only in terms of the RI factor, but with other statistical techniques such as ANCOVA, the mlgs method was used for most applications of the RI assay presented below.

Applications of the RI assay. The development of an RI factor provides the basis of a quantitative assay detailing the RI strength of an antifeeze protein solution. To illustrate the usefulness of this assay and to determine further optimal assay parameters, we proceeded to explore several different applications of such an assay. First, we examined the effect of different sample annealing temperatures on the relative RI strength of a purified Tm 12.86 solution and T. molitor hemolymph solution from the perspective of RI factor determinations.

a) Effects of different annealing temperatures on RI factors for THP solutions. As presented previously, the RI factor derived from the dilution profile of 25 mg/ml Tm 12.86 in 0.9% NaCl was estimated at 4.88±0.147 (RI factor±95% C.I.) for samples annealed at −6° C. An identical RI factor analysis was performed for Tm 12.86 in 0.9% NaCl (25 mg/ml starting concentration) at an annealing temperature of −2° C. Recall from previous ANOVA results that loss of statistically significant RI activity first occurs at a lower dilution level for Tm 12.86 samples annealed at −2° C. (5 μg/ml) versus samples annealed at −6° C. (0.1 μg/ml); therefore, a significant difference in RI factors was also expected. Linear regression analysis of this data is presented in FIG. 8.20 with the corresponding RI factor determinations. For samples annealed at −2° C. (Tm 12.86 concentrations ranging from 25 μg/ml to 5 μg/ml), an RI factor of 3.88±0.100 is estimated. FIG. 8.20 also shows a comparison of RI factors between Tm 12.86 samples annealed at −2° C. and −6° C., indicating an approximately ten-fold increase in relative RI sensitivity for samples annealed at −6° C. as compared to those annealed at −2° C. This corresponds fairly well with ANOVA results presented earlier, though a comparison of RI factors seems to provide a more conservative outcome (a difference in sensitivity of approximately ten times using RI factors rather than approximately fifty times using ANOVA results).

Estimates of RI factors for Tm 12.86 samples in H₂O annealed at −6° C. and −2° C. were 4.87±0.229 and 5.049±0.184 respectively (FIG. 8.21), with both the −6° C. and −2° C. plots exhibiting fairly strong linear character (R²=0.890 and 0.953 for −6° C. and −2° C. regressions respectively). Overlapping RI factors suggest that the relative RI sensitivity of a 25 mg/ml Tm 12.86 in H₂O solution is similar for annealing at both −6° C. and −2° C. Therefore, the removal of NaCl would appear to eliminate the significant difference in RI potency observed when comparing Tm 12.86/0.9% NaCl solutions at −2° C. and −6° C.

Finally, we conducted a similar experiment with T. molitor hemolymph, whereby dilution profiles (in 0.9% NaCl) were obtained for a winter-acclimated T. molitor hemolymph sample displaying a T.H. of 3.17° C. All samples were diluted in 0.9% NaCl, then frozen and annealed at −6° C. and −2° C. Linear regression analyses of the results are presented in FIG. 8.22. The RI factor for the profile at −6° C. was estimated at 4.60±0.102 (R²=0.961). The RI factor for the profile at −2° C. was estimated at 4.36±0.076 (R^{2=0.94}). Like Tm 12.86 in 0.9% NaCl a decrease in RI potency at −2° C. also occurs for the hemolymph in this case; however, the gap between potencies at −2° C. and −6° C. appears much smaller than that observed for Tm 12.86/0.9% NaCl.

Thus from these experiments, we generally observed only very minor changes in RI strengths with changes in annealing temperature. The single important exception to this observation involved the difference between Tm 12.86/0.9% NaCl at −2° C. and −6° C. Because of the significant loss of RI capability for Tm 12.86 in 0.9% NaCl at −2° C., we chose to use −6° C. annealing temperatures for most of the remaining studies of RI dilution profiles. It is also interesting to note RI strengths remained comparable for both Tm 12.86 in 0.9% NaCl and Tm 12.86 in H₂O at −6° C. and −2° C. (RI factors ˜4.9).

b) Comparisons of insect hemolymph and Tm 12.86 RI dilution profiles. The inherent linearity observed for most RI dilution profiles provided the basis for a statistical comparison of several different dilution profiles at the same time. Dilution profiles for T. molitor hemolymph obtained from the single summer conditions-acclimated larva and the single winter conditions-acclimated larva were compared to the dilution profile for 25 mg/ml Tm 12.86, all samples diluted in 0.9% NaCl and annealed at −6° C. for 30 minutes (FIG. 8.23). RI factors for the summer conditions-acclimated T molitor profile, winter conditions-acclimated, and Tm 12.86 profile were estimated as previously described at 3.49±0.086, 4.60±0.102 and 4.88±0.147 respectively. T.H. measurements for the summer and winter conditions acclimated T. molitor hemolymph samples were 0.97° C. and 3.17° C. respectively; while a mean T.H. value for the 25 mg/ml Tm 12.86 samples was ˜1.75° C. (n=2). No overlap in RI factor intervals are apparent for any of the three profiles, though RI activities (as defined by the RI factors) for the winter conditions-acclimated T._molitor profile and Tm 12.86 profile are very similar. In addition, a significant shift in RI factors is apparent when comparing the summer-acclimated T. molitor profile (T.H.=0.97° C.) to the winter-acclimated T. molitor profile (T.H.=3.17° C.).

Regression coefficients for the two hemolymph profiles and Tm 12.86 profile were also compared using an analysis of covariance (ANCOVA). No statistically significant difference in regression slopes between the three lines was detected based on an ANCOVA test for homogeneity of slopes (p>0.25). A significant difference in elevation was detected by a subsequent ANCOVA test for homogeneity of elevations (p<0.001). A post hoc pairwise test (Tukey's) revealed significant differences in elevations between all three lines (p<0.001). Therefore, although the summer and winter hemolymph, and Tm 12.86 dilution profile least squares lines are essentially parallel, each line remains statistically distinct as reflected in the differences occurring for elevations and the non-overlapping RI factors.

To estimate the concentrations of Tm 12.86 (in 0.9% NaCl) that produce RI effects similar to those observed for the winter and summer conditions-acclimated T. molitor hemolymph, a series of theoretical regression lines reflecting the predicted linear profiles for various starting concentrations of Tm 12.86 was plotted FIG. 8.24 shows that the winter conditions hemolymph profile corresponds to approximately 10 mg/ml Tm 12.86, while the summer conditions hemolymph profile corresponds to approximately 1 mg/ml Tm 12.86.

As a confirmation of results comparing regression lines for hemolymph samples derived from individual larvae and Tm 12.86, dilution profiles from multiple larvae were determined. In this case, six larvae each were selected from winter conditions-acclimated and summer conditions-acclimated T. molitor populations respectively. Selection of the winter acclimated group was performed on the basis of relatively strong hemolymph RI activity, while selection of the summer acclimated group was performed on the basis of relatively weak hemolymph RI activity. Single dilution profiles for each of the six individuals of each group were consolidated into winter and summer dilution profiles. Regression analyses were performed in addition to T.H. measurements for each hemolymph sample (FIG. 8.25). RI factors for the winter and summer groups were estimated at 4.48±0.090 (R²=0.941) and 3.25±0.120 (R²=0.865) respectively, while corresponding T.H. values were 3.03±0.10° C. (range=2.7° to 3.24° C.) and 0.67±0.10° C. (range=0.45° to 1.0° C.) respectively. ANCOVA tests of regression coefficients were also performed using the hemolymph and Tm 12.86 arcsine(mlgs)^{0.5} data. The results again show that all three regression lines are essentially parallel (p>0.25), but arise from three statistically distinct populations with different line elevations (p<0.001). Similar to the single individual hemolymph samples analyzed previously, Tm 12.86 concentrations producing equivalent RI profiles as the multiple individual summer and winter-acclimated T. molitor hemolymph samples were determined using FIG. 8.26. The winter conditions-acclimated hemolymph profile corresponds to a Tm 12.86 concentration of approximately 10 mg/ml, while the summer-acclimated hemolymph profile corresponds to a Tm 12.86 concentration of approximately 1 mg/ml.

To determine if THPs other than those of T. molitor produce similar RI dilution profiles, hemolymph samples were tested from two different D. canadensis individuals. One individual with a thermal hysteresis of 0.5° C. was collected in July and the other individual, with a thermal hysteresis of 2.1° C., was collected in February. Dilution profiles resulted in estimated RI factors of 3.27±0.078 (R^{2=0.987}) and 4.21±0.073 (R²=0.939) for the July and February individuals respectively (FIG. 8.27). An ANCOVA analysis using the D. canadensis February and July individual, and T. molitor winter and summer individual hemolymph profiles in addition to Tm 12.86 (25 mg/ml) revealed a difference in regression slopes among the five lines (p<0.001). A post hoc pairwise comparison (Tukey's) of the regression line slopes detected no significant difference in slopes at the alpha=0.05 test level (p>0.10) with the exception of the D. canadensis February hemolymph profile. Significant differences were detected between the D. canadensis February hemolymph profile regression slope and the T. molitor winter and summer hemolymph and Tm 12.86 profile regression slopes (p<0.01). Curiously, the pairwise difference between slopes for D. canadensis winter and summer hemolymph profiles was not significant at the α=0.05 level (p˜0.06). However, pairwise comparisons of the D. canadensis summer hemolymph profile with the T. molitor hemolymph profiles or Tm 12.86 profiles revealed no significant difference in slopes (p>0.20). This discrepancy may be due to the small number of data points representing the D. canadensis summer hemolymph profile.

A further, less restrictive comparison of winter conditions-acclimated and summer conditions-acclimated T. molitor was also performed. Ten larvae each were selected at random from winter conditions-acclimated and summer conditions-acclimated T. molitor populations and hemolylnph samples collected for RI dilution profile and T.H. analysis. The winter acclimated T. molitor exhibited a mean±SEM T.H. of 3.07±0.30° C. (range=1.6° C. to 4.1° C.), while summer acclimated T. molitor exhibited a mean±SEM T.H. of 1.15±0.23° C. (range=0.2° C. to 2.62° C.). Thus, the randomly sampled larvae displayed a much greater range of T.H. levels than those described previously for the non-randomly selected larvae. R.I. factors were estimated at 4.47±0.143 (R^{2=0.747}) and 4.33±0.457 (R²=0.408) for the winter acclimated and summer acclimated T. molitor hemolymph profiles respectively (FIG. 8.28). The randomly sampled, winter acclimated RI factor corresponds fairly well with previous RI factors estimated for selected winter acclimated T molitor (R.I. factors of 4.6 and 4.49 for single and multiple selected, winter conditions-acclimated larvae respectively). However, a rather large discrepancy in RI factors exists for the randomly selected summer T. molitor as compared to the non randomly selected summer T. molitor (R.I. factors of 3.49 and 3.32 for single and multiple selected, summer conditions-acclimated larvae respectively). Yet a definite shift in RI dilution profiles is apparent in FIG. 8.28 between summer acclimated and winter acclimated profiles. An ANCOVA of the random summer and winter acclimated hemolymph RI profiles, and Tm 12.86 R.I. profile reveals a difference in regression line slopes (p<0.001). The post hoc multiple comparisons test reveals that the winter hemolymph profile remains essentially parallel to the Tm 12.86 profile (p>0.50); however, the summer hemolymph profile slope differs from both the winter hemolymph and Tm 12.86 profiles (p<0.025). At −2 C annealing temperature the slope profiles of Tm 12.86 and winter Tenebrio hemolymph differ significantly (P<0.05) (FIG. 8.29).

c) Detection of possible THP activity in solutions with no measurable T.H. activity using the RI assay: T. molitor fat body cell culture, cold-hardy frog blood plasma, recombinant protein from a cloned AFP gene. A distinct advantage of using RI effects to characterize THP activity is the high sensitivity of recrystalhzation inhibition to low concentrations of THPs. To demonstrate the use of RI profiles under these conditions, we first examined a T. molitor fat body cell culture system that given the scaled down numbers of cells within such an in vitro culture, detection of thermal hystersis is marginal. Thus, the culture media supernatant sample was subjected to two replicate dilution series and the arcsine(mlgs)^{0.5} plotted to estimate an RI factor (FIG. 8.30). In this case, a regression analysis provided an RI estimate of 1.94±0.18. For the same undiluted sample, no measurable thermal hysteresis was apparent, therefore demonstrating the inherent sensitivity of the RI assay. The equivalent Tm 12.86 concentration producing an RI profile similar in potency to that observed for the cell culture was determined from FIG. 8.31 as approximately 0.025 to 0.05 mg/ml. Also shown for comparison in FIGS. 30 and 31 are additional winter and summer conditions-acclimated hemolymph dilution profiles from single T. molitor larvae. RI factors of 4.96±0.180 and 3.41±0.140 were calculated for the winter and summer conditions acclimated larvae respectively, corresponding to T.H. measurements of 3.60° C. and 1.2° C.

The inherent sensitivity of THP-induced RI was further applied to the detection of possible low THP activity in frog plasma and bacterial lysate. In each case, a primary concern was the ability to distinguish RI effects induced by THPs from those produced by non-thermal hysteresis proteins, varying salt concentrations, and other possible factors. Therefore, in each case, the use of an appropriately selected control becomes essential in the accurate determination of THP RI activity.

Plasma from the freeze tolerant frog R. sylvatica (collected in early spring) was tested for the possible presence of THPs using an RI evaluation. As a control, the R. sylvatica plasma was compared to plasma obtained from R. pipens, a non-freeze tolerant frog not expected to synthesize thermal hysteresis proteins. Because higher concentrations of non-thermal hysteresis proteins such as BSA have been shown to induce RI effects, while variations in NaCl content also influence mean largest grain size during recrystalization, an effort was made to equalize total protein and ionic contents between R. sylvatica and control R. pipens samples. Total protein contents and osmolarities of each undiluted plasma sample (the total osmolarity of each sample was calculated as an approximation of ionic content) were determined. A mean osmolarity of all R. sylvatica (n=10) and R. pipens (n=10) samples was determined to be approximately equivalent to a 0.406% NaCl solution.

Subsets consisting of five samples each of the R. sylvatica and R. pipens samples were subjected to dilution series in 0.406% NaCl such that total protein contents were equalized to 10 mg/ml, 1 mg/ml and 0.1 mg/ml. All dilution samples, in addition to undiluted plasma samples, were subjected to splat cooling followed by annealing at −6° C. for 30 minutes. All samples were evaluated for composite mean largest grain sizes and compared using an analysis of variance (ANOVA). The results of the ANOVA indicate no significant difference in mean largest grain size existing within pairwise comparisons of R. sylvatica and R. pipens at undiluted, 10 mg/ml, 1 mg/ml, and 0.1 mg/ml concentration levels (p>0.86). Thus no THP activity is apparent for the R. sylvatica plasma. However, probable non-THP RI effects are apparent for the undiluted and 10 mg/ml plasma samples (FIG. 8.32).

A similar analysis was used in an effort to detect possible antifeeze activity of the recombinant form of T. molitor antifreeze protein, Tm 13.17. Bacteria containing the Tm 13.17 cDNA clone in an expression vector were induced to synthesize the recombinant form of Tm 13.17, then lysed to release all bacterial proteins. An identical procedure was performed on the same bacterial strain lacking the Tm 13.17 cDNA to produce a control lysate. Lysates from both bacteria types were dialyzed exhaustively against water, then lyophilized and resuspended in 0.9% NaCl in an effort to equalize ion concentrations between the samples. Protein determinations were also performed on each sample, followed by the preparation of dilution series of each sample such that total protein contents were equalized to 3.2, 1, and 0.1 mg/ml. Samples were then splat-cooled and annealed at −6° C. for 30 minutes. Photographic analysis showed that ice grain sizes between the lysate containing recombinant Tm 13.17 and the control lysate (lacking recombinant Tm 13.17) appear identical at each protein dilution level. Therefore, no THP-induced RI activity was readily apparent for recombinant Tm 13.17 for both lysate samples, however, there does appear to be some non-thermal hysteresis protein influence on recrystallization at the higher total protein concentration of 3.2 mg/ml and possibly at 1 mg/ml as well. This data was obtained prior to when the focus was shifted to the isolation of recombinant histagged products from the bacterial inclusion bodies. FIGS. 6.2 and 6.3 show that the recombinant Tm 13.17 isolated from bacterial inclusion bodies displays significant RI activity with an RI factor of 1.93.]

Of all the purified Tm 12.86 and T. molitor and D. canadensis hemolymph RI dilution profiles determined so far, only two appeared to differ significantly in terms of profile slope: purified Tm 12.86 in 0.9% NaCl at −2° C. annealing temperature, and winter acclimated D. canadensis hemolymph in 0.9% NaCl at −6° C. annealing temperature. The most pronounced difference was noted for the purified Tm 12.86/0.9% NaCl at −2° C. Here the loss in RI strength (as measured using RI factors) was calculated at ˜10-fold when compared to the relative RI strength of Tm 12.86/0.9% NaCl samples annealed at −6° C. As noted previously, a major difficulty encountered with 0.9% NaCl samples annealed at −2° C. is the presence of a consistent ice grain size heterogeneity as shown in FIG. 8.1b. To compensate for this heterogeneity, the maximum/minimum deformation method of composite mlgs determination was devised. However, we suspected that such a sampling method might be contributing to the unusual Tm 12.86/0.9% NaCl profile observed at the −2° C. annealing temperature.

To determine if this was the case, we repeated both the purified Tm 12.86/0.9% NaCl profile at −2° C. and a T. molitor hemolymph/0.9% NaCl profile at −2° C. using a random sampling method of composite mlgs determination (see Example 8, Section F1). Briefly, the random sampling method uses a grid consisting of squares approximately ˜1.5 mm by 1.5 mm in dimension onto which ice samples are placed for annealing at −2° C. The grid allows for the determination of mlgs values at random locations within the sample rather than at specific sites such as “maximum deformation” or “minimum deformation” locations. Using the random sampling method, composite mean largest ice grain sizes were obtained for dilutions of both purified Tm 12.86 (25 mg/ml starting concentration) and T. molitor hemolymph (T.H.=6.15° C.) in 0.9% NaCl. The resulting dilution profiles are shown in FIG. 8.33. Based on the graphed results of mlgs data, the regression lines appear very similar in slope and elevation and, in fact, are essentially coincident. ANCOVA results indicate this to be the case: the two dilution profiles are statistically indistinguishable (p>0.20). These results are very different from those obtained previously using the maximum/minimum deformation method of mlgs detemination in which a significant slope difference between the Tm 12.86 and T. molitor hemolymph profiles also annealed at −2° C. was detected. With respect to regression line slopes, the randomly determined mlgs data at −2° C. do correspond well with mlgs data previously obtained for samples annealed at −6° C. and sampled using the “center+mid-sample” FIG. 8.1a) technique. For purified Tm 12.86 and the several T. molitor hemolymph dilution profiles at −6° C., all regression line slopes were also found to be statistically equivalent. In addition, the mean RI factors computed for both the 25 mg/ml starting concentration Tm 12.86 dilution profiles at −6° C. (“center+mid-sample” sampling technique) and −2° C. (random sampling technique) are very similar: 4.88 at −6° C. and 4.82 at −2° C. (computed RI factors with 95% confidence intervals for the Tm 12.86 and T. molitor hemolymph profiles are 4.82±0.35 and 4.78±0.29 respectively).

Another unexpected outcome of the experiment was the unusually high hemolymph T.H. (6.15° C.) detected for the individual T. molitor larva used in this case. Based on the results presented in FIG. 8.33, the hemolymph has an RI strength equivalent to 25 mg/ml Tm 12.86 (both the Tm 12.86 and hemolymph dilution profiles are statistically equivalent). Such a high RI potency for the hemolymph sample is not unexpected based upon its T.H. value, though a similar RI potency was also detected for a hemolymph sample with a T.H. of 3.60° C. (samples annealed at −6° C.; see FIG. 8.31).

e) Confirmation of R.I. dilution profile slope differences: D. canadensis hemolymph/0.9% NaCl at −6° C. The second R.I. dilution profile which exhibited a significant difference in slope compared to most of the other Tm 12.86 and hemolymph profiles presented in this study was derived from a winter acclimated D. canadensis larva hemolymph sample with T.H.=2.1° C. Because winter-acclimated D. canadensis larvae are capable of producing much higher hemolymph thermal hysteresis values (as high as 8-10° C.), and since higher hemolymph T.H. values are probably associated with higher THP levels (at least in part), we sought out such high T.H. activity larvae in an attempt to confirm the previous R.I. dilution profile slope results. An R.I. dilution profile was developed for one such individual with a hemolymph T.H. value of 6.1° C. As presented in FIG. 8.34, the profile slope remains similar to both the purified Tm 12.86 and summer-acclimated (T.H.=0.5° C.) D. canadensis profiles, but appears to differ from the winter acclimated (T.H.=2.1° C.) D. canadensis profile (the Tm 12.86, summer acclimated D. canadensis (T.H.=0.5° C.), and winter acclimated D. canadensis (T.H.=2.1° C.) profiles are identical to those shown previously in FIG. 8.27). ANCOVA analysis confirms this assertion: the D. canadensis T.H.=6.1° C. profile slope is statistically indistinguishable from the Tm 12.86 or the D. canadensis T.H.=0.5° C. profiles (p>0.50), but is different from the profile slope for D. canadensis T.H.=2.1° C. (p<0.001). In addition, although the Tm 12.86 and D. canadensis T.H.=6.1° C. profiles result in similar RI factors, the two profiles remain statistically distinct based on a comparison of profile elevations (p<0.001).

f) Summary of RI factors vs. T.H. evaluations. From our hemolymph RI results, it is apparent that shifts in hemolymph RI dilution profiles appear to be related to changes in hemolymph thermal hysteresis. We therefore compared RI factors with corresponding T.H. values derived through this study. For the hemolymph and cell culture samples, a roughly positive correlation exists between RI factors and T.H. values. The purified Tm 12.86 profiles represent an exception to this rule, however the Tm 12.86 (25 mg/ml) T.H. values remain somewhat lower with respect to R.I. factors when compared to the T.H. values of hemolymph samples exhibiting similar RI factors. To better characterize the relationship between T.H. and R.I. activity, a graph comparing RI factors to T.H. values in T. molitor and D. canadensis hemolymph as well as T. molitor fat body cell culture is provided in FIG. 8.35 (purified Tm 12.86 was not included in this case). The relationship between RI factors and T.H. activity appears to be logarithmic, a result not altogether unexpected since the RI factor itself is defined in terms of the logarithm of a dilution factor.

Experimental results concerning the RI behavior of purified Tm 12.86, T. molitor and D. canadensis hemolymph, and T. molitor fat body cell culture generally support the assertion that recrystallization inhibition characteristics can be used to develop a quantitative, fairly specific, and sensitive assay for THPs.

The first step in the development of a quantitative R.I. assessment technique was to determine whether or not significant ice grain size variations occur within splat-cooled samples. An assessment of mean largest grain size homogeneity focused on three areas defined by FIG. 8.1a: (1) sample center, (2) mid-sample, and (3) sample edge. The boundary line which separates the sample center area from the mid-sample and edge areas in is believed to represent a wave front of the sample droplet returning toward the sample center after spreading on the plate. Two concerns involving grain size homogeneity were considered One concern involved the possible freezing out of solutes resulting in an uneven distribution of solutes within the sample, thus producing concomitant differences in mean largest grain sizes between the center and edge of the sample. The other concern involved possible variations in sample thickness arising from the presence of a wave front, since sample thickness has been shown previously to influence recrystallization rates in metals.

A study of mean largest grain sizes within the zones defined by revealed that mean ice grain sizes remain fairly homogeneous across the sample in most cases, despite the presence of both THPs and non-THPs. Cases where grain size heterogeneity were problematic occurred for BSA and α-lac in H₂O at concentrations above 0.1 mg/ml; however, in these specific cases the heterogeneities did not appear to be position-related within samples. A single, apparently position-related heterogeneity did occur for 0.01 mg/ml a-lac in H₂O at −6° C., with smaller ice grain sizes detected in the mid-sample area as compared to the sample center area. This effect may be due to subtle differences in sample thickness occurring between the center and mid-sample areas in this case or may simply be a statistical anomaly due to the low number of samples studied (n=3). However, no such difference was detected for the majority of the other H₂O-based samples at both −2° C. or −6° C., nor did any detectable differences occur for any of the 0.9% NaCl-based samples at −6° C.

The one circumstance where a position-based ice grain size heterogeneity was consistently a factor did not appear to be related to sample thickness or solute distribution. For samples annealed at −2° C. and containing 0.9% NaCl, smaller ice grain sizes were consistently found at a sample area defined relative to the sample support (see FIG. 8.1b), and seemingly independent of the sample zones defined by FIG. 8.1a. This phenomenon appears to be related to the gravity-induced deformation of the samples, resulting in smaller ice grain sizes at the area of greatest deformation. Since this same phenomenon does not occur for H₂O samples at −2° C., the presence of NaCl is probably indirectly responsible for the decrease in mechanical stiffness of the sample: the colligative melting point depression induced by the NaCl in solution would create additional sample liquid volume, which in turn would cause additional deformation or sagging of the sample. Increasing deformation may contribute to a pooling of liquid at the area of greatest deformation, therefore resulting in the smaller ice grain sizes required to accommodate the increase in intergrain liquid volume. A subsequent check for the deformation effect in 0.9% NaCl samples annealed at −6° C. for 30 minutes failed to reveal statistically significant variations in ice grain sizes, hence the volume of intergrain liquid present at −6° C. apparently remains small enough, and the sample mechanical stiffness sufficient, to prevent the significant grain size heterogeneity observed at −2° C. Information concerning ice grain size homogeneity was used to develop a single, representative composite mean largest grain size for each sample. We then applied the composite mean largest grain size evaluations toward the development of quantitative, sensitive, and THP-specific R.I. assays for Tm 12.86, insect hemolymph, and other THP solutions.

A random sampling technique for use with samples containing 0.9% NaCl and annealed at higher temperature (−2° C.) which appears to provide a more “robust” measurement of RI acitivity in the cases (FIG. 8.33) was developed. Such a method could also be used for all other sample and annealing conditions though our results concerning grain size distribution within samples indicate that, for most all cases other than 0.9% saline solutions at −2° C., the center+mid sample and random sampling methods of mlgs determination would probably produce similar results.

Non-THPs and Recrystallization Inhibition. The specificity of an RI assay with respect to THPs can be influenced by factors such as annealing temperature and the presence of non-THP solutes. As water solutions of proteins such as bovine serum albumin (68 kDa) and α-lactalbumin (14.4 kDa) induce RI effects for ice samples annealed at −6° C. The addition of sodium chloride at 0.9% w/v or an increase in annealing temperature to −2° C. can help to eliminate the non-THP RI effect while maintaining the RI activity of THPs. However, both of these methods are still limited by the concentration of non-THPs present in solution. Of the two methods, the use of saline solutions (0.9% NaCl) to eliminate non-THP RI effects appears much more effective. Results indicate that for samples in 0.9% NaCl annealed at −6° C. for 30 minutes, non-THP RI effects do not appear to be significant for concentrations less than or equal to 1.0 mg/ml. This mass/volume concentration limit was identical for the two non-THPs used in this study, BSA and α-lactalbumin, despite the obvious differences in amino acid compositions and molecular weights. For non-THP samples annealed at −2° C. for two hours, threshold concentrations for the elimination of non-THP RI effects were approximately 10-fold smaller: our data showed the non-THP concentration limit to be ˜0.01 mg/ml. Interestingly, a slight variation in concentration limit was observed when comparing BSA and α-lac in H₂O at −6° C. annealing temperatures: for a-lac, the threshold concentration was lower at 0.005 mg/ml, while for BSA the limit remained at 0.01 mg/ml. Despite this single difference between a-lac and BSA, it seems likely that other non-THPs would exhibit similar concentration limits with respect to the elimination of RI For both H₂O and NaCl solutions of BSA and a-lac, the similarities in mass/volume limits between the two non-THP species seems to indicate that non-THP induced RI is probably a steric phenomenon rather than a protein-specific effect.

Indeed, the physical explanation of how non-THPs can cause RI under certain circumstances and how this effect can be eliminated presumably relate to the fact that the addition of sodium chloride increases the volume of liquid between ice grain boundaries, thus allowing greater mobility of non-THPs within the boundary layer. This assertion is supported by the smaller ice grain sizes observed for 1.8% NaCl samples as compared to 0.9% NaCl samples, both annealed at −6° C. for 30 minutes. The higher salt concentration would most likely increase the total liquid to solid ratio within the sample and, since the total volume of the sample remains fairly constant, accommodation of the additional liquid phase between ice grains would require smaller ice grain sizes. A similar explanation may apply to the effects of higher annealing temperatures on non-THPs in water. The non-THPs are predicted to occupy the thin zones existing between ice grains and with them some liquid H₂O would also be present (due to colligative freezing point depression). Higher annealing temperatures would tend to increase the liquid volume between ice grains, again increasing the mobility of the non-THPs within this boundary layer. Mobility of the non-THPs would be important with respect to migration of ice grain boundaries; freezing around an immobile non-THP molecule would result in an additional increase in surface area not required if the non-THP was simply pushed ahead of an advancing ice front. Since the increase in ice grain surface area would be energetically unfavorable, the grain growth would be inhibited. Despite the presence of liquid inclusions resulting from higher annealing temperatures or NaCl, higher concentrations of non-THPs within these inclusions may result in some decrease of non-THP mobility. This may explain the non-THP R.I. effect observed for higher concentrations of non-THPs in NaCl solution, or in H₂O at higher annealing temperatures. In addition, both non-THP and salt concentration effects may also explain the RI effects seen in control samples containing higher concentrations of M. sexta hemolymph, frog plasma, or TMG cell culture medium.

The accelerating effect of certain NaCl solutions with respect to recrystallization is more difficult to explain, although it too is probably related to the presence of liquid inclusions between ice grains. The migration of H₂O molecules between adjacent ice grains probably requires that each H₂O molecule leaving one grain must reorient itself to match the lattice orientation of the second grain (which in all probability differs from that of the first ice grain). The molecules in liquid phase would presumably be fairly mobile, thus the presence of liquid H₂O would, in effect, decrease the “activation energy of reorientation” of migrating molecules. For H₂O samples lacking NaCl or protein solutes, liquid inclusions would not be expected to occur (although impurities may still allow very small quantities of liquid to exist). In this case higher annealing temperatures would act as the facilitator of intergrain H₂O migration, hence the acceleration of recrystallization observed for H₂O samples at −2° C. With respect to the elimination of non-THP R1, the greater efficacy of NaCl over higher annealing temperatures may be the result of much larger liquid volumes generated, thus allowing for increased non-THP mobility.

The accelerated grain growth occurring for 0.01 mg/ml BSA/H₂O at −2° C. and 0.005 mg/ml α-lac/H₂O at −6° C. as compared to H₂O controls (see FIG. 8.8) could be related somehow to the NaCl effect. This particular phenomenon is most peculiar, however, since no significant accelerating effects were evident for the other non-THP/H₂O solutions such as 0.1 mg/ml BSA, 0.01 mg/ml BSA (at −6° C. only), 0.1 mg/ml α-lac, 0.01 mg/ml α-lac, or 0.005 mg/ml α-lac (at −2° C. only). Another possible explanation is that a change in experimental conditions, such as the possible influence of ambient temperature and/or humidity during transfer of the sample to the cold stage, may have played a role. Further investigation is certainly required, since this effect may have influenced the recrystallization behavior of other samples in this study.

For our purposes, however, the non-THP effect is most important in regard to the use of the RI assay as a diagnostic indicator of antifreeze activity. Two examples of this application included the testing of cold-hardy frog blood plasma and a recombinant protein for antifreeze activity. In each case, the general strategy for antifreeze testing was to maximize the protein concentrations used for RI detection (to gain the best possible chance of detecting activity) while minimizing or accounting for non-THP RI effects. These examples demonstrated that it is possible to exceed the protein limits described previously (based on BSA and α-lac concentrations) and still obtain an accurate assessment of antifreeze activity provided that proper controls to account for non-THP RI effects are included for comparative purposes.

Sensitivity of the RI Assay. Concerning the sensitivity of the RI assay, quantification of RI effects using mean largest ice grain sizes reveals that the presence of Tm 12.86 in 0.9% NaCl at a −6° C. annealing temperature is detectable down to concentrations between 0.5 μg/ml and 0.1 μg/ml. In contrast, thermal hysteresis measurements of Tm 12.86 are detectable only to ˜100 μg/ml (FIG. 1.9); thus the detection of Tm 12.86 by RI effects is at least 200 times more sensitive than detection by thermal hysteresis. The sensitivity of the RI assay is most striking with respect to insect hemolymph: the hemolymph of one winter conditions-acclimated T. molitor individual produced a detectable RI effect even after diluted 40,000 times in 0.9% NaCl (see FIG. 8.30). The sensitivity of the RI assay with respect to Tm 12.86 in 0.9% NaCl initially appeared to be influenced substantially by annealing temperature. Using the maximum/minimum deformation method of nilgs determination, Tm 12.86 samples annealed in 0.9% NaCl at −2° C. for 30 minutes produced detectable RI effects down to concentrations between 10 and 5 μg/ml, a sensitivity at least ten times weaker than that observed for samples annealed at −6° C. The sensitivity of RI detection for Tm 12.86 samples in H₂O, however, was similar for both −6° C. and −2° C. annealing temperatures. Both exhibited sensitivities less than 1 μg/ml, similar to the sensitivity of Tm 12.86 in 0.9% NaCl at −6° C. In addition, at both −6° C. and −2° C. annealing temperatures, RI sensitivities for winter acclimated T. molitor hemolymph are much more similar than the −6° C. and −2° C. sensitivities observed for purified Tm 12.86 (see FIG. 8.20).

Because of these discrepancies, we suspected that the unusual results obtained for Tm 12.86/0.9% NaCl at −2° C. might be due to the method of composite mlgs determination used in this case (maximum/minimum deformation method). To test this assertion, a second Tm 12.86/0.9% NaCl dilution profile was repeated at −2° C. using a random sampling method for composite mlgs determination (FIG. 8.33).The results support this assertion: the RI sensitivity of the Tm 12.86/0.9% NaCl solution (25 mg/ml starting concentration) at −2° C. as determined via the random sampling technique was ˜1 μg/ml, similar to the 0.5 μg/ml limit obtained for Tm 12.86/0.9% NaCl samples annealed at −6° C. Therefore, the sudden loss of R.I. activity observed for Tm 12.86/0.9% NaCl at −2° C. as shown in FIG. 8.15 would appear to be an inaccurate representation of actual physical behavior. Although the particular composite mlgs determination method used in this instance may be largely responsible, other factors could be involved. For instance, previous work has shown that Tm 12.86 is a fairly labile protein, and thus protein degradation could have played a role in the abrupt loss of RI activity. Changes in solution pH could also be a factor, since our study did not make use of buffers (mainly to eliminate additional solutes which might affect ice recrystallization in an unknown way).

Alternatively, RI analysis and the RI assay have been implemented with a more physiological buffered solution (PBS, phosphate buffered saline) to maintain pH values, that may otherwise influence the behavior of protein solutes. RI profiles generated and parameters used under these circumstance are essentially identical to those described with 0.9% NaCl annealed at −6° C. for 30 min. We feel the use of PBS to be a more natural solution in which to obtain assessments of AFP specific RI behavior and the elimination of non-THP induced RI effects, therefore this is currently the approach that is now routinely being implemented.

Quantification of RI: dlution profiles and RI Factors. Quantification of RI effects has also revealed an approximately linear relationship between the logarithm of Tm 12.86 concentration or hemolymph dilution and mean largest grain size. This linear relationship is strengthened further by the conversion of mean largest grain size data through the function arcsine[(mlgs)^{0.5}]. The ability to associate THP concentration with ice grain size through linear regression provides a basis for the development of the RI factor, a single numerical value which provides a more systematic measurement of the RI sensitivity or capability of a THP sample. A particular advantage of the RI factor is its use in comparisons of RI potency for ice samples of different THP composition and concentration, different salt contents, and annealed at different temperatures, since it is a dimensionless quantity calculated relative to baseline control samples lacking THPs.

For the selected T. molitor hemolymph samples represented in FIGS. 8.23, 8.25 and 8.30, the increase in hemolymph RI factor associated with the acclimation of T. molitor from summer to winter conditions is observed as leftward shifts of the regression lines This leftward shift of the dilution profiles was accompanied by a ˜2 to 2.5° C. increase in hemolymph T.H. values. As previously noted, the RI factor can be influenced by translation of a regression line along the x-axis or by changes in slope of the regression line. In the case of regression lines for selected winter-acclimated T. molitor hemolymph, summer acclimated hemolymph, and purified Tm 12.86, ANCOVA results revealed no significant differences in slope, but discerned significant differences in elevation corresponding to profile shifts. Therefore, in this instance, the increase in RI factor occurring as summer acclimated larvae are subjected to winter acclimation is due primarily to a translational shift in the dilution profile regression line. The basis for the translational shifts observed in FIGS. 8.23, 8.25, and 8.30 is probably related, at least in part, to an increase in hemolymph THP concentrations. The theoretical dilution profile lines based on different starting concentrations of Tm 12.86 in 0.9% NaCl are shown in FIGS. 8.24, 8.26, and 8.31. Steadily increasing concentrations of Tm 12.86 result in a leftward shift of the Tm 12.86 dilution profile. It is interesting to note that for the selected T. molitor hemolymph samples, the relative RI strength (as measured by RI factors) increases, on average, by a factor of ten as acclimation conditions change from summer to winter. The RI factor 10 fold increase is associated with only a ˜3 to 3.5 fold increase in T.H. values, a characteristic which may have important implications with respect to freeze tolerant organisms.

In addition, the increase in T. molitor hemolymph relative RI strength from summer to winter conditions can also be expressed in terms of the equivalent Tm 12.86 concentrations described previously (FIGS. 8.24, 8.26, and 8.31). Concentrations corresponding to ˜1.0 mg/ml Tm 12.86 for summer-acclimated individuals are increased to greater than ˜10 mg/ml Tm 12.86 for winter-acclimated individuals. Western blot information quantifying Tm 12.86 levels in T. molitor hemolymph, however, indicate that concentrations of only 2-3 mg/ml exist for winter acclimated individuals. Therefore, the translational shift in R.I. profile observed for winter acclimated T. molitor must be due to more than just a simple increase in Tm 12.86 hemolymph concentration to 2 to 3 mg/ml. One possible explanation for the greater than expected RI shift could be the influence of other THPs in T. molitor hemolymph, such as an increased presence of the Type II THP forms. Another possibility could be the apparent influence of an activator to Tm 12.86 (FIG. 1.12).

If the translation of the RI profile regression lines can be related, at least in part, to changes in THP concentration, the physical interpretation of the slope of the regression lines becomes more difficult based on the results of this study. It seems reasonable to conjecture that the slope of RI regression profiles might be related to the particular species of THP involved, and therefore may be an indicator of the ice-binding properties of the THP. [A cautionary note concerning slope comparisons must also be included here, since the particular selection of THP sample dilution ranges for linear regression can influence dilution profile slopes. For best results the ideal dilution range should start just beyond saturating levels of THPs (resulting in ice grain sizes no smaller than ˜0.0004 mm² based on the quantification methods presented here), and end at the very limit of RI detection. Although empirical, these guidelines seemed to work fairly well for the study presented here based on R² determinations of regression line estimates.]

ANCOVA results comparing Tm 12.86 and T. molitor hemolymph dilution profiles showed that for most of the regression lines tested (samples in 0.9% NaCl and −6° C. annealing temperature), slopes were homogeneous. Assuming different THPs would result in RI profiles with significantly different slopes, this result would not be immediately expected. Since T. molitor hemolymph contains several different THP species, T. molitor hemolymph dilution profiles would be expected to exhibit different slopes as compared to purified Tm 12.86. However, this result was generally not observed. With respect to T. molitor hemolymph, only two such slope differences were detected. The first of these involved randomly sampled, summer-acclimated T. molitor hemolymph dilution samples annealed at −6° C. (FIG. 8.28). In this case, however, the regression line slope seems to be somewhat of an anomaly caused by the widespread range of data points and does not appear to be a physical manifestation of different THP composition. The second slope difference involved a comparison of a T. molitor hemolymph dilution profile and purified Tm 12.86 profile, both at −2° C. annealing temperature. As discussed previously concerning the R.I. sensitivity of Tm 12.86/0.9% NaCl at −2° C., this slope difference can probably be dismissed as well on the basis of results presented in FIG. 8.33. Using a random sampling technique to determine composite mlgs rather than the maximum/minimum deformation method, FIG. 8.33 shows that no true slope difference is probably occurring between Tm 12.86/0.9% NaCl and T. molitor hemolymph dilution profiles at −2° C. In terms of RI factors, the random sample Tm 12.86/0.9% NaCl profile at −2° C. is very similar to the Tm 12.86/0.9% NaCl profile at −6° C., thus confirming the assertion that the RI potency of Tm 12.86 in 0.9% NaCl remains relatively unchanged with changes in annealing temperature.

In summary, most all of the T. molitor hemolymph and Tm 12.86 dilution profiles display a remarkable similarity with respect to regression line slope. The general homogeneity of slopes encountered for Tm 12.86 and T. molitor hemolymph could be due to the predominance of Tm 12.86 in hemolymph over other THPs, or could simply mean that dilution profile slopes for the different THP species are not significantly different. To further investigate the possible physical significance of dilution profile slopes, hemolymph RI dilution profiles for D. canadensis larvae were used in a comparison with T. molitor hemolymph and purified Tm 12.86 profiles to determine if slope differences might occur. Results indicated that the D. canadensis profiles exhibit many of the same characteristics as the T. molitor and Tm 12.86 profiles, including a strong degree of linearity after an arcsine[(mlgs)^{0.5}] transformation, and a general increase in RI factors with increasing T.H values. One significant slope difference was detected for a winter acclimated D. canadensis hemolymph/0.9% NaCl compared to the purified Tm 12.86/0.9% NaCl profile. However, a summer acclimated D. canadensis profile (T.H.=0.5° C.) slope was determined through ANCOVA to be indistinguishable from the Tm 12.86 and selected T. molitor hemolymph profile slopes. These results initially indicated the possibility that winter acclimated D. canadensis THPs might be influencing the R.I. dilution profile slope in a manner different than that observed for T. molitor THPs.

Since winter acclimated D. canadensis larvae are capable of producing hemolymph T.H. values of 8-10° C., we attempted to confirm the slope results obtained for the specimen with hemolymph T.H.=2.1° C. by producing an RI profile for a second winter acclimated individual with hemolymph T.H.=6.1° C. ANCOVA results detected no significant differences occurring between this profile and the Tm 12.86/0.9% NaCl profile. Thus it seems unlikely that any true slope difference exists between D. canadensis and Tm 12.86 or T. molitor hemolymph profiles. The difference occurring for the T.H.=2.1° C. D. canadensis individual may have been due to sample degradation, possibly the result of pH changes or freeze/thaw cycles.

Remarkably, all the Tm 12.86, T. molitor hemolymph, and D. canadensis hemolymph dilution profile data, including the profile data at −2° C. annealing temperatures, demonstrates the complete equivalency of R.I. dilution profile slopes despite differences in THP species composition. The possible influence of THP type on RI dilution profile slopes cannot be completely dismissed by these results, however, since only insect THPs have been included in this study. Also, examination of the D canadensis RI levels used hemolymph as the source, not purified AFP. Therefore, until other purified AFPs are subject to such RI analysis, one can not rule out the potential for RI dilution profile slope differences beween different AFP types. In particular, given that there is a substantial difference in TH activity between the insect and fish AFPs, it would be quite interesting to compare the insect RI dilution profiles with fish RI dilution profiles to determine if any slope differences occur.

The discovery of a activator associated with Tm 12.86 is also of interest with respect to influence on RI dilution profile slopes and translational shifts. Previous work on D. canadensis has demonstrated that activators interact directly with THPs but not with ice surfaces (activators exhibit no thermal hysteresis activity by themselves). Therefore, according to the hypothesis presented here, the presence of THP activators might not influence a THP dilution profile slope but could still induce a profile translational shift as described previously. Ice surfaces may ‘see’ THP+activator complexes covering a greater surface area as equivalent to an increase in THP concentration.

Relationship between T.H. and RI. As described previously, our study of T. molitor and D. canadensis hemolymph dilution profiles has shown that, in most cases, a positive correlation exists between hemolymph T.H. values and RI factors. The correlation does not appear to be linear, however. RI dilution profiles for a T. molitor hemolymph sample with T.H.=6.15° C. and a D. canadensis hemolymph sample with T.H.=6.1° C. were difficult to distinguish (in terms of profile translation) compared to T. molitor hemolymph samples with T.H. values of only −3 to 3.6° C. Because of this particular RI behavior for higher T.H. values, we sought to better characterize the relationship between T.H. and RI factors by graphing all hemolymph T.H. and RI data. In many cases this required the development of RI factors using single series dilution data obtained from the multiple selected and multiple random hemolymph dilution profile studies. However, this data does provide a general idea of the relationship between RI factors and T.H. as shown in FIG. 8.35, which appears to correspond best to a logarithmic curve. This may be due in large part to the definition of the RI factor as the logarithm of a THP dilution. FIG. 8.35 also demonstrates that the best resolution of the RI assay, as presented in this study, occurs for THP solutions with lower T.H. activities. For example, the assay is probably more likely to distinguish between hemolymph samples with T.H. values of 0.2° C. and 0.6° C. than it would between hemolymph samples with T.H. values of 6° C. and 8° C. Based on ANCOVA results, it appears that the RI assay functions best for distinguishing different insect hemolymph samples with T.H. activities less than ˜3.0 to 3.5° C.

If the RI factor can be interpreted roughly as a measure of RI potency or strength, then FIG. 8.35 also has implications for organisms producing only very low levels of thermal hysteresis activity, such as certain cold hardy plants. Here, a simple increase in T.H. from 0.1° C. to 0.5° C. would result in a ten-fold increase in R.I. strength (in terms of the RI factor), which may contribute to the reduction of ice recrystallization-induced tissue damage. It should be noted that the Tm 12.86 dilution profile data was not included in FIG. 8.35. R.I. factors for these profiles were among the highest at ˜4.8 to 4.9; however, the corresponding T.H. values for the undiluted samples (25 mg/ml starting concentrations) were only ˜1.6° C. to 1.75° C., values much lower than those predicted by FIG. 8.35. This apparent discrepancy may be due in large part to the fact that Tm 12.86 at 25 mgmml is well within the saturation area of the T.H. activity curve for Tm 12.86. Since this situation is quite different from insect hemolymph, these values were excluded from FIG. 8.35.

Mathematical modelling of recrystallization and RI. A better understanding of the experimental RI dilution profiles observed may ultimately be derived from mathematical modeling of ice recrystallization and recrystallization inhibition processes. The driving force for recrystallization is the minimization of surface free energy created at the boundaries of individual ice crystals. As the average crystal size within a sample increases, the total interfacial surface area decreases resulting in a concomitant decrease in the driving force for recrystallization. A decrease in the driving force for recrystallization also results in a corresponding decrease in the rate of recrystallization. For a hypothetical circular ice crystal with cross sectional area A(t) and circumference C(t), an equation describing this behavior is,
dA/dt=[1/C(t)]·K₁(K1=constant) (1)
If the circumference is expressed in terms of cross sectional area, the equation becomes,
dA/dt=[1/A^{1/2}]·K₂ (K₂=constant=K₁·0.5π) (2)
Solving for cross sectional area as a function of time,
A(t)=K₃·t^{2/3}+A₀(K₃=constant=(3/2K₂)^{2/3}; A₀=starting area at time=0) (3)

Assuming the starting crystal size A₀ is very small (˜0), equation (3) resembles curve fits for experimentally derived data (FIG. 8.36). Equation (3) does not account for the presence of THPs or other solutes, which may influence the constant K₃, the time exponent (2/3), or both. Equation (3) also predicts that for experimental data resembling the theoretical time course as shown in FIG. 8.36b, a logarithmic conversion of both ice grain area and time should result in an approximately linear plot. A log/log conversion of the experimental time course data shown in FIG. 8.37a appears to support this assertion, with slopes of the regression lines (as determined by the exponents of the approximating power curves) decreasing with concomitant decreases in the rates of recrystallization over time. The statistical advantages of applying linear estimates to recrystallation kinetics could be extremely important with respect to characterization of THP-induced recrystallization inhibition effects

Another interesting feature of FIG. 8.37a is that the regression lines representing Tm 12.86 solutions both in 0.9% NaCl and H₂O solutions are readily distinguishable from the controls (0.9% NaCl and H₂O) on the basis of slope. The slopes of the regression lines appear to be independent of the type of solvent used (either 0.9% NaCl or H₂O), but are clearly influenced by the presence of Tm 12.86. The slope change is probably Tm 12.86 concentration-dependent, however, the type of solvent involved does influence the elevations of the regression lines, however.

Physically, FIG. 8.37a indicates that the R.I. potency of Tm 12.86 is similar in both H₂O and 0.9% NaCl. This result is confirmed by previous dilution profile experiments showing similar RI factors for Tm 12.86 at 25 mg/ml diluted in H₂O and 0.9% NaCl. The differences in elevation between the H₂O and 0.9% NaCl regression lines probably occur due to the recrystallization-accelerating effects of NaCl.

The invention method has shown that RI can be applied in a quantitative, THP-specific way as an extremely sensitive means of THP detection and characterization. The RI assay expands the range of THP concentrations or hemolymph dilutions that can be detected to those that exhibit very low antifreeze activity, as exemplified by its application to T. molitor fat body cell culture. However the assay does have limitations, including an inability to distinguish between THP solutions with higher T.H. activities and difficulties with non-THP induced RI under certain conditions. Characterization of different THP species may also be possible using RI dilution profile slopes, although results from the selected samples used here show that RI dilution profile slopes remain surprisingly constant despite changes in THP solution content.

Preferably, a phosphate-buffered saline solution is used when developing dilution profiles for hemolymph, THP and non-THP solutions to eliminate possible variations in pH. Also, it is probably advisable (for the sake of consistency) to employ the random sampling technique of mlgs determination to all samples in order to avoid possible problems with grain size heterogeneities, although our results indicated that grain size heterogeneities were problematic only for 0.9% NaCl solutions annealed at −2° C.

The discrepancy in dilution profiles observed for the purified Tm 12.86/0.9% NaCl dilutions annealed at −2° C. and subjected to two different sampling techniques may be due to one or a combination of several different possible explanations. Among these is the explanation detailed previously, that the “maximum/minimum” deformation method does not provide a very accurate representation of composite mean largest grain size due to the presence of a grain size heterogeneity in each sample. The placement of samples on a flat grid may help to eliminate the grain size heterogeneity simply by providing more uniform support across the sample (and thus helping to prevent excessive sample deformation. A second explanation relates to the preparation of the cold stage between ice samples while collecting data using the “maximurn/mrninimum” deformation method. In the case of samples containing 0.9% NaCl and annealed at −2° C., the support ring was generally removed from the cold stage between samples and rinsed with ddH₂O before being placed back into the cold stage. This procedure was performed since the samples appeared to “stick” to the support ring, presumably due to the higher annealing temperature (the same procedure was generally not used for the −6° C. samples, though the support ring was usually rinsed between dilution series). Although the support ring was allowed to cool in the cold stage for several minutes before an additional ice sample was added, the elapsed time period may not have been sufficient to allow the ring to reach −2° C. Since the thermocouple was not in contact with the ring, any temperature deviations would have been difficult to detect. Temperatures higher than −2° C. might create the effect seen for the Tm 12.86 dilution series. When using the grid with random sampling technique, greater care was taken to ensure proper maintenance of temperature by carefully removing ice samples from the cold stage without removing the grid between each sample. Finally, a third explanation for the unusual slope observed in the case of the original Tm 12.86 dilution profile at −2° C. may have simply involved excessive degradation of the purified Tm 12.86 in solution. This may have occurred for Tm 12.86 samples during dilution profile assessment using the “maximum/minimum” deformation method. Purified Tm 12.86 is fairly labile and thus degradation is always a concern. However, this explanation seems less likely since the same effect apparently did not occur for samples annealed at −6° C., nor was the same effect observed for Tm 12.86/H₂O samples annealed at −2° C. (in both cases using the center+mid-sample sampling technique).

Concerning the similarity in R.I. potency observed for the T. molitor hemolymph sample with T.H.=6.15° C. to that of the hemolymph sample with T.H.=3.60° C., explanations become more difficult One possibility could be an erroneously low T.H. measurement in the case of the hemolymph with T.H.=3.60° C. However, the error would have to be an especially large one, and therefore this explanation seems unlikely. A more likely possibility could be an error in volume assessment for one of the hemolymph samples. Still another reason could be a non-linear association between hemolymph T.H. and RI potency. Though most of the data presented here seems to indicate that a positive correlation exists between T.H. and RI potency, nearly all of this data relates to T.H. values between 0.0° C. and ˜3.5° C. Clearly further data at higher hemolymph T.H. values is required, possibly by using D. canadensis hemolymph (known to reach T.H. values of up to 9° C.).

Automation of the R.I. assay. Although the recrystallization inhibition assay provides a highly sensitive means of THP activity detection, one notable drawback of the assay in its present form is the relatively extensive time required to complete an RI analysis. For most of the applications of the RI assay presented here, multiple samples within multiple dilution series are required to assess the RI activity of a THP solution. Currently the cold stage is able to accommodate only one splat-cooled sample at a time, thus multiple samples require a considerable amount of time and effort to complete. In addition, mean largest grain size determinations also contribute significantly to the time required for R.I. assessment.

The invention also details the use of light scattering as a means of quantifying RI as an alternative to mean largest grain size measurements, and is very amenable to automated processes (FIG. 8.3). As detailed, this method would be intended for more of a rapid screening technique with a moderately high level of quantitation. However, in instances (including samples identified throught the light scattering method) that require a high degree of quantitative accuracy, would then need evaluation via the milgs RI dilution profile and RI factor analyses.

The second problem associated with automation of the R.I. assay involves the ability to create and anneal multiple ice samples at the same time. A possible solution to this problem might be involve the use of an air gun system which creates multiple “splat-cooled” samples simultaneously by expelling liquid samples onto a −80° C. aluminum plate. The samples would then be transferred to a cold microscope stage or chamber capable of holding multiple samples at a constant annealing temperature. The extent of RI could then be assessed either by evaluating mlgs or light scattering characteristics.

A somewhat simpler method that combines the ability to freeze and anneal multiple samples at the same time, followed by RI assessment using light scattering characteristics, was recently attempted with some positive results. The method is based on work of Knight, C. and J. G. Duman ([1986] Cryobiology 23: 256-262) in which ice samples were created by nucleating a thin, supercooled liquid sample “sandwiched” between two glass microscope slides. In our case multiple, small volume (0.1 μl to 0.2 μl) samples were “sandwiched” between two circular 10 mm diameter coverglasses, then frozen by placing the coverglass “sandwich” on an aluminum plate at ˜−80° C. for 10 minutes. The small sample volumes used with 10 mm diameter coverglasses were necessitated by the small viewing area available within the cold microscope stage. The “sandwich” was then transferred to the cold stage using chilled forceps (−20° C.), where the samples were annealed at −6° C. for up to 12 hours. A schematic of this procedure is presented in FIG. 8.38.

Photographs of two samples prepared using this method are shown FIG. 8.39. The samples consisted of 1/50 T. molitor hemolymph in 0.9% NaCl and a 0.9% NaCl sample annealed simultaneously at −6° C. and photographed after 30 minutes and 12 hours of annealing time. From these low magnification photographs, it is apparent that noticeably greater light scattering occurs for the 1/50 hemolymph sample as compared to the 0.9% NaCl sample, especially at the much extended annealing time of 12 hours. The contrast observed between samples in this case could easily allow for subsequent quantitation through light scattering.

Somewhat less positive results were obtained for a second set of samples consisting of 1/500, 1/1000, 1/2000, and 1/5000 T. molitor hemolymph dilutions in 0.9% NaCl, and a 0.9% NaCl control sample. The low magnification view of the samples shown in FIG. 8.40 after 12 hours annealing time reveals little apparent contrast between different samples. Higher magnification views of the same samples in FIG. 8.41 confirm that only slight increases in average ice grain size occur with changes in hemolymph dilution from 1/500 to 1/5000. Rather striking differences are observed, however, between each hemolymph sample and the 0.9% NaCl control.

In general, these preliminary results indicate that recrystallization rates appear to be slower for the “sandwich” samples as compared to splat-cooled samples, thus longer recrystallation times may be required to provide better contrast between samples. Longer annealing times are not especially desirable for the purposes of RI assay automation; however, in the case of the sandwich method, the increase in annealing time is offset by the ability to analyze multiple samples at the same time. In addition, unusual and highly heterogeneous ice growth was observed for the 0.9% NaCl samples, especially after the 12 hours of annealing time required to elicit greater contrast between the controls and hemolymph samples. The extensive heterogeneity may cause difficulty with respect to quantification of RI effects, though this difficulty may be less severe with the use of light scattering rather than milgs determination for quantitation.

Although the longer annealing times involved with the “sandwich” method might be considered a disadvantage in certain respects, from a different perspective this characteristic may actually be desirable. As previously described, the hemolymph and 0.9% NaCl samples analyzed were subjected to comparatively long annealing times of up to 12 hours. Using the splat-cooling method, such lengthy annealing times would not be possible due to extensive sample sublimation. Our particular equipment configuration and methods involving splat-cooled samples allowed for maximum annealing times of only 5 to 6 hours before sample volume loss became significant. With the sandwich method, maximum annealing times could easily surpass 24 hours depending upon the starting volume of a given sample (though some 1 sublimation is evident with this method also, as seen in FIG. 8.39). Thus, long range RI studies are possible using the “sandwich” method, a capability which may help to elucidate possible differences between THP and non-THP induced R.I. behavior. Longer annealing times may also contribute to an increase in sensitivity with respect to detectable, THP-induced R.I. effects. Evidence of this is observed in FIG. 8.41 when comparing the 1/5000 dilution hemolymph sample to the 0.9% NaCl control. The contrast between the two samples is substantial, much more than would be expected using the splat-cooling method at 30 minutes annealing time. However, much more experimental work is required to confirm whether an increase in sensitivity indeed exists for the “sandwich” method over the splat-cooling method. Other factors such as evaporation of water from samples while preparing the “sandwich” (which may be significant in our case because of the extremely small volumes (0.1 to 0.2 μl) involved) may be contributing to the stronger RI effect observed for hemolymph using the this method.

Finally, by far the most promising advantage of the “sandwich” method over our current RI assay procedure involving splat-cooling lies in its adaptability to automated techniques. This could be accomplished most easily, with the fewest modifications or technical difficulties, by using an automated microplate reader with a temperature-controlled plate chamber. The standard 96-well microtiter plate generally used with this type of reader could be replaced by two flat plates of the same dimensions as the microtiter plate, with “sandwiched” RI samples occupying positions corresponding to those of the standard 96 well locations. Between the two plates, the samples would be frozen rapidly by placing them on a −80° C. aluminum plate for 10 minutes as described previously, then transferred to the microplate reader for annealing at a constant temperature and time. After annealing, optical density measurements would then be taken and recorded for each sample using the microplate reader. This procedure is quite feasible, although the relatively high cost of a microplate reader, and the added expense of modifying the reader to maintain constant, sub-zero temperatures is a distinct disadvantage (currently temperature-controlled, automated plate readers are available that sustain plate temperatures to only +5° C.). Further experiments using the method presented here with the cold stage are first required to determine the reliability of light scattering detection in conjunction with the “sandwich” method to adequately quantify RI.

Another level of automation of the RI assay is directed toward image capturing of the ice crystals viewed through the microscope for monitoring ice crystal growth over time and to report quantitative data based on what is observed in the field of view. This can be readily accomplished through either video recording using a CCD camera or through image capturing via a digital camera. Additionally, standalone image analysis software that will monitor ice crystal growth within a 256 gray-scale (or through more upscaled color monitoring) and perform size calculations on the resultant data, with particular reference to the foundation studies based on the mean largest grain size, RI dilution profiles, linear regression analyses for RI factors and ANCOVA slope analyses, will provide meaningful, reliable, biologically relevent calibration references. Therefore at least two modes for identifying ice crystals need to be employed. The first is to monitor the largest five ice grains (possibly from 2-3 separate fields of view) over time, for assessment of composite mlgs. The second mode could monitor all the grains that are in the field of view over time. Operator-editable parameters will allow choice of measurement frequency and selection of ice grain assessment characteristics evaluated. The advantages of all this over typical generic image analysis software will be the ability to relate computer assisted images and measurement parameters to true documented foundation studies on RI behavior of a purified AFP under a variety of different assaying conditions (as detailed in this invention). Moreover, it can facilitate implementation and testing of the mathematical modeling equations described and thereby also allow for a systems level approach and predictive theories to recrystallization behavior of solutions and the impact of ice growth suppressing peptides.

Importantly, the detail specifications of the RI assay as provided in this invention, and the sensitive and statistically reliable quantification analyses of composite mlgs provide the necessary framework with which to ensure that upscaled image capturing and analyses of camera ready fields of ice grains, and the computer generated area units and demarcation limits detected by the computer software instructions, have true biologically relevant meaning.

With a combination of upscaling for multiple samples and image capturing and analysis of their ice crystal grain sizes, this invention is likely to have numerous industrial and commercial uses for detecting and quantifying ice recrystallization, and also provide the impetus for reducing or eliminating deleterous ice with addition of AFPs. To name just a few examples, it is envisioned that the frozen food industry and ice cream manufacturers could better monitor and improve shelf life of their products, and the meat and poultry industries which also requires extended storage of partially thawed meats and poultry would be particularly suited for such implementation. Additionally, similar monitoring would provide important improvements for gauging the longevity of tissue cryopreservation and extended storage of synthetically engineered tissues, while predictive rates and therefore, selected control of localized ice crystallization would improve implementation in cryosurgury. Moreover, this would enable more large scale screening of the effectiveness of current and newly designed de-icing solutions, including those containing natural or recombinant and/other organically synthesized AFPs.

Other considerations. As described within the present invention, native Tm 12.86 has been found to display enhanced antifreeze activity in the presence of a partially purified fraction derived from cold hardy T. molitor larvae. The nature of this endogenous compound(s) one moderately characterized. It is within the scope of this invention to envision implementation of more extensive and further purification of these active compounds through HPLC and other means necessary for biochemical characterization. If the activator is confirmed to be proteinaceous, then both partial sequencing and generation of specific antibodies will be performed to subsequently allow for probes to screen our existing cDNA libraries to isolate the full length clones. It is also within the scope presented here to examine whether this activator is capable of enhancing the RI or TH activity all recombinant proteins generated by expression of the clones detailed in this invention. Similarly, it is foreseeable that such responses would also been observed using by antisera and/or isolated immunoglobulins originally generated against native Tm 12.86. Moreover, this polyclonal antiserum has numerous applications, similar to those employed in our western analyses studies and as a principle tool to screening the cDNA library for positive clones, regarding detection of other members of the Tm 12.86 family of products, both among other species, as well as in biotechnological applications

FIGS. 8.43 and 8.44 are also included for description and illustration of the regions of the clones (examples given for Tm 13.17 and 2-2) designated and used as DNA probes for Examples 4, 5 and in rtPCR studies.

The invention is illustrated by the following examples. These examples illustrate procedures, including the best mode for practicing the invention. These examples are offered by way of illustration, not by way of limitation.

### EXAMPLE 1

Purification of Tenebrio molitor Type m AFPs

Acclimation of Animals: Tenebrio molitor larvae were purchased from Carolina Biological Supply Company and were immediately subjected to a 21-day stepwise cold-acclimation (weekly steps at 15° C., 10° C. and 5° C.) while being maintained under a short-day (8 hour light, 16 hour dark) photoperiod. These conditions cause the larvae to display a significant elevation of hemolymph thermal hysteretic activity.

Purification. The first steps of protein extraction involved homogenizing 75 grams of whole cold-acclimated larvae in 150 ml of 4° C. 50% ethanol for 5 minutes in a blender. Whole larvae were used because antifreeze proteins in T. molitor are present in both hemolylnph, and within the fat body within discrete protein granules. The homogenized suspension was centrifuged at 4500 g for 15 minutes in a Sorvall RC-3 Refrigerated Centrifuge (4° C.). The layer of lipid on top was removed, the remaining supernatant was carefully decanted off, and the pellet was discarded. This 50% ethanol supernatant was placed in Spectrapor dialysis tubing (6,000-8,000 MW cutoff) and dialyzed at 4° C. for 72 hours against at least 10 changes of distilled water. The dialyzed supernatant was concentrated by lyophilization in a Virtis Model 10-145MR-BA freezedryer.

The lyophilized preparation was then subjected to ion exchange chromatography. The sample was dissolved in 25 mM Tris-Cl buffer (pH 9.0) at a concentration of 50 mg/ml and chromatographed on a DEAE-Sepharose CL-6B (Pharmacia) ion exchange column (2.5×20 cm), flow rate 2.5 ml/min. Fractions were eluted using stepwise increases in sodium chloride (0.03 M, 0.06 M, 0.09 M, 0.1 M, 0.2 M, 0.3 M, 0.5 M, 0.7 M, and 1.00 M) (8 ml/tube) and monitored at 230 nm (peak wavelength absorption of the peptide backbone) in an LKB Ultraspec II at 22° C. Elution peaks were pooled, lyophilized, suspended in 4° C. distilled water, dialyzed exhaustively against 4° C. distilled water, and lyophilized again. Freeze-dried peaks were dissolved in 4° C. double distilled water at a concentration of 50 mg/ml and screened for thermal hysteretic activity.

Peak II, one of two highly active fractions from the DEAE-Sepharose CL-6B column, was lyophilized, suspended in a 25 mM Tris-Cl buffer (pH 7.5) containing 0.1 M NaCl at a concentration of 50 mg/ml and subjected to further purification using size-exclusion chromatography. The protein peak was chromatographed on a Sephadex G-75 Superfine (Pharmacia) gel filtration column (1.2×60 cm) containing 25 mM Tris-Cl buffer (pH 7.5) containing 0.1 M NaCl at a flow rate 3.9 ml/hr (1.4 ml/tube). The eluant was monitored at 280 nm (the peak wavelength absorption of aromatic side chain amino acids). Protein peaks were collected, dialyzed against 4° C. distilled water, lyophilized, and tested for thermal hysteretic activity at a concentration of 25 mg/ml.

Peak 3 from the Sephadex G-75 gel filtration column was the only active fraction of ion exchange Peak II and was subjected to further purification on preparatory non-denaturing polyacrylamide gel electrophoresis at 4° C. Following electrophoresis, the antieeze band was visualized immediately (without fixation) with bromophenol-blue because bromophenol-blue was found to be reversibly associated with the antifreeze protein. The major band was excised, sliced (in order to increase surface area for the electro-elution process), and electro-eluted off the gel fragment in a Bio-Rad Electro-Eluter Model 432 for 12 hours at 5 mA and 4° C. in non-denaturing reservoir buffer (5 mM Tris-Cl base and 38 mM glycine). Once the elution was complete, eluates were combined and placed into dialysis bags (MW cutoff=6,000-8,000 Da) and dialyzed against distilled water for 72 hours at 4° C. with frequent changes of water. The dialyzed samples were lyophilized and tested for thermal hysteretic activity at 6 mg/ml.

Gel Electrophoresis. Aliquots (25 ug of protein) of samples from the ion exchange column, gel filtration column, and electro-elutions were run on both non-denaturing gel electrophoresis and SDS-polyacrylamide gel electrophoresis. For the native gels, samples were prepared in 20 ul of sample buffer (5 mM Tris-Cl base, 38 mM glycine, and 0.58 M sucrose) and run on 9%, 0.8 mm vertical slab gels (1.25 mm with a 10 cm trough for preparatory gels) under constant current (20 mA) at 4° C.

For the SDS-gel electrophoresis, samples were added to equal volumes of 2× sample buffer (0.125 M Tris-Cl base, pH 6.8, 20% glycerol, 10% b-mercaptoethanol, and 4.6% SDS) and sufficient volume of 1×SDS sample buffer to achieve a total volume of 25 ul. This preparation was then boiled for 5 minutes and electrophoresed on 15%, 0.8 mm vertical slab gels under constant current conditions (15 mA through the stacking gel and 20 mA through the running gel). Molecular weight standards (phosphorylase b, 94 kDa; bovine serum albumin, 67 kDa; ovalbumin, 43 kDa; carbonic anhydrase, 30 kDa; soybean trypsin inhibitor, 20.1 kDa; and alphα-lactalbumin 14.4 kDa: Pharmacia) were boiled in SDS sample buffer and co-electrophoresed with the experimental samples. To increase retention of peptides less than 10 kDa in size, 49.5% T/6% C SDS-polyacrylamide slab gels (0.8 mm) using a tricine buffer (0.1 M Tris-Cl base pH 8.25,0.1 M Tricine, and 0.1 M SDS) were also run. Samples were treated in standard SDS-sample buffer and electrophoresed (20 ug total protein/lane) at constant voltage (105 V). Additionally, when indicated, some samples run on SDS-PAGE were prepared without b-mercaptoethanol in the sample buffer.

Unless indicated otherwise, all gels were fixed in 50% methanol solution and stained with Coomassie brilliant blue for protein band detection. Additionally, some gels were subject to alternative protein stains, including silver stains and copper chloride, or screened for the presence of a carbohydrate moiety with Periodic Acid-Schiff's Base reagent.

Biochemical Characterization and N-Terminal Sequencing of Purified AFP. To confirm the purification of a single thermal hysteresis protein species following characterization on gel electrophoresis, the Cornell University Analytical and Synthesis Laboratory was contracted out to perform High Performance Liquid Chromatography (HPLC) of gel filtration Peak 3. One hundred micrograms of gel filtration Peak 3 was dissolved in ddH₂O and run on a Waters 900 HPLC with a Vydac C-18 Reverse Phase column for over 40 minutes at 1 ml/min using a 10-50% methyl cyanide gradient containing 0.1% TFA. Peaks were monitored at 210 nm and 280 nm. The major peak at 30 minutes was collected and was subjected to mass spectrometry and compositional analyses.

Mass spectrometry was performed on a Matrix Assisted Laser Desorption Mass Spectrometer at Cornell University. Sample treatment and instrument calibration were as specified by Cornell's Analytical and Synthesis Laboratory. Compositional analyses of the 30-minute HPLC peak included amino acid analysis on a Waters LC at the Cornell University Analytical and Synthesis Laboratory. Two separate analyses were performed. The standard, acid hydrolyzed amino acid determination was carried out using hydrolysis conditions for 90 minutes at 150° C. However, because cysteine and methionine are not adequately determined by this method, a second analysis using performic acid oxidation was conducted in hydrolysis conditions for 85 minutes at 150° C. Final amino acid composition involved normalizing the picomoles recovered between the standard acid hydrolyzed amino acid analysis and the oxidized amino acid analysis. To do so, the values for cysteine and methionine of the acid hydrolysis analysis were omitted and the values for tyrosine, phenolalanine and glycine of the oxidized analysis were omitted, since these are destroyed by performic acid oxidation.

Amino-terminal sequence analysis was also performed for the 30-minute HPLC peak. This was conducted using automated Edman degradation. Sample treatment and instrument calibration were as specified by Cornell's Analytical and Synthesis Laboratory.

Generation of an Antibody Against the Purified Antifreeze Protein. Since gel filtration Peak 3 was found to contain a single antifreeze protein species during gel electrophoretic assessment, we proceeded to run preparatory non-denaturing polyacrylamide gels to obtain a sufficient amount of antifreeze protein as an antigen (in an acrylamide matrix) for antibody production. Gel filtration Peak 3 at a concentration of 25 mg/ml was run on two preparatory (1.25 mm) non-denaturing polyacrylamide gels according to the method detailed earlier. Following electrophoresis, the gels were fixed and stained with Coomassie. For each gel, only one major band was observed. The first preparatory gel contained 1200 ug of total protein. This band was sliced out and split into eight equal sections. The excised band was used for the first four injections (approximately 150 ug of total protein per injection) for each of the two rabbits. For the second perparatory gel, a 1000 ug of total protein was run. The excised band was split into four equal sections and used for the final two injections (approximately 250 ug of total protein per injection) for each of the two rabbits. Bethyl Laboratories (Montgomery, Tex.) was contracted to inject each of the two rabbits with an antigen sample every two weeks for fourteen weeks. At this time, terminal bleeds of approximately 120 ml sera were taken and stored at −20° C.

Western Blot Analysis. Antibody specificity and sensitivity were examined by Western blot analysis. All Western blot analysis used 15% SDS-polyacrylamide gels (procedures detailed earlier) for protein separation. Pre-stained low-range molecular weights (phosphorylase B; 139.9 kDa, bovine serum albumin; 86.8 kDa, ovalbumin; 47.8 kDa, carbonic anhydrase; 33.3 kDa, soybean trypsin inhibitor; 28.6 kDa; lysozyme; 20.7 kDa: Bio-Rad.) or (myosin; 203.0 kDa, b-galatosidase; 135.0 kDa, bovine serum albumin; 81.0 kDa, carbonic anhydrase; 44 kDa soybean trypsin inhibitor, 32.3 kDa, lysozyme; 17.4 kDa, aprotinin; 7.5 kDa: Pharmacia) were used to provide a visual marker for transfer and an approximate molecular weight standard. All samples including hemolymph, tissue and purified antifreeze were run in the absence of b-mercaptoethanol.

Following electrophoresis, the gel and a 0.2 um nitrocellulose membrane were separated and soaked in ddH₂O for 5 minutes before their equilibration to Towbin's transfer buffer for 20-30 minutes. Proteins were electroblotted to the nitrocellulose membrane overnight (approximately 12 to 16 hours) at 40V in a CBS Scientific blotting tank at 4° C. Following transfer, the nitrocellulose membrane was treated as detailed below and the gel was stained with Coomassie to verify the efficiency of transfer.

The nitrocellulose membrane was rinsed in staining buffer (0.1 M PBS, pH 7.4) for 15 minutes with three changes of solution. The membrane was then blocked with fresh 5% nonfat dry milk in PBS with 0.1% NaN₃ for 2 hours and rinsed in PBS. Endogenous peroxidases were blocked with fresh 0.5% H₂O₂ for 20 minutes and the membrane was rinsed again. Next, a 100 ml dilution of 1:5000 primary antibody serum in staining buffer (determined in a related study to be an optimal antibody titer for this antiserum) was introduced to the membrane for 2 hours and the membrane was rinsed. Then, the membrane was treated with a 1:500 dilution of a peroxidaseqonjugated goat-anti-rabbit secondary antibody (Sigma) for 2 hours and rinsed again. Finally, the nitrocellulose membrane was stained with a 15 ml DAB solution (3,3′-Diaminobenzidine Tetrahydrochloride; Fast Dab: Sigma) until the bands were visualized (approximately 1-5 minutes with shaking). The DAB reaction was stopped by several rinses of PBS and the membrane was air dried under a weight

Determination of Thermal Hysteresis. Samples to be tested for thermal hysteresis (2-5 ul) were drawn into a 10 ul capillary tube. The end opposite to that which contains the sample was sealed under a flame. Finally, the sample was centrifuged down toward the sealed end and the open end of the capillary tube was sealed with mineral oil using a drawn out Pasteur pipette. These samples were then assayed for thermal hysteretic activity by what is commonly referred to as the micro-capillary method.

The capillary tube was placed into a refrigerated alcohol bath chamber and was viewed with a dissection microscope through a viewing port. The bath was set just below the sample melting point temperature (determined empirically). A small seed crystal (0.25 mm) was sprayed with spray-freeze (Cryowick, Fisher#12-645-20) and the capillary tube was placed into the alcohol bath chamber. The temperature was raised 0.02° C./5 minutes until the crystal disappeared. This temperature was taken as the colligative melting point temperature. The temperature of the refrigerated alcohol chamber was then lowered just below that of the melting point and another seed crystal was sprayed in the capillary sample. The temperature was lowered 0.1° C./2 minutes until the crystal began to grow. This temperature was taken as the freezing point. Samples without antifreeze proteins exhibit melting and freezing points that were virtually identical (within 0.02° C.). However, samples containing proteinaceous antifreezes will generate a thermal hysteresis, i.e. thermal hysteretic activity is defined as a non-colligative depression of the freezing point below that of the sample's colligative melting point. The amount of thermal hysteresis observed reflect the type of AFP (e.g. fish verses insect AFP, insect AFPs being more potent) and the concentration of AFP is solution.

An advantage of the microcapillary method is that if offers great consistency of thermal hysteresis measurements due to controlled measurement parameters, and can detect thermal hysteresis activity as small as 0.02 C, corresponding to the estimated resolution of the method. However, this method is subject to seed crystal size variation, which can influence thermal hysteresis measurment, and requires experimenter skill to minimize this effect.

The other common method for determining thermal hysteresis behavior is by using an nanoliter osmometer (Clifton Technical Physics, Marftord, N.Y.). The device is a thermal electric cooling module that can be set up on a microscope stage, such that the growth and melt behaviors of ice crystals can be observed. The sample holder contains a few small holes (about 0.35 mm in diameter) that can be filled with immersion oil. The test sample (1-5 nl volume) is then inserted into the oil. The sample is then subject to rapid freezing to −4° C. followed by rapid rewarming to observe the melting point temperature. Another crystal is then formed in the sample again through rapid freezing and rewarming. Just prior to when the last crystal would melt, the temperature is again lowered until the freezing temperature is reached. The presence of AFPs will generate a hysteretic gap in this instance, while non-AFPs do not. This device also requires experimenter skill to achieve reproducibility.

The two assays are not directly equitable, given differences in cooling rates and size of seed crystals. However, it should be clear to the reader that the native and synthetic proteins of this invention are not limited to being screened via the capillary method, and can be readily evaluated in in the nanoliter osmometer method, or even other less common methods (differential scanning caloritmetry, temperature gradient osmometry) used to assess non-colligative freezing point depressive activity.

However, whenever detection of antifreeze protein specific activity is required at dilute solutions of AFPs, subthreshold for being capable of generated thermal hysteresis, a more sensitive screening approach and antifreeze protein specific assay is required. The quantitative recrystallization inhibition assay detail later in this invention fulfills this need.

Screening and Partial Purification for an Antifreeze Enhancing “Activator. Each of the nine peaks obtained from the original ion exchange column were screened for the presence of any factor capable of enhancing the thermal hysteretic activity of purified Tm 12.86 antifreeze protein. Gel filtration Peak 3 was introduced to each peak from the DEAE-Sepharose column such that the antifreeze protein concentration equaled 6 mg/ml and the ion exchange peak concentration equaled 25 mg/ml. Ion exchange Peak IV was determined to enhance thermal hysteretic activity relative to what would be expected from simply a solution of pure antifreeze protein at 6 mg/ml. Therefore, ion exchange Peak IV was further purified by gel filtration chromatography following the protocol used for purifying the antifreeze protein. Peaks off the gel filtration column of ion exchange Peak IV were collected, dialyzed, lyophilized and suspended in distilled water at a concentration of 25 mg/ml for thermal hysteresis determination. Elution peaks that did not exhibit thermal hysteretic activity were then screened for activator activity in the manner described above. Gel filtration Peak 4 of ion exchange Peak IV was the only fraction from the gel filtration column to display activator activity. Therefore, this peak was subjected to gel electrophoresis (as detailed earlier) and its absorption spectrum was recorded on a Gilford Response UV-Vis Spectrophotometer.

### EXAMPLE 2

Insects and fat body collection. T. molitor larvae were acclimated as detailed in Example 1, Section I. The larvae raised under the above cold-acclimation were then used for isolation mRNA of whole body or fat body since the latter has been shown to be a key source for THPs. Fat body was isolated under sterile procedures. The larvae were sterilized on the surface by 70% ethanol and cut longitudinally in a dissection plate while emerged in Tenebrio modified saline. Under a dissecting microscope the body was opened and fixed in the plate by pins. Then the malpighian tubules were gently removed as cleanly as possible. The fat body was smoothly separated from the tracheae and immediately collected into a 15 ml Falcon tube that was immersed in liquid nitrogen. Following dissection, the collected fat body was stored at −80° C. until use.

RNA isolation. Total RNA was isolated by the following. Approximately 1.2 g of the intact larvae of T. molitor or an equal amount of fat body dissected from larvae was quickly immersed in liquid nitrogen and ground in a mortar. After grinding, the fine dry powder was immediately suspended in 7.5 ml of tissue guanidinium solution (590.8 g guanidinium isothiocyanate; 25 ml of 2 M Tris. Cl, pH 7.5; 20 ml of 0.5 M Na₂EDTA, pH 8.0; add DEPC-dH2O to 950 ml; final 50 ml of B-mercaptoethanol) and mixed thoroughly. The solution was homogenized by sonication of 10 sec bursts for 4-6 times. Debris were removed by centrifugation in a SM-24 rotor (Sorvall RC 5B plus, Dupont) at 10,000 rpm (12,000×g),12° C. for 10 min. The supernatant was transferred into a new 15 ml Falcon tube and 0.1 ml volume of 20% Sarkosyl solution was added. After the incubation at 65° C. for 2 min, CsCl was added to the final concentration of 0.3 g CsCl/ml. After the CsCl was dissolved the sample was layered over 1 ml of 5.7 M CsCl in a polycarbonate thick wall centrifuge tube and ultracentrifuged in the TLA-100, 4 rotor (Optima TM TLX Ultracentrifuge 120,000 rpm, Beckman) at 80,000 rpm (267,000×g) at 22° C. for 2 or 2.5 hours. The supernatant was carefully discarded with a Pasteur pipette. The remaining liquid was drained off by inverting the tube on a paper towel. The pellet was redissolved in diethylpyrocarbonate (DEPC) treated water and then transferred to an RNase-free tube. The solution was sequentially extracted with equal volume of 25:24:1 phenol/chloroform/isoamyl alcohol, and of 24:1 chloroform/isoamyl alcohol. Then the mixture was centrifuged for 5 min at maximal speed, and the supernatant of the upper phase was carefully transferred into a new RNase-free tube. The same operation with 24:1 chloroform/isoamyl alcohol was repeated. The supernatant of the upper phase was carefully transferred into a new RNase-free tube. The RNA was precipitated by adding 0.1 volume of 3 M sodium acetate, pH 5.2 and 2.5 volume of 100% ethanol. The RNA pellet was resuspended in DEPC water and stored at −80° C. until further use.

Messenger RNA (mRNA) isolation. PolyATtract mRNA isolation system from Promega was chosen for use and the procedure was followed according to the instruction provided by the manufacturer. Briefly, 500 μl of total RNA solution (concentration between 600-1000 μg) was incubated at 65° C. for 10 min and then 3 μl of biotinylated-oligo(dT) probe and 13 μl of 20×SSC (3 M NaCl, 0.3 M sodium of citric acid) were added. The solution was gently mixed and incubated at room temperature for no more than 10 min. The solution was transferred to a tube containing the washed SA-PMPS (StrptAvidin ParaMagnetic Particles) and incubated at room temperature for 10 min. Next, the SA-PMPS was captured with a magnetic stand, and the supernatant was carefully discarded. The captured particles were washed with 0.1×SSC (0.3 ml per wash) four times. To elude the mRNA, the final washed SA-PMP pellets were resuspended in 0.1 ml of the DEPC water and mRNA was released into the solution. The aqueous phase of mRNA was transferred to a sterile, RNAse-free tube. The SA-PMPS pellets were resuspended in 0.15 ml of RNase-free water. The capture step was repeated and combined with the eluded mRNA from the first elution in a new mRNA-free tube with total volume of 0.25 ml. The solution was stored at −80° C.

mRNA concentration and purity was determined by measuring the 260/280 absorbance with spectrophotometer. The mRNA for secondary applications was handled respectively as the following (protocol from Promega “PolyATtract mRNA isolation systems”):

1. For cDNA cloning: add 0.1 volume of 3M sodium acetate and 1.0 volume of isopropanol to the elude and incubate at −20° C. overnight.

2. For translation in vitro: add 0.1 volume of 3M potassium or ammonium acetate and 1.0 volume of isopropanol to the elude and incubate at −20° C. overnight. After the treatments as described in step 1 and 2 the samples were centrifuged at full speed for 10 min and then washed with 75% ethanol. The pellet was resuspended in DEPC water and stored at −70° C.

Electrophoresis of RNA and mRNA. Electrophoresis of RNA and mRNA in native agarose gel was performed according to routine procedures. Agarose (1 gm) was melted in 100 ml of 1×TAE buffer (1×: 0.04 M Tris-acetate; 0.001 M EDTA, pH 8.0) with 2 μl of 0.1 μg/ml of ethidium bromide (EtBr). After cooling down, about 30 ml of agarose solution was poured into a mini-gel tray with a six well comb. When the gel was solidified RNA (2 μg) or mRNA (2 μg) sample was loaded in individual wells and electrophoresed in 1×TAE buffer under 70V for 2 h. Lambda DNA/Hind III markers (eight DNA fragments with molecular weight range from 0.125 to 23.130 Kb, purchased from Promega, Madison, Wis.) were used as molecular weight standards. The electrophoresis products were visualize under the UV light and pictures were taken using Polaroid 667 pack film or on a Gel Documentation System (UVP Imagestore 5000, San Gabriel CA) following the procedure provided by the manufacturer.

Electrophoresis of RNA and mRNA under denaturing conditions was performed on quick formaldehyde RNA gel following the protocol from the Stratagene cDNA synthesis kit. A 0.33 g of agarose powders were added into 3.3 mL of 10×MOPS buffer {0.2 M mops [3-(N-morpholino) propanesulfonic acid]; 0.05 M of sodium acetate; 0.01 M EDTA, pH 8.0} and 28.3 mL of sterile water and melted in a microwave oven. After it was cooled down to about 50° C., 1.8 mL of 37% (V/) formaldehyde was added in a fume hood and mixed well. It was then poured into a mini-gel tray with a six well comb. After the gel became solid, the gel was immersed in 1×MOPs running buffer. 2-3 μl RNA or mRNA (about 5 μg) was mixed with 3 μl of 25 mM EDTA containing 0.1% SDS and 10 μl of loading buffer [48% (v/v) formamide; 160 ml of 10×MOPS buffer; 260 ml of 37% formaldehyde; 100 ml of sterile water, 100 μl of EtBr (10 mg/ml); 80 ml of sterile glycerol; 80 ml of saturated bromphenol blue in sterile water]. The mixture was incubated at 67° C. for 10 min, and then loaded into a well. The gel was electrophoresed in 1×MOPS buffer at 50 to 70V. A single-stranded RNA molecular weight standard (the range from 0.28 to 6.58 Kb, purchased from Promega, Madison, Wis.) was co-electrophoresed following a similar treatment to that described above. Gels were visualized under UV light and photographed.

In vitro translation. Isolated mRNA was subjected to in vitro translation using an in vitro translation kit (Stratagene) and following the procedure provided by manufacturer. In general, 2 μl (1 μg/μl) mRNA isolated from T. molitor was incubated at 68° C. for 30 seconds, then 2 μl ³⁵S-methionine-1200 Ci/mmole (DuPont NEN) was immediately added. DEPC water (1 μl) was added to the final volume of 51 μl. Then 20 μl of thawed and well-mixed lysate of rabbit reticulocyte was added to the reaction, which was mixed thoroughly and incubated in a 30° C. heat block for 1 hr. The translation products were precipitated by TCA precipitation assay. The pellet was resuspended in electrophoresis buffer and loaded onto a SDS-PAGE gel for electrophoresis and autoradiography. If it was necessary to store the in vitro translation products, electrophroesis buffer was added and the samples were boiled for 5 min, then frozen at −80° C.

TCA (trichloracetic acid) precipitation assay. The method for TCA precipitation was that detailed in protocol from Stratagene cDNA synthesis kit and Promega. This consisted of adding 2 μl of translation product into 500 μl of glass distilled water. The solution was mixed with 250 μl of 1.0 M NaOH containing 0.5 M H2O2 and 1 mg/mL unlabeled methionine and incubated at 37° C. for 15 min to decolorize sample. The protein was precipitated by the addition of 1 ml of ice-cold 25% TCA. After the incubation in ice for 30 min, the reaction mixture was filtrated on glass fiber discs Whatman (GF/C). The filter was rinsed with 1 ml of ice-cold 8% TCA four times, then dried and the precipitated radioactivity was counted with a liquid scintillation counter. The translated products from different samples were pooled and stored at −20° C.

Immunoprecipitation. The in vitro translation products were subjected to immunoprecipitation using an antiserum generated against purified Tm 12.86 AFP (See Example 1). The protocol for immunoprecipitation was that initially developed for immunoprecipitation of in vitro translation products generated from wheat germ cell-free systems. Thus, to adopt the protocol to a rabbit reticulocyte lysate cell-free system used in this study, some modifications were necessary as detailed below.

The procedure consisted of taking 8 μl of 25% SDS, added to 42 μl of translation reaction mixture. The sample was heated at 100° C. for 4 min, then diluted with the same volume (50 μl) of dH2O. Then, 4× volume of dilution buffer was added (2.5% Triton X-100, 190 mM NaCl, 6 mM EDTA, 50 mM Tris-HCl, pH 7.4 and 10 units of Trasylol [same as aprotinin, Sigma] per milliliter). After adding 15 μl of the nondiluted Tm 12.86 AFP antisera, the reaction mixture was incubated at 4° C. overnight Next morning, 30 μl of Immobilized Protein A™ (Repli Gen Corporation) (in place of protein A-Sepharose CL4B as in wheat germ system) was added and the sample incubated with end-over-end mixing at room temperature for 2 hours. The agarose beads were pelleted in a microcentrifuge at 10,000 rpm for 10-second. The supernatant was discarded. The beads were washed for 3 times with 1 ml (per wash) of buffer solution (0.1% Triton X-100, 0.02% SDS, 150 mM NaCl, 50 mM Tris-HCl, pH 7.5, 5 mM EDTA, 10 units of Trasylol per ml) at room temperature with vortexing and pelleted at 12,000 rpm for 1 min. The beads were finally washed with the buffer solution, but with no detergent. The supernatant was removed as completely as possible. SDS-gel sample buffer (30 μl) was then added to the beads, and the suspension heated for 4 min in the boiling water bath. Free-SH groups were blocked by adding 10 μl of 1.0 M iodoacetamide in sample buffer and incubated for 45 min at 37° C. The beads were pelleted at 14,000 rpm at a microcentrifuge for 4 min and the eluded antibody bound proteins were transferred to a new tube, and stored at −20° C. until eletrophoresis.

Electrophoresis analysis on SDS-PAGE gel and Fluorography. Translation products and immunoprecipitation products were analysized by electrophoresis on 0.8 mM of SDS-PAGE polyacrylamide gel following the protocol detailed in Example 1, Section 3 using either a 15%; 17% or 20% resolving gel in conjunction with a 5% stacking gel. The gel was fixed and stained in the 10% methanol, 10% glacial acetic acid solution with 0.1 μg/ml Coomassie brilliant blue (R-250) for one hour and then destained in the 10% glacial acid and 50% methanol solution. The destain solution was changed after 5, 10 and 60 minutes. After destaining was complete, the gel was transferred into the enhance solution (EN₃HANCE™, Biotechnology System, NEN Research Product) for one hour and then washed with distilled water. Finally, the gel was placed onto a piece of filter paper and dried under heat (60-70° C.) and vacuumed on a slab gel drying apparatus. The dried gel was exposed to Kodak X-ray film (Biomax, MR or X-omat RP) overnight or longer depending on the count of the radioactivity from TCA incorporation result. The film was developed according to the instructions provided.

Construction of cDNA Libraries of T. molitor

Synthesis of cDNA. mRNA isolated from winter-acclimated whole animal and fat body of T. molitor were used as starting material to construct cDNA libraries. The ZAP express cDNA synthesis kit purchased from Stratagene was used for synthesis of cDNA. The detailed protocols suggested by the manufacturer were followed. Briefly, the protocol for cDNA library construction is described as follows: The first strand synthesis was primed with hybrid oligo(dT) linker-primer which contains an xhoi site and transcribed using reverse transcriptase (MMLV-RT) and 5-methyl dCTP. After hemimethylation, the second single strands of cDNA were synthesized and blunted with DNA polymerase I and RNAse H. Then, EcoR I adaptors were ligated by using T4 DNA ligase to make a cohesive end of the cDNAs. Finally, XhoI restriction enzyme was used to digest the XhoI site, thus, each strand has a XhoI site on one end and EcoRI site on the other end.

Ligation of cDNA into ZAP expression vector. The above cDNAs were applied to the Sephacryl S-500 spin column to get rid of small pieces and uncomplete cDNA. Fractions were collected after each spin. Then each fraction was precipitated and ligated to the ZAP express vector arms, which generated libraries with different size of cDNA inserts. The ligated ZAP express vector was packaged into lambda phage particles using ZAP express cDNA Glgapack Gold Cloning Kit (Stratagene), i.e. packageing the vector with lambda coat protein to have viable phage activity. The cDNA libraries were amplified by plating on NZY plates with XL1-blue MRF′ strain (Stratagene).

Screening of Tm 12.86 clone from libraries.

Phage plaque lift. Phages were plated at high density with 5.0×10⁴ pfu (plague forming unit) per plate (150 mm) as recommended by Stratagene in the PicoBlue™ immunoscreening kit. Briefly, the XL1-blue MRF′ cells were cultured overnight in NZY medium [5 g NaCl, 2 g MgSO4.7H2O, 5 g yeast extract, 10 g NZ amine (casein hydrolysate), 15 g agar per liter at pH 7.5] supplemented with 10 mM MgSO₄ and 0.2% (v/v) of maltose. When the cell density reached OD600 of 1.0 the cells were pelleted and resuspended with sterilized 10 mM MgSO4 and diluted to a final OD600 of 0.5. A portion of this XL1-Blue MRF′ cell suspension was mixed with phages and incubated for 15 minutes at 37° C., then the melted NZY top agar [5 g NaCl, 2 g MgSO4.7H2O, 5 g yeast extract, 10 g NZ amine and 0.7% (v/v) agarose, pH 7.5] was added and mixed. The mixture was immediately poured onto the surface of a pre-prepared agar plates and left to solidify at room temperature. The agar plates were then incubated at 42° C. for 5 hours. During incubation the nitrocellulose membranes (Stratagene) were submerged in 10 mM IPTG (isopropyl-1-thio-β-D-galactopyranoside) solution. After completely wetting the nitrocellulose membranes, they were placed on Whatman 3 mm paper to air dry. When small plaques became visible in plates, the plates were covered with the treated nitrocellulose membranes and incubated for another 3-5 hours or overnight at 37° C. The expression of cDNA in the vector is induced by IPTG absorbed in the membrane and the expressed proteins would be transferred to the membrane via plaque lift process. The lifted nitrocellulose membrances were washed in PBS buffer and subjected to immunoblot screening.

Immunoblotting of cDNA clones. Methods used for immunoblot screening of the plaques were similar to the approach detailed in Example 1, with some modification. In brief, the nitrocellulose membranes obtained during the phage lift were washed in PBS (phosphate Buffer Saline: 0.002 M kCl, 0.14 M NaCl, 0.01 M Na2HPO₄, 0.0015M KH₂PO₄, pH 7.2) after lifting. The wash was usually carried out for 3 times with shaking, each time for 5 min. The membrane was first blocked with fresh 5% nonfat dry milk in PBS buffer for one hour with gentle agitation and then washed with PBS as described above. To block the possible endogenous perixodases in the membrane, the membrane then was incubated with firsh 0.5% H₂O₂, for 5-30 min and followed by washing with PBS for three times. Next, the membrane was incubated in the primary antibody against Tm12. 86 kD antifreeze protein (primary antibody serum was diluted at 1:1000 with PBS) for one to two hours with gentle shaking at room temperature, then washed with PBS for three times. The membrane was incubated with a 1:500 dilution second antibody (peroxidase-conjugate goat-anti-rabbit, Sigma) for one to two hours and washed with PBS as above. Finally, the membrane was colorized with 15 ml of DAB solution (3,3′-Diaminobenzidine Tetrahydrochloride; Fast Dab: Sigma) with gentle agitation until purple dots (positive clones) were visualized. The DAB reaction was stopped by washing the membrane with PBS. The membrane was dried in air for preservation. Plaques corresponding to positive dots in the membrane were marked for further evaluation including purification and isolation.

In vivo excision of the pBK-cmv phagemid vector from the isolated single positive plaques. Several single immunologically positive plaques from each of the two cDNA libraries [F5+6 (WB) and F3 . . . 6 (FB)] containing small cDNA fragments were used for excision following the single-clone excision protocol described in the ZAP express cDNA synthesis kit (Stratagene). Individual positive plaques obtained from initial screening were further purified and isolated in low concentration of pfu from NZY agar plates and stored in a tube containing 500 μl of phage stock buffer (SM buffer) (0.1M NaCl; 0.017 M MgSO4.7H₂O, 10, 0.05M Tris —HCl, pH 7.5; 1% (W/V) gelatin, 201 μl of chloroform). XL1-Blue MRF′ and XLOLR cells were grown separately overnight in NZY broth [5 g of NaCl; 2 g of MgSO4.7H2O; 5 g of yeast extract; 10 g of NZ amine with deionized H₂O added to a final volume of 1 liter, and pH to 7.5 with NaOH] at 30° C. Then cells were pelleted and resuspend in 10 mM MgSO4 at a concentration of 1.0 determined spectrophotometry at OD600. First, 200 μl of XL1-Blue MRF′ cells were mixed with 250 μl of the phage stock and 1 μl of ExAssist helper phage and the mixture was incubated in a Falcon polypropylene tube at 37° C. for 15 minutes, then 3 ml of NZY broth was added and the solution was incubated for 2.5-3 hours at 37° C. with shaking. Next, the solution was heated at 65-70° C. for 20 minutes and spun down at 1000×g for 15 minutes. The supernatant containing the excised pBK-CMV ss DNA phagemid packaged as filamentous phage particles was saved.

To get colonies from the phagemid, 200 μl of freshly grown XLOILR cells were mixed with 10 μl of the excised phagemids. After incubation at 37° C. for 15 minutes, 300 μl of NZY broth was added and incubated at 37° C. for another 45 minutes. 200 μl of the cell mixture was plated on each LB (loria broth))-kanamycin agar plate and incubated overnight at 37° C. Next day many colonies would appear on the plates which contain the pBK-CMV double-stranded phagemid vector with the cloned cDNA insert

Plasmid DNA isolation. cDNA was isolated from phagemid using the “plasmid boiling miniprep protocol” from Stratagene. Briefly, a single excised colony was grown overnight in 3 ml of LB broth with kanamycin (50 μg/ml). The next day the cells were pelleted in a microcentrifuge and resuspend in 110 μl of STETL buffer [8% sucrose, 0.5% Triton X-100, 50.0 mM Tris (pH 8.0), 50.0 mM EDTA, 0.5 mg/ml lysozyme]. The sample was placed in a boiling water bath for 30 seconds, immediately spun done at 4° C. for 15 minutes and the supernatant was saved. Then, 1 μl of “RNAse-it Ribonuclease cocktail” (Stratagene) was added to the supernatant and the tube was incubated at 37° C. for 30 minutes in order to get rid of RNA. The plasmid DNA was precipitated by adding an equal volume of isopropanol to the tube and spun for 15 minute. The pellet was resuspended in 100 μl of TE buffer [10 mM Tris-HCl (pH 7.5), 1 mM EDTA]. The DNA solution was extracted twice with same volume of phenol-chloroform and once with chloroform. An equal volume of 7.5 M ammonium acetate was added and the DNA was precipitated with 2.5 volumes of ethanol during incubation at −20° C. for 15 minutes, followed by a spin at 4° C. for 20 minutes. The pellet was washed by adding 1 ml of 75% (VIV) ethanol and spun briefly (for a few seconds). Finally, the pellet was vacuum dried and resuspended in 15 μl of TE buffer and stored at 4° C.

Digestion of DNA with restriction enzymes. In general, the method for DNA digestion was as follows. A certain amount (˜2 μg) of plasmid DNA was added to a 1.5 ml microcentrifuge tube containing 3 μl of universal buffer (Stratagene) was added and then appropriate amount (following recommendation by Stratagene) of restriction enzymes of Xhol and EcoRI were added. The final volume was brought to 20 μl with dH₂O and incubated at 37° C. for 1 hour. The digested DNA solution was subjected to electrophoresis in 1.0% agarose gel or stored at −20° C.

DNA sequencing and its analysis. Seven out of 30 recombinant plasmids detected by antiserium against Tm 12.86, each containing about 500 bps following digestion by XLo I and Eco R I were selected for nucleotide sequencing. These clones were initially sequenced by the dideoxy chain termination method using the Sequenase sequencing kit (version 2.0) from U.S. Biochemical Corp. (Cleveland, Ohio); and a −³⁵S-dATP from Du pont NEN (Boston Mass.). Both T7 and T3 primers, complementary to the sequence of the vector were used. The purified plasmid DNA was denatured with 0.2 M NaOH containing 0.2 mM EDTA, then neutralied with 0.6 M sodium acetate, pH 5.2 and precipitated with ethanol prior to sequencing. Sequence reaction followed the instruction provided by USB and sequence reaction products (about 3 μl) were loaded on 6% polyacrylamide gel (Life technologies. GiBcoBRL) for electrophoresis at a constant power (1500V). After the blue dye reached the bottom of the plate, the gel was placed onto a piece of filter paper and dried under heat (80° C.) and vacuumed on a slab gel drying apparatus. The dried gel was exposed to Fuji X-ray film overnight or longer depending on the count of the radio-activity from the monitor. The film was developed according to the instructions provided. After DNA sequence was read, DNA and predicted protein sequences were analyzed with FASTA and Genetics Computer Group version 7.1 programs. Subsequent sequencing was obtained via an automated DNA sequencer (detailed in Example 4).

Western analysis. Protein products expressed from the colonies were screened with anti-Tm12.86 in western blot analysis, via the method detailed in Example 1. Collection of products involved culturing a single colony containing the cDNA insert (FW1) plus kanamycin (50 μg/ml), or just host cell XLOLR (for the control) plus tetracycline 12.5 μg/ml in 3 ml LB broth with 250 rpm agitation at 37° C. When OD600 reached about 0.2-0.5, IPTG (1-2 mM) was added to the culture. The medium was incubated as before for 5 hours and pelleted in 1500 g for 10 min. The pellet was resuspended in 200 μl protein exaction buffer (0.0625 M Tris-HCl, pH 6.8, 0.001 M phenylmethylsulfonylfluoride, 1% Nonidet P-40) and sonicated for 50 seconds with pulse of each 10 seconds, then the sample was centrifuged at 12,000 g for 5 min and the liqiud was transferred to a new tube. 2 μl of the each solution was used to determine the total concentration of protein. Then about 30 μg of total protein was subjected to electrophoresis in 16.5% SDS-PAGE gel (detailed in Example 1.)

After electrophoresis, electroblotting was performed as detailed in Example 1, with minor modification. The membrane was incubated in the primary antisera against the Tm 12.86 kD antifreeze protein (primary anti-serum was diluted at 1:2000 with PBS) for one to two hours, then the membrane was washed with PBS for three times. The membrane was blocked with a 1:500 dilution second antibody (peroxidase-conjugate goat-anti-rabbit, Sigma) for one to two hours and washed with PBS as above. Bands were detected on the membrane with 15 ml of DAB solution (3,3′-Diaminobenzidine Tetrahydrochloride; Fast Dab: Sigma) using gentle agitation until bands were visualized. The DAB reaction was stopped by washing the membrane with PBS. The membrane was dried in air.

Preparation of protein samples from positive clone. To test whether the recombinent protein expressed from the positive clones had antifreeze activity, protein was extracted from clones grown in 100 ml of LB containing kanamycin (50 μg/ml) with agitation (250 rpm) at 37° C. When OD600 reached about 0.2-0.5, ITG (1-2 μM/ml) was added to the culture to induce the expression of recombinent protein. The culture was incubated for additional 5 hours and then pelleted in 1500 g for 10 min. The pellet (about 1 gram) was resuspended in 5 ml protein extraction buffer (50 mM Tris, pH 8.0, 1 mM of EDTA, 100 mM NACl). Then, 4 μL of 0.1 M PMSF (phenylmethylsulfonylfluoride), and 80 μl of lysozyme (10 mg/ml) was added and the sample was stirred 20 minutes at room temperature. 4 mg of deoxycholate was added and incubated at 37° C. until the solution became very viscous (approximately for 15 minutes). Then 20 μl of DNase I (1 mg/ml) was added and stirred at room temperature for about 30 minutes (until the solution was no longer viscous). The solution was centrifuged for 15 minutes at 10 K rpm. The pellet was washed with the extraction buffer plus 0.5% Triton and 10 mM EDTA, and then incubated for 10 minutes at room temperature, and centrifuged for 15 minutes at 10 k rpm. The pellet was dissolved in teflon homogenizer containing 2.5 ml solubilization buffer (8 M urea deionized, 50 mM tris, pH 8.0, 0.01% Triton, 200 mM NaCl) and incubated for 1.5 hours at room temperature with shaking. The solution was then centrifuged for 15 minutes at 10 K rpm, and supernatant was diluted to approximately 500 μg/ml protein with renaturation buffer (6 M urea deionized, 50 mM Tris, pH 8.0, 0.01% Triton, 0.20 M NaCl, 1 mg reduced glutathione, and 0.05 mM oxidized glutathione) and stirred for 1.0 hour at 4° C. The renatured sample was then changed for 12 hours, and then 6 hours against each 300 ml of 50 mM tris at pH 8.0, 0.01% Tween 80,200 mM NaCl, 1 mM of reduced glutathione, and 0.05 mM of oxidized glutathione. Then in order to get rid of the salt the solution was further dialyzed against dH2O with changing water every six hours for three times. Finally, the solution was lyophilized and resuspended in a small amount of dH2O (about 20 μl).

Antifreeze protein activity assay. Two methods were used for the detection of antifreeze protein activity of the prepared sample above. 1. Determination of thermal hysteresis activity via the microcapillary method (detailed in Example 1). 2. Screening for recrystallization inhibition behavior (See Example 8).

### EXAMPLE 3

Five cDNA libraries were developed as detailed in Example 2, two from fat body-derived cDNAs, designated F₁₊₂ (FB) (corresponding to larger cDNAs) and F_{3 . . . 6} (FB) (corresponding to smaller cDNAs). Likewise, three fractions were derived from “whole body” cDNAs, designated F₁₊₂ (WB), F₃₊₄ (WB) and F₅₊₆ (WB), with F₁₊₂ (WB) representing the largest cDNAs, etc. Example 3 involves screening a different cDNA library from those used in Example 2, and the subsequent isolation and characterization of two other members (clones 2-2 and 2-3) of the Tm 12.86 family of genes.

Immunoscreening of the T. molitor cDNA library. Screening of the cDNA library was performed using the F₁₊₂ (FB) fraction phages. The choice of F₁₊₂ (FB) for this screening was based on observations that Tm 12.86 is found stored in fat body protein granules, and the possibility that a storage form of Tm 12.86 may occur as a polyprotein derived from larger mRNAs.

This cDNA library was screened as in Example 2, Section X with certain modifications. The F₁₊₂ (FB) phages were first diluted to ˜5×10⁶ pfu/ml (pfu =“plaque forming units”, or more roughly, the number of phages) using sterile SM buffer. The starting concentration of phages in the libraries was assumed (based on previous results) to be ˜10⁸ pfu/ml. Next the XL1-Blue MRF′ strain of E. coli culture was prepared by inoculating 6 ml of sterile NZY medium supplemented with 0.2% maltose in a sterile Falcon 2059 tube (cap loosened) with bacteria transferred from a XL1-Blue MRF′ culture plate. The XL1-Blue MRF′ culture was incubated with shaking for ˜10 hours (overnight), reaching a final O.D.₆₀₀ reading of 0.77. The cells were then pelleted by centrifugation at 500 g for 10 minutes (2000 rpm using an SS-34 rotor; the Falcon tubes were placed in 50 ml VWR Scientific polypropylene tubes before centrifuging). After centrifugation, the cells were diluted to O.D.₆₀₀=˜0.5 using sterile 10 mM MgSO₄ (˜2 to 3 ml MgSO₄ in this case, corresponding to about one-half the original culture volume). At this point, the cells were stored at 4° C. and used up to 48 hours later during the screening process.

The prepared XL1-Blue MRF′ cells were then “infected” with the diluted phages by mixing 10 μl of 5×10⁶ pfu/ml F₁₊₂ (FB) suspension with 600 μl XL1-Blue MRF′ cells (O.D.₆₀₀=˜0.5), resulting in a final phage density of 50,000 pfu/ml. The phages and bacteria were then incubated at 37° C. for 15 minutes (with gentle shaking). NZY top agar was prepared and cooled to 48° C. after autoclaving. A volume of 6.5 ml of NZY top agar was transferred to a sterile VWR Scientific 50 ml polypropylene tube with lid and maintained at 48° C. in a water bath until ready for use. At the same time, an NZY agar plate (150 mm diameter) was also incubated at 42° C. for ˜30 minutes in preparation for the spreading of the top agar. The phages and bacteria were then added to the top agar in the 50 ml tubes (still immersed in the water bath at 48° C.) and mixed gently for 2-3 seconds. The top agar mixture was immediately poured onto the warmed (i.e. 42° C.) NZY agar plate, and spread as evenly as possible over the agar surface. This procedure was performed as quickly as possible to ensure that (1) bacteria are not destroyed by 48° C. temperatures and (2) the top agar (after pouring) does not solidify too quickly before spreading on the plate is complete. After allowing the top agar to cool at room temperature for 10 minutes, the plate was incubated (inverted) at 42° C. for 5 hours.

While allowing the bacteria and phages to incubate, an IPTG-nitrocellulose filter was prepared by soaking the filter (cut to fit the circular 150 mm plate) in a 10 mM IPTG solution, then allowing the filter to air dry on a Whatman 3 mm (or other blotting) paper. After the five hour incubation period, the filter was carefully placed on the agar. The plate with nitrocellulose (NC) overlay was incubated for another 5 hours at 37° C. The IPTG in the filter induces translation of the cDNA within infected bacteria, which release recombinant protein onto the filter by export or upon phage-induced lysis. Upon completion of the second five-hour incubation, the plate with filter was allowed to dry by removing the lid and incubating for an additional 20 minutes at 37° C. followed by cooling the plate at 4° C. for 30 minutes to facilitate removal of the NC filter from the top agar. Before removing the NC filter, a pin was used to mark patterns at the edge of the plate to ensure that the filter could be aligned properly with the agar at a later step. The filter was then removed and placed in phosphate buffered saline (PBS) in preparation for immunoscreening.

Immunoblot development. The NC membrane was screened with anti-Tm 12.86 antiserum using procedures outlined in Example 2, Section X). Briefly, the NC membrane was first blocked with dry milk proteins and treated with hydrogen peroxide to neutralize possible peroxidases on the membrane (which may produce false positive results). Since peroxidase activity was evident in this case (gas bubbles were produced in the presence of hydrogen peroxide), the hydrogen peroxide concentration was increased from 0.5% to 3%, and exposure time increased to 20-30 minutes. The membrane was then exposed to primary rabbit antibody (polyclonal antibody containing anti-Tm 12.86) at 1:2000 dilution in PBS, then washed leaving primary antibodies bound only to specific immunoreactive proteins. The next step involved exposure of the membrane with bound primary antibody to a secondary goat anti-rabbit antibody-peroxidase conjugate, allowing the formation of primary-secondary antibody complexes. These complexes were detected using DAB (3,3′-Diaminobenzidine tetrahydrochloride) solution which reacted to the presence of peroxidase. Positive results appeared as small (˜1-2 mm) brown-colored dots on the membrane.

A replicate of the membrane was constructed using a transparency overlay, marking all immunopositive locations, along with the orienting pin-hole positions along the edge of the membrane, on the transparency. The original NZY agar plate with plaques was then aligned properly with the transparency and positive plaques removed as agar “plugs”. In this case, the plugs were removed with a short section (˜3 cm) of a polyethylene transfer pipette (Fisher, 5 ml) sterilized in 70% ethanol. The plugs were immediately transferred to microfuge tubes containing 1 ml SM buffer+20 μl chloroform (as a preservative) and stored at 4° C. Phages eluted from the plugs were subjected to two more screenings to ensure isolation of single cDNA clones.

Excision of pBK-CMV phagemid (plasmid) vectors from ZAP Express vectors. As described some in Example 2, Section XI, excision of lambda-phage vector DNA was required to allow for expression of cDNA-encoded recombinant proteins in E. coli. XL1-Blue MRF′ cells were prepared by incubation in NZY broth with 0.2% maltose (˜6 ml) at 37° C. for 4 to 5 hours (times varied considerably) until an O.D.₆₀₀=0.2 to 0.5 is reached. The cells were pelleted (500 g for 10 minutes; 2000 rpm in SS 34 rotor) and resuspended in 10 mM MgSO₄ to an O.D.₆₀₀ of 1.0. The XL1-Blue MRF′ cells were then coinfected with the single clone lambda phages (from plaque cores) and ExAssist helper phages (M13) by combining 200 μl of XL1-Blue MRF′ cells (at O.D.₆₀₀˜1.0), 250 μl of phage stock (containing at least 10⁵ phages), and 1 μl of the helper phage stock (containing at least 10⁶ pfu/μl) and incubating at 37° C. for 15 minutes. An additional 3 ml of NZY medium (without maltose) was then added to the E. coli+phage mixture and incubated further for 2.5-3 hours at 37° C. with gentle shaking.

The helper phages generate proteins that recognize a specific site within the lambda vector DNA, initiating synthesis of a circular ssDNA phagemid (pBK-CMV) containing cDNA from the linear lambda DNA template. The circular phagemid is then packaged as a filamentous phage particle and released from the bacterium. To recover the filamentous phages from the bacterial culture, the culture was heated to 65°-70° C. for 20 minutes and centrifuged at 1000 g for 15 minutes (3000 rpm using an SS 34 rotor). The supernatant containing filamentous phage particles was then saved, to be used for subsequent infection and recovery of pBK-CMV plasmids within a second strain of E. coli. This particular strain, designated XLOLR, was prepared by inoculated NZY medium (without maltose) and growing to mid-log phase (O.D.₆₀₀˜0.2 to 0.5), then pelleting and resuspending the bacteria in 10 mM MgSO₄ to an O.D.˜1.0.

A 20 μl volume of filamentous phage supernatant (Stratagene recommends 10 μl; however, due to the low number of pBK-CMV positive XLOLR E. coli recovered during this procedure, the volume was increased) was added to 200 μl of XLOLR cells, then incubated at 37° C. for 15 minutes with gentle shaking. Additional NZY medium was then added (300 μl) and the mixture incubated at 37° C. for an additional 45 minutes. After incubation, 200 μl of the mixture was spread evenly on the surface of an LB-kanamycin plate that was then dried for several minutes under a sterile hood (with lid removed). Finally, the plates were placed in an incubator for up to 48 hours at 37° C.

Infection of the XLOLR bacteria by filamentous phages results in conversion of the ssDNA pBK-CMV phagemid to a dsDNA pBK-CMV phagemid (plasmid) within the bacterium, which is replicated as the bacterium divides. Since the pBK-CMV phagemid contains an antibiotic resistance gene, only those XLOLR bacteria that have been successfully infected by filamentous phages will survive plating on the LB-kanamycin medium. In addition, the filamentous phages lack the genes required to replicate in XLOLR, therefore infected cells are not destroyed. The surviving XLOLR bacteria with pBK-CMV phagemids containing the putative Tm 12.86 cDNA insert should produce colonies visible after incubation (over 24 hours incubation time was usually required to observe colonies).

pBK-CMV phagemid vector (plasmid) isolation from E. coli. Two different plasmid isolation methods were applied in this example. Both represent variations of the alkaline lysis method. The first method was primarily used for restriction endonuclease studies of the pBK-CMV phagemid with cDNA insert. Cultures (5 ml each) containing XLOLR E. coli with cDNA clones were grown in LB-kanamycin medium at 37° C. until reaching an O.D.₆₀₀˜1.0 (usually requiring at least 8-10 hours in sterile 50 ml tubes). The bacteria were then separated into 1 ml aliquots (using 1.5 ml microfuge tubes), and pelleted in a microfuge at 10,000 r.p.m. (Eppendorf 5415C) for ˜1 minute. The supernatant was removed from each tube, and individual pellets resuspended in 100 μl ice cold GTE buffer (glucose/Tris/EDTA). After resuspension, a cell lysis reagent consisting of 200 μl of 1% SDS/0.2 NaOH was added to each tube, which were then mixed by inverting the tubes five times each. The tubes were then placed on ice for five minutes to allow completion of the E. coli (XLOLR) lysis. After the five minute incubation period, 150 ml of an ice cold potassium acetate/acetic acid buffer solution was added to each tube to neutralize the NaOH. Each tube was again inverted five times to mix, and then placed on ice for five minutes. A white precipitate of cellular lysis debris was formed at this point in the procedure (cell membranes, cell walls, genomic DNA).

The tubes were then microfuged for five minutes at 14,000 r.p.m. (Eppendorf 5415C) to pellet the precipitate. A volume of 400 μl of supernatant was saved from each tube and transferred individually to new 1.5 ml microfuge tubes. Isopropanol (400 μl) was added to each tube and each tube then inverted 5 times to mix. The tubes were then incubated for exactly two minutes at room temperature to precipitate phagemid DNA. The incubation time in this case was very important since contaminating proteins also begin to precipitate out of the solution (though not as quickly as phagemid DNA) over time. After the two minute incubation period, the microfuge tubes were spun at 14,000 r.p.m. for five minutes to pellet the phagemid DNA, followed by the removal of supernatant. Ethanol (200 μl of 95% v/v) was added to each tube, then “flicked” to wash the pellets. The tubes were microfuged again at 14,000 r.p.m. for five minutes, and most of the supernatant removed by pipetting. The pellets were then dried by leaving the tubes uncapped in a 37° C. incubation chamber. Following the drying period, the pellets were dissolved in 15 μl T.E. (Tris/EDTA) buffer and stored at 4° C. Prior to restriction enzyme digests and gel electrophoresis, 1 μl of RNase-It ribonuclease cocktail (Stratagene) was added to the DNA/T.E. mixture to help remove contaminating RNA.

The second method of phagemid isolation used a Bio101 RPM mini-prep kit to isolate plasmid DNA. This method was preferentially used when sequencing cDNA clones, since purity of the isolated phagemid DNA was of greater concern. The kit protocol was very similar to the previously described alkaline lysis procedure. However, in place of the isopropanol precipitation of phagemid DNA as described for the previous procedure, the RPM kit uses a silica fiber suspension in a spin column which tends to preferentially bind the phagemid DNA. Washing of the silica matrix with bound DNA removes much of the remaining impurities, allowing the phagemid DNA to be eluted with water (as is required for subsequent sequencing) or T.E. buffer at the final purification step.

Restriction endonuclease digests of plasmid DNA. To confirm the presence and size of selected cDNA inserts, EcoRI and XhoI double digests were performed on pBK-CMV phagemids isolated from E. coli (XLOLR) as described in Example 2, section XIII. Enzyme digest reaction mixtures consisted of ˜10 to 35 μg total plasmid DNA, reaction mixture buffer (usually 2 μl of 10× buffer per reaction), enzyme added to a final concentration of at least 1.0 U/mL, and water added to a final reaction volume of 20 μl. The restriction enzyme (R.E.) digest reaction mixtures were then incubated at 37° C. for one hour. Gel electrophoresis was performed on the enzyme digest products using 0.9% NaCl gels.

Sequencing of cDNA clones. Nucleotide sequences for cDNA inserts were obtained using a variation of the Sanger dideoxy method with an ABI Prism 310 Genetic Analyzer. Phagemid DNA (˜0.5 to 0.7 μg total) containing cDNA was added to a reaction mixture with 3.2 pmole T3 or T7 primer (two separate reaction mixture were created: one contained T3 primer only, the other contained T7 primer only), a terminator premix containing dye-conjugated ddNTPs, dNTPs, buffer, and DNA polymerase, and finally water to bring the reaction mixture to 20 μl total volume.

The reaction mixtures were then subjected to thermal cycling on an MJ Research PTC-200 Peltier Thermal Cycler, creating dye-terminated complementary DNA extension strands. The thermal cycler first heat samples to 96° C. for 30 seconds (denatures dsDNA into single strands), followed by cooling to 50° C. for 15 seconds (allows primers to bind ssDNA), then heating to 60° C. for 4 minutes (primer extension: polymerization of complentary DNA strands). These three steps are repeated in sequence 25 times. After thermal cycling, the newly synthesized DNA extension strands were purified using Centri-sep spin columns (Princeton Separations) which function as gel filtration columns to remove unused nucleotides from the reaction mixtures. Briefly, the spin columns were prepared according to the manufacturer's recommendations by hydrating the gel beads in 0.8 ml H₂O for 30 minutes, then allowing the liquid to drain from the column by gravity. Liquid remaining in the column was drained by centrifuging the column at 750 g (3000 rpm using the Eppendorf Model 5415C) for two minutes. The 20 μl reaction mixture volume was pipetted onto the top of the gel matrix, followed by placement of the column into a collection tube and centrifugation at 750 g for 2 minutes. The resultant liquid expelled into the collection tube (containing purified DNA strands) was saved and then dried using a Savant Speed-vac for 20 to 30 minutes. Care was taken not to excessively dry the DNA, since this might interfere with subsequent rehydration steps. The collection tube with DNA was then wrapped in aluminum foil (to avoid exposing the nucleotide-conjugated dyes to light) and stored at −20° C. in preparation for analysis using the ABI Genetic Analyzer.

The dried DNA samples were each resuspended in 25 μl of Template Suppression Reagent (ABI) followed by heating of the sample at 95° C. for two minutes to separate any renatured single-stranded DNA molecules. The samples were then placed on ice until loaded onto the Genetic Analyzer. The ABI Genetic Analyzer functions much like an automated version of gel electrophoresis to separate the dye-terminated strands according to size (in the total number of bases). A laser-based detection system identifies the 3′ base of each migrating strand according to the particular dye conjugated to that base (four different fluorescent dyes corresponding to A, G, T, and C bases were used). Software associated with the analyzer converts the strand detection data into a full DNA sequence usually most accurate up to ˜300 to 350 bases downstream from the end of the primer. For this reason, primers corresponding to pBK-CMV sequences at either end of the cDNA insert (T3 and T7) were used to sequence the full ˜500 bp. cDNAs of this particular study from both the 5′ ends and 3′ ends, thus creating overlapping sequences.

Analysis of sequence data. The computer program DNASTAR was used to help develop full nucleotide sequences based on data obtained from the ABI Genetic Analyzer. The Analyzer data consisted of a “+” strand sequence (T3 primed) and “−” strand sequence (T7 primed), both exhibiting a certain amount of sequence due to the relatively small sizes of the cDNA inserts studied (˜500 bp.). The DNASTAR programs facilitate the construction of a full nucleotide sequence by aligning overlapping strands (creating a “contiguous” sequence as shown in FIGS. 3.0 and 3.1). Conflicting base determinations do occur, especially for locations furthest from the primers where sequencing tends to become less accurate. Where conflicts arise, the “correct” base is more likely to correspond to the one closest to a primer. However, a confirmation of the nucleotide determination based on fluorescent peak raw data is also desirable, especially where distances from primers is about the same for both strands.

Expression of recombinant protein in bacteria containing the cDNA of interest. A modification of a protein granule isolation procedure detailed in Example 2, Section XVI was used to isolate recombinant proteins from bacterial clones containing pBK-CMV phagemids with cDNA inserts. Using this procedure, 3 ml cultures of the bacterial clones were grown in LB+kanamycin medium to an O.D.₆₀₀ of 0.2 to 0.5. To induce recombinant protein synthesis by the bacteria, 300 μl of 20 mM IPTG stock solution was added to each 3 ml culture, resulting in a final concentration of ˜1.8 mM. The cultures were then incubated for an additional 5 hours. After incubation, the cultures were pelleted at 1500 g for 10 minutes (SS 34 rotor at ˜3500 rpm) and supernatant removed. The pellets were resuspended in 200 μl of 0.0625 M Tris-Cl pH=6.8, 1% (v/v) non-det P40, and 0.001M PMSF (the resuspension buffer just described was prepared immediately before use by adding 10 μl 0.1 M PMSF stock (in 100% isopropanol) to 990 μl 0.0625 M Tris-Cl pH=6.8, 1% non-det P40, since PMSF degrades fairly rapidly in water solution). Each tube with resuspension buffer was sonicated using ten one-second pulses and repeating the procedure 5 times for each tube (for a total of 50 seconds sonication time). The lysed cells were then transferred to 1.5 ml microfuge tubes and centrifuged at 12,000 g for 5 minutes (Eppendorf 5415C Microfuge at 14,000 rpm). The supernatant containing soluble bacterial proteins was then transferred to new 1.5 ml microfuge tubes.

The liquid samples were then frozen and concentrated using a Labconco freeze drier in order to decrease the liquid volume by at least one-half. The concentrated samples were then assessed for protein content using the Bradford assay. Finally, the samples were evaluated for the presence of recombinant proteins immunoreactive with the anti-Tm 12.86 polyclonal antibody by performing SDS-PAGE followed by Western blotting.

### EXAMPLE 4

Presented here are procedures for further analyses of the Tm 12.86 AFP multigene family, including through Southern analyses detection for the presence and number of additional homologous genes, consideration of their arrangement in the genome (e.g. tandemly linked or scattered), PCR generation of genomic DNA fragments, and further immunoscreening of the cDNA library whereby three additional clones (designated 3-4, 3-9, and 7-5) have been identified and characterized as additional members of the Tm 12.86 gene family.

Part A: Southern Blot Analysis

Isolation of Genomic DNA. DNA was isolated from T. molitor larvae, which had been subject to prior dissection and gut removal. Approximately 20 grams of larval tissue was pulverized in liquid nitrogen using a mortar and pestle, and the powdered tissue was immediately transferred to centrifuge tubes containing 10 mls of resuspension buffer (0.1 M Tris-HCl, 0.01 M NaCl, 0.1 M EDTA, pH 8.0), and gently mixed to suspend the cells. The original suspension was then carefully placed on top of a 15 ml cushion of 0.88 M sucrose in a 45 milliliter centrifuge tube, and spun at 2500×g for 5 minutes to separate the nucleus from the dense protein granules which are difficult to break down and can lead to contamination of DNA. The top layer of sucrose containing the protein granules was discarded, while an equal volume of cell lysis buffer (0.1 M Tris-HCl, 0.1 M EDTA, 0.01 M NaCl, 1% SDS, pH 8.0), was added to the nuclei in the bottom of the centrifuge tube to break open the nuclear membrane. Proteinase K (Boehringer Mannheim, Indianapolis, Ind.) was added to the solution (150 mg/ml) and incubated at 55 C for 2 hours to break down any remaining protein. Then, 6 M NaCl was added to a final concentration of 1.5 M. The solution was vortexed vigorously, chilled on ice for 10 minutes, then centrifuged at 1200×g for 30 minutes. If the supernatant was not yet clear, it was necessary to transfer the solution and centrifuge for an additional 15 minutes in a clean tube. The supernatant was then transferred to a new 45 ml centrifuge tube containing an equal volume of 100% isopropanol and inverted several times to precipitate the DNA. The long strands of DNA were then pelleted by centrifuging at 1200×g for 15 minutes. The pellet was washed in 70% ethanol, dried moderately, and resuspended in TE buffer (10 mM Tris-HCl, 1 mM EDTA, pH 8.0). The DNA was then quantitated using a Pharmacia Ultrospec 3000 TM spectrophotometer (Pharmacia Biotech Inc., Piscataway, N.J.). On average, the amount of DNA obtained from one isolation procedure was >500 micrograms, with a 260/280 ratio between 1.8 and 1.9. Whenever 260/280 ratios were less than 1.8, suggesting further protein contamination in the final genomic DNA solution, it was again subject to treatment with additional Proteinase K and additional isopropanol precipitation until optical density ratios were acceptable.

Later, a second, more efficient protocol was brought into use. This protocol was more effective in allowing isolation of large amounts of genomic DNA. with very little protein contamination. Twenty grams of larvae, gut removed, were pulverized in liquid nitrogen, and the powdered tissue was placed in a 45 ml plastic centrifuge tube resuspended in 15 milliliters of 0.1 M Tris-HCl, 0.01 M NaCl, 0.1 M EDTA, pH 8.0. To this, 15 milliliters of cell lysis buffer was added (0.1 M Tris-HCl, 0.1 M EDTA, 0.01 M NaCl, 1% w/v SDS, pH 8.0). The solution was inverted gently to mix, and 150 g/ml Proteinase K was added. The solution was then incubated at 55 C for one hour. Next, 10 milliliters of 6 M NaCl was added for a final concentration of 1.5 M. This salting-out of the proteins proved adequate to precipitate protein granules. The solution was mixed well, and spun in a chilled centrifuge for 30 minutes at 1200×g. If the supernatant was not clear after this time, it was transferred to a clean tube and spun for an additional 15 minutes. The supernatant was then removed and divided between two clean 45 ml centrifuge tubes. One and a half volumes of ice cold ethanol were added to each tube to precipitate the DNA. The DNA was pelleted by spinning for 5 minutes at 1200×g, and resuspended in TE buffer, pH 8.0. The DNA was then re-precipitated and resuspended twice more, or until the pellet looked clean and the OD 260/280 ratio of the resuspended DNA was between 1.8 and 2.0. These final steps of re-precipitating the DNA removed any residual protein contamination. Yields of pure DNA were up to 500 μg.

Restriction Enzyme Digestion. The genomic DNA of T. molitor has a high percentage (more than 50%) of satellite DNA, which is a series of short, repeated sequences containing no genes. Because nearly half of the total genomic DNA extracted from the larvae is therefore this non-coding DNA, the amount isolated and loaded onto a gel for a Southern blot would have to be doubled in order to have adequate copies of the target gene for detection with the cDNA probe. T. molitor genomic DNA samples of known mass and purity were aliquoted into 1.5 ml centrifuge tubes. Restriction enzymes were obtained from New England Bio Labs (Beverly, Mass.) and were chosen on the basis of whether or not they cut within the cDNA sequences of interest, and therefore presumably the genomic copy of Tm 13.17 or 2-2/2-3 clones. Digests were carried out in the supplied buffers, at the temperature recommended for the particular enzyme, in volumes of at least 500 microliters. Digests took place from one hour to two days, depending on the amount of DNA to be digested. For larger amounts (i.e. >60(ug) more enzyme was added halfway through the digestion, and the reaction took place for at least one day. Alternatively, DNA was aliquoted into 10 microgram amounts for digestion. The separate digestions were then combined into the total amount of DNA desired in the sample. After digestion with the restriction enzyme, the DNA was spun in a Savant Speed-Vac Concentrator vacuum centrifuge in order to reduce the volume to less than 40 μl, so that the entire digestion could be loaded in one lane of the agarose gel to be used for the Southern blot. Gel electrophoresis of the DNA was used to confirm that it had been effectively cut by the restriction enzyme. The phagemid vector containing the cDNA insert to be used as a probe was cut with EcoRI and XhoI for one hour at 37 C to release the insert from the vector.

Gel Electrophoresis. The restriction enzyme digested samples of genomic DNA were run on a 1% or 1.5% agarose gel made with TBE buffer, at 80-85 volts in 1×TBE. After electrophoresis, the gels were stained for 20 minutes 0.1 μg/ml ethidium bromide solution and photographed under UV light using the UVP Image Store 5000 Gel Documentation System (UVP Inc., San Gabriel, Calif.) to visualize and photograph the DNA. The DNA was then denatured and neutralized in the gel by washing the entire gel twice for 15 minutes in 0.5 M NaOH, 1.5 M NaCl, then twice for 15 minutes in 1 M Tris-HCl, 1.5 M NaCl, pH 7.5, and finally rinsing in distilled water. Some of the gels were also depurinated prior to transfer, however this step did not seem to effect the subsequent transfer of large pieces of DNA to the membrane. Gels stained and viewed after the transfer showed that no DNA remained in the gel. DNA still stained with ethidium bromide could be seen on the membrane under UV light after transfer. Non-depurinated DNA transferred equally well.

Southern Blotting. Southern blots were prepared according to standard protocols. The prepared gels containing 20-100 μg of digested, denatured genomic DNA were inverted on a blotting apparatus containing 20×SSC buffer. A positively charged nylon membrane from Boehringer Mannheim was placed over the gel and covered with two pieces of 3 mm Whatman “1” paper, then a stack of absorbent paper towels and a weight of approximately 500 grams. The capillary blotting of the DNA onto the membrane was allowed to proceed overnight. The next day, the membrane was removed and immediately crosslinked on both sides using a Fisher Biotech FB-UVXL-1000™ UV crosslinker. The gel and the membrane were then observed under UV light to be sure the DNA was successfully transferred to the membrane.

Probe Labeling and Detection. Probes used in hybridization to Southern blots: Tm 13.17, 2-2, and 2-3. These cDNA inserts were amplified by PCR using the T3 and T7 primers sites contained in the PBk-CMV phagemid vector, or primers from the termini of the cDNA inserts themselves. The cDNA primers resulted in a slightly shorter probe, as some of the cDNA ends on the outside of the primers were not amplified. Several methods were explored to label the Tm 13.17, 2-2, and 2-3 cDNA probes, in order to achieve appropriately high level of probe sensitivity and specificity. These methods consisted of 1) Psoralen-biotin labeling, 2) DIG labeling, and 3) 32P labeling.

Psoralen Biotin Labeling, Hybridization, and Detection: We used the BrightStar Psoralen-Biotin probe labeling kit obtained from Ambion, Inc. (Austin, Tex.) which makes use of a molecule, Psoralen, that upon exposure to UV light, intercalates into the DNA molecule and becomes covalently bound. This molecule can subsequently be detected chemiluminescently using a biotin-aviden conjugate. Probe labeling was conducted as per directions of the manufacture. Under dim lights, one microliter of the Psoralen-Biotin reagent was added to 10 microliters of the nucleic acid solution (Tm 13.17 PCR amplified cDNA) in an eppendorf tube and mixed. This solution was transferred to a well of a microtiter plate placed on ice. A 365 nm ultraviolet light source was placed directly over the sample, and it was irradiated for 45 minutes. The sample was then diluted to 100 microliters by adding 89 microliters of TE buffer and transferred to a clean micrcentrifuge tube. Two hundred microliters of ddH2O-saturated n-butanol were then added. The sample was vortexed and centrifuged for one minute at 7,000×g. The top layer of n-butanol was removed with a pipette, and the labeled probe was stored at −80C until needed for hybridization and detection.

At that time, the nylon membrane containing the genomic DNA was wetted with 0.25 M disodium phosphate. Prehybridization was at 65 C for one hour in hybridization buffer (1 mM EDTA, 7% SDS, 0.25 M disodium phosphate, pH 7.2) with constant agitation. The labeled probe was denatured by boiling for five minutes and then diluted to 100 ng/ml in 8 mls of hybridization buffer and added to the membrane in a sealed plastic bag. Hybridization took place overnight at 65 C in a water bath with constant agitation. The membrane was then washed 2×15 minutes in 2× concentrated sodium citrate buffer (2×SSC) and 1% SDS, 2×15 minutes in 1×SSC, 1% w/v SDS at 65 C, and 2×5 minutes in 1×.

Detection of the Psoralen-Biotin labeled probe was with Sigma's Chemiluminescent DNA Detection Kit as per manufacture's instructions. The membrane was washed 2×5 minutes in blocking buffer (200 mls PBS, 4 gm I-Block™, 10 mls 10% SDS), and then incubated in blocking buffer for 10 minutes. The streptavidin phosphatase conjugate was diluted 1:5000 in blocking buffer, and the membrane was incubated with the conjugate solution for 20 minutes with constant agitation. The membrane was then washed for five minutes in blocking buffer, and 3 times for 5 minutes in Detection Wash Buffer (1×PBS, 0.5% SDS), and 2 times for 2 minutes in Assay Buffer (0.1M diethanolamine, 1 mM magnesium chloride). The Chemiluminescent Substrate Solution was then diluted (25 microliters in 4 mls) and added to the membrane with agitation for 5 minutes. The membrane was then sealed in plastic and exposed to Kodak BioMax™ film for three hours or as otherwise stated.

DIG Labeling, Hybridization, and Detection. We also used a digoxygenin (DIG) labeling kit from Boehringer Mannheim (Indianapolis, Ind.), specifically, the PCR Dig Probe Synthesis Kit. In this case, the detectable DIG molecule was attached to a dUTP nucleotide, which became incorporated in the Tm 13.17, 2-2, or 2-3 cDNAs upon PCR amplification. Probe labeling via the PCR DIG Probe Synthesis kit was conducted as per manufacture's instructions. Briefly, digoxygenin-11-dUTP (DIG dUTP) is incorporated by Taq polymerase during PCR. The cDNA probes (Tm 13.17, 2-2, and 2-3) were labeled with DIG UTP in a PCR reaction volume of 50 μl and containing: Five μl of PCR buffer (100 mM Tris-HCl, 500 mM KCl; pH 8.3), 5 μl MgCl2 stock solution (25 mM MgCl2), 5 μl PCR DIG probe synthesis mix (2 mM dATP, 2 mM dCTP, 2 mM dGTP, 1.3 mM dTTP, 0.7 mM alkai-labile DIG-11-dUTP; pH 7.0), 0.8 μl Taq DNA polymerase (5 U/μl), T3 and T7 primers (0.2 mM final concentration), cDNA template (0.1 ng), and ddH2O to a total volume of 50 μl The PCR reaction conditions were: 95 C for 45 seconds, 55 C for one minute, 72 C for two minutes, for 40 cycles. The average concentration of probe after 40 cycles of PCR was about 70 ng/μl. Labeled probes and unlabeled controls were run on a gel to confirm successful incorporation of the DIG label, via a labeled probe (being slightly larger), running at slightly higher on the gel than unlabeled probe. Labeling was also ascertained with dot blots of the labeled probe, using chemiluminscent detection.

Pre-hybridization and hybridization of the membranes was carried out in either a standard buffer [5×SSC, 0.1% (w/v) N-lauroylsarcosine, 0.02% (w/v) SDS, and 1% Blocking Reagent (provided with detection kit)], or a formamide buffer [50% formamide, 5×SSC, 0.1% (w/v) N-lauroylsarcosine, 0.02% (w/v) SDS, and 2% Blocking Reagent]. Hybridization in the formamide buffer was carried out at room temperature, whereas hybridization temperatures in the standard buffer were usually 37 C or higher. Pre-hybridization was for one to two hours, and temperatures used ranged from 20 to 65 C with constant agitation. Hybridizations were carried out over night at the same temperature as pre-hybridization, also with constant agitation. Probe concentration in the hybridization buffer was 5-25 ng/ml.

After hybridization, the membranes were washed twice for five minutes in 2×SSC, 0.1% SDS, and twice for fifteen minutes in 0.1×SDS, 0.1% SDS, at hybridization temperature. Chemiluminescent detection of DIG labeled probes was with alkaline phosphatase conjugated Anti-DIG, as per manufacture's instructions. Membranes were washed five minutes in washing buffer (100 mM Tris-HCl, 150 mM NaCl, pH 7.5; 0.3% v/v Tween 20) and incubated for 30 minutes in 1× blocking buffer (1% w/v Blocking Reagent dissolved in 100 mM Tris-HCl, 150 mM NaCl buffer, pH 7.5) with gentle agitation. This was followed by a 30 minute incubation in a 1:100,000 (75 mU/ml) dilution of anti-DIG alkaline phosphatase conjugate in 1× blocking buffer. The membranes were then washed twice for fifteen minutes in washing buffer, and equilibrated for five minutes in detection buffer (100 mM Tris-HCl, 100 mM NaCl, pH 9.5). The CSPD^{R} chemiluminescent substrate was diluted 1:100 in 20 mls of detection buffer, and was incubated in a sealed bag with a membrane for fifteen minutes. The excess was then blotted off with filter paper, and the damp membrane was sealed in a plastic bag. The membrane was then exposed to film (Kodak Biomax™) at 37 C for at least fifteen minutes, and up to 24 hours.

32P Labeling, Hybridization, and Detection. The final method for probe labeling was 32P, since 32P labeling is reputed to be significantly more sensitive than chemiluminescent detection methods. Therefore we proceeded to also label cDNA probes using the RTS RadPtime DNA Labeling System from Life Technologies (Gaithersburg, Md.) as per manufacture's instructions. This kit uses the random primer labeling method. Twenty five nanograms of cDNA PCR product, as determined by spectrophotometric readings at 260 nm, was dissolved in 45 microliters of TE buffer (10 mM Tris-HCl, pH 7.5; 1 mM EDTA). The cDNA was then added to the pre-mixed reaction components: 50 mM Tris acetate (pH 6.8), 5 mM magnesium acetate, 1 mM dithiothreitol, 60 ug/ml random octamer primers, 10 uM dATP, 10 uM dGTP, 10 uM dTTP, and 3-6 U/ul Klenow fragment. After mixing thoroughly, 5 ul [(a-32 P] dCTP (3000 Ci/mmol, 10 uCi/ul), obtained from New England Nuclear (Boston, Mass.), was added, and the microfuge tube was centrifuged for 30 seconds. The reaction was allowed to take place at 37° C. for 10 minutes. The reaction was stopped by the addition of 5 ul of 0.2 M EDTA. The entire reaction volume was then immediately added to the Southern blot in a plastic bag and sealed for hybridization.

For hybridization and detection, membranes were prehybridized at the appropriate temperature (42° C. to 68° C.) in 6×SSC, 5× Denhardt's reagent, 0.5% SDS, and 100 microgram per milliliter denatured herring sperm DNA with constant agitation. Hybridization was carried out at the same temperature as pre-hybridization also with constant agitation, either in the identical buffer, or without the herring sperm DNA, in order to increase the likelihood of probe binding. The entire reaction volume of probe (50 microliters) was added to each hybridization. Each probe was re-used several times after boiling to denature double stranded probe. After washing, the membranes were blotted dry on Whatmampaper and sealed in plastic bags. The membranes were exposed to Kodak Biomaxfilm at −70° C. in cartridges wrapped in plastic, for the appropriate length of time, from one hour up to fifteen days. Some of the exposures used the Kodak Trans-screen LE™ intensifying screen for ³²P isotopic detection.

Part B: PCR Amplification of Genomic DNA Fragments

Isolation of Genomic DNA. The method used to isolate T. molitor genomic DNA for PCR is also the protein salting-out method used for Southern blotting. DNA used in subsequent PCR reactions had a ratio of OD at 260/280 nm of 2.0, and was at a concentration of 300 ng/μl.

PCR Amplification of Genomic DNA. Several protocols for the PCR amplification of the Tm 12.86 family of genes were used initially. PCR reactions were set up on ice to contain between 500 ng and 5 ug of genomic DNA, 10 mM dNTPs from Boehringer Mannheim, 20 mM MgCl2 buffer, 0.25 uM final concentration of each primer (forward and reverse), various amounts of sterile ddH2O to 50 ul total reaction volume, and 5 units of Taq polymerase from Boehringer Mannheim. Reactions were run with a primer annealing temperature of between 25 C and 55 C. The primers for these reaction were sequence from both termini of the Tm 13.17 cDNA clone. Since results from this initial procedure showed no PCR products visible on ethidium bromide stained agarose gels, new protocols were then implemented.

Thus, primers were designed which encompassed regions at either terminus of the Tm 13.17 clone which have the greatest degree of conservation between all known cDNA sequences which may belong to the Tm 12.86 gene family (FIG. 4.6). The melting temperature of both Primers is 44° C. The TaqPlus-Long PCR System kit was purchased from Stratagene. This kit contained a mixture of Taq DNA polymerase and cloned Pfu DNA polymerase to optimize the synthesis of long or difficult to amplify target sequences. Reactions were run as per manufacture's instructions. Various salt concentrations, amounts of template, annealing and elongation temperatures and times, and primer combinations were used, but as with the previous approach, no product was observed with ethidium bromide staining.

The third approach taken used the kit and protocol from the Sigma AccuTaq LA-DNA polymerase mix. This mix incorporates Taq DNA polymerase and a thermostable proofreading enzyme to increase the length and accuracy of amplification. The kit also included dimethyl sulfoxide (DMSO), and a protocol for its use in PCR. The reactions were carried out as per manufacture's instructions, incorporating between 1% and 5% DMSO. Cycling parameters included a 15 second denaturation at 94° C., primer annealing at various temperatures (25-65 C) for 20 seconds, and extension for 20 minutes at 68 C. Primers for the PCR were those described in FIG. 4.6. Since PCR products were successfully obtained with this approach, they were then subject to further detection and cloning steps.

Detection of PCR Products. Twenty microliters of each PCR reaction was run on a 0.8% agarose gel made with TBE, and stained with ethidium bromide to see if any products were visible with ethidium bromide staining. The gel was then blotted onto a Boehringer Mannheim positively charged nylon membrane for later hybridization with a labeled cDNA probe. The remainder of each reaction was reserved for ligation into a vector and subsequent transformation of the bacterial host for cloning and selection.

Cloning of PCR Generated Fragments.

a) Ligation of Fragments into a Vector. PCR products were purified using a Centispin spin column from Princeton Separations as per manufacture's instructions to remove unincorporated dNTPs, polylmerase, and primers. The PCR products were recovered from the column in TE buffer, pH 8.0. Several methods were used to try to clone the PCR generated fragments.

b) Blunt—End Ligation. The ligation of the DNA fragments into a vector was accomplished with the Prime PCR Cloner Cloning System from 5 Prime→3 Prime, Inc. In a 0.65 microcentrifuge tube, 4.5 ul molecular biology grade water, 2 ul 10× Prime PCR Cloning Reagent, 1 ul Prime PCR Cloner Nucleotide Stock, 1 ul 0.1 M DTT Solution, and 10 ul of column processed PCR product were combined. The contents of the tube were mixed briefly by vortexing, then 1.5 ul of the Prime PCR Modification Reagent was added to the tube. The contents were again mixed by vortexing, spun briefly, then incubated at 16 C for 15 minutes. The tube was then heated at 75 C for 15 minutes. After heating, the cloning reaction was set at room temperature until needed in the ligation protocol. In another 0.65 ml microcentrifuge tube, 5 μl molecular biology grade water, 1 μl 0.1 M DTT Solution, 2 μl of 10× Prime Efficiency Ligation Buffer, 10 μl of the cloning reaction from above, and 1 μl of pNoTA vector DNA were combined. The contents of the tube were mixed briefly, then 1 μL of T4 DNA Ligase was added. The solution was mixed well and spun briefly. The tube was then incubated at 25 C for 30 minutes. The ligation mixture was then heated at 65 C for 2 minutes. The tube was then set aside at room temperature until needed in the transformation protocol. Blue/white selection was used to identify recombinant clones.

c) Direct Ligation into pGEM Cloning Vector. PCR products were again column purified using Centri-Sep spin columns. Both the pGEM sequencing vector (provided with the Perkin Elmer DNA sequencing kit) and the purified PCR product were digested in separate reactions with EcoRI for 1 hour. The digested PCR product and vector were then combined with T4 DNA Ligase (Boehringer Mannheim) as per manufacture's instructions, and allowed to ligate for 24 hours at room temperature. Clones were differentiated by blue/white selection.

Transformation of Bacterial Host

a) Making Competent Cells. The bacterial host used for cloning of the PCR fragments was the E. coli strain DH5a. The bacterial cells were grown overnight and diluted to an OD 600 of 0.5. Forty milliliters of cells were placed in a 45 ml conical plastic centrifuge tube, and spun for 10 minutes at 1000×g. The supernatant was sterilely removed, and the cells were resuspended in 5 mls of 50 mM CaCl₂. The cells were then placed on ice for 20 minutes, and respun for ten minutes at 1000×g. The supernatant was again sterilely removed, and the cells were resuspended in 1 ml of 50 mM CaCl₂. The cells were stored on ice in a 4 C refrigerator until needed.

b) Transformation and Selection of Recombinant Clones. Two hundred microliters of competent cells were added directly to the ligation reaction, and mixed gently by tapping the tube. A control tube with no DNA was also prepared. The tubes were then incubated for 20 minutes on ice, after which they were heat shocked for 90 seconds at 42 C, and then returned to the ice. For blue/white selection, all 200 μl of the transformation reaction was then spread onto an LB plate containing 100 μg/ml ampicillin, 100 μl of 0.6 mM isopropyl-1-thio-(-D-galactophyranoside (IPTG) solution, and 40 μl of 20 mg/ml X-gal (5-Bromo-4-Chloro-3-indolyl-(-D-galactoside) solution. The plate was incubated overnight at 37 C. The following day, recombinant colonies were identified by their white color. Some of these colonies were selected, inoculated into LB broth, and grown overnight at 37 C for subsequent plasmid isolation.

TOPO™ XL PCR Cloning. Both the blunt-end ligation and ligating into the p-GEM cloning vector did not appear to be sufficiently effective, therefore a third method was used. The TOPO™ XL PCR Cloning Kit was purchased from Invitrogen (Carlsbad, Calif.). The procedure was as per manufacture's instructions. In brief, several PCRs were run on a 0.8% agarose gel containing 40 μl of 2 mg/ml Crystal Violet solution. Eight μl of 6× Crystal Violet Loading Buffer was added to each PCR amplification, and each PCR was loaded into one well of the gel. The gel was run at 80 volts until the crystal violet in the gel had run one quarter of the way up the gel. PCR products appeared as a thin blue band. The bands were excised from the gel with a razor blade, cut up into small pieces, and transferred to a sterile 1.5 ml centrifuge tube. The volume of the agarose pieces was estimated, and 2.5 times the volume of 6.6 M sodium iodide was added. The tube was mixed by vortexing, and then incubated at 50 C to melt the agarose. At room temperature 1.5 volumes of Binding Buffer was added and mixed. All of the mixture was then loaded onto a S.N.A.P. purification column. The column was centrifuged at 3,000×g for 30 seconds, then the liquid was poured back onto the column and respun two more times to make sure all of the DNA was bound to the column. After the third spin, 400 μl of 1× Final Wash was added to the column, and it was centrifuged as before. The column was dried by centrifuging at >10,000×g for at least one minute, and then 40 μl of TE buffer was added, and the column was incubated at room temperature for one minute. The column was centrifuged at >10,000×g for one minute to elute the DNA into the microcentrifuge tube. Concentration of the isolated PCR product was estimated by ethidium bromide agarose gel electrophoresis.

For the cloning reaction, 4 μl of gel purified PCR product and 1 μl of the pCRrXL-TOPO vector were mixed together in a sterile microfuge tube and incubated at room temperature for 5 minutes. Then, 1 ul of the 6×TOPO Cloning Stop Solution was added and mixed. Two uls of the cloning reaction were added to a vial of One Shot TOP10 chemically competent cells and mixed gently, then incubated on ice for 30 minutes. After the incubation, the cells were heat shocked at 42° C. for 30 seconds, and incubated on ice for an additional two minutes. Next, 250 μl of SOC medium was added, and the tube was incubated at 37° C. for one hour with shaking. Then 150 μl of the transformation reaction was then spread on a prewarmed LB plate. The plate was incubated overnight at 37° C. The next day, positive clones (any colonies growing on the plate) were selected and grown overnight in LB broth for plasmid isolation and further analysis.

Isolation of Plasmid DNA. Bacterial cells containing the recombinant plasmids of interest were grown overnight in Luria—Bertani (LB) broth. The cells were spun down in a 1.6 ml centrifuge tube for one minute, then the supernatant was poured off. One hundred microliters of ice cold GTE (50 mM glucose, 25 mM Tris, 10 mM EDTA) solution was added and the cells were resuspended by pipetting up and down. Then 5 μl of 5 mg/ml RNase (Boehringer Mannheim) and 200 μl 1% SDS/0.2 N NaOH solution were added and the tubes were mixed by rapidly inverting them five times. After standing on ice for five minutes, 150 μl ice cold KOAc solution (60 ml 5 M potassium acetate, 11.5 ml glacial acetic acid, 28.5 ml distilled water) was added to each tube. The tubes were again mixed by inverting five times, and incubated on ice for five minutes. The tubes were then spun for five minutes to pellet the precipitate. The supernatant was transferred to a clean 1.6 ml tube, and 400 μl of isopropanol was added. The solution was mixed by rapidly inverting the tubes, and then incubated at room temperature for 90 seconds. The tubes were then spun for five minutes to pellet the nucleic acids. The pellets were washed with 200 μl of 95% ethanol, re-spun, and allowed to air dry. When dry, the nucleic acid pellets were resuspended in 15-20 μl TE buffer (10 mM Tris, 1 mM EDTA). Plasmids were run on a 0.8% agarose gel and viewed by ethidium bromide staining.

Sequencing of Clones. Plasmids believed to have an insert based on their larger size were chosen for DNA sequencing. In a 0.5 ml PCR reaction tube, 5 ug of plasmid DNA was added to 3.2 picomoles of M13 Universal Primer, and 8.0 microliters of the Terminator Ready Reactions Mix from the Perkin Elmer DNA sequencing kit. The tube was spun briefly, then subject to PCR under the following conditions: 1.0 C/second thermal ramp to 96° C. for 30 seconds, then 1.0 C/second thermal ramp to 50° C. for 15 seconds, then 1.0 C/second thermal ramp to 60° C., 60° C. for 4 minutes. This was repeated for a total of 25 cycles. After PCR, the samples were filtered through a CentriSep™ spin column (Princeton Separations) to remove unincorporated dye and primers, then the sequence was read by the ABI Prism Model 310 DNA Sequencer.

Part C: Cloning Additional Homologous cDNAs

Screening the T. molitor cDNA Ubrary. The cDNA library generated from winter acclimated T. molitor larval total mRNA as detailed in Example 2 using the Stratagene Zap Express™ cDNA synthesis and cloning kit (Stratagene, La Jolla, Calif.) was used for screening of additional Tm 12.86 homologues. The vector used for cloning the cDNAs was the PBk-CMV phagemid (FIG. 2.4).

Screening of the cDNA library was done as per the cDNA cloning kit manufacture's instructions as detailed in Example 3. In this case, six hundred microliters of the phage and bacteria mixture was prepared and added to 6.5 ml of top agar at 48° C., and poured onto the warm NZY plate. The agar was allowed to harden at room temperature, and was then incubated at 42° C. for five hours. After five hours of incubation small phage plaques were visible. Nitrocellulose membranes (Stratagene) cut to fit the plates were submerged in 10 mM IPTG (isopropyl-1-thio-fl-D-galactopyranoside) until completely wet, then air dried on Whatman 3 mm paper. The plates were covered with the IPTG treated membranes and incubated overnight at 37° C. The next day, the plates were chilled at 4° C. for two hours to prevent the top agar from sticking to the membranes. The membranes were marked for orientation on the plates, then carefully lited and washed in PBS buffer (0.002 M KCl, 0.14 M NaCl, 0.01 M Na2HPO4, 0.0015 M KH2PO4, pH 7.2), three times for five minutes each time, with shaking. The membranes were then blocked with 5% (w/v) nonfat dry milk in PBS for one hour with gentle agitation, then washed with PBS as above. The membranes were then incubated in 3% H2O2 for 30 minutes to block endogenous peroxidases, and then washed in PBS three more times for five minutes each time. Next, the membranes were incubated in a 1:2000 dilution in PBS of the primary antibody serum (rabbit anti-Tm 12.86) for two hours with gentle shaking, then washed with PBS again as above. Then, the membranes were incubated with a 1:500 dilution (in PBS) of the secondary antibody (peroxidase-conjugate goat-anti-rabbit [Sigma]), for two hours and washed with PBS as above. The positive plaques were colorized with 15 ml of DAB solution (3,3í—diaminobenzidine tetrahydrochloride; Fast DABTM: Sigma) in PBS with gentle agitation until positive clones were visualized as dark colored spots. The reaction was stopped by washing the membrane with PBS. The membranes were air dried to preserve them.

Excision of Positive Clones. (as in Example 3 with slight modification). Membranes were lined up with the original plates, and positive plaques were cored and stored in 500 μl of SM buffer (5.8 g NaCl, 2.0 g MgSO4*7H2O, 50.0 ml of 1M Tris-HCl {pH 7.5}, and 5.0 ml of 2% {w/v} gelatin) and 20 μl of chloroform. Positive plaques too close to background plaques were subjected to re-plating and secondary screening.

E. coli strains XLI-Blue MRF′ and XLOLR were grown overnight in NZY broth at 37° C. The next day, the cells were pelleted at 2000 rpm for 10 minutes and resuspended 10 mM MgSO4 at an OD 600 of 1.0. Then 200 μl of XI-Blue MRFí cells were mixed with 1 μl of the ExAssist helper phage (Stratagene), and incubated in a 15 ml Falcon tube for 15 minutes at 37° C. Three milliliters of NZY broth were added, and the tubes were incubated for 3 hours at 37° C. with shaking. The solution was subsequently heated to 65-70° C. for 20 minutes, and spun at 1000×g for 15 minutes. At this point, the supernatant contained the excised pBK-CMV phagemid, packaged as filamentous phage particles. Two hundred microliters of the XLOLR cells were added to two microcentrifuge tubes. To one tube, 100 μl of the phage—containing supernatant was added, and 10 μl was added to the other. These solutions were incubated for 15 minutes at 37° C., then 300 μl of NZY broth was added and the tubes were incubated for an additional 45 minutes at 37° C. After incubation, 200 μl of each mixture was plated on LB-Kanamycin plates (10 g NaCl, 10 g tryptone, 5 g yeast extract, 20 g agarose, ddH2O to one liter, pH 7.0, 50 μg/ml Kanamycin) and incubated overnight at 37° C. Colonies growing on the plates the next day contained the phagemid. Several colonies from each plate were selected and grown over night in 3 mls of LB broth with Kanamycin (10 g NaCl, 10 g tryptone, 5 g yeast extract, to 1 liter with ddH2O, pH 7.0, 50 μg/ml Kanamycin).

Phagemid Isolation. In this case, the protocol followed for plasmid isolation was from Laboratory DNA Science. The bacterial cells grown overnight were transferred to a 1.6 ml microcentrifuge tube, and spun for one minute to pellet the cells. The supernatant was poured off and more cells were added and spun down, until most of the 3 ml overnight culture had been pelleted. The pellet was then resuspended in 100 μl of ice cold GTE solution (50 mM glucose, 25 mM Tris, 10 mM EDTA {ethylene diamine tetraacetic acid}) until no clumps remained. Then 200 μl of room temperature SDS/NaOH solution [1% (w/v) SDS (sodium dodecyl sulfate), 0.2 N NaOH] was added to each tube, and the solutions were mixed by rapidly inverting the tubes about 5 times. The tubes were incubated on ice for 5 minutes, then 150 μl of ice cold KoAc solution [60 mls of 5 M KoAc (potassium acetate), 11.5 mls of glacial acetic acid, and 28.5 mls dd H2O] was added. The solutions were again mixed by rapid inversion, and incubated on ice for 5 minutes. They were then spun for 5 minutes to pellet the precipitate. The supernatants containing the plasmid were transferred to clean microcentrifuge tubes, and 400 μl of isopropanol was added to each tube. The solution was mixed by inverting the tubes, and they were incubated at room temperature for 2 minutes, then spun for 5 minutes to pellet the nucleic acids. The isopropanol was removed, and the pellets were washed with 95% ethanol, then dried. The DNA pellets were resuspended in 15 μl TE buffer (10 mM Tris, 1 mM EDTA). Isolated plasmid DNA was cut with appropriate restriction enzymes and viewed on an agarose gel.

Sequencing. Phagemids believed to have an insert based on their larger size were chosen for DNA sequencing, by the method detailed in Example 4, Part B.

Part D: Sequence Comparision to Examine Relationships within the Tm12.86 Multigene Family

Sequence Data. DNA sequence data from T. molitor was obtained from cDNA clones selected from a T. molitor cold acclimated cDNA library with an antibody to the T. molitor AFP Tm 12.86. Several positive clones were sequenced using the ABI Prism model 310 DNA sequencer. The clones concentrated on are Tm 13.17 (Example 2), 2-2 and 2-3 (Example 3), and 3-4, 3-9, and 7-5 (Example 4, Part C). Also available were the N-terminal amino acid sequence of Tm 12.86 (Example 1), and the nucleotide sequence and predicted amino acid sequence of AFP-3, B 1 and B2, and other sequence data obtained from GenBank (www.ncbi.nlm.nih.gov).

Alignments. Alignments of nucleotide and amino acid sequences was done using the computer program DNASTAR (DNASTAR Inc, Madison Wis.). The Clustal method of multi-sequence alignment with a weighted residue table generated by the computer was used. Sequence similarity tables were also produced by DNASTAR, using the Megalign Program.

### EXAMPLE 5

Part A: Effect of Bacterial Proteins on Antifreeze Activity

In order to evaluate the effect of endogenous bacterial proteins on the antifreeze activity of recombinant proteins, purified Tm 12.86 was tested in the presence of bacterial proteins. The bacterial strain XLOLR-1 used in this experiment is identical to the strain used in the cloning and expression of the T. molitor cDNA library (detailed in Example 2). As a negative control, the antifreeze activity of endogenous bacterial proteins were tested. Antifreeze activity of samples was tested by Recrystallization Inhibition assay (R1) (Described in Example 8).

Purification of bacterial proteins. A stab from a frozen stock of XLOLR [D(mcrA)183-(mcrCB-hsdSMR-mrr)173endAlthi-1 recAlgyrA96relAl lac{FproABlacIqZ-M15Tn10(Tetr)]^{c} (Obtained from Stratagene, CA) was innoculated in 3 ml of LB media (Luria-Bertani medium, 10 g bacto-tryptone; 5 g bacto-yeast extract; 10 g NaCl with deionized H20 added to a final volume of 1 liter, and pH adjusted to 7.0 with NaOH; autoclaved for 20 minutes at 15 lb/sq.in. in liquid cycle) containing 12.5 (g/ml of tetracycline. The cultures were grown over-night in a loosely capped 15 ml conical tube (Fisher, Cat. No. F2054) for 13-15 hrs with 250 rpm agitation at 37 C. The resulting culture was centrifuged in two 1.5 ml microcentrifuge tubes at 12,000×g for 5 minutes. The supernatant was discarded and the pellet was resuspended in 0.2 ml of protein extraction buffer (10 mM Tris-HCl at pH 8.0, 1 mM EDTA and 1% SDS). Samples were briefly sonicated (10 seconds, 90% output) and centrifuged at 12,000×g for 5 minutes. The supernatant was transferred to a new tube and quantitated for total proteins. Two microlitres of the supernatant was used to determine the protein concentration of the lysate by using the Bradford assay.

Sample Preparation. A working concentration of 0.25 mg/ml of Tm 12.86 (initially at 25 mg/ml) was prepared by diluting 1 ul of the protein in 99 ul of protein extraction buffer. Purified Tm 12.86 was provided using protocols detailed in Example 1. The concentration of the lysate was determined to be 2.5 mg/ml. Serial dilutions of 0.025 mg/ml and 0.0025 mg/ml were made in 0.9% NaCl. Similarly, a working concentration of 0.25 mg/ml of Tm12.86 and 2.3 mg/ml of XLOLR lysate was prepared by diluting 1 ul of the working concentration with 9 ul of XLOLR lysate (2.5 mg/ml). Serial dilution of 0.025 mg/ml and 0.0025 mg/ml of this sample was prepared in 0.9% NaCl solution.

Recrystallization Inhibition (RI) Activity. The protocols used to measure the RI activity in these samples reflecting a measure of the antifreeze activity are detailed Example 8.

Part B: Generation of Signal Plus and Signal Minus His-Tagged Inserts Cloning and Protein Purification of Signal Peptide Deleted AFP

Signal Peptides. The lack of antifreeze activity of the recombinant products may be attributed to the presence of a uncleaved signal peptide. Therefore, a region of the cDNA that encodes for the signal peptide was deleted and the remaining insert expressed in E. coli in order to generate signal-minus recombinant proteins.

Histidine-Tag Expression System. As part of this approach, we introduced a system to facilitate rapid purification of the AFP recombinant protein, i.e. a histidine-tag purification system. This involves cloning the gene of interest in an expression vector pET 28a, which is capable of linking a hexamer of histidine amino acids to the protein of interest (FIG. 5.0, Novagen Catalog 1998). During purification, the negatively charged histidine tag becomes coordinated to the positively charged Ni++ resin and subsequent elutions allow for the selective purification of the histidine tagged recombinant AFP. Thus, purification of recombinant AFP is based on selective affinity chromatography. Following this, the eluted protein is dialyzed, lyophilized and tested for activity. If necessary, the histidine-tag can be cleaved proteolytically (FIG. 5.1).

Cloning in pET-28a Vector

A. Transformation of DH5a′. DH5a.[F-¢80dlac ZdM15 d(lac ZYA-argF)U169 deo R rec AendA1 hsd R17 (rk−, mk+) pho A supE441-thi-1gyr96relA1] is a strain of E. coli that is routinely used for sub-cloning plasmids (Stratagene Catalog, 1998). Mutation in the end A1 bacterial gene greatly increases plasmid yield and quality, while a mutation in the deo R gene permits stable transformation of large plasmids. The presence of the lacZ gene supports blue/white screening of colonies. The cloning vector pET28a and plasmids pBK-CMV: 2-2, 2-3 and Tm13.17 were transformed in DH5a. Competent cells of DHSa were prepared by standard procedures. Fifty microlitres of competent cells were incubated with 150 ng of plasmid DNA for 30 minutes at 4° C. in 1.5 ml micro-centrifuge tubes. The tubes were transferred to a water-bath at 42° C. for 45 seconds and immediately followed by incubation on ice for 2 minutes. Five hundred microlitres of LB media was added to the cells and incubated at 37° C. for 30 minutes with shaking. The cells were spread on LB-Agar plates with kanamycin at 50 mg/ml and incubated in a chamber at 37° C. for 12-15 hours.

B. Plasmid Purification. Individual colonies were selected and grown in 5 ml of LB media with kanamycin (50 mg/ml) in polypropylene tubes with loosely fitted caps. The tubes were agitated at 250 rpm at 37° C. for 8-10 hours. The culture was transferred to 100 ml LB-kanamycin (50 mg/ml) in a 500 ml Erlenmeyer flask and grown in identical conditions. The bacterial growth rate was monitored every 30 minutes until the culture reached an OD600 between 1.0-1.5 when 2 ml of this was then measured in plastic cuvettes at 600 nm. The bacterial culture was transferred to a polycarbonate centrifuge tube and centrifuged in a Sorvall RC 5B+ centrifuge at 4° C. in a Sorvall GSA for 15 min at 5000×g. The bacterial pellet was saved after discarding the supernatant. [Note: At this point the pellet was stored at −8° C. if it could not be processed immediately]. A plasmid purification kit was purchased from Qiagen (Valencia, Calif.) and used according to the manufacturer's protocol with slight modifications. The pellet was resuspended in 4 ml of pre-chilled resuspension buffer (P1: 50 mM Tris-Cl pH 8.0, 10 mM EDTA and 100 ug/ml RNase). The resuspended pellets were then transferred to 30 ml COREX [symbol] centrifuge tubes. Upon transfer, 4 ml of lysis buffer (P2: 200 mM NaOH and 1% SDS, at room temperature) was added to the re-suspension. The solution was gently, but thoroughly mixed by inverting 4-6 times and incubated at room temperature for no more than 3 minutes. Four ml of pre-chilled neutralization buffer (P3: 3.0 M Potassium acetate pH 5.5) was added to the lysate and mixed gently by inversion and stored for an additional 15 minutes on ice. The sample was centrifuged at 20,000×g for 30 minutes at 4° C. in a Sorvall SS-34 rotor. The supernatant was transferred to a clean COREX [symbol] tube and re-centrifuged for additional 30 minutes at the above setting. Without disturbing the pellet, the supernatant was carefully transferred to a 15 ml polypropylene bottle. A Qiagen-tip was equilibrated with 4 ml of equilibration buffer (Buffer QBT: 750 mM NaCl, 50 mM MOPS, pH 7.0; 15% isopropanol) that emptied the column by gravity flow. The plasmid supernatant was added to the Qiagen-tip and once again emptied by gravity flow. The column was washed twice with 20 ml wash buffer (Buffer QC: 1.0 m, NaCl; 50 mM MOPS, pH 7.0; 15% isopropanol). Following this, 5 ml of elution buffer pre-warmed at 50° C., was added to the Qiagen tip and the elutant was saved in a COREX [symbol] tube. Three and a half ml of isopropanol was added to the elutant and incubated on ice for 20 minutes. The sample was centrifuged at 15,000×g for 30 minutes at 4° C. Following this, the supernatant was discarded and the minute glassy pellet was re-suspended in 70% ethanol in order to dissolve the excess salt. The sample was re-centrifuged under identical conditions and the resulting supernatant was discarded. The plasmid was airdried and re-suspended in 100 ul of ddH2O.

C. Restriction Analysis of pET 28a, pBK-CMV: [2-2, 2-3 and Tm 13.17]. The purified plasmids were analyzed for quantity and quality. Samples for DNA content were prepared in a 1.5 ml micro-centrifuge tube at a dilution of 1:200 (5 ul of plasmid DNA in 995 ul of ddH2O). DNA content was measured in quartz cuvettes at 260 and 280 nm in a UKB Spectrophotometer. Content was interpreted with the formula: (dilution factor)×(absorbance at 260 nm)×(50, a constant for double stranded DNA) mg(ml. In order to ensure that the correct plasmids were purified, restriction digestion of the samples were performed and analyzed on an agarose gel. The final volume of the restriction digest was 30 ul that contained 2 ul of plasmid DNA (0.5ug/ul), 3 ul of 10×BamHI buffer, 0.3 ul of 100× Bovine Serum Albumin, 1 ul of BamlI and XhoI at 20 units/ul and 22.7 ul of ddH2O. All restriction enzymes and buffers were purchased from New England Biolabs (Beverly, Mass.). The samples were incubated at 37° C. for 2 hours. In addition, double-digested pET-28a was dephosphorylated with Calf ALkaline Phosphatase (CIP). In this procedure, 2 ul of CIP was added to the restriction mixture and incubated for 1 hour at 37 C and followed with heat-inactivation at 70° C. for 20 minutes. Meanwhile, a 1% low melting point agarose solution was prepared in a 250 ml Erlenmeyer flask (0.5 gm of agarose and 50 ml of TAE buffer [0.04 M Tris-acetate and 0.001 M EDTA, pH 8.0]) and melted in a microwave at high power for 50 seconds. The solution was cooled until the flask was warm to touch and 2.5 ul of ethidium bromide at 10 mg/ml was mixed into the solution. The agarose gel was cast in a standard DNA electrophoresis apparatus (Bio Rad Sub-cell system GT). Restriction digested samples were prepared for electrophoresis by adding 5 ul of 6× sample loading buffer (40% w/v sucrose in water and 0.25% bromophenol blue). The running buffer was TAE and the apparatus was set at a constant voltage of 80V for 50 minutes. The gel was visualize under long-wave UV and photographed using the Gel Documenting System (UVP Imagestore 5000, San Gabriel, Calif.) following the procedure outlined by the manufacturer. The 500 bp AFP fragments and the digested pET-28a fragment of 5.5 kB were excised from the gel and extracted by the gel-purification technique (described later).

D. Generation of signal-peptide minus cDNA fragments. Primers were designed downstream of the signal peptide and upstream of the stop codon. Additionally, primers were designed to encode BamHI and XhoI sites on the 5′ and 3′ terminal ends of the inserts. Oligonucleotides (primers) were synthesized by BioSynthesis Inc. Other parameters such as melting temperature (tm), annealing temperature and primer stability was checked using DNA Strider. Based on these parameters, a PCR condition was designed. The primer sequences and PCR condition as described below, should result in the generation of a 350 bp fragment.

### Primer Sequences

2-2 and 2-3:

5′-tail -BamHI-sequence-3′:
CGC GGATCCCTCACCGACGAACAG-3′
3′-tail -XhoI-sequence-3′:
CCG CTCGAG TTAATCAATAGGAGAG-5′

Tm13.17:

5′-tail -BamHI-sequence-3′:
CGC GGATCC CTGACCGAGGCACAA-3′
3′-tail -XhoI-sequence-3′:
CCG CTCGAG TCAATCAACTGGTGAG-5′

PCR Conditions:

| | |
|---|---|
| Step 1: | 95° C. for 2 minutes. |
| Step 2: | 94° C. for 1 minute. |
| Step 3: | 60° C. for 1 minute. |
| Step 4: | 72° C. for 1 minute. |

Repeat Steps 2 to 4 for 35 cycles.

| | |
|---|---|
| Step 5: | 72° C. for 5 minutes. |
| Step 6: | 4° C. indefinitely. |

A PCR kit was purchased from Promega. Reaction conditions were determined for a total volume of 25 ul. Reaction was performed in thin-wall PCR tubes and overlaid with 25 ul of sterile mineral oil.

| | |
|---|---|
| DNA template: | 50 ng (2 ul at 25 ng/ul) |
| Primers: | 80 pmoles (5 ul at 16 pmol/ul for each direction) |
| Buffer (10×): | 2.5 ul |
| 10 mM dNTP: | 2.5 ul |
| Taq. Polymerase: | 0.5 ul (5 units/ul) |
| ddH2O: | 7.5 ul |

The entire PCR amplified product was analyzed on a 1.5% low-melting point agarose gel and visualized with a short exposure of UV radiation. Bands of the amplified inserts were excised and DNA was extracted by agarose-removing techniques.

E. Restriction digestion of Signal-minus PCR products. The end product of a PCR amplification results in fragments that are blunt ended. To recreate the “sticky-end” restriction sites, Bam.Hi and XhoI were used to double digest the fragments under previously described conditions. The fragments were again run on a 1% low-melting point agarose gel and the bands were excised and purified from the agarose.

F. Gel Purification of Signal-plus AFP fragments, Signal-minus PCR products and Restriction Digested pET 28a. A Gene Clean II kit was purchased from Bio 101. One ml of sodium iodide (NaI) was added to the cut bands and incubated at 55° C. for 10-15 minutes till completely dissolved. Following this, 10 ul of glass-beads was added and mixed gently to avoid shearing of the DNA. This was incubated on ice for 15 minutes while mixing occasionally and finally centrifuged at 14,000×g for 30 seconds. The NaI solution was discarded and the pellets were washed three times with wash solution initially stored at −20° C. Next, the glass-bead pellet was re-suspended in ddH₂O and incubated for 5 minutes at 55° C. to elute the DNA from the glass-beads. Lastly, the mixture was spun again at the above speed and the eluted DNA was removed with the water.

G. Ligation of PCR fragments and pET-28a. A 1:1 molar ratio of insert and vector was determined and ligated in a total volume of 20 ul (1 ul pET-28a, 12 ul of insert, 2 ul of T4 DNA ligase buffer, 1 ul of T4 DNA ligase and 4 u of ddH20). A control ligation was set up similarly with pET-28a only (1 ul pET 28a, 2 ul of ligase buffer, 1 ul of ligase and 16 ul of ddH20). The ligation mixtures were incubated overnight at 16° C. and heat inactivated at 70° C. for 15 minutes. This step ensures the inactivation of T4 DNA ligase. The ligation product was vacuum-died and transformed in DH5a bacterial cells. The transformed bacteria were plated on LB-agar plates with kanamycin (50 mg/ml) and grown overnight.

H. Analysis of Transformed Clones. Individual clones were carefully picked and grown overnight in 5 ml of LB-kanamycin inl4 ml Falcon polypropylene round bottom tube with loose cap. Once grown to saturation, 0.8 ml of the culture was thoroughly vortexed with 0.6 ml of 80% glycerol and stored at −80° C. The remaining cultures were spun at 4000 rpm at 4° C. for 15 minutes. The supernatant was discarded and the pellet was thoroughly re-suspended in 200 ul of cold Glucose Tris EDTA buffer (50 mM glucose, 25 mM Tris-HCl pH 8.0, 10 mM EDTA), transferred to 1.5 ml micro-centrifuge tubes and incubated at room temperature for 5 minutes. The bacteria were lysed with 400 ul of freshly prepared lysis buffer (0.2 M NaOH, 1% SDS). The buffer was mixed by inverting the tubes several times and incubated on ice for 10 minutes. The solution was then neutralized with 300 ul of acetate buffer (3M potassium and 5M acetate) and mixed by inverting several times and incubated on ice for 10 minutes. Following neutralization a cloudy precipitate was formed. The solution was centrifuged for 10 minutes at 14,000×g. The supernatant was carefully removed and transferred to a new 1.5 ml micro-centrifuge tube. The contaminating protein was removed from the supernatant by adding 550 ul of phenol:chloroform:isoamyl alcohol at a ratio of 25:24:1. The mixture was thoroughly vortexed and centrifuged at 14,000×g for 5 minutes. Following this, the top aqueous layer was transferred to a fresh 1.5 ml micro-centrifuge tube and 600 ul of isopropanol was added. The solution was mixed and the DNA was precipitated at −20° C. for 1 hour. The precipitate was centrifuged at 14,000×g for 10 minutes and the resulting supernatant was carefully discarded. The white pellet at the bottom was washed with cold 70% ethanol and re-centrifuged for 1 minute at 14,000×g. The ethanol was discarded and the pellet was air-dried for 15 minutes and resuspended in 100 ul of TE buffer containing 10 ug of RNase.

I. Restriction Analysis of Mini-Prep DNA. In order to determine the clones with the AFP insert, the samples were restriction digested. Initially, the DNA was tested with Bamli and XhoI to visualize the release of a 350 bp fragment. The digestion was performed with 5 ul of the mini-prep DNA, 5 ul of BamHI buffer, 0.5 ul of 100×BSA, 0.5 ul of BamHI and XhoI each and 38.5ul of ddH20. The digestion was performed for 1 hour at 37° C. and the entire amount was analyzed on a 1% agarose gel. Promising clones were further tested with PvuI enzyme under similar digestion conditions.

J. PCR Confirmation with Internal and External Primers. PCR was used to further confirm for the presence of 2-2, 2-3 and Tm13.17 in the pET-28a vector. In this process, internal and external primers were used to amplify the product. Internal primers are primers that are specific to each clone used. The sequences of these primers were specified earlier. External primers are specific to regions of the vector flanking the inserts. The upstream external primer generated against the T7 promoter and the downstream external primer is against the T7 terminator region. The PCR reaction and conditions were the same as described earlier.

K. DNA Sequencing. The clones were sequenced to check for random and insertional mutations resulting in a shift of the reading frame. Using the ABI Prism Sequencing kit from Perkin Elmer, the clones were sequenced by adding 200 ng of plasmid DNA, 20 ng of T7 primer and 8 ul of Big Dye Terminator dNTP mix with AmpliTaq. The total reaction volume was 50 ul and the sequences were amplified using the PCR conditions determined earlier. The samples were de-salted with Centricon 100 and processed by ABI Prism automated sequencer.

Protein Expression and Purification

L. Transformation of Clones in BL21 DH3 cells. Potential clones were transformed in a strain of bacteria, BL21 that is conducive to protein expression and purification. Fifty microlitres of competent bacteria was used for transforming 50 ng of DNA by heat-shock method. The bacteria were then plated on LB-Agar kanamycin plates and grown overnight. Individual colonies were selected for growth in 5 ml of IB-kanamycin and grown overnight. Aliquots of the culture (0.8 ml bacteria and 0.6 ml 80% glycerol) were frozen for future use. The remaining 4 ml were used for alkaline lysis mini-prep.

M. Testing Optimal Conditions for Protein Expression. In order to establish optimal conditions for fusion protein expression, a stab of the frozen stock was inoculated in 2 ml of LB media with kanamycin and grown overnight. The resulting culture was centrifuged and the pellet was re-suspended in 20 ml of LB media with kanam ycin (50 mg/ml) and 0.2 gm of D-glucose in a 250 ml Erlenmeyer flask. The glucose serves to reduce proteolytic degradation. The flask was incubated with shaking at 37° C. until the OD600 reached 0.5-0.6. At this time 1 ml of the culture was removed, centrifuged at 14,000×g, and the pellet stored at −20° C. The remaining culture was induced for protein expression with isopropyl-1-thio-(-D-galactopyranoside (IPTG) (Sigma, Cat. No. I-5502) to a final concentration of 1 mM. The culture was monitored every hour for 5 hours and 1 ml of culture was removed periodically and the pellet was stored at −20° C. A final sample was removed after 24 hours from IPTG induction and processed in a similar fashion. To analyze the content of secreted bacterial proteins, an aliquot of the LB-media was collected 24 hours from IPTG induction and centrifuged for 5 minutes at 14,000×g. The supernatant was saved and mixed with 50 ul of trichloroacetic acid (TCA) and incubated on ice for 15 minutes. The protein precipitate was centrifuged again at 14,000×g for 10 minutes and the pellet was washed twice with acetone. Following this procedure, the pellet was re-suspended in 75 ul of PBS. All protein samples were analyzed by SDS-PAGE.

N. Gel Electrophoresis. When all the time points were collected, the frozen pellets were re-suspended in 50 ul of ddH20 and 2× sample loading buffer (0.125 M Tris-HCl, pH 6.8, 20% glycerol, 10 mM DTT and 4.6% SDS), boiled for 5 minutes and analyzed on SDS-PAGE. A 15% acrylamide gel of 0.75 mm thickness was made in a vertical mini-electrophoretic apparatus and the samples were electrophoresed under constant current conditions (9 mA through stacking gel and 15 mA through the running gel). Five microlitres of pre-stained molecular weight standards (Gibco-BRL) were electrophoresed along with the experimental samples. Gels were fixed and stained in a solution containing 50% methanol, 5% acetic acid and 0.025% (w/v) Coomassie brilliant blue for protein detection.

O. Thrombin Cleavage. The histidine tag was cleaved from the recombinant proteins by utilizing thrombin protease purchased from Novagen. To determine the optimal conditions for thrombin mediated cleavage, the time and number of units of thrombin were varied. 10 ug of recombinant protein was resuspended in 1× thrombin digestion buffer with 0.004 and 0.001 units of thrombin for 1, 4 and 16 hours at 20° C. Additionally, control protein provided by the company was also subjected to proteolytic cleavage. The different time-points were then electrophoresed on a 15% SDS-PAGE and visualized with Coomassie stain.

P. Large Scale Purification of Fusion Proteins. In order to produce a sufficient amount of proteins for biochemical and functional analysis, a 100 ml bacterial culture was induced under identical conditions as above. The purification protocol is specified in the literature manual for pET vectors from Novagen (Madison, Wis.). The culture was grown for 5 hours from IPTG induction and centrifuged at 5000×g for 20 minutes. The pellet was re-suspended in 4 ml of lysis buffer (5 mM imidazole, 0.5 M NaCl, 20 mM Tris-HCl, pH 7.9) and 4 ug of fresh lysozyme. The lysate was incubated for 15 minutes at room temperature and followed by two freeze-thaw cycles in liquid nitrogen. This procedure in combination with the lysozyme serves as a powerful method to break open the tough bacterial cell wall. The viscous chromosomal DNA was sheared by sonicating for 45 seconds at 90% output and under pulse setting. The samples remained in contact with ice throughout the entire procedure. Following sonication, each lysate was split into three 1.5 ml micro-centrifuge tubes and centrifuged at 14,000×g for 15 minutes at 4° C. The supernatant was carefully removed and pooled in a 15 ml conical tube. The His-Bind resin was purchased from Novagen and 0.5 ml of the resin was used for each sample. Prior to its usage, the resin was centrifuged at 700×g for 30 seconds. It was then serially processed by first washing with 2 ml of sterile deionized water, followed by 2.5 ml of charge buffer (50 mM NiSO4) and lastly with 2 ml of binding buffer (5 mM imidazole, 0.5 M NaCl, 20 mM Tris-HCl, pH 7.9). The charged resin was re-suspended in the protein supernatant and incubated with shaking for 30 minutes at 4° C. Columns were purchased from Novagen and equilibrated with 5 ml of binding buffer. The supernatant-resin mix was added to the column and allowed to pack under gravity. Upon releasing the nozzle of the column, the supernatant began to flow under gravity. The column was washed with 10 ml of binding buffer and 10 ml of wash buffer (60 mM imidazole, 0.5 M NaCl, 20 mM Tris-HCl at pH 7.9). The protein was eluted with 5 ml of elution buffer (1 M imidazole, 0.5 M NaCl, 20 mM Tris-HCl at pH 7.9) and the elutant was captured in a clean tube. The elutant was dialyzed overnight in sterile deionized water in a dialysis cassette (Slide-A-Lyser, Pierce). The de-salted histidine tagged protein was lyophilized and re-suspended in 50 ul of ddH20. Thrombin digested recombinant proteins, along with insect hemolymph, column purified Tm12.86 and whole bacterial lysate were electrophoretically analyzed on a 15% SDS-PAGE. The gel was fixed in 50% methanol and stained with Coomassie blue.

Q. Western Blot Analysis. A western blot analysis of the recombinant proteins was performed with Tm 12.86 antibodies. Pre-stained molecular weight markers (Gibco-BRL) were used along with recombinant proteins, hemolymph, Tm12.86 and whole bacterial lysate. Following electrophoresis, a PVDF membrane of dimensions similar to the gel was activated in 100% methanol. The membrane was then soaked in transfer buffer (39 mM glycine, 48 mM Tris base, 0.037% SDS and 20% methanol) for 10 minutes. Sponge and Whatman paper were additionally soaked in the transfer buffer. Proteins were transferred overnight in a CBS Scientific blotting tank at 4° C. at 10 volts. The membrane was briefly reactivated in methanol and blocked with 2.5% BSA and 0.05% Tween for 1 hour. The membrane was rinsed three times with PBST buffer (0.1 M PBS, 0.1% Tween and 0.5% H2O2). Next, a 100 ml dilution of 1:2000 primary antibody serum was incubated with rocking for 2 hours and followed by three rinses of PBST. Then, a 100 ml dilution of 1:500 peroxidase-conjugated goat-anti-rabbit secondary antibody was incubated for 2 hours and rinsed thrice with PBST for 5 minutes each. Lastly, the membrane was stained with a 15 ml DAB solution (3.3í-Diaminobenzidine Tetrahydrochloride, Fast DAB: Sigma) with 12 ul of 30% hydrogen peroxide and incubated for 2-3 minutes until the bands were visualized. The reaction was stopped with three rinses of PBST and the membrane was air-dried.

R. Determination of Thermal Hysteresis. The recombinant proteins (folded and unfolded) were tested for thermal hysteresis by the ice capillary method (Example 1). Proteins were tested at varying concentrations (50 mg/ml, 25 mg/ml, 5 mg/ml, and 1 mg/ml). It is important to note that presence of imidazole will interfere in protein determination. Positive control for this procedure was column purified Tm 12.86 and negative control was PBS.

S. Recrystallization Inhibition. Antifreeze activity was analyzed by recrystallization inhibition, performed by the procedures detailed in Example 8. The concentrations of the samples were 1 ug/ml, 10 ug/ml, 100 ug/ml and 1 mg/ml. Positive control for the experiment was Tm12.86 and negative control was PBS.

T. Refolding of Recombinant Protein(s). Denaturing conditions were utilized to facilitate the proper folding of the recombinant proteins. The denaturing conditions of protein purification are similar to the non-denaturing conditions described earlier. The crucial difference was the addition of urea to the binding, wash and elute buffers to a final concentration of 6 M. Urea unfolds the three-dimensional conformation of the protein. Serial dialyzes with decreasing concentrations of urea enables the protein to refold into its conformation. Recombinant proteins purified by this procedure were dialyzed in a Pierce dialysis cassette for over 48 hours with decreasing concentrations of urea (6M, SM, 4M, 3M, 2M, 1M, 0.5M and ddH₂O). The samples were lyophilized and re-suspended in 50 ul of ddH₂O and tested for activity with capillary tube method and recrystallization assay.

### EXAMPLE 6

Purification and Refolding of Inclusion Body Proteins

Phase Two for establishing antifreeze activity of the recombinant proteins involved a redirection of focus from the soluble protein fraction to the recombinant products packaged in the bacterial inclusion bodies. The methods detailed below involve modifications of several of the procedures used in Example 5, which generated His-tagged recombinant products isolated from the bacterial supematent.

Construction of pET-28a-2-2 and Tm 13.17 cloning vector. Details of the procedures for cloning signal minus and signal plus inserts of 2-2, 2-3 and T 13.17 into the pET-28a cloning vector for expression of His-tagged recombinant products are as stated in Example 5.

The procedures to recover and refold recombinant products from the bacterial inclusion bodies are different from those detailed in Example 5 for obtaining recombinant proteins from the soluble bacterial fraction. The procedures for isolating and refolding inclusion body proteins are described below.

Expression and purification of His-tag Tm 13.17 recombinant proteins. A single BL 21 strain bacteria colony with insert was put into 5 ml LB medium containing 30 ug/ml kanamycin, and incubated with shaking at 260 rpm at 37 C overnight. Then 1 ml culture solution from overnight 5 ml culture was added into 50 ml LB medium containing kanamycin for further incubation until OD600 reached 0.6. IPTG was added and continued incubation for 5 hr. Bacteria cells were harvested by centrifugation at 6500×g for 15 min at 4 C. The cell pellet was thoroughly resuspended in 1× IB wash buffer (20 mM Tris-HCL pH 7.5, 10 mM EDTA, 10% Triton X-100). Freshly prepared lysozyme was added to bacterial solution and the solution was treated with sonication. The inclusion bodies were collected by centrifugation. The inclusion bodies were resuspended in solublization buffer (500 mM CAPS, pH 11.0) with 6 M urea and 1 mM DTT added. After centrifugation, the supernatant containing solublized protein was transferred into a clean tube.

His-Bind resin chromatography and Thrombin cleavage. His-Bind kit was purchased from Novagen. The solubilized inclusion body suspension was loaded onto a prepacked and equilibrated Ni2+ column. Recombinant proteins were then purified and eluted according to the manufacturer's instruction. The histidine-tag was cleavaged from the recombinant protein by utilizing biotinylated thrombin purchased from Novagen. A 1:2000 wt:wt ratio of thrombin to target protein was used for cleavage in 1× thrombin cleavage buffer at room temperature for 16 hours.

Protein refolding by dialysis. A protein refolding kit was purchased from Novagen. The procedure was modified. The thrombin cleaved protein was dialyzed against 1× dialysis buffer and supplement with 0.1 mM DTT for 4 h at 4° C., two times changed. Next, the protein solution was dialysed with 1× dialysis buffer lacking of DTT for 4 h at 4° C., with two times change in buffer. Finally, the protein solution was dialyzed in 1× dialysis buffer containing 1 mM reduced glutathione and 0.2 mM oxidized glutathione for 24 h at 4° C.

Detailed Procedures for Purification and Refolding of Inclusion Body Proteins includes: 1. Preparation for induction; 2. Preparation of inclusion bodies; 3. Solubilization and refolding; 4. His.Bind resin chromatography; 5. Thrombin cleavage and 6. Dialysis protocol for protein refolding.

1. Preparation for induction A single colony is isolated from spread plate and inoculated into 5 ml LB medium containing 30 μg/ml kanamycin in a 15 ml Falcon tube. Incubation was done with shaking 250 rpm at 37° C. overnight. Later, 1 ml of overnight bacteria culture was then added into 50 ml LB medium containing 30 μl/ml kanamycin in a 250 ml Erlenmeyer flask. Incubation occurred with shaking at 250 rpm at 30° C. until OD600 reached 0.6. Then 0.5 ml stock (100 mM) of IPTG was added and the incubation continued for 5 hrs. The flask was placed on ice for 5 min and proceed to lysis step.

2. Preparation of inclusion bodies—Protein Refolding Kit from Novagen. (1) The induced culture was harvested by centrifugation at 6500×g for 15 min at 40° C. Supernatant removed and discarded; (2) Cell pellet was thoroughly resuspended in 0.1 culture volume of 1× IB wash buffer. Mixing may be necessary for full resuspension; (3) The suspension was cooled on ice to 4° C. to prevent heating during cell breakage; (4) Lysozyme added to a final concentration of 100 ug/rnl from a freshly preparedly mg/ml stock in water. Incubation at 300° C. for 15 min.; (5) It was then mixed by swirling and sonicated on ice with an appropriate tip until cells were lysed and the solution no longer viscous; (6) The inclusion bodies were collected by centrifugation at 10000×g for 10 min.; (7) Supernatant removed and the pellet thoroughly resuspended in 0.1 culture volume of 1×IB wash buffer; (8) Centrifugation repeated as in step 7 and the pellet saved. (9) Again, the pellet was thoroughly resuspended in 0.1 culture volume of 1×IB wash buffer, then the suspension was transferred to a clean centrifuge tube with known weight; (10) Inclusion bodies were collected by centrifugation at 10000×g for 10 min. Supematent decanted and the last traces of liquid removed by tapping the inverted tube on a paper towel; (11) The tube and substrate weighed, the tare weight to obtain the weight of the inclusion bodies

3. Solubilization and refolding—Protein Refolding Kit from Novagen 6 M urea was added to following solution for solublization of inclusion bodies: (1) From the wet weight of the inclusion bodies to be processed, the amount of 1×IB solublization buffer necessary to resuspend the inclusion bodies at a concentration of 10 mg/ml was calculated; (2) At room temperature, preparation of the calculated volume of solublization buffer supplemented with 0.3% N-lauroylsarcosine and imM DTT was done; (3) The calculated amount of 1× solubilization buffer from step 2 was added to the inclusion bodies and gently mix. Large debris was broken up by repeated pipetting; (4) Incubation performed at room temperature for 15 min.; (5) Then centrifugation at room temperature at 10000×g for 10 min. Supernatant containing the solublized protein was transferred into a clean tube while carefully avoiding the pellet debris because the pellet may be soft and loose.

4. His.Bind resin chromatography—His-Bind Kitfrom Novagen

Resin Preparation

(1). Appropriate amounts of the supplied stocks of charge buffer, binding buffer, wash buffer and elute buffer were diluted to 1× with sterile deionized water before use. Approximately 5 bed volumes of Charge buffer, 13 bed volumes of Binding buffer, 6 bed volumes of Wash buffer, and 6 bed volumes of Elute buffer were needed. (Note: For the purification of inclusion body protein, the binding, wash and elute buffers needed to be supplemented with 6M urea.); (2). The bottle of HisBind resin was gently mixed by inversion until completely suspended. Using a wide-mouth pipet, the desired amount of slurry was transferred to a column. The resin was allowed to pack under gravity flow; 3) When the level of storage buffer dropped to the top of the column bed, the following sequence of washes to charge and equilibrate the column was used.
a. 3 volumes of sterile deionized water
b. 5 volumes of 1× charge buffer
c. 3 volumes of binding buffer.

Column Chromatography

(1). Binding buffer was allowed to drain to the top of the column bed and then the column was loaded with the prepared extract. A flow rate of about 10; column volume per hour was optimal for efficient purification; (2) The column was washed with 10 volumes of 1× binding buffer; (3) The column was washed with 6 volume of 1× wash buffer; (4) The bound protein was eluted with 6 volumes of 1× elute buffer, (5) The eluted protein was stored in −80° C. refrigerator.

5. Thrombin cleavage—Thrombin Kit from Novagen

(1) 1:100 dilution was made of Biotinylated Thrombin in Thrombin dilution buffer. The dilution contained 0.01 U enzyme per ul; (2) The following components were assembled in a clean tube

| | |
|---|---|
| 5 ul | 10× Thrombin cleavage buffer |
| 10 ug | target protein |
| 1 ul | Diluted Thrombin |
| X ul | deionized water |
| Total volume | 50 ul |

(3) The reaction was incubated at room temperature (20-21 C) for 16 hours; (4) The extent of cleavage of the sample was determined by SDS-PAGE analysis. (After the cleavage reaction, Biotinylated Thrombin was able to be quantitatively removed with Streptavidin Agarose using a ratio of 16 μl settled resin per unit of enzyme.); (5). The Streptavidin Agarose beads were ensured to be evenly suspended by gently mixing by inversion before removing an aliquot; (6) The desired amount of agarose was transferred to the reaction with a wide-bore pipet tip. A minimum of 25 μl Streptavidin Agarose slurry was recommended because smaller resin volumes are difficult to manipulate; (7) Incubation was at room temperature for 30 min with gentle shaking; (8) The entire reaction was transferred to the sample cup of a spin filter, (9) Centrifugation was done at 500×g for 5 min. The filtrate in the collect tube contained the cleavaged protein, free of Biotinylated Thrombin.

6. Dialysis protocol for inclusion body protein refolding—Protein Refoling Kit from Novagen

(1). The required volume of dialysis buffer was prepared supplemented with 0.1 mM DTT for Thrombin cleavaged protein. In general, dialysis was performed with at least two buffer changes of greater than 50 times the volume of the sample; (2) Dialyis occurred for at least 4 h at 40° C. The buffer changed and dialysis continued for an additional 4 or more hours; (3). Additional dialysis buffer was prepared as determined in step 1, but DTT omitted; (4) Continuing the dialysis through two additional changes (4 h each) with the dialysis buffer lacking DTT; (5) Further dialysis buffer was prepared containing 1 mM reduced glutathione and 0.2 mM oxidized gutathione in 1× dialysis buffer. The volume was 25 times greater than the volume of the protein sample. Chilled to 4° C.; (6) Refolded protein was dialyzed 12-16 hr at 4° C.; (7) The sample of the dialyzed protein assayed for target protein activity.

Additionally, some aspects of the following were in some instances implemented for potential enhancement of protein activity levels.

The protein eluted in urea-containing buffer was diluted gradually with washing buffer, pH 7.8 containing 0.2 mM (-mercaptoethanol until the final protein concentration was about 10 ug/ml. The solution was dialyzed against buffer A (100 mM NaCl, mM CaCl2, 50 mM Tris-HCl, pH 8.0) for 48 h and against 0.1 NH4HCO3 for 72 h at 80° C. Then protein was freeze-dried.

Facilitate slow reoxidation by opening the cover of the microcentrifuge tube, letting the purified protein solution be exposure to air in 4° C. refrigerator for 10 days. Protein activity was tested after 3, 5, 7 and 10 days.

In all cases, refolded recombinant products were screened for antifreeze activity through assessment of RI (see Example 8) and thermal hysteresis (Example 1).

### EXAMPLE 7

Derivation of Concensus Sequences for the Tm 12.86 Gene and Protein Families

In developing concensus sequences for the genes and proteins of the Tm 12.86 family (cladistic tree shown in FIG. 4.20), careful attention was made to the types of substitutions and the chemistry involved Both a full generic concensus sequence was identified for the entire Tm 12.86 gene family encoded proteins, and consensus sequences for the nested genes within the family are also described (i.e. concensus sequence for Tm 12.84-6 like, consensus sequence expanded to include Tm 13.17 like, concensus sequence expanded to include B1/B2 like, and concensus expanded to include AFP3 like, genes and their encoded proteins (SEQ ID NO. 44-48).

The following letter designations used in deriving these concensus sequences are as specified below. In the concensus gene sequences, (FIG. 7.2) there are the letters for the four bases, A, G, C, and T. In addition, N is used to designate “any nucleotide”, Y is used to designate “any pyrimadine” (C or T), and R is used to designate “any purine” (A or G). In the concensus, we have included the designations AfF, T/A, G/C, and C/G. These reflect the special relation these pairs of bases have in the anitparallel strands of DNA. In fact, if a T is substituted for A, for example, the opposite strand then must be substituted A for T. The base pair at that position is retained, though the precise sequence has changed and may affect the protein when translated. Conversely, if C is substituted for T (pyrimadine substitution), then the opposite strand must substitute G for A (purine substitution). The sequence is changed and the original pair is completely elimintated. The chemistry of the strands also changes since the G-C bond is stronger than the A-T bond. The concensus sequences listed for each grouping (among Tm 12.84; expanded to include Tm 13.17 like, then B1 like, and then AFP-3 like) list the most representative concensus sequence and positions and types of substitutions occurring or deemed acceptable (FIG. 7.2).

With protein sequences, convention assigns a three letter abbreviation or a single letter to each amino acid. The three letter abbreviation is more tractable to describing substitutions and fits nicely with each three letter codon in the gene sequence, yet it's bulk in generating column groupings was undesirable, so single letter assignments for each amino acid was chosen for generating the protein concensus for the Tm 12.86 family (FIG. 7.3). However, no convention has been developed for describing substitutions in the single letter system. We have chosen to designate substitutions as to chemical class and hydrophobicity classifications. Refer to FIG. 7.1 for one and three letter designations of amino acids, and their chemical class and other key characteristics. The concensus protein sequences listed for each grouping (among Tm 12.84; expanded to include Tm 13.17 like, then to B 1 like, then AFP-3 like, and then an identified general concensus peptide) lists the most representative concensus sequence to encompass that grouping and positions and types of substitutions occurring or deemed acceptable.

### EXAMPLE 8

Development and Use of the Recrystailization Inhibition Assay for Antifreeze Protein Activity

Acclimation of insect larvae. T. molitor larvae originally purchased from Carolina Biological Supply were maintained and then acclimated to winter conditions as detailed in Example 1. The acclimated larvae were used as a source of “winter hemolymph” and for purification of Tm 12.86. A second population of T. molitor larvae used as a source for “summer hemolymph” were maintained at 21 and long-day photoperiod conditions (16 hours light: 8 hours dark) for at least three weeks. D. canadensis larvae used for hemolymph R.I. studies from natural populations were collected from the Binghamton University Nature Preserve in July, 1996 and February, 1997. Manduca sexta larvae used as non-antifreeze protein-producing insect controls for the hemolymph R.I. study were maintained at 25° C. under 12 hours light: 12 hours dark photoperiod conditions on standard artificial diet.

Purification of Tm 12.86. Purification of the THP Tm 12.86 from T. molitor was performed as described as detailed in Example 1. Testing for the presence of Tm 12.86 was performed using a Western blot and thermal hysteresis (T.H.) activity measurement via the capillary method (see Example 1).

Collection of hemolymph samples from insect larvae. Hemolymph samples from T. nolitor, D. canadensis, and M. sexta larvae were collected by puncturing the cuticle between anterior segments with a needle, then collecting the droplet with a 10 μl capillary pipette. For T.H. measurements, hemolymph was sealed in the capillary. For RI studies, the hemolymph volume was estimated by measuring the length fraction of the capillary occupied by hemolymph and multiplying by the total 10 μl capillary volume. Each hemolymph sample was expelled into an appropriate volume of 0.9% NaCl, usually to produce a hemolymph dilution of 1/50 (more concentrated dilutions were created in the case of M. sexta hemolymph). In general, two capillary samples were obtained from each larva, one for RI studies and the other for measurement of thermal hysteresis.

Measurement of thermal hysteresis. Hemolymph, purified Tm 12.86 solutions, and T. molitor fat body cell culture supernatant were assayed for thermal hysteresis activity using the micro-capillary method Example 1).

Assessment of recrystallization inhibition (R.I.) The “splat cooling” technique (Knight, C. A. et al. [1988] Cryobiology 24: 55-60) was used to produce fine-grained ice samples for recrystallization inhibition studies. For this procedure, 10 μl sample droplets were released approximately 2.6 m above a polished aluminum plate maintained at dry ice temperature (about −78° C.). Upon contact with the surface of the aluminum plate, a thin, fine-grained ice wafer about 8 mm in diameter was formed. The wafer was immediately transferred to the cold stage holding chamber (preset to −2° C. or −6° C.) using a metal weighing spatula maintained at −78° C. The cold stage, consisting of a brass extension piece drilled through to form the ice sample holding area, was cooled via a Peltier device (Laboratory Instruments Services). Heat transferred by the Peltier was absorbed by a second brass head cooled via a circulating alcohol bath at −8° C.

A coverglass was placed at the bottom of the cold stage to form a holding chamber, ice samples were positioned on a small polypropylene ring (cut from the top of a 0.5 ml microfuge tube) at the bottom of the chamber, which was then sealed with a second coverglass. The temperature within the chamber was monitored using a Type T thermocouple needle microprobe (Physitemp type MT-26/2) with digital thermometer (Physitemp BAT-10RLOP) and a second Type T thermocouple sensor (Physitemp type MT-4) immersed in an ice-water bath for differential temperture measurements.

Ice samples were sealed within the holding chamber and allowed to anneal at a constant temperature for a specified time period. Annealing temperatures of −2° C. and −6° C. and annealing times of 30 minutes and two hours were primarily studied, with the chamber temperature adjusted manually by varying electric current supply to the Peltier unit. The Peltier current was monitored with a Fluke SP-7 digital multimeter.

Following placement of a sample within the refrigerated holding chamber and allowing for a specified annealing time, the top coverglass was removed from the chamber and the cold stage placed between crossed polarizing filters for microscopic observation of an ice sample. A stereoscope (Bausch and Lomb Stereozoom 7) with eyepiece mounted camera (Nikon N6006 or Canon AE-1 35 mm, with camera adaptors from Carolina Biological Supply) was used to photograph each ice sample, with crossed polaroids producing contrast between individual ice grains by birefringence. Kodak 400 speed color film was used for photographs, and 4 to 8 second exposure times were required with the light source used (Bausch and Lomb incandescent). Generally, a stereoscopic total magnification of 44.5× was used for most of the ice samples.

Quantification of Ice Recrystallization

Mean largest grain size. Our principle method used to quantify the recrystallization process involved the assessment of an average of the largest ice grain sizes for each sample. For this purpose, we photographed two different areas on each sample as defined by FIG. 8.1a: the first area corresponded to the sample “center” region and the second to the “mid-sample” region. The sampling of these two particular areas is based upon our observations of putative sample thickness variation across the sample and its possible effects on ice grain size homogeneity. This particular sampling protocol was generally applied to all samples of varying composition and annealing temperatures with the exception of samples diluted in 0.9% NaCl and annealed at −2° C. In this case, the two photographs taken per ice sample included a sample “maximum deformation” and “minimum deformation” region as defined FIG. 8.1b. These samples show a definite ice grain size heterogeneity which appears to be the result of a gravity-induced sagging of the sample (putatively due to higher liquid content). This effect does not appear to be significant for samples diluted in H₂O and annealed at −2° C. or −6° C., or for samples diluted in 0.9% NaCl and annealed at −6° C.

Photographs used to assess ice grain size were taken at high (44.5×) magnification. In each photograph, the five largest ice grains (largest in cross sectional area) were each assessed for cross sectional area. Grain cross sectional area was approximated as an elliptical area, with the largest linear dimension across the grain serving as the major elliptical axis, and the largest linear grain dimension orthogonal to the major axis serving as the minor elliptical axis. A schematic representation of a typical ice sample photograph at high magnification and the process of mean largest grain size determination is presented in FIG. 8.2. Since two photographs for each test sample were obtained, a total of ten elliptical area measurements were calculated and averaged to produce a single, composite mean largest grain size (mlgs.) for each sample.

Random sampling method for mlgs evaluation of recrystallized ice samples. As detailed in the specifications, one of the difficulties encountered during the study of recrystallized samples in NaCl solution at higher annealing temperatures is the presence of an apparent ice grain size heterogeneity, presumably due to gravity-induced sagging of the sample. One method used to determine a representative mean largest grain size for samples annealed at higher temperatures (−2° C.) involved sampling two different areas of each ice sample: the first at an area of maximum deformation, the second at an area of minimum sample deformation (FIG. 8.1b). These two areas were then averaged to produce a composite sample mean largest grain size. Using this method, RI dilution profiles were developed for purified Tm 12.86 in 0.9% NaCl and T. molitor hemolymph in 0.9% NaCl, with all sample dilutions annealed at −2° C. A slope difference was detected between linear regression estimates of the purified Tm 12.86 and T. molitor profiles, due primarily to a rapid loss of RI potency with increasing dilution observed for the purified Tm 12.86 solutions. Because of the presence of an ice grain size heterogeneity for samples containing 0.9% NaCl and annealed at −2° C., an attempt was made to confirm the previous Tm 12.86 and T. molitor hemolymph results using a modified sampling method. A random sampling technique was specifically developed for this purpose. The random sampling method appears to eliminate any bias which might be expected to occur using the “maximum/minimum deformation” method, and as described below may help to minimize sample deformation altogether. The random sampling method uses a grid consisting of common window screening material (in this case plastic material) cut to fit the cold stage holding area, with grid squares approximately 1.5 mm by 1.5 mm in size. Grid square numbers were then assigned as shown in FIG. 8.42. A splat-cooled ice sample was prepared as described previously, then placed directly on the grid in the cold stage holding chamber. Upon completion of the annealing time period, two grid squares were chosen at random for photographs and subsequent mean largest grain size measurements. The selected grid squares were determined using a random number generator function of a Casio fx-300 Scientific calculator. The random number generator produces a random decimal between 0.000 and 1.000, which was then multiplied by 30 (corresponding to the total number of grid squares) and rounded to the nearest whole number to obtain the final square number. In some cases where a grid square was unoccupied or occupied by only a small portion of an ice sample, another random number was chosen. This process was repeated until an occupied square was obtained. A total of two squares per ice sample were selected using this process. In the event that the second square number chosen was identical to the first, an additional random number was generated. Thus, two different sample areas were always photographed per ice sample.

Light scattering. A second method used to quantify the extent of recrystallization involved a light scattering method which approximates the amount of light passing through a given ice sample. Generally, ice samples with smaller ice grains were found to exhibit greater light scattering capability, apparently due to the presence of a greater number of intergrain boundaries. The result is less light flux reaching the photographic film for samples with smaller ice grains.

To measure the extent of the light scattering behavior of an annealed ice sample, the top coverglass of the holding chamber was removed and a single polarizing filter placed between the stereoscope light source and the ice sample. A full view photograph of each sample was obtained by setting the stereoscope magnification to a constant 1.85× (FIG. 8.3A). Kodak black and white TMAX 100 film was used for photographs, and a camera exposure time of 1/2000 second was set for all samples (shorter exposure times provided the best results). Full magnification (44.5×) photographs of each sample were also obtained (FIG. 8.3B) in order to compare both the light scattering and mlgs determination methods of R.I. assessment.

Quantitation of light transmitted through each ice sample was accomplished using laser densitometry scans of the resultant film negative. Scanning was performed using a Pharmacia LKB Ultrascan 2000 laser densitometer. The resultant densitometry absorbance peak for each sample was then evaluated for maximum amplitude (FIG. 8.3C).

The light scattering method of RI quantitation was not generally used in the specifications and applications of the R.I. assay, rather those evaluations were performed using the mlgs measurement approach. The light scattering method appears to be better suited for implement on a large scale, detailed further in the invention specifications.

Assessment of recrystallization inhibition (RI) behavior for insect hemolymph and purified Tm 12.86. A general strategy for the study of RI behavior in both insect hemolymph and Tm 12.86 solutions as presented here consisted of the development of RI dilution profiles. Starting with a known hemolymph dilution or Tm 12.86 concentration, a series of dilutions was prepared using either 0.9% NaCl or H₂O followed by the preparation and annealing of fine-grained ice samples as described previously. The mean largest grain size calculated for each sample was plotted as a function of the logarithm of hemolymph or Tm 12.86 dilution to obtain a dilution profile. Replicate dilution series were usually created for a given purified Tm 12.86 sample (at a starting concentration of 25 mg/ml) or for individual hemolymph samples (usually at a 1/50 starting dilution), depending upon the application. In some instances, single series dilution profiles from multiple independent hemolymph samples were combined to form a replicate profile group. Dilution profiles for Tm 12.86 were derived from two independently prepared 25 mg/ml stock samples by dissolving lyophilized Tm 12.86 in H₂O, designated Tm 12.86(a) and (b) stocks. Profiles based on Tm 12.86 diluted in 0.9% NaCl were created from both 25 mg/ml stock samples (five dilution series from Tm 12.86(a) and three dilution series from Tm 12.86(b) for both −2° C. and −6° C. annealing temperatures), with the exception of 250 μg/ml, 25 μg/ml and 0.1 μl/ml samples at −6° C. annealing temperature which were evaluated later. Profiles based on Tm 12.86 in H₂O were derived from a single 25 mg/ml stock sample (Tm 12.86(b)) for both −2° C. and −6° C. annealing temperatures.

For best results, hemolymph or Tm 12.86 dilution profile samples were tested for RI activity in order of most dilute to least dilute. This was done due to possible degradative effects on the THPs, since only one sample at a time could be evaluated for RI. In addition, all sample solutions were maintained on ice as much as possible during RI testing. Sample dilutions produced for RI dilution series studies were generally non-serial in origin. Preliminary results suggest that the method of dilution (i.e. serial or non-serial dilutions) may affect dilution profile graphs somewhat, though the cause of such an effect remains unknown. Based on differences in recrystallization rates, samples diluted in H₂O were annealed at −2° C. or −6° C. for two hours, while samples diluted in 0.9% NaCl were annealed at −2° C. or −6° C. for 30 minutes.

Preparation of T. molitor fat body cell culture. The T. molitor fat body cell culture was prepared using the method developed in the Horwath lab. Briefly, fat body (4 lobes) dissected from T. molitor maintained at 26° C. under long day conditions (16 h light: 8 h dark) was placed in sterile Tenebrio saline and washed twice, followed by washing in Tenebrio Modified Grace's cell culture medium (TMG). After washing, 100 ml droplets of fat body cells and tissue were transferred to well culture plates which were then transferred to an incubator maintained at 26° C. Three days after initial culturing, 200 ml of fresh TMG medium was added to each culture well. At the end of the seventh day after initial culturing, the culture supernatant (designated here as “C1” for culture after one week) was sampled and used for RI and T.H. measurements.

Expression of Tm 13.17 cDNA and preparation of. E. coli lysate. The expression and preparation of a recombinant, putative thermal hysteresis protein in E. coli was performed using methods detailed in Examples 2-6. Recombinant products from each of these Examples were screened for antifreeze specific inhibition of ice recrystallization. Identical procedures were used to isolate and refold proteins derived from control E. coli lacking AFP cDNA inserts. These bacterial protein extracts were used as a control n the RI assay.

Blood plasma from R. sylvatica and R. pipens. Blood plasma from the cold hardy frog Rana sylvatica and the non-cold hardy species Rana pipens was obtained from the laboratory of Dr. Richard Lee (Miami University of Ohio) for the possible detection of THPs using RI assessments.

Water source. 0.9% NaCl solutions were prepared using double-distilled (glass distilled) water, and subsequently used to dilute hemolymph, Tm 12.86, and other samples for RI evaluation. Samples requiring dilution in water alone were prepared using nanopure H₂O (Barnstead) filtered with Schleicher and Schuell Uniflow-25 0.2 μm filters. The additional filtration step was required due to the discovery of an unusual effect with respect to recrystallization for H₂O samples during the course of RI experiments. A pronounced difference in recrystallization behavior was noted for both unfiltered double-distilled (glass distilled) H₂O and unfiltered nanopure H₂O (Barnstead) as compared to filtered (Uniflow-25, Schleicher and Schuell) samples for both H₂O sources when subjected to splat cooling followed by annealing at −6° C. for 2 hours. There appeared to be unusual ice crystal morphology (roughened surfaces) in addition to some inhibition of recrystallization in the case of the unfiltered samples.

Statistics. Differences between multiple categories of THP and non-THP solution mean largest grain sizes were tested for statistical significance using one way analysis of variance (ANOVA) with Tukey's HSD post hoc multiple comparisons test and critical test statistic calculated at the α=0.05 level. Testing for differences in mean largest grain sizes between only two treatment categories was performed using Student's t-test with a critical test statistic calculated at the α=0.05 level. Regression analyses were also performed for dilution profiles of purified Tm 12.86 and insect hemolymph followed by analysis of covariance (ANCOVA) for differences in regression line slopes and elevations. Linear regression, ANOVA, and t-test calculations were performed using SYSTAT version 5.2 (SYSTAT, Inc.); ANCOVA calculations were performed using Microsoft Excel version 4.0 spreadsheet software. Linear regression analysis was also performed on a dilution profile for T. molitor hemolymph in 0.9% NaCl using light scattering data. In this case, relative absorbances replaced mlgs values for each sample. A linear regression analysis was then performed using SYSTAT as described previously.

### Sequence Listing Data

The written Sequence Listings for SEQ ID NO's 1-48 (pages 166-221) are attached herein with the Submission of the Computer Readable Copy.

The invention now being fully described, it will be apparent to one of ordinary skill in the art that many changes and modifications can be made thereto without departing from the spirit or scope of the invention as set forth herein.

1. A cDNA polynucleotide comprising a nucleotide sequence for encoding thermal hysteresis proteins derived from the Tenebrionoidea Superfamily.

2. The cDNA according to claim 1, wherein said thermal hysteresis proteins are Type III anti-freeze proteins.

3. The cDNA according to claim 1, wherein said thermal hysteresis proteins are from the group consisting of Tm 12.86, Tm 2.2, Tm 3.4, Tm 3.9, Tm 7.5, Tm 2.3, Tm 13.17, Tm 12.84 and isoforms thereof.

4. The cDNA according to claim 1, wherein said nucleotide sequences are from the group consisting of SEQ ID NO.'s 2, 5, 6, 9, 12, 15, 16, 18, 20, 22, 24, 26, 28, 30, 32, 34, 36, 38, 44-47 and their respective complements.

5. The cDNA according to claim 1, wherein said nucleotide sequence further includes a 5′ end selected from the group consisting of non-his/signal plus, non-his/signal minus, his/signal plus and his/signal minus.

6. A m-RNA polynucleotide comprising a nucleotide sequence for encoding thermal hysteresis proteins derived from the Tenebrionoidea Superfamily transcribed from said cDNA in claim 1.

7. The mRNA according to claim 6, wherein said thermal hysteresis proteins are Type III anti-freeze proteins.

8. The mRNA according to claim 6, wherein said thermal hysteresis proteins are from the group consisting of Tm 12.86, Tm 2.2, Tm 3.4, Tm 3.9, Tm 7.5, Tm 2.3, Tm 13.17, Tm 12.84 and isoforms thereof.

9. The mRNA according to claim 6, wherein said nucleotide sequences comprise the fully complementary sequences from the group consisting of SEQ ID NO.'s 2, 5, 6, 9, 12, 15, 16, 18, 20, 22, 24, 26, 28, 30, 32, 34, 36, 38 and 44-47.

10. The mRNA according to claim 6, wherein said nucleotide sequence further includes a 5′ end selected from the group consisting of non-his/signal plus, non-his/signal minus, his/signal plus and his/signal minus.

11. A DNA probe having a sequence complementary or identical to a sequence of contiguous nucleotides for at least a portion of said cDNA sequences of claim 1.

12. A RNA probe having a sequence complementary or identical to a sequence of said nucleotides of said nucleotide sequences of claim 6.

13. A recombinant vector containing the cDNA of claim 1.

14. A thermal hysteresis protein derived from the Tenebrionoidea Superfamily which lowers the freezing point of a solution without effecting the melting point of the solution.

15. The thermal hysteresis protein according to claim 14, wherein said protein has an amino acid sequence from the group consisting of SEQ ID NO.'s 1 to 39 and 44 to 48.

16. A consensus sequence with a nucleotide sequence from the group consisting of SEQ ID NO.'s 44 to 47.

17. A consensus sequence with an amino acid sequence from the group consisting of SEQ ID NO.'s 44 to 47.

18. A consensus sequence with amino acid sequences as in SEQ ID NO. 48.

19. A primer having a nucleotide sequence selected from the group consisting of SEQ ID NO.'s 40 to 43.

20. A method for producing a polypeptide having antifreeze properties comprising: forming a cloning vector with a Tm 12.86 family member gene encoding an antifreeze polypeptide; transferring genes of said cloning vector into DNA of host cell to create a transformed cell; expressing a mRNA sequence and a translated amino acid sequence from said recombinant expression vector, said sequence being isoforms of said Tm 12.86 T.molitor antifreeze polypeptide.

21. The method according to claim 20, further comprising isolating said amino acid sequence and establishing antifreeze protein activity for said amino acid sequence.

22. The method according to claim 20, wherein said amino acid sequences are from set forth SEQ ID NO.'s 2-39 and 44-48.

23. The method according to claim 20, wherein said polypeptide has an apparent molecular weight from about 11,000 to 25,000 Daltons.

24. The method according to claim 20, wherein said isolating said amino acid sequence comprises extraction from inclusion bodies within said transformed host bacterial cell.

25. The method according to claim 20, wherein establishing activity further comprises denaturing and extracting proteins from said transformed cells followed by renaturizing and purifying said polypeptide, followed by further denaturing and refolding.

26. The method according to claim 25, wherein said activity step provides antifreeze polypeptide activity as measured by thermal hysteresis or antifreeze specific recrystallization inhibition.

27-40. (canceled)


SEQUENCE LISTING (48 sequences)

SEQ ID NO: 1 - protein, 19 aa, Tenebrio molitor
  N-terminal sequence of protein Tm 12.86
LTDEQIQKRNKISKECQQV

SEQ ID NO: 2 - DNA, 576 bp, Tenebrio molitor
  Non-his-tagged, signal plus, Tm 13.17
gtggatccaaagaattcggcacgagactactaagatgaagttgctctgttgtctaatctccctcattctgttggtcacagttcaggccctgaccgaggcacaaattgagaaactgaacaagatcagcaaaaaatgtcaaaatgaaagtggagtgtcgcaagagatcataaccaaagctcgcaacggtgactgggaggacgatcctaaactgaaacgccaagttttttgcgtggccaggaacgccggtctggccacggaatcgggagaggtggtggtcgacgtgttgagggagaaggtgaggaaggtcactgacaacgacgaagaaactgagaaaatcatcaataagtgcgccgtcaagagagatactgttgaagagacggtgttcaatactttcaaatgtgtcatgaaaaacaagccaaagttctcaccagttgattgaaccaccacgactagtagatggttcaaatggtgtgctttacatataaaaataaagtgtttctgatgtaaaaaaaaaaaaaaaaaaaaaaaaaactcgagagtattctagagcggccgcgggcccatcgttttccaccc

SEQ ID NO: 3 - protein, 134 aa, Tenebrio molitor
  Precursor Protein for Tm 13.17
MKLLCCLISLILLVTVQALTEAQIEKLNKISKKCQNESGVSQEIITKARNGDWEDDPKLKRQVFCVARNAGLATESGEVVVDVLREKVRKVTDNDEETEKIINKCAVKRDTVEETVFNTFKCVMKNKPKFSPVD

SEQ ID NO: 4 - protein, 116 aa, Tenebrio molitor
  Mature Protein for Tm 13.17
LTEAQIEKLNKISKKCQNESGVSQEIITKARNGDWEDDPKLKRQVFCVARNAGLATESGEVVVDVLREKVRKVTDNDEETEKIINKCAVKRDTVEETVFNTFKCVMKNKPKFSPVD

SEQ ID NO: 5 - DNA, 481 bp, Tenebrio molitor
  Non-His-tagged, Signal plus, Clone 2.2
ggcacgagcaaaaatgaaactcctcttgtgctttgcgttcgccgccatcgtcatcggagctcaggctctcaccgacgaacagatacagaaaaggaacaagatcagcaaagaatgccagcaggtgtccggagtgtcccaagagacgatcgacaaagtccgcacaggtgtcttggtcgatgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaagtgtatttacgacagtaaacctgatttctctcctattgattaattgttttgtatttgactgaattttgacaataaaggtaatatcgttatgtaaaaaaaaaaaaaaaaa

SEQ ID NO: 6 - DNA, 482 bp, Tenebrio molitor
  Non-His-tagged, Signal plus, Clone 2.3
ggcacgagcaaaaatgaaactcctcttgtgctttgctttcgccgccatcgtcatcggagctcaggctctcaccgacgaacagatacagaaaaggaacaagatcagcaaagaatgccagcaggtgtccggagtgtcccaagagacgatcgacaaagtccgcacaggtgtcttggtcgacgatcccaaaatgaagaagcacgtcctctgcttctcgaagaaaactggagtggcaaccgaagccggagacaccaatgtggaggtactcaaagccaagctgaagcatgtggccagcgacgaagaagtggacaagatcgtgcagaagtgcgtggtcaagaaggccacaccagaggaaacggcttatgacaccttcaagtgtatttacgacagtaaacctgatttctctcctattgattaattgttttgtatttgactgaattttgacaataaaggtactatcgttatgaaaaaaaaaaaaaaaaaaa

SEQ ID NO: 7 - protein, 133 aa, Tenebrio molitor
  Precursor Protein for Tm 12.84, Clones 2.2, 2.3, and 7.5
MKLLLCFAFAAIVIGAQALTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKCIYDSKPDFSPID

SEQ ID NO: 8 - protein, 115 aa, Tenebrio molitor
  Mature Protein for Tm 12.84, Clones 2.2, 2.3, and 7.5
LTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKCIYDSKPDFSPID

SEQ ID NO: 9 - DNA, 481 bp, Tenebrio molitor
  Non-His-tagged, Signal plus, Clone 3.4
ggcacgagcaaaaatgaaactcctcttgtgctttgctttcgccgccatcgtcatcggagctcaggctctcaccgacgaacagatacagaaaaggaacaagatcagcaaagaatgccagcaggtgtccggagtgtcccaagagacgatcgacaaagtccgcacaggtgtcttggtcgacgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaaggttatttacgacagtaaacctgatttctctcctattgattaattgttttgtatttgactgaattttgacaataaaggtactatcgttatgtaaaaaaaaaaaaaaaaa

SEQ ID NO: 10 - protein, 133 aa, Tenebrio molitor
  Precursor Protein for Clone 3.4
MKLLLCFAFAAIVIGAQALTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKVIYDSKPDFSPID

SEQ ID NO: 11 - protein, 115 aa, Tenebrio molitor
  Mature Protein for Clone 3.4
LTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKVIYDSKPDFSPID

SEQ ID NO: 12 - DNA, 482 bp, Tenebrio molitor
  Non-His-tagged, Signal plus, Clone 3.9
ggcacgagcaaaaatgaaactcctcttgtgctttgctttcgccgccatcgtcatcggagctcaggctctcaccgatgaacagatacagaaaaggaacaagatcagcaaagaatgccagcaggagtccggagtgtcccaagagacgatcgacaaagtccgcacaggtgtcttggtcgacgatcccaaaatgaagaagcacgtcctctgcttctcgaagagaactggagtggcaaccgaagccggagacaccaatgtggaggtactcaaagccaagctgaagcatgtggccagcgacgaagaagtggacaagatcgtgcagaagtgcgtggtcaagaaggccacaccagaggaaacggcttatgacaccttcaagtgtatttacgacagtaaacctgatttctctcctattgattaattgttttgtatttgactgaattttgacaataaaggtactatcgttatgaaaaaaaaaaaaaaaaaaa

SEQ ID NO: 13 - protein, 133 aa, Tenebrio molitor
  Precursor Protein for Clone 3.9
MKLLLCFAFAAIVIGAQALTDEQIQKRNKISKECQQESGVSQETIDKVRTGVLVDDPKMKKHVLCFSKRTGVATEAGDTNVEVLKAKLKHVASDEEVDKIVQKCVVKKATPEETAYDTFKCIYDSKPDFSPID

SEQ ID NO: 14 - protein, 115 aa, Tenebrio molitor
  Mature protein for Clone 3.9
LTDEQIQKRNKISKECQQESGVSQETIDKVRTGVLVDDPKMKKHVLCFSKRTGVATEAGDTNVEVLKAKLKHVASDEEVDKIVQKCVVKKATPEETAYDTFKCIYDSKPDFSPID

SEQ ID NO: 15 - DNA, 481 bp, Tenebrio molitor
  Non-his-tagged, Signal plus, Clone 7.5
ggcacgagcaaaaatgaaactcctcttgtgctttgcgttcgccgccatcgtcatcggagctcaggctctcaccgacgaacagatacagaaaaggaacaagatcagcaaagagtgccagcaggtgtccggagtgtcccaagagacgatcgacaaagtccgcacaggtgtcttggtcgacgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaagtgtatttacgacagtaaacctgatttctctcctattgattaattgttttgtatttggctgaattttgacaataaaggtactatcgttatgtaaaaaaaaaaaaaaaaa

SEQ ID NO: 16 - DNA, 681 bp, Tenebrio molitor
  His-tagged, Signal plus, Clone 2.2
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccgaattcgcacgagcaaaaatgaaactcctcttgtgctttgcgttcgccgccatcgtcatcggagctcaggctctcaccgacgaacagatacagaaaaggaacaagatcagcaaagaatgccagcaggtgtccggagtgtcccaagagacgatcgacaaagtccgcacaggtgtcttggtcgatgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaagtgtatttacgacagtaaacctgatttctctcctattgattaattgttttgtatttgactgaattttgacaataaaggtaatatcgttatgtaaaaaaaaaaaaaaaaactcgagcaccaccaccaccaccactgagat

SEQ ID NO: 17 - protein, 173 aa, Tenebrio molitor
  Precursor Protein with His-tag, Clone 2.2
MGSSHHHHHHSSGLVPRGSHMASMTGGQQMGRGSEFARAKMKLLLCFAFAAIVIGAQALTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKCIYDSKPDFSPID

SEQ ID NO: 18 - DNA, 543 bp, Tenebrio molitor
  His-tagged, signal minus, Clone 2.2
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccctcaccgacgaacagatacagaaaaggaacaagatcagcaaagaatgccagcaggtgtccggagtgtcccaagagacgatcgacaaagtccgcacaggtgtcttggtcgatgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaagtgtatttacgacagtaaacctgatttctctcctattgattaactcgagcaccaccaccaccaccactgagat

SEQ ID NO: 19 - protein, 149 aa, Tenebrio molitor
  Mature Protein with His-tag, Clone 2.2
MGSSHHHHHHSSGLVPRGSHMASMTGGQQMGRGSLTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKCIYDSKPDFSPID

SEQ ID NO: 20 - DNA, 682 bp, Tenebrio molitor
  His-tagged, Signal Plus, Clone 2.3
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccgaattcgcacgagcaaaaatgaaactcctcttgtgctttgctttcgccgccatcgtcatcggagctcaggctctcaccgacgaacagatacagaaaaggaacaagatcagcaaagaatgccagcaggtgtccggagtgtcccaagagacgatcgacaaagtccgcacaggtgtcttggtcgatgatcccaaaatgaagaagcacgtcctctgcttctcgaagaaaactggagtggcaaccgaagccggagacaccaatgtggaggtactcaaagccaagctgaagcatgtggccagcgacgaagaagtggacaagatcgtgcagaagtgcgtggtcaagaaggccacaccagaggaaacggcttatgacaccttcaagtgtatttacgacagtaaacctgatttctctcctattgattaattgttttgtatttgactgaattttgacaataaaggtactatcgttatgaaaaaaaaaaaaaaaaaaactcgagcaccaccaccaccaccactgagat

SEQ ID NO: 21 - protein, 173 aa, Tenebrio molitor; identical to SEQ ID NO: 17
  Precursor Protein with His-tag, Clone 2.3

SEQ ID NO: 22 - DNA, 543 bp, Tenebrio molitor
  His-tagged, Signal minus, Clone 2.3
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccctcaccgacgaacagatacagaaaaggaacaagatcagcaaagaatgccagcaggtgtccggagtgtcccaagagacgatcgacaaagtccgcacaggtgtcttggtcgatgatcccaaaatgaagaagcacgtcctctgcttctcgaagaaaactggagtggcaaccgaagccggagacaccaatgtggaggtactcaaagccaagctgaagcatgtggccagcgacgaagaagtggacaagatcgtgcagaagtgcgtggtcaagaaggccacaccagaggaaacggcttatgacaccttcaagtgtatttacgacagtaaacctgatttctctcctattgattaactcgagcaccaccaccaccaccactgagat

SEQ ID NO: 23 - protein, 149 aa, Tenebrio molitor; identical to SEQ ID NO: 19
  Mature Protein with His-tag, Clone 2.3

SEQ ID NO: 24 - DNA, 776 bp, Tenebrio molitor
  His-tagged, Signal plus, Tm 13.17
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccgaattctggatccaaagaattcggcacgagactactaagatgaagttgctctgttgtctaatctccctcattctgttggtcacagttcaggccctgaccgaggcacaaattgagaaactgaacaagatcagcaaaaaatgtcaaaatgaaagtggagtgtcgcaagagatcataaccaaagctcgcaacggtgactgggaggacgatcctaaactgaaacgccaagttttttgcgtggccaggaacgccggtctggccacggaatcgggagaggtggtggtcgacgtgttgagggagaaggtgaggaaggtcactgacaacgacgaagaaactgagaaaatcatcaataagtgcgccgtcaagagagatactgttgaagagacggtgttcaatactttcaaatgtgtcatgaaaaacaagccaaagttctcaccagttgattgaaccaccacgactagtagatggttcaaatggtgtgctttacatataaaaataaagtgtttctgatgtaaaaaaaaaaaaaaaaaaaaaaaaaactcgagagtattctagagcggccgcgggcccatcgttttccacccctcgagcaccaccaccaccaccactgagat

SEQ ID NO: 25 - protein, 174 aa, Tenebrio molitor
  Precursor Protein with His-tag, Tm 13.17
MGSSHHHHHHSSGLVPRGSHMASMTGGQQMGRGSEFARAKMKLLCCLISLILLVTVQALTEAQIEKLNKISKKCQNESGVSQEIITKARNGDWEDDPKLKRQVFCVARNAGLATESGEVVVDVLREKVRKVTDNDEETEKIINKCAVKRDTVEETVFNTFKCVMKNKPKFSPVD

SEQ ID NO: 26 - DNA, 543 bp, Tenebrio molitor
  His-tagged, Signal minus, Tm 13.17
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggcctgaccgaggcacaaattgagaaactgaacaagatcagcaaaaaatgtcaaaatgaaagtggagtgtcgcaagagatcataaccaaagctcgcaacggtgactgggaggacgatcctaaactgaaacgccaagttttttgcgtggccaggaacgccggtctggccacggaatcgggagaggtggtggtcgacgtgttgagggagaaggtgaggaaggtcactgacaacgacgaagaaactgagaaaatcatcaataagtgcgccgtcaagagagatactgttgaagagacggtgttcaatactttcaaatgtgtcatgaaaaacaagccaaagttctcaccagttgattgactcgagcaccaccaccaccaccactgagat

SEQ ID NO: 27 - protein, 149 aa, Tenebrio molitor
  Mature Protein with His-tag, Tm 13.17
MGSSHHHHHHSSGLVPRGSHMASMTGGQQMGRGLTEAQIEKLNKISKKCQNESGVSQEIITKARNGDWEDDPKLKRQVFCVARNAGLATESGEVVVDVLREKVRKVTDNDEETEKIINKCAVKRDTVEETVFNTFKCVMKNKPKFSPVD

SEQ ID NO: 28 - DNA, 681 bp, Tenebrio molitor
  His-tagged, Signal plus, Clone 3.4
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccgaattcgcacgagcaaaaatgaaactcctcttgtgctttgctttcgccgccatcgtcatcggagctcaggctctcaccgacgaacagatacagaaaaggaacaagatcagcaaagaatgccagcaggtgtccggagtgtcccaagagacgatcgacaaagtccgcacaggtgtcttggtcgacgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaaggttatttacgacagtaaacctgatttctctcctattgattaattgttttgtatttgactgaattttgacaataaaggtactatcgttatgtaaaaaaaaaaaaaaaaactcgagcaccaccaccaccaccactgagat

SEQ ID NO: 29 - protein, 173 aa, Tenebrio molitor
  Precursor protein with His-tag, Clone 3.4
MGSSHHHHHHSSGLVPRGSHMASMTGGQQMGRGSEFARAKMKLLLCFAFAAIVIGAQALTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKVIYDSKPDFSPID

SEQ ID NO: 30 - DNA, 543 bp, Tenebrio molitor
  His-tagged, Signal minus, Clone 3.4
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccctcaccgacgaacagatacagaaaaggaacaagatcagcaaagaatgccagcaggtgtccggagtgtcccaagagacgatcgacaaagtccgcacaggtgtcttggtcgacgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaaggttatttacgacagtaaacctgatttctctcctattgattaactcgagcaccaccaccaccaccactgagat

SEQ ID NO: 31 - protein, 149 aa, Tenebrio molitor
  Mature Protein with His-tag, Clone 3.4
MGSSHHHHHHSSGLVPRGSHMASMTGGQQMGRGSLTDEQIQKRNKISKECQQVSGVSQETIDKVRTGVLVDDPKMKKHVLCFSKKTGVATEAGDTNVEVLKAKLKHVASDEEVDKIVQKCVVKKATPEETAYDTFKVIYDSKPDFSPID

SEQ ID NO: 32 - DNA, 682 bp, Tenebrio molitor
  His-tagged, Signal plus, Clone 3.9
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccgaattcgcacgagcaaaaatgaaactcctcttgtgctttgctttcgccgccatcgtcatcggagctcaggctctcaccgatgaacagatacagaaaaggaacaagatcagcaaagaatgccagcaggagtccggagtgtcccaagagacgatcgacaaagtccgcacaggtgtcttggtcgacgatcccaaaatgaagaagcacgtcctctgcttctcgaagagaactggagtggcaaccgaagccggagacaccaatgtggaggtactcaaagccaagctgaagcatgtggccagcgacgaagaagtggacaagatcgtgcagaagtgcgtggtcaagaaggccacaccagaggaaacggcttatgacaccttcaagtgtatttacgacagtaaacctgatttctctcctattgattaattgttttgtatttgactgaattttgacaataaaggtactatcgttatgaaaaaaaaaaaaaaaaaaactcgagcaccaccaccaccaccactgagat

SEQ ID NO: 33 - protein, 173 aa, Tenebrio molitor
  Precursor Protein with His-tag, Clone 3.9
MGSSHHHHHHSSGLVPRGSHMASMTGGQQMGRGSEFARAKMKLLLCFAFAAIVIGAQALTDEQIQKRNKISKECQQESGVSQETIDKVRTGVLVDDPKMKKHVLCFSKRTGVATEAGDTNVEVLKAKLKHVASDEEVDKIVQKCVVKKATPEETAYDTFKCIYDSKPDFSPID

SEQ ID NO: 34 - DNA, 543 bp, Tenebrio molitor
  His-tagged, Signal minus, Clone 3.9
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccctcaccgatgaacagatacagaaaaggaacaagatcagcaaagaatgccagcaggagtccggagtgtcccaagagacgatcgacaaagtccgcacaggtgtcttggtcgacgatcccaaaatgaagaagcacgtcctctgcttctcgaagagaactggagtggcaaccgaagccggagacaccaatgtggaggtactcaaagccaagctgaagcatgtggccagcgacgaagaagtggacaagatcgtgcagaagtgcgtggtcaagaaggccacaccagaggaaacggcttatgacaccttcaagtgtatttacgacagtaaacctgatttctctcctattgattaactcgagcaccaccaccaccaccactgagat

SEQ ID NO: 35 - protein, 149 aa, Tenebrio molitor
  Mature Protein with His-tag, Clone 3.9
MGSSHHHHHHSSGLVPRGSHMASMTGGQQMGRGSLTDEQIQKRNKISKECQQESGVSQETIDKVRTGVLVDDPKMKKHVLCFSKRTGVATEAGDTNVEVLKAKLKHVASDEEVDKIVQKCVVKKATPEETAYDTFKCIYDSKPDFSPID

SEQ ID NO: 36 - DNA, 681 bp, Tenebrio molitor
  His-tagged, Signal plus, Clone 7.5
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccgaattcgcacgagcaaaaatgaaactcctcttgtgctttgcgttcgccgccatcgtcatcggagctcaggctctcaccgacgaacagatacagaaaaggaacaagatcagcaaagagtgccagcaggtgtccggagtgtcccaagagacgatcgacaaagtccgcacaggtgtcttggtcgacgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaagtgtatttacgacagtaaacctgatttctctcctattgattaattgttttgtatttggctgaattttgacaataaaggtactatcgttatgtaaaaaaaaaaaaaaaaactcgagcaccaccaccaccaccactgagat

SEQ ID NO: 37 - protein, 173 aa, Tenebrio molitor; identical to SEQ ID NO: 17
  Precursor Protein with His-tag, Clone 7.5

SEQ ID NO: 38 - DNA, 543 bp, Tenebrio molitor
  His-tagged, Signal minus, Clone 7.5
ttgttagcggatggaattccctcgtaggggataattttgtttactttaagaaggagatataccatgggcagcagccatcatcatcatcatcacagcagcggcctggtgccgcgcggcagccatatggctagcatgactggtggacagcaaatgggtcgcggatccctcaccgacgaacagatacagaaaaggaacaagatcagcaaagagtgccagcaggtgtccggagtgtcccaagagacgatcgacaaagtccgcacaggtgtcttggtcgacgatcccaaaatgaagaagcacgtcctctgcttctcgaagaaaactggagtggcaaccgaagccggagacaccaatgtggaggtactcaaagccaagctgaagcatgtggccagcgacgaagaggtggacaagatcgtgcagaagtgcgtggtcaagaaggccacaccagaggaaacggcttatgacaccttcaagtgtatttacgacagtaaacctgatttctctcctattgattaactcgagcaccaccaccaccaccactgagat

SEQ ID NO: 39 - protein, 149 aa, Tenebrio molitor; identical to SEQ ID NO: 19
  Mature protein with His-tag, Clone 7.5

SEQ ID NO: 40 - DNA, 24 bp, Tenebrio molitor
  Tm 12.84 upper primer with Bam-H1 site (Clones 2.2, 2.3, 3.4, 3.9, and 7.5)
cgcggatccctcaccgacgaacag

SEQ ID NO: 41 - DNA, 25 bp, Tenebrio molitor
  Tm 12.84 lower primer with Xho1 site (Clones 2.2, 2.3, 3.4, 3.9, and 7.5)
gagaggataactaattgagctcgcc

SEQ ID NO: 42 - DNA, 24 bp, Tenebrio molitor
  Tm 13.17 upper primer with Bam-H1 site
cgcggatccctgaccgaggcacaa

SEQ ID NO: 43 - DNA, 25 bp, Tenebrio molitor
  Tm 13.17 lower primer with Xho1 site
gagtggtcaactaactgagctcgcc

SEQ ID NO: 44 - DNA, 481 bp, Tenebrio molitor
  misc_feature: Consensus of the Tm 12.84 Isoforms, n = any nucleotide, Xaa = any amino acid.
ggcacgagcaaaaatgaaactcctcttgtgctttgcnttcgccgccatcgtcatcggagctcaggctctcaccgaygaacagatacagaaaaggaacaagatcagcaaagartgccagcaggngtccggagtgtcccaagagacgatcgacaaagtccgcacaggtgtcttggtcgaygatcccaaaatgaagaagcacgtcctctgcttctcgaagaraactggagtggcaaccgaagccggagacaccaatgtggaggtactcaaagccaagctgaagcatgtggccagcgacgaagargtggacaagatcgtgcagaagtgcgtggtcaagaaggccacaccagaggaaacggcttatgacaccttcaagnntatttacgacagtaaacctgatttctctcctattgattaattgttttgtatttgrctgaattttgacaataaaggtantatcgttatgnaaaaaaaaaaaaaaaaa

SEQ ID NO: 45 - DNA, 484 bp, Tenebrio molitor
  misc_feature: Consensus of Seq ID #44 with Tm 13.17, n = any nucleotide, Xaa = any amino acid
ggcanrnnnnaaratgaarytnctctnntgyytnryntycnycryyntnntnrtcrnagytcaggcyctnaccgangnacaratnnagaaanngaacaagatcagcaaarartgycarnangnrnnyggagtgtcncaagagaynatnrncaaagyycgcannggtgnctnggnngaygatccyaaantgaarnrncangtyytytgcntnncnargarnrcyggnntggcnacngaancnggaganrynrnngtngangtrytnarrgnnaagntgargnangtnrcyrrcaacgacgaagarrynganaaratcrtnnanaagtgcgyngtcaagarrgnyacnnyngargaracggyntnyrayacyttcaarnntrtynnnranaryaarccnranttctcnccnrttgattranynnyynnnaytngnnnrnrnttyranaataaagnnnntntnrtnnnrnaaaaaaaaaaaaaaaaa

SEQ ID NO: 46 - DNA, 484 bp, Tenebrio molitor
  misc_feature: Consensus of Seq ID #45 with B1/B2, n = any nucleotide
ggcanrnnnnaaratgaarytnctctnntgyytnryntyynycryyntnntnrtcnnagytcaggcyntnacynangnananntnnagnnanngnncnaraycagcrnarartgynarnnngnrnnyggagtgtcnnaaganrynatnrnnaragyycgcannggtgnctnggnngaygayccyaaantgaarnnncanntyytytgcntnnynargrnnnyygrnntrgynrcngaancnggaganrynrnngynganryrytnarrgnnaagntgangnrnnnnnnnrnnannrnnrarrarrynrrnarrntnnynnrnarnnnnnnnnnnnngarnrnnnynnnnrarrnrnnnnnntnnrannynyynaannnnnnynnnrrnannarnccnrnntyytyncnnrytrnttrnnynnnnnnnnynngnnnrnrnttyranaataaagnnnytntnrtnnnrnaaaaaaaaaaaaaaaaa

SEQ ID NO: 47 - DNA, 484 bp, Tenebrio molitor
  misc_feature: Consensus of SEQ. ID #46 with AFP-3, n = any nucleotide
ggcnnrnnnnaaratgaarytnctcynntgyytnrynyyynyyryyntnntnrycnnrryyyangcyntnacynanrnannnnnnnagnnrnngnnynarnncagcrnnrnntgynarnnngnrnnyggagtrtcnnaagannynntnrnnarrgyycgcannngtgnnnnrgnngaygayccyaaantgaarnnncannyyytytgcntnnynargrnnnyygrnntnrynrnngnnnnnggngannynnnnnyngannnnntnarrrnnaarntnangnrnnnnnnnrnnrnnnnnrarrarrynrrnrrnntnnynnnnarnnnnnnnnnnnngarnrnnnynnnnnarnnnnnnnnnnnnrannynyynaannnnnnynnnrrnannarnycnnnntnnnnncnnnynrnntrnnnnnnnnnnnynnrnnnnnnnnnnnnnaataaannnnnnnnnnnnnnnnaaaaaaaaaaaaaaaaa

SEQ ID NO: 48 - protein, 136 aa, Tenebrio molitor
  misc_feature: General Consensus of Clones, B1, B2 and AFP-3, Xaa = any amino acid
MKLLXCXXXXXXXXXXXAXTXXXXZXXXXXSXXCXXXSGXSZXXXXXXRXXXXXDDPKXKXXXXCXXXXXXXXXXXGXXXXXXXXXKXXXXXXXXEXXXXXXXKCXVXXXTXZXXXXXXXKXXXXXXXXFXXXXXX